SEQUENCE LISTING (56 sequences)

SEQ ID NO: 1 - DNA, 4181 bp, synthetic construct
  misc_feature 1..4181: note = vector genome ITR.CB.CBI.IL2-V1.Trastuzumab-coGW.SV40.ITR
  repeat_region 1..108: note = 5' ITR
  misc_feature 45..46: note = 22 bp deletion
  regulatory 172..448: note = promoter - chicken beta-actin promoter; regulatory_class = promoter
  misc_feature 451..1516: note = Intron - chicken beta-actin intron (rev)
  misc_feature 1523..1528: note = Kozak
  sig_peptide 1529..1588: note = IL-2 signal sequence
  misc_feature 1589..2935: note = Trastuzumab Heavy Variable-Constant
  misc_feature 2945..2998: note = T2A
  sig_peptide 2999..3058: note = IL-2 V1 mut
  misc_feature 3059..3706: note = Trastuzumab Light Variable-Constant
  regulatory 3769..3890: note = polyA_signal - SV40 polyA signal; regulatory_class = polyA_signal_sequence
  repeat_region 4052..4181: note = 3' ITR
  source 1..4181: mol_type = other DNA
ctgcgcgctcgctcgctcactgaggccgcccgggcaaagcccgggcggcctcagtgagcgagcgagcgcgcagagagggagtggccaactccatcactaggggttccttgtagttaatgattaacccgccatgctacttatctacgtagccatgctctaggaagatcttggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatccggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 2 - DNA, 3719 bp, synthetic construct
  misc_feature 1..3719: note = expression cassette CB.CBI.IL2.V1.Trastuzumab-coGW.SV40
  regulatory 1..278: note = promoter - chicken beta-actin promoter; regulatory_class = promoter
  misc_feature 280..1345: note = Intron - chicken beta-actin intron
  misc_feature 1352..1357: note = Kozak
  sig_peptide 1358..1417: note = IL-2 signal sequence
  misc_feature 1418..2764: note = Trastuzumab Heavy Variable-Constant
  misc_feature 2774..2827: note = T2A
  misc_feature 2828..2887: note = IL-2 V1 mut signal peptide
  misc_feature 2888..3535: note = Trastuzumab Light Variable Constant
  regulatory 3598..3719: note = polyA_signal - SV40 polyA signal; regulatory_class = polyA_signal_sequence
  source 1..3719: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatccggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 3 - DNA, 1347 bp, synthetic construct
  misc_feature 1..1347: note = Trastuzumab-coGW heavy chain (without leader)
  source 1..1347: mol_type = other DNA
gaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggc

SEQ ID NO: 4 - protein, 449 aa, synthetic construct
  REGION 1..449: note = Trastuzumab-coGW heavy chain (without leader)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - DNA, 648 bp, synthetic construct
  misc_feature 1..648: note = Trastuzumab-coGW light chain (without leader)
  source 1..648: mol_type = other DNA
gatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataa

SEQ ID NO: 6 - protein, 214 aa, synthetic construct
  REGION 1..214: note = Trastuzumab-coGW light chain (without leader)
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - DNA, 60 bp, synthetic construct
  misc_feature 1..60: note = nucleic acid sequence IL-2 signal peptide
  source 1..60: mol_type = other DNA
atgtaccggatgcagctgctgagctgtatcgccctgtctctggccctcgtgaccaacagc

SEQ ID NO: 8 - protein, 20 aa, synthetic construct
  REGION 1..20: note = amino acid sequence IL-2 signal peptide
MYRMQLLSCIALSLALVTNS

SEQ ID NO: 9 - DNA, 60 bp, synthetic construct
  misc_feature 1..60: note = nucleic acid sequence IL-2 signal peptide V1
  source 1..60: mol_type = other DNA
atgtaccggatgcagctgctgctgctgatcgccctgtctctggccctcgtgaccaacagc

SEQ ID NO: 10 - protein, 20 aa, synthetic construct
  REGION 1..20: note = amino acid sequence IL-2 signal peptide V1
MYRMQLLLLIALSLALVTNS

SEQ ID NO: 11 - DNA, 57 bp, synthetic construct
  misc_feature 1..57: note = nucleic acid sequence IL-2 signal peptide V2
  source 1..57: mol_type = other DNA
atgcggatgcagctgctgctgctgatcgccctgtctctggccctcgtgaccaacagc

SEQ ID NO: 12 - protein, 19 aa, synthetic construct
  REGION 1..19: note = amino acid sequence IL-2 signal peptide V2
MRMQLLLLIALSLALVTNS

SEQ ID NO: 13 - DNA, 1407 bp, synthetic construct
  misc_feature 1..1407: note = nucleic acid sequence Trastuzumab-coGW heavy chain (with leader)
  source 1..1407: mol_type = other DNA
atgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggc

SEQ ID NO: 14 - protein, 469 aa, synthetic construct
  REGION 1..469: note = amino acid sequence Trastuzumab-coGW heavy chain (with leader)
MYRMQLLSCIALSLALVTNSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - DNA, 705 bp, synthetic construct
  misc_feature 1..705: note = nucleic acid sequence Trastuzumab-coGW light chain (with leader)
  source 1..705: mol_type = other DNA
atgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctga

SEQ ID NO: 16 - protein, 234 aa, synthetic construct
  REGION 1..234: note = amino acid sequence Trastuzumab-coGW light chain (with leader)
MYRMQLLLLIALSLALVTNSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 2175 bp, synthetic construct
  misc_feature 1..2175: note = nucleic acid sequence Trastuzumab (coGW) (with leader)
  misc_feature 1..60: note = IL-2 signal peptide
  misc_feature 61..1407: note = Trastuzumab heavy Variable-Constant
  misc_feature 1417..1470: note = T2A
  misc_feature 1531..2175: note = Trastuzumab Light Variable-Constant
  source 1..2175: mol_type = other DNA
atgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctga

SEQ ID NO: 18 - protein, 724 aa, synthetic construct
  REGION 1..724: note = amino acid sequence Trastuzumab (coGW) (with leader)
  REGION 1..20: note = IL-2 signal peptide
  REGION 21..469: note = Trastuzumab heavy Variable-Constant
  REGION 470..472: note = GSG linker
  REGION 473..490: note = T2A
  REGION 491..510: note = IL-2 V1 signal peptide
  REGION 511..724: note = Trastuzumab Light Variable-Constant
MYRMQLLSCIALSLALVTNSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGEGRGSLLTCGDVEENPGPMYRMQLLLLIALSLALVTNSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 382 bp, synthetic construct
  regulatory 1..382: note = CMV IE enhancer; regulatory_class = enhancer
  source 1..382: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattac

SEQ ID NO: 20 - DNA, 282 bp, synthetic construct
  regulatory 1..282: note = chicken beta-acton promoter; regulatory_class = promoter
  source 1..282: mol_type = other DNA
tggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 21 - DNA, 1729 bp, synthetic construct
  misc_feature 1..1729: note = CB7 hybrid promoter
  source 1..1729: mol_type = other DNA
  regulatory 1..380: note = CMV IE enhancer; regulatory_class = enhancer
  regulatory 382..659: note = chicken beta actin promoter; regulatory_class = promoter
  misc_feature 660..1729: note = chimeric intron (chimera between introns from chicken beta-actin and rabbit beta-globin)
  misc_feature 753..1725: note = chicken beta actin intron
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 22 - DNA, 133 bp, synthetic construct
  misc_feature 1..133: note = chimeric intron (promega intron)
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 23 - DNA, 122 bp, synthetic construct
  regulatory 1..122: note = SV40 polyA signal; regulatory_class = polyA_signal_sequence
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 24 - DNA, 1212 bp, synthetic construct
  misc_feature 1..1212: note = Ubiquitin C promoter (UbC)
  source 1..1212: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagac

SEQ ID NO: 25 - DNA, 4400 bp, synthetic construct
  misc_feature 1..4400: note = vector genome ITR.UbC.PI.IL2_Furin_V1.Trastuzumab-coGW.SV40.ITR
  misc_feature 1..130: note = ITR
  regulatory 191..1402: note = promoter - UbC promoter; regulatory_class = promoter
  misc_feature 1513..1645: note = Intron - chicmeric intron
  misc_feature 1690..1708: note = T7
  misc_feature 1730..1735: note = Kozak
  sig_peptide 1736..1795: note = IL-2 signal sequence
  misc_feature 1796..3142: note = Trastuzumab heavy Variable-Constant
  misc_feature 3164..3217: note = T2A
  misc_feature 3218..3277: note = IL-2 V1 mut
  misc_feature 3278..3925: note = Trastuzumab light variable constant
  misc_feature 3143..3154: note = Furin
  regulatory 3988..4109: note = polyA_signal - SV40 polyA signal; regulatory_class = polyA_signal_sequence
  misc_feature 4271..4400: note = ITR
  source 1..4400: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagaattcgccaccgcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggccgtaagcgccgcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatcgggtacatctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 26 - DNA, 3919 bp, synthetic construct
  misc_feature 1..3919: note = expression cassette UbC.PI.IL2_Furin_V1.Trastuzumab-coGW.SV40
  regulatory 1..1212: note = promoter - UbC promoter; regulatory_class = promoter
  misc_feature 1323..1455: note = Intron - chimeric intron
  misc_feature 1500..1518: note = T7
  misc_feature 1540..1545: note = Kozak
  sig_peptide 1546..1605: note = IL-2 signal sequence
  misc_feature 1606..2952: note = Trastuzumab Heavy Variable-Constant
  misc_feature 2953..2964: note = Furin
  misc_feature 2974..3027: note = T2A
  misc_feature 3028..3087: note = IL-2 V1 mut
  misc_feature 3088..3735: note = Trastuzumab Light Variable-Constant
  regulatory 3798..3919: note = polyA_signal - SV40 polyA signal; regulatory_class = polyA_signal_sequence
  source 1..3919: mol_type = other DNA
ggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagaattcgccaccgcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggccgtaagcgccgcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatcgggtacatctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 27 - DNA, 4600 bp, synthetic construct
  misc_feature 1..4600: note = vector genome ITR.CB7.CI.IL2_Furin_V1.Trastuzumab-coGW.SV40.ITR
  misc_feature 1..130: note = ITR
  regulatory 191..572: note = enhancer - CMV IE Promoter; regulatory_class = enhancer
  regulatory 191..1923: note = promoter - CB7 hybrid promoter; regulatory_class = promoter
  regulatory 575..856: note = promoter - CB promoter; regulatory_class = promoter
  misc_feature 829..832: note = TATA
  misc_feature 951..1923: note = chicken beta-actin intron
  misc_feature 1930..1935: note = Kozak
  sig_peptide 1936..3995: note = IL-2 signal peptide
  misc_feature 1996..3342: note = Trastuzumab heavy Variable-Constant
  misc_feature 3343..3354: note = Furin site
  misc_feature 3364..3417: note = T2A
  misc_feature 3418..3477: note = IL-2 V1 mut
  misc_feature 3478..4125: note = Trastuzumab light Variable-Constant
  regulatory 4188..4309: note = polyA_signal - SV40 polyA signal; regulatory_class = polyA_signal_sequence
  misc_feature 4471..4600: note = ITR
  source 1..4600: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatctctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggccgtaagcgccgcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatccggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 28 - DNA, 4119 bp, synthetic construct
  misc_feature 1..4119: note = expression cassette CB7.CI.IL2_Furin_V1.Trastuzumab-coGW.SV40
  regulatory 1..382: note = enhancer - CMV IE enhancer; regulatory_class = enhancer
  regulatory 1..1733: note = promoter - CB7 hybrid promoter; regulatory_class = promoter
  regulatory 385..666: note = promoter - CB promoter; regulatory_class = promoter
  misc_feature 639..642: note = TATA
  misc_feature 761..1733: note = chicken beta-actin intron
  misc_feature 1740..1745: note = Kozak
  sig_peptide 1746..1805: note = IL-2 signal sequence
  misc_feature 1806..3152: note = Trastuzumab heavy variable-constant
  misc_feature 3153..3164: note = Furin Site
  misc_feature 3174..3227: note = T2A
  misc_feature 3228..3287: note = IL-2 V1 mut
  misc_feature 3288..3935: note = Trastuzumab Light Variable-Constant
  regulatory 3998..4119: note = polyA_signal - SV40 polyA signal; regulatory_class = polyA_signal_sequence
  source 1..4119: mol_type = other DNA
ctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggccgtaagcgccgcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatccggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 29 - DNA, 2187 bp, synthetic construct
  misc_feature 1..2187: note = nucleic acid sequence Trastuzumab (coGW) (with furin)
  misc_feature 1..60: note = IL-2 signal peptide
  misc_feature 61..1407: note = Trastuzumab heavy variable-constant
  misc_feature 1408..1419: note = furin site
  misc_feature 1429..1482: note = T2A
  misc_feature 1483..1542: note = IL-2 V1 Mut signal peptide
  misc_feature 1543..2187: note = Trastuzumab light variable-constant
  source 1..2187: mol_type = other DNA
atgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggccgtaagcgccgcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctga

SEQ ID NO: 30 - protein, 728 aa, synthetic construct
  REGION 1..728: note = amino acid sequence Trastuzumab (coGW) (with furin)
  REGION 1..20: note = IL-2 signal peptide
  REGION 21..469: note = Trastuzumab heavy variable-constat
  REGION 470..473: note = Furin
  REGION 474..476: note = GSG linker
  REGION 477..494: note = T2A
  REGION 495..514: note = IL-2 V1 mut signal peptide
  REGION 515..728: note = Trastuzumab light variable-constant
MYRMQLLSCIALSLALVTNSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGRKRRGSGEGRGSLLTCGDVEENPGPMYRMQLLLLIALSLALVTNSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 12 bp, synthetic construct
  misc_feature 1..12: note = furin recognition sequence
  source 1..12: mol_type = other DNA
cgtaagcgccgc

SEQ ID NO: 32 - DNA, 54 bp, synthetic construct
  misc_feature 1..54: note = T2A sequence
  source 1..54: mol_type = other DNA
gaaggcagaggcagcctgcttacctgtggcgacgtggaagagaaccccggacct

SEQ ID NO: 33 - DNA, 75 bp, synthetic construct
  misc_feature 1..75: note = Furin with T2A
  source 1..75: mol_type = other DNA
cgtaagcgccgcggctctggcgaaggcagaggcagcctgcttacctgtggcgacgtggaagagaaccccggacct

SEQ ID NO: 34 - DNA, 973 bp, synthetic construct
  misc_feature 1..973: note = Chicken beta-actin intron
  source 1..973: mol_type = other DNA
gtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaag

SEQ ID NO: 35 - protein, 450 aa, synthetic construct
  REGION 1..450: note = amino acid sequence of heavy chain of humanized monoclonal antibody Trastuzumab (DrugBank: DB00072)
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 214 aa, synthetic construct; identical to SEQ ID NO: 6
  REGION 1..214: note = amino acid sequence of light chain of humanized monoclonal antibody Trastuzumab (DrugBank: DB00072)

SEQ ID NO: 37 - DNA, 2211 bp, adeno-associated virus hu68
  source 1..2211: mol_type = other DNA
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 38 - DNA, 2211 bp, synthetic construct
  source 1..2211: mol_type = other DNA; note = adeno-associated virus hu68 engineered nucleic acid sequence
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgacggcagtcaggcggtgggccgctcatccttctactgcctggagtacttcccttcgcagatgctgaggactggcaacaacttccagttcagctacgagttcgagaacgtccctttccacagcagctacgcccacagccagagtttggaccgcttgatgaaccctctgatcgaccagtacctgtactacctgtcaaagacgatcaacggttctggccagaaccagcagacgctgaagttcagcgtggccgggcctagcaacatggccgtccagggcagaaactacatccctgggcccagctaccggcagcagagagtctcaaccactgtgactcagaacaacaacagtgagttcgcctggcctggcgccagctcttgggccctcaacggccgcaactcgctgatgaacccaggcccagccatggccagtcacaaggagggcgaggaccgtttcttccctttgtctggctctctgatcttcggcaagcaggggaccggcagagacaacgtggacgcggacaaggtcatgatcacgaacgaggaggagatcaagaccaccaaccctgtggcaaccgagtcctacggccaggtggcaaccaaccaccagagcgcccaggcacaggcgcagactggctgggtccagaaccaggggatcctgcctggcatggtgtggcaggaccgtgacgtgtacctgcagggccctatctgggcaaagatccctcacacggacggcaacttccacccttctcctctgatgggcggcttcggcatgaagcacccgcctcctcagatcctcatcaagaacactccggtcccggcagaccctccgacggccttcaacaaggacaagctgaactcattcatcactcagtactccactggccaggtcagcgtggagatcgagtgggagctgcagaaggagaacagcaagcgttggaacccagagatccagtacacttccaactactacaagtctaacaacgtggagttcgccgtcaacactgagggtgtgtacagtgagcctcgccctatcggcacccggtacctcacccgaaacttgtga

SEQ ID NO: 39 - protein, 736 aa, adeno-associated virus hu68
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 40 - DNA, 2211 bp, adeno-associated virus rh91
  source 1..2211: mol_type = other DNA
atggctgccgatggttatcttccagattggctcgaggacaacctctctgagggcattcgcgagtggtgggcgctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaaccttttggtctggttgaggaagcagctaagacggctcctggaaagaaacgtccggtagagcagtcgccccaagaaccagactcctcctcgggcattggcaaatcaggccagcagcccgccaaaaagagactcaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtacggctacctaacgctcaacaatggcagccaggccgtaggacgttcatccttttattgcctggaatatttcccatctcaaatgctgagaacgggcaacaactttaccttcagctacacctttgaagatgtgcctttccacagcagttacgcgcacagccagagcctggacaggctaatgaatcctctaatcgaccagtacctgtattacctaaacagaactcagaatcaatccggaagtgcacaaaacaaggacttgctgtttagccgggggtctccagctggcatgtctgttcagcccaaaaactggctacccgggccctgttaccgacagcagcgtgtttctaaaacaaaaacagacaacaacaacagcaactttacctggactggtgcctccaaatacaatctgaacggacgtgaatccatcattaaccctggcaccgctatggcatcccacaaggacgacgaagacaaattttttcccatgagcggtgttatgatttttggcaaagaaaatgcaggagcatcaaacactgcattagacaatgttatgattacagatgaagaggaaattaaagctaccaaccccgtggccaccgagagatttggaactgtggcagtcaatctccaaagcagcaatacagaccctgcaacaggagacgtgcatgtcatgggggctttacctggcatggtgtggcaagacagagacgtgtacctgcagggtcccatttgggccaagattcctcacacggatggacactttcacccgtctcctcttatgggcggctttggacttaagcacccgcctcctcagatcctcatcaaaaacacgcctgttcctgcgaatcctccggcagagttttcggctacaaagtttgcttcattcatcacccagtactccacaggacaagtgagcgtggaaattgaatgggagctgcagaaagaaaacagtaagcgctggaatcctgaagtgcagtacacctccaactacgcgaaatctgccaacgttgatttcactgtggacaacaatggactttatactgagcctcgccccattggcacccgttaccttacccgtcccctttaa

SEQ ID NO: 41 - DNA, 2211 bp, synthetic construct
  source 1..2211: mol_type = other DNA; note = adeno-associated virus rh91 engineered nucleic acid sequence
atggctgctgacggttatcttccagattggctcgaggacaacctttctgaaggcattcgtgagtggtgggctctgaaacctggagcccctaaacccaaagcgaaccaacaaaagcaggacgacggccggggtcttgtgcttccgggttacaaatacctcggacccttcaacggactcgacaaaggagagccggtcaacgcggcggacgcggcagccctcgaacacgacaaagcttacgaccagcagctcaaggccggtgacaacccgtacctccggtacaaccacgccgacgccgagtttcaggagcgtcttcaagaagatacgtcttttgggggcaaccttggcagagcagtcttccaggccaaaaagagggttcttgagccttttggtctggttgaggaagcagctaaaacggctcctggaaagaagaggcctgtagagcagtctcctcaggaaccggactcatcatctggtattggcaaatcgggccagcagcctgccaaaaaaagactaaatttcggtcagactggcgactcagagtcagtccccgaccctcaacctctcggagaacctccagaaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacctgggcccttcctacctacaacaaccacctctacaagcaaatctccagcgcttcaacgggggccagtaacgacaaccactactttggctacagcaccccctgggggtattttgatttcaacagattccactgccacttctcaccacgtgactggcagcgactcattaacaacaactggggattccggcccaagagactcaacttcaagctcttcaacatccaggtcaaggaggtcacgacgaatgatggcgtcacaaccatcgctaataaccttaccagcacggttcaagtgttctcggactcggagtaccagctgccgtacgtcctcggttctgcgcaccagggctgcctccctccgttcccggcggacgtattcatgattcctcagtatggatacctcaccctgaacaacggaagtcaagcggtgggacgctcatccttttactgcctggagtacttcccttcgcagatgctaaggactggaaataacttcaccttcagctataccttcgaggatgtaccttttcacagcagctacgctcacagccagagtttggatcgcttgatgaatcctcttattgatcagtatctgtactacctgaacagaacgcaaaatcaatctggaagtgcacaaaacaaggacctgctttttagccgggggtctcctgctggcatgtctgttcagcccaaaaattggctacctgggccctgctaccggcaacagagagtttcaaagactaaaacagacaacaacaacagtaactttacctggacaggtgccagcaaatataatctcaatggccgcgaatcgatcattaatccaggaaccgctatggccagtcacaaggacgatgaagacaaatttttccctatgagcggcgttatgatatttggcaaagaaaatgcaggagcaagtaacactgcattagataatgtaatgattacggatgaagaagagattaaagctaccaatcctgtggcaacagagagatttggaactgtggcagtcaacttgcagagctcaaatacagaccccgcaactggagacgtccatgtcatgggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcctcatcaaaaatactccggtaccggcaaatcctccggcagagttcagcgctacaaagtttgcttcatttatcactcagtactccactggacaggtcagcgtggaaattgagtgggagctacagaaagaaaacagcaaacgttggaatccagaggtgcagtacacttccaactacgcgaagtctgccaatgtggactttactgtagacaacaatggtctttatactgaacctcgccctattggaacccggtatctcacacgacccttgtaa

SEQ ID NO: 42 - protein, 736 aa, adeno-associated virus rh91
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEAAKTAPGKKRPVEQSPQEPDSSSGIGKSGQQPAKKRLNFGQTGDSESVPDPQPLGEPPETPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKENAGASNTALDNVMITDEEEIKATNPVATERFGTVAVNLQSSNTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 43 - DNA, 2211 bp, adeno-associated virus hu95
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagtttcaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaataatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgcttcgtaccggaaacaactttaccttcagctacgagtttgagaacgtacctttccatagcagctatgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagttagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 44 - DNA, 2211 bp, synthetic construct
  source 1..2211: mol_type = other DNA; note = adeno-associated virus hu95 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagcgggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccagctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 45 - protein, 736 aa, adeno-associated virus hu95
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 46 - DNA, 2211 bp, adeno-associated virus hu96
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgccgcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgagggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagttatcaccaccagcacccgaacctgggccctgcccacctacaacaaccacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgcttcgtaccggaaacaactttaccttcagctacgagtttgagaacgtacctttccatagcagctacgctcacagccaaagcctggaccgactaatgaatccactcatcgaccaatacttgtactatctctcaaagactattaacggttctgggcagaatcaacaaacgctaaaattcagtgtggccggacccagcaacatggcagtccagggaagaaactacatacctggacccatctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccggtagcaacggagtcctatggacaagtggccacaaaccaccagagtgcccaagcacaggcgcaaacaggctgggttcaaaaccaaggaatacttccgggtatggtttggcaagacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 47 - DNA, 2211 bp, synthetic construct
  source 1..2211: mol_type = other DNA; note = adeno-associated virus hu96 engineered nucleic acid sequence
atggctgctgacggttatcttccagattggctcgaggacaacctttctgaaggcattcgtgagtggtgggctctgaaacctggagcccctcaacccaaagcgaaccaacaacaccaggacaacgctcggggtcttgtgcttccgggttacaaatacctcggacccggtaacggactcgacaaaggagagccggtcaacgcagcggacgcggcagccctcgaacacgacaaagcctacgaccagcagctcaaggccggtgacaacccgtacctcaagtacaaccacgccgacgccgagtttcaggagcgtcttaaagaagatacgtcttttgggggcaaccttggcagagcagtcttccaggccaaaaagaggcttcttgagcctcttggtctggttgaggaagcagctaaaacggctcctggaaagaagaggcctgtagagcagtctcctcaggaaccggactcatcagagggtattggcaaatcgggcgcacagcctgccaaaaaaagactaaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtatggatacctcaccctgaacgatggaagtcaagcggtgggacgctcatccttttactgcctggagtacttcccttcgcagatgctaaggactggaaataacttcaccttcagctatgagttcgagaacgtaccttttcacagcagctacgctcacagccagagtttggatcgcttgatgaatcctcttattgatcagtatctgtactacctgtcaaagacgattaacggttctggacagaaccaacaaacgctgaaatttagcgtggctgggcctagcaacatggctgtccagggaagaaattacatacctgggcccatctaccggcaacagagagtctcaaccactgtgactcaaaacaacaacagtgaatttgcttggcctggagccagctcttgggctctcaatggccgcaattcgctgatgaatccaggaccagctatggccagtcacaaggaaggagaagaccgttttttccctttgtctggctctctaatatttggcaaacaagggacaggaagagacaacgtggatgcggataaagtaatgattacgaacgaagaagagattaaaaccaccaatcctgtggcaacagagtcctatggacaagtggcaacaaaccaccagagcgcccaagcacaggcgcagactggctgggtccaaaaccaggggatattacctggcatggtgtggcaagatcgtgacgtgtaccttcaaggacctatctgggcaaagattcctcacacggatggaaactttcatccttctcctctgatgggaggctttggaatgaaacatccgcctcctcaaatcctcatcaaaaatactccggtaccggcagatcctccgacggccttcaacaaggacaagctgaactcatttatcactcagtactccactggacaggtcagcgtggaaattgagtgggagctacagaaagaaaacagcaaacgttggaatccagagattcagtacacttccaactactacaagtctaataatgtggaatttgctgtaaatactgaaggtgtttatagtgaacctcgccctattggaacccggtatctcacacgaaacttgtaa

SEQ ID NO: 48 - protein, 736 aa, adeno-associated virus hu96
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSEGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFTFSYEFENVPFHSSYAHSQSLDRLMNPLIDQYLYYLSKTINGSGQNQQTLKFSVAGPSNMAVQGRNYIPGPIYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 49 - protein, 4 aa, synthetic construct
  REGION 1..4: note = RKRR linker
RKRR

SEQ ID NO: 50 - DNA, 4613 bp, synthetic construct
  misc_feature 1..4613: note = vector genome ITR.CB7.CI.IL2_Furin_V1.Trastuzumab-coGW.SV40.ITR (V2)
  repeat_region 1..130: note = ITR
  regulatory 204..585: note = enhancer - CMV IE promoter; regulatory_class = enhancer
  regulatory 588..869: note = promoter - CB promoter; regulatory_class = promoter
  misc_feature 842..845: note = TATA
  misc_feature 964..1936: note = Intron - Chicken beta-actin intron
  misc_feature 1943..1948: note = Kozak
  sig_peptide 1949..2008: note = IL-2 signal sequence
  misc_feature 2009..3355: note = Trastuzumab Heavy Chain
  misc_feature 3356..3367: note = furin site
  misc_feature 3377..3430: note = T2A
  misc_feature 3431..3490: note = IL2 V1
  misc_feature 3491..4138: note = Trastuzumab Light Chain
  regulatory 4201..4322: note = polyA_signal - SV40 polyA; regulatory_class = polyA_signal_sequence
  rep_origin 4484..4613: note = ITR
  source 1..4613: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaaggcggccgccaccatgtaccggatgcagctgctgagctgtatcgccctgtctctggccctcgtgaccaacagcgaagtgcagctggtggaaagcggcggaggactggtgcagcctggcggatctctgagactgagctgtgccgccagcggcttcaacatcaaggacacctacatccactgggtgcgccaggcccctggcaagggactggaatgggtggccagaatctaccccaccaacggctacaccagatacgccgacagcgtgaagggccggttcaccatcagcgccgacaccagcaagaacaccgcctacctgcagatgaacagcctgcgggccgaggacaccgccgtgtactactgtagtagatggggaggcgacggcttctacgccatggactattggggccagggcaccctcgtgacagtgtctagtgcgtcgaccaagggaccttcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgaaccaaagagctgcgacaagacccacacgtgtcccccctgccctgcccctgaactgctgggaggccccagcgtgttcctgttccccccaaagcccaaggacaccctgatgatcagccggacccccgaagtgacctgcgtggtggtggacgtgtcccacgaggaccctgaagtgaagtttaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagcctcaggtctacacactgccccccagccgggaagagatgaccaagaaccaggtgtccctgacctgcctggtcaagggcttctaccccagcgacatcgccgtggaatgggagagcaacggccagcccgagaacaactacaagaccaccccccctgtgctggacagcgacggctcattcttcctgtatagcaagctgaccgtggacaagagccggtggcagcagggcaacgtgttcagctgcagcgtgatgcacgaggccctgcacaaccactacacccagaagtccctgagcctgagccccggccgtaagcgccgcggctctggcgaaggcagaggcagcctgcttacctgtggcgacgtggaagagaaccccggacctatgtaccggatgcagctgctgctgctgatcgccctgtctctggccctcgtgaccaacagcgatatccagatgacccagagccccagcagcctgtctgccagcgtgggcgacagagtgaccatcacctgtagagccagccaggacgtgaacaccgccgtggcctggtatcagcagaagcctggcaaggcccccaagctgctgatctacagcgccagcttcctgtacagcggcgtgcccagcagattcagcggcagcagatccggcaccgacttcaccctgaccatcagctccctgcagcccgaggacttcgccacctactactgccagcagcactacaccaccccccccacatttggccagggcaccaaggtggaaatcaagcgtacggtggccgccccaagcgtgttcatcttcccaccaagcgatgagcagctgaagagcggaaccgccagcgtggtgtgcctgctgaacaacttctacccacgggaggccaaggtgcagtggaaggtggataacgccctgcagagcggaaacagccaggagagcgtgaccgagcaggatagcaaggatagcacctacagcctgagcagcaccctgaccctgagcaaggccgattacgagaagcacaaggtgtacgcctgcgaggtgacccaccagggactgagcagcccagtgaccaagagcttcaaccgcggagagtgctgataatgaggatccggtacctctagagtcgacccgggcggcctcgaggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 51 - DNA, 1070 bp, synthetic construct
  source 1..1070: mol_type = other DNA; note = Chimeric Intron comprising Chicken Beta Actin Intron
ggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 52 - protein, 18 aa, synthetic construct
EGRGSLLTCGDVEENPGP

SEQ ID NO: 53 - protein, 25 aa, synthetic construct
RKRRGSGEGRGSLLTCGDVEENPGP

SEQ ID NO: 54 - DNA, 2208 bp, adeno-associated virus 9
  source 1..2208: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 55 - protein, 736 aa, adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 56 - protein, 8 aa, synthetic construct
DTYIHWVR

(19) United States
(12) Patent Application Publication
(10) Pub. No.: US 2025/0215111 A1
(43) Pub. Date: Jul. 3, 2025

(54) COMPOSITIONS AND METHODS FOR TREATING HER2 POSITIVE METASTATIC BREAST CANCER AND OTHER CANCERS

(71) Applicant: The Trustees of the University of Pennsylvania, Philadelphia, PA (US)

(72) Inventors: Jenny A. Sidrane, Phoenixville, PA (US); Marcela Salazar Werner, West Chester, PA (US)

(73) Assignee: The Trustees of the University of Pennsylvania, Philadelphia, PA (US)

(21) Appl. No.: 18/853,071

(22) PCT Filed: Apr. 6, 2023
(86) PCT No.: PCT/US2023/065423

§ 371 Date: Sep. 30, 2024

Related U.S. Application Data
(60) Provisional application No. 63/328,225, filed on Apr. 6, 2022.
(60) Provisional application No. 63/343,326, filed on May 18, 2022.
(60) Provisional application No. 63/344,905, filed on May 23, 2022.

(51) Int. Cl. C07K 16/32; A61K 39/00; A61P 35/00; C12N 15/86

(52) U.S. Cl. CPC C07K 16/32; A61P 35/00; C12N 15/86; A61K 2039/5256; C07K 2317/24; C07K 2317/732; C12N 2750/14143; C12N 2750/14145; C12N 2830/15

(57) ABSTRACT

Provided herein is a recombinant AAV (rAAV) comprising an AAV capsid and a vector genome packaged therein, wherein the vector genome comprises an AAV 5′ inverted terminal repeat (ITR), an engineered nucleic acid sequence encoding an anti-Her2 antibody (i.e., trastuzumab) comprising heavy chain and light chain operably linked to regulatory control sequences which direct expression of trastuzumab in a target cell, and an AAV 3′ ITR. Also provided are a pharmaceutical composition comprising a rAAV as described herein in a formulation buffer, nucleic acid molecule, packaging host cells, rAAV production system, and a method of treating HER2-positive metastatic cancer in the brain, optionally a metastatic breast cancer in the brain.

### REFERENCE TO AN ELECTRONIC SEQUENCE LISTING

The electronic sequence listing filed herewith named “UPN-22-9834PCT_Seq_List.xml” with size of 186,012 bytes, created on date of Mar. 30, 2023, and the contents of the electronic sequence listing (e.g., the sequences and text therein) are incorporated herein by reference in entirety.

### BACKGROUND OF THE INVENTION

Brain metastases are a common and devastating sequelae of breast cancer for which treatment options are few and inadequate. 6-16% of breast cancer patients develop central nervous system (CNS) metastases. These patients have a 20% one-year and 1.3% five-year median survival from the time of diagnosis. DiStefano A, et al., Cancer. 1979; 44:1913-1918; Takakura K, et al., Metastatic tumors of the central nervous system. Tokyo: Igaku-Shoin, 1982; Hall W A, et al., Long-term survival with metastatic cancer to the brain. Med Oncol. 2000 Nov; 17(4):279-86; Pieńkowski T, Zielinski C C. Trastuzumab treatment in patients with breast cancer and metastatic CNS disease. Ann Oncol. 2010 May; 21(5):917-24. Surgical resection of brain metastases is often infeasible, and chemotherapeutic agents are mostly excluded from the CNS by the blood brain barrier (BBB) [Nakayama A, et al., Antitumor Activity of TAK-285, an Investigational, Non-Pgp Substrate HER2/EGFR Kinase Inhibitor, in Cultured Tumor Cells, Mouse and Rat Xenograft Tumors, and in an HER2-Positive Brain Metastasis Model, J Cancer. 2013 Aug. 16; 4(7)]. Alternative therapies to treat breast cancer brain metastases are needed.

Breast cancers that overexpress the HER2 receptor tyrosine kinase have a high propensity to metastasize to the CNS and comprise 25-30% of all breast cancer cases [Bendell J C, et al., Central nervous system metastases in women who receive trastuzumab-based therapy for metastatic breast carcinoma. Cancer. 2003 Jun. 15; 97(12):2972-7]. Trastuzumab (Herceptin®) is a first-line therapeutic immunoglobulin G (IgG) monoclonal antibody (mAb) directed toward HER2; this antibody has been reported to significantly improve survival of patients with HER2 positive disease [Lin N U, et al., Brain metastases: the HER2 paradigm. Clin Cancer Res. 2007 Mar. 15; 13(6):1648-55; Palmieri D, et al, Her-2 overexpression increases the metastatic outgrowth of breast cancer cells in the brain. Cancer Res. 2007 May 1; 67(9):4190-8]. However, patients that benefit from trastuzumab often experience simultaneous progression of CNS disease because mAbs do not cross the BBB [Nakayama, cited above]. Injecting trastuzumab directly into the CNS has been proven to be safe, and intrathecal administration of trastuzumab to patients with leptomeningeal carcinomatosis has been reported to increase overall survival from 2 to 13.5 months [Zagouri F, et al., Intrathecal administration of trastuzumab for the treatment of meningeal carcinomatosis in HER2-positive metastatic breast cancer: a systematic review and pooled analysis. Breast Cancer Res Treat. 2013 May; 139(1):13-22]. Leptomeningeal carcinomatosis is associated with an impaired, rather than an intact, blood-brain barrier. Park, E J, et al., J Controlled Release, 163 (2012), 277-284 report that focused ultrasound bursts combined with circulating microbubbles can temporarily permeabilize both the blood brain barrier and the blood tumor barrier for trastuzumab.

While current therapies have led to an improved control of the systemic disease, treatment of metastatic dissemination of human breast cancer into the CNS is a great therapeutic challenge.

### SUMMARY OF THE INVENTION

Provided herein is a therapeutic, recombinant, and replication-defective adeno-associated virus (rAAV) comprising an adeno-associated virus (AAV) capsid containing a vector genome, wherein the vector genome comprises: (a) an AAV-5′ inverted terminal repeat (ITR), (b) an expression cassette comprising a coding sequence for an anti-Her2 antibody having a heavy chain and a light chain, said expression cassette comprising: (i) a nucleic acid sequence encoding an IL2 leader peptide operably linked to an anti-Her2 antibody heavy chain, (ii) a nucleic acid sequence comprising SEQ ID NO: 3 or a sequence at least 95% identical to SEQ ID NO: 3 encoding an anti-Her2 antibody heavy chain, (iii) a furin cleavage site, (iv) a T2A element linker, (v) a nucleic acid sequence encoding an IL2 leader peptide operably linked to an anti-Her2 antibody light chain, (vi) nucleic acid sequence comprising SEQ ID NO: 5 or a sequence at least 95% identical to SEQ ID NO: 5 encoding an anti-Her2 antibody light chain, and regulatory control sequences operably linked to the sequences in the expression cassette, wherein the regulatory sequences comprise a ubiquitin C (UbC) promoter, an optional enhancer, an intron, and a polyadenylation (polyA) sequence, and (c) an AAV-3′ITR. In certain embodiments, the nucleic acid sequence encoding a leader peptide operably linked to an anti-Her2 antibody heavy chain comprises SEQ ID NO: 7, and the nucleic acid sequence encoding a leader peptide operably linked to an anti-Her2 antibody light chain comprises SEQ ID NO: 9. In certain embodiments, the trastuzumab coding sequence comprises nucleic acid sequence of SEQ ID NO: 29, or a sequence at least 95% identical to SEQ ID NO: 29. In certain embodiments, the expression cassette comprises UbC promoter having a nucleic acid sequence of SEQ ID NO: 24. In certain embodiments, the expression cassette comprises SV40 polyA having a nucleic acid sequence of SEQ ID NO: 23. In certain embodiments, the expression cassette comprises a chimeric intron having nucleic acid sequence of SEQ ID NO: 22. In certain embodiments, the expression cassette comprises nucleic acid sequence of SEQ ID NO: 26. or a nucleic acid sequence at least 99% identical to SEQ ID NO: 26. In certain embodiments, the vector genome comprises nucleic acid sequence of SEQ ID NO: 25, or a nucleic acid sequence at least 99% identical to SEQ ID NO: 25.

In certain embodiments, provided herein is a recombinant adeno-associated virus (rAAV) comprising an adeno-associated virus (AAV) capsid containing a vector genome, wherein the vector genome comprises: (a) an AAV-5′ inverted terminal repeat (ITR), (b) an expression cassette comprising a coding sequence for an anti-Her2 antibody having a heavy chain and a light chain, said expression cassette comprising: (i) a nucleic acid sequence encoding an IL2 leader peptide operably linked to an anti-Her2 antibody heavy chain, (ii) a nucleic acid sequence comprising SEQ ID NO: 3 or a sequence at least 95% identical to SEQ ID NO: 3 encoding an anti-Her2 antibody heavy chain, (iii) a furin cleavage site, (iv) a T2A element linker, (v) a nucleic acid sequence encoding an IL2 leader peptide operably linked to an anti-Her2 antibody light chain, (vi) nucleic acid sequence comprising SEQ ID NO: 5 or a sequence at least 95% identical to SEQ ID NO: 5 encoding an anti-Her2 antibody light chain, and regulatory control sequences operably linked to the sequences in the expression cassette, wherein the regulatory sequences comprise one or more of: a promoter which is a chicken beta actin promoter or a CB7 hybrid promoter comprising cytomegalovirus immediate early (CMV IE) enhancer, a chicken beta actin promoter, and a chimeric intron comprising chicken beta actin intron, and a polyA sequence which is a SV40 polyA sequence. In certain embodiments, the CB7 hybrid promoter has nucleic acid sequence of SEQ ID NO: 21. In certain embodiments, the expression cassette comprises nucleic acid sequence of SEQ ID NO: 28, a nucleic acid sequence at least 99% identical to SEQ ID NO: 28, a nucleic acid sequence of SEQ ID NO: 2 or a nucleic acid sequence at least 99% identical to SEQ ID NO: 2. In certain embodiments, the vector genome comprises nucleic acid sequence of SEQ ID NO: 27, a sequence at least 99% identical to SEQ ID NO: 27, SEQ ID NO: 50, a sequence at least 99% identical to SEQ ID NO: 50, SEQ ID NO: 1, or a sequence at least 99% identical to SEQ ID NO: 1. In certain embodiments, the capsid is an AAVhu68 capsid, an AAV9 capsid, an AAVhu95 capsid, AAVhu96, or AAVrh91 capsid.

In one aspect, provided herein is a composition and pharmaceutical composition comprising a rAAV as described herein and an aqueous suspension media. In certain embodiments, the rAAV or the composition thereof is for use in treating metastatic cancer in the brain, wherein the metastatic cancer is from HER2-positive primary tumor, optionally for use in treating metastatic breast cancer in the brain. In certain embodiments, the composition or a pharmaceutical composition is formulated for a central nervous system (CNS) delivery, optionally wherein the CN S delivery is optionally intrathecal delivery, optionally via intracerebroventricular (ICV) injection, intracisterna magna (ICM) injection, intraparenchymal injection, direct injection into the tumor or tumor bed, or via an Ommaya device. In certain embodiments, the rAAV or a composition thereof as described herein is for use in preparing a medicament for treatment of metastatic HER2-positive cancer in brain, optionally wherein the metastatic HER2-positive cancer in brain is a metastatic breast cancer in the brain. In certain embodiments, the composition may be delivered via a systemic route and/or directly into a Her2-positive tumor located outside of the CNS (e.g., in the breast or a metastatic Her2-positive cancer).

In a further aspect, provided herein is a recombinant nucleic acid molecule comprising (a) an AAV-5′ inverted terminal repeat (ITR), (b) an expression cassette comprising at least one open reading frame (ORF) comprising an anti-Her2 antibody heavy chain and an anti-Her2 antibody light chain and nucleic acid sequences operably linked thereto which regulate expression of the anti-Her2 antibody heavy chain and anti-Her2 antibody light chain, and (c) an AAV-3′ ITR, wherein the expression cassette comprises: (i) a promoter which is: (A) a ubiquitin C (UbC) promoter, or (B) a CB7 hybrid promoter comprising a CMV IE enhancer, a chicken beta-actin promoter, and a chimeric intron comprising chicken beta actin splicing donor including chicken beta actin intron and rabbit beta globin splicing acceptor, and/or (ii) an intron, which is a chimeric intron, and (iii) at least one ORF which comprises: a nucleic acid sequence encoding a leader peptide operably linked to an anti-Her2 heavy chain, (iv) a nucleic acid sequence comprising SEQ ID NO: 3 encoding an anti-Her2 heavy chain, (v) a furin cleavage site, (vi) a T2A linker, (vii) a nucleic acid sequence encoding a leader peptide operably linked to an anti-Her2 light chain, (viii) nucleic acid sequence comprising SEQ ID NO: 5 encoding an anti-Her2 light chain, (ix) an SV40 polyadenylation (polyA) sequence, and wherein the expression cassette further comprises spacer sequences. In certain embodiments, the nucleic acid molecule comprises expression cassette comprising nucleic acid sequence of SEQ ID NO: 26. In certain embodiments, the nucleic acid molecule comprises the nucleic acid sequence of SEQ ID NO: 25. In certain embodiments, the recombinant nucleic acid molecule is a plasmid.

In a yet further aspect, provided herein is a packaging host cell comprising recombinant nucleic acid molecule as described herein, which further comprises AAV rep coding sequences operably linked to sequences which express rep protein in the packaging host cell, an AAV capsid coding sequences operably linked to sequences which express AAV capsid proteins in the packaging host cell, and helper virus functions necessary to permit packaging of the expression cassette and AAV ITRs into the AAV capsid.

In another aspect, provided herein is an rAAV production system useful for producing the rAAV as described herein, wherein the production system comprises a cell culture comprising: (a) a nucleic acid sequence encoding a AAV capsid protein; (b) a vector genome; and (c) sufficient AAV rep functions and helper functions to permit packaging of the vector genome into the AAV capsid.

In yet another aspect, provided herein is a method for treating metastatic HER2-positive cancer in the brain. In certain embodiments, provided herein is a method for treating metastatic breast cancer in the brain. The method comprises administrating an effective amount of a rAAV as described herein, or a composition, a pharmaceutical composition or a suspension thereof to a subject in need thereof. In certain embodiments, a suspension is formulated for intrathecal administration, intra-cisterna magna administration or intracerebroventricular administration. In certain embodiments, provided herein is an anti-neoplastic regimen comprising administering rAAV as described herein, a composition, or a pharmaceutical composition thereof, and in combination with a biologic drug, a small molecule, anti-neoplastic agent, radiation, and/or chemotherapeutic agent.

These and other aspects of the invention are apparent from the following detailed description of the invention.

### BRIEF DESCRIPTION OF THE DRAWINGS

FIG. 1A shows a schematic representation for a designed study examining preclinical activity in mice. FIG. 1B shows results demonstrating preclinical activity of the engineered trastuzumab (anti-tumor activity against breast cancer brain metastasis) shown as a plot of the probability of survival in mice after tumor implantation.

FIG. 2A shows results of the ELISA with plotted measurements of trastuzumab concentration (ng/mg protein) in the perfused brain tissue samples collected post administration with AAVrh91.CB-CI.IL2_V1_Trastuzumab-coGW.SV40 and AAVrh91.UbC.CI.IL2_V1_Trastuzumab-coGW.SV40 (Promega intron) in mice as compared to AAVhu68.CMV.PJ.Trastuzumab.SV40 (previously examined construct) and PBS. FIG. 2B shows results of the ELISA with plotted measurements of trastuzumab concentration (g/mL) in the serum samples collected post administration with AAVrh91.CB-CI.IL2_V1_Trastuzumab-coGW.SV40 and AAVrh91.UbC.CI.IL2_V1_Trastuzumab-coGW.SV40 (Promega intron) in mice as compared to AAVhu68.CMV.PI.Trastuzumab.SV40 (previously examined construct) and PBS. Mean peak serum concentration in patients receiving 500 mg (highest dose) were 377 μg/mL.

FIG. 3A shows results of a biodistribution study with plotted measurements of DNA concentration (GC/gg) in the collected liver and brain tissue. FIG. 3B shows results of a biodistribution study with plotted measurements of RNA concentration (copies/100 ng of RNA) in the collected liver and brain tissue.

FIG. 4A shows further data of measurements of trastuzumab concentration in collected serum samples at day 28 post administration with AAVrh91.CB-CJ.IL2_V1_Trastuzumab-coGW.SV40, AAVrh91.UbC.PI.IL2_V1_Trastuzumab-coGW.SV40 and AAVrh91.UbC.PI.IL2_V2_Trastuzumab-coGW.SV40 in mice as compared to AAVhu68.CMV.PJ.Trastuzumab.SV40 (previously examined construct) and PBS.

FIG. 4B shows further data of measurements of trastuzumab concentration in collected perfused brain tissue samples at day 28 post administration with AAVrh91.CB-CI.IL2_V1_Trastuzumab-coGW.SV40, AAVrh91.UbC.PI.IL2_V1_Trastuzumab-coGW.SV40 and AAVrh91.UbC.P.IL2_V2_Trastuzumab-coGW.SV40 in mice as compared to AAVhu68.CMV.PI.Trastuzumab.SV40 (previously examined construct) and PBS.

FIG. 4C shows further data of measurements of DNA biodistribution in collected brain and liver tissue samples at day 28 post administration with AAVrh91.CB-CI.IL2_V1_Trastuzumab-coGW.SV40, AAVrh91.UbC.PI.IL2_V1_Trastuzumab-coGW.SV40 and AAVrh91.UbC.P.IL2 V2 Trastuzumab-coGW.SV40 in mice as compared to AAVhu68.CMV.PI.Trastuzumab.SV40 (previously examined construct) and PBS.

FIG. 5A shows expression levels of trastuzumab (g/mL) as measured in serum samples at day −1, 7, 14, and 28 post administration with AAVhu95.CB.CJ.IL2_V1.Trastuzumab-coGW.SV40, AAVrh91.CB.CI.IL2_V1.Trastuzumab-coGW.SV40 in comparison with capsid control and PBS.

FIG. 5B shows expression levels of trastuzumab (gg/mL) as measured in brain tissue samples at day −1, 7, 14, and 28 post administration with AAVhu95.CB.CI.IL2_VJTrastuzumab-coGW.SV40, AAVrh91.CB.CI.IL2_V1.Trastuzumab-coGW.SV40 in comparison with capsid control and PBS.

FIG. 6 shows vector biodistribution (GC/diploid cell) samples at day −1, 7, 14, and 28 post administration with AAVhu95.CB.CI.IL2_V. Trastuzumab-coGW.SV40, AAVrh91.CB.CI.IL2_V1.Trastuzumab-coGW.SV40 in comparison with capsid control and PBS.

FIG. 7 shows quantified results of the tumor bioluminescence assessment in mice xenograft (MDA-MB-453 (ER−/PR−/HER2+)) post treatment with AAVhu95.CB.CI.IL2.V1.Trastuzumab-coGW.SV40 in comparison with isotype control.

FIG. 8A shows that ICV Injection of AAV Resulted in Sustained Expression of Trastuzumab in Rag1 KO Mice as measured with ELISA in serum samples. FIG. 8B shows that ICV Injection of AAV Resulted in Sustained Expression of Trastuzumab in Rag1KO Mice as measured with ELISA in brain homogenate (perfused) samples.

FIG. 9 shows Kaplan-Meier survival analysis (disease remission) of probability of survival in tumor bearing mice (MDA-MB-453 (ER−/PR−/HER2+) xenografts) treated with AAVhu95. CB.CI.IL2.V1.Trastuzumab-coGW.SV40.

FIG. 10 Kaplan-Meier survival analysis (prophylactic treatment) of probability of survival in tumor bearing mice (BT-474 (ER+/PR+/HER2+) brain xenografts) treated with AAVhu95.CB.CI.IL2.V1.Trastuzumab-coGW.SV40.

FIGS. 11A and 11B show a representative western blot confirming expression of the Trastuzumab heavy (FIG. 11A) and light (FIG. 11B) chains in brain lysates post administration of AAVrh91.CB.CI.IL2_V1.Trastuzumab-coGW.SV40 and AAVhu68.CMV.PI.Trastuzumab.SV40 (column 2921 and 2922) (e.g., FIGS. 2A and 2B).

FIG. 12A shows a schematic representation for an experimental design of a study examining preclinical activity in mice. FIG. 12B shows quantified results of the tumor bioluminescence assessment in mice xenograft (BT-474 Clone 5 trastuzumab-resistant (ER+/PR+/HER2+) xenograft) post treatment with AAVhu95.CB.CI.IL2.V1.Trastuzumab-coGW.SV40 in comparison with isotype control. FIG. 12C shows Kaplan-Meier survival analysis of probability of survival in tumor bearing mice (BT-474 Clone 5 trastuzumab-resistant (ER+/PR+/HER2+) xenograft) treated with AAVhu95.CB.CI.IL2.V1.Trastuzumab-coGW.SV40

FIG. 13A shows a schematic representation for an experimental design of a study examining preclinical activity in mice. FIG. 13B shows Kaplan-Meier survival analysis of probability of survival in tumor bearing mice (MDA-MB-231^{HER2/low} tumors) treated with AAVhu95.CB.CI.IL2.V1.Trastuzumab-coGW.SV40. FIG. 13C shows HER2 expression levels in MDA-MB-231 cells as measured via flow cytometry following surface staining with isotype control antibody. FIG. 13D shows HER2 expression levels in MDA-MB-231 cells as measured via flow cytometry following surface staining with HER2 antibody.

FIG. 14A shows a schematic representation for an experimental design of a study examining preclinical activity in mice.

FIG. 14B shows quantified results of the tumor burden assessment (bioluminescence assessment) in mice xenograft (BT-474 (ER+/PR+/HER2+) xenograft) post treatment with AAVhu95.CB.CI.IL2.V1.Trastuzumab-coGW.SV40 in comparison with PBS, and isotype control.

FIG. 14C shows Kaplan-Meier survival analysis of probability of survival in tumor bearing mice (BT-474 Clone 5 Trastuzumab Resistant (ER+/PR+/HER2+) Xenograft) treated with AAVhu95. CB. CI.IL2.V1.Trastuzumab-coGW.SV40.

FIG. 15A shows a schematic representation for an experimental design of a study examining long-term transgene expression after a single or double (re-dose) administration of AAV.Trastuzumab in MDA-MB-453 (ER−/PR−/HER2+) xenograft model.

FIG. 15B show trastuzumab expression levels measured by ELISA using the serum samples harvested on day 98, plotted as trastuzumab g/mL in MDA-MB-453 (ER−/PR−/HER2+) xenograft model.

FIG. 15C show trastuzumab expression levels measured by ELISA using the brain samples (perfused brain homogenate) harvested on day 98, plotted as trastuzumab g/mL in MDA-MB-453 (ER−/PR−/HER2+) xcnograft model.

FIG. 16 shows trastuzumab expression levels in serum following administration of AAVhu95M199.CB7.CI.IL2_Trastuzumab-coGW.SV40, AAVhu95M199.IE.CB7.CI.IL2_Furin_V1 Trastuzumab-coGW.SV40, AAVhu95M199.UbC.PI.IL2 Furin_V1_Trastuzumab-coGW.SV40, AAVhu95M199.UbC.PI.IL2_Trastuzumab-coGW.SV40 in mice.

FIG. 17 shows trastuzumab expression levels in brain (perfused) following administration of AAVhu95M199.CB7.C.IL2_Trastuzumab-coGW.SV40, AAVhu95M199.IE.CB7.CJ.IL2 Furin_V1 Trastuzumab-coGW.SV40, AAVhu95M199.UbC.PI.IL2_Furin_V1_Trastuzumab-coGW.SV40, AAVhu95M199.UbC.PI.IL2_Trastuzumab-coGW.SV40 in mice.

FIG. 18A shows a schematic of the experimental design of the study for assessing tumor burden. Briefly, seven days before cell implantation (D-7) a guide screw was implanted, on day 0 MDA-MB-453 cells were implanted (2.5×10⁵ cells/mouse), on day 3 post cell implantation mice were administered withrAAV.trastuzumab at a dose of 1×10¹¹ GC/mouse intracranially (ICV), and imaging was performed at 2, 4, and 6 weeks post cell implantation.

FIG. 18B shows results of the tumor burden assessment plotted as Total Flux (p/s) at 2, 4 and 6 weeks post tumor cell implantation in mice administered with AAVhu95M199.UbC.CJ.IL2.3bnc117.SV40, AAVhu95M199.IE.CB.CJ.3bnc117.SV40, AAVhu95M199.IE.CB7.CJ.IL2_Furin_V1.Trastuzumab-coGW.SV40, AAVhu95M199.UbC.PI.IL2_Furin_V1.Trastuzumab-coGW.SV40. These results confirm therapeutic effect of rAAV.Trastuzumab vectors.

FIG. 18C shows Kaplan-Meier survival curve in mice following administration with AAVhu95M199.UbC.CJ.IL2.3bnc117.SV40, AAVhu95M199.IE.CB.CJ.3bnc117.SV40, AAVhu95M199.IE.CB7.CJ.IL2_Furin_V1.Trastuzumab-coGW.SV40, AAVhu95M199.UbC.PI.IL2_Furin_V1.Trastuzumab-coGW.SV40.

FIG. 19A shows a schematic of the experimental design of the study for assessing antitumor activity. Briefly, seven days before cell implantation (D-7) a guide screw was implanted, on day 0 E2 pellets with BT474 clone 5 cells were implanted (1.5×10⁵ cells/mouse), on day 3 post cell implantation mice were administered with rAAV.trastuzumab at a dose of 1×10¹¹ GC/mouse intracranially (ICV), and imaging was performed at 2 and weeks post cell implantation.

FIG. 19B shows results of the tumor burden assessment plotted as Total Flux (p/s) at 2, 4 and 6 weeks post tumor cell implantation in mice administered with AAVhu95M199.UbC.CJ.IL2.3bnc117.SV40, AAVhu95M199.UbC.PI.IL2_Furin_V1.Trastuzumab-coGW.SV40.

FIG. 19C shows Kaplan-Meier survival curve in mice (with BT474 Xenografts) following administration with AAVhu95M199.UbC.CI.IL2.3bnc117.SV40, AAVhu95M199.IE.CB.CI.3bnc117.SV40, AAVhu95M199.IE.CB7.CI.IL2_Furin_V1.Trastuzumab-coGW.SV40, AAVhu95M199.UbC.PI.IL2_Furin_V1.Trastuzumab-coGW.SV40.

FIG. 20 shows results of the vector titration study plotted as trastuzumab expression levels a as measured by ELISA and Mass Spectrometry in mice administered with rAAV.Trastuzumab at a dose of 1×10¹¹ GC/mouse and 1×10¹⁰ GC/mouse.

FIG. 21A shows a schematic of the experimental design of the study for assessing tumor challenge.

FIG. 21B shows tumor growth plotted as measured total flux (p/s) at 2, 4, and 5 weeks post tumor implantation in mice administered with AAVhu95M199.UbC.CI.IL2.3bnc117.SV40, AAVhu95M199.UbC.PI.IL2_Furin_V1. Trastuzumab-coGW.SV40 at a dose 1×10¹¹ GC/mouse.

FIG. 22A shows a schematic of the experimental design of the study for assessing tumor challenge. FIG. 22B shows tumor growth plotted as measured total flux (p/s) at 2, 4, and 5 weeks post tumor implantation in mice administered with AAVhu95M199.UbC.CI.IL2.3bnc117.SV40, AAVhu95M199.UbC.PI.IL2_Furin_V1. Trastuzumab-coGW.SV40 at a dose 1×10¹⁰ GC/mouse.

FIG. 23A shows results of tumor burden assessment as examined by imaging plotted as total flux (p/s) in mice administered with AAVhu95M199.UbC.CI.IL2.3bnc117.SV40, AAVhu95M199.UbC.PI.IL2_Furin_V1.Trastuzumab-coGW.SV40 at a dose 1×10¹⁰ GC/mouse and 1×10¹¹ GC/mouse. FIG. 23B shows results of Kaplan-Meier survival analysis plotted as probability of survival in mice administered with AAVhu95M199.UbC.CI.IL2.3bnc117.SV40, AAVhu95M199.UbC.PI.IL2_Furin_V1.Trastuzumab-coGW.SV40 at a dose 1×10¹⁰ GC/mouse and 1×10¹¹ GC/mouse.

FIG. 24A shows trastuzumab expression levels in serum following administration with rAAV.Trastuzumab with and without IVIG pre-treatment. FIG. 24B shows trastuzumab expression levels in brain (perfused) at day 30 following administration with rAAV.Trastuzumab with and without IVIG pre-treatment.

FIG. 25A shows trastuzumab expression levels in CSF as measured by ELISA on days 0. 3, 7, 14, 21 and 36-37 following rAAV administration in NHPs of cohort 1b ((AAVhu95M199.IE.CB7.CI.IL2_Furin_V1.Trastuzumab-coGW.SV40)), cohort 2 (AAVhu95M199.UbC.PI.IL2_Furin_V1.Trastuzumab-coGW.SV40), and negative control on day 14. FIG. 25B shows levels of anti-drug antibodies as measured in collected samples of CSF in NHPs on days 0. 3, 7, 14, 21 and 36-37 following rAAV administration (AAVhu95M199.TE.CB7.CI.IL2_Furin_V1.Trastuzumab-coGW.SV40 and AAVhu95M199.UbC.PI.IL2_Furin_V1. Trastuzumab-coGW.SV40.

FIG. 26A shows trastuzumab expression levels in serum as measured by ELISA on days 0. 3, 7, 14, 21 and 36-37 following rAAV administration in NHPs of cohort 1b ((AAVhu95M199.IE.CB7.CI.IL2_Furin_V1.Trastuzumab-coGW.SV40)), cohort 2 (AAVhu95M199.UbC.PI.IL2_Furin_V1.Trastuzumab-coGW.SV40).

FIG. 26B shows levels of anti-drug antibodies as measured in collected samples of serum in NHPs on days 0. 3, 7, 14, 21 and 36-37 following rAAV administration (AAVhu95M199.IE.CB7.CI.IL2_Furin_V1.Trastuzumab-coGW.SV40 and AAVhu95M199.UbC.PI.IL2_Furin_V1.Trastuzumab-coGW.SV40.

FIG. 27 shows quantification of trastuzumab protein in NHP brain tissue following ICM administration of AAV.Trastuzumab (AAVhu95M199.IE.CB7.CLIL2_Furin_V1.Trastuzumab-coGW.SV40 and AAVhu95M199.UbC.PI.IL2_Furin_V1.Trastuzumab-coGW.SV40).

FIG. 28A shows quantification of trastuzumab protein in NHP spinal cord following ICM administration of AAV.Trastuzumab (AAVhu95M199.IE.CB7.CI.IL2_Furin_V1.Trastuzumab-coGW.SV40 and AAVhu95M199.UbC.PI.IL2_Furin_V1.Trastuzumab-coGW.SV40).

FIG. 28B shows quantification of trastuzumab protein in NHP Dorsal Root Ganglion (DRG) following ICM administration of AAV.Trastuzumab (AAVhu95M199.IE.CB7.CI.IL2_Furin_V1.Trastuzumab-coGW.SV40 and AAVhu95M199.UbC.PI.IL2_Furin_V1.Trastuzumab-coGW.SV40).

FIGS. 29A to 29D show results of trastuzumab mRNA Detection in NHP brain tissues (cerebellum and occipital lobe cortex) by 10×GENOMICS (final sequencing depth average reads −883×10⁶ reads/sample). There was observed no background detection of trastuzumab in untreated monkeys. FIG. 29A shows results of trastuzumab mRNA Detection in cerebellum in NHP 18-032 administered with AAVhu95M199.UbC.PI.IL2_Furin_V1.Trastuzumab-coGW.SV40. FIG. 29B shows results of trastuzumab mRNA Detection in cerebellum in NHP 20-198 administered with AAVhu95M199.UbC.PI.IL2_Furin_V1.Trastuzumab-coGW.SV40. FIG. 29C shows results of trastuzumab mRNA Detection in occipital lobe cortex in NHP 18-032 administered with AAVhu95M199.UbC.PI.IL2 Furin_V1.Trastuzumab-coGW.SV40. FIG. 29D shows results of trastuzumab mRNA Detection in occipital lobe cortex in NHP 20-198 administered with AAVhu95M199.UbC.PI.IL2_Furin_V1. Trastuzumab-coGW.SV40.

FIGS. 30A and 30B show biodistribution of AAV.Trastuzumab (AAVhu95M199.IE.CB7.CJ.IL2_Furin_V1.Trastuzumab-coGW.SV40 and AAVhu95M199.UbC.PI.IL2 Furin_V1. Trastuzumab-coGW.SV40) in NHP in various tissues following ICM administration. FIG. 30A shows DNA biodistribution of AAV.Trastuzumab in NHP in various tissues following ICM administration. FIG. 30B shows RNA biodistribution of AAV.Trastuzumab in NHP in various tissues following ICM administration.

FIGS. 31A-31H shows single ICV administration of AAV9 vector encoding engineered version of trastuzumab resulted in robust transgene expression in RAG1 KO Mice. FIG. 31A shows a schematic of AAV vector genome. FIG. 31B shows schematic for evaluation of in vivo transgene expression following ICV delivery of AAV9 vector encoding trastuzumab (1×10¹¹ GC/mouse) in healthy adult Rag1 KO female mice. FIG. 31C shows longitudinal quantification of trastuzumab by ELISA in serum from Rag1 KO mice treated ICV with AAV9.UbC.trastuzumab vector (1×10¹¹ GC/mouse; n=10) or PBS (n=10). FIG. 31D shows longitudinal quantification of trastuzumab by ELISA in perfused brain tissue from Rag1 KO mice treated ICV with AAV9.UbC.trastuzumab vector (1×10¹¹ GC/mouse; n=10) or PBS (n=10). FIG. 31E shows DNA biodistribution and FIG. 31F shows RNA biodistribution analysis by qPCR of brain and liver tissue from Rag1 KO mice treated ICV with AAV9.UbC.Trastuzumab or PBS.

FIG. 31G shows Western blot analysis of Trastuzumab heavy and light chains in brain lysates from Rag1 KO mice treated ICV with AAV9.UbC.trastuzumab (n=4) or PBS (n=2). Purified trastuzumab (10 ng/lane) and f-actin were used as a positive control and loading control, respectively. FIG. 31H shows trastuzumab identity was confirmed by LC-MS using brain homogenates from Rag1 KO mice treated ICV with AAV9.UbC.trastuzumab (n=4) or PBS (n=2). Data shown as individual data points and mean±SEM.

FIGS. 32A to 32C show AAV9.UbC.trastuzmnab CNS transduction efficiency is not affected by pre-treatment with intravenous immunoglobulin (IVIg) containing broad neutralizing antibodies against AAV9. Adult female Rag1 KO mice were pre-treated with IVIg or control serum from C57BL6/J donor mice 2 hours prior to ICV administration of AAV9.UbC.Trastuzumab (1×10¹¹ GC/mouse; n=5). Mice treated ICV with PBS (n=3) were used as controls. FIG. 32A shows trastuzumab levels in serum over time and FIG. 32B shows trastuzumab levels in perfused brain tissue (day 30) from ICV treated mice were measured by ELISA. FIG. 32C shows DNA biodistribution analysis of AAV9.UbC.Trastuzumab vector by qPCR in brain, liver and heart 30 days post-vector administration. Data shown as individual data points and mean±SEM. ***p<0.0001; ns, not significant.

FIGS. 33A to 33E show that a single dose ICV administration of AAV9.UbC.Trastuzumab led to tumor regression in xenograft mouse models of HER2+ breast to brain metastases. Three days post-intracranial implantation of BT-474 (ER+/PR+/HER2+) or MDA-MB-453 (ER−/PR−/HER2+) human breast cancer cell lines, adult female Rag1 KO mice were ICV injected with 1×10¹¹ GC/animal of AAV9.UbC.trastuzumab or AAV9.isotype control.

FIG. 33A shows quantification of total photon flux via bioluminescent in vivo imaging system (IVIS) and FIG. 33B shows a Kaplan-Meier survival curve of mice bearing BT-474 cell line-derived orthotopic brain tumors and treated ICV with AAV vectors (AAV9.Isotype control: n=9; or AAV9.UbC.trastuzumab: n=12). FIG. 33C shows representative bioluminescent IVIS images (at week 4, left panel), total photon flux signal quantification (right panel), and FIG. 33D shows a Kaplan-Meier survival curve of mice bearing MDA-MB-453 cell line-derived orthotopic brain tumors following ICV treatment (AAV9.isotype control: n=8; or AAV9.UbC.trastuzumab: n=7).

FIG. 33E shows a long-term trastuzumab expression quantified in perfused brain tissue by ELISA in a subset of mice showcased in FIG. 33C and FIG. 33D (n=5) at day 104 post-ICV administration of AAV9.UbC.trastuzumab.***p<0.0001 FIGS. 34A to 34E show detection of trastuzumab in the CSF of female NHPs following intra-cisterna magna (ICM) administration of AAV9 vectors encoding trastuzumab. FIG. 34A sows schematic design of experimental ICM delivery of 3×10¹³ GC/animal of AAV9.UbC.trastuzumab (n=2) vector in NHPs to inform in-life blood and CSF collection schedules and scheduled end-of-study time points. NHP #3 was injected ICM with 3×10¹³ GC of an AAV9 vector expressing GFP and was used as a negative control. Detection of trastuzumab, a humanized IgG1 antibody, in the CSF (FIG. 34B) and serum (FIG. 34C) of treated animals was determined by ELISA following quantification of human IgG and anti-drug antibodies against trastuzumab in CSF (FIG. 34D) and serum (FIG. 34E).

FIGS. 35A, 35B, and 36A to 36D show CNS-wide gene delivery and transgene transcription following ICM treatment with 3×10¹³ GC/animal of AAV9.UbC.trastuzumab. FIG. 35A shows absolute quantification of AAV vector GC in NHPs on day 36-37 post-ICM administration of AAV9.UbC.Trastuzumab (n=2) vector. FIG. 35B shows transcripts in selected tissues by qPCR in NHPs on day 36-37 post-ICM administration of AAV9.UbC.Trastuzumab (n=2) vector.

FIGS. 36A to 36D show results of single-nuclei RNA sequencing showing the presence of trastuzumab mRNA transcripts in the cerebellum and occipital cortex from animals injected ICM with AAV9.UbC.Trastuzumab vector.

FIGS. 37A to 37F show that ICM delivery of 3×10¹³ GC/animal with AAV9.UbC.trastuzumab results in transgene protein expression in NHP CNS tissues at levels sufficient to induce complete antitumor responses in tumor bearing mice. Experimental scheme is shown in FIG. 34A. FIG. 37A shows results of LC-MS analysis to detect a unique AAV-encoded, trastuzumab-derived peptide (DTYIHWVR; SEQ ID NO: 56) in NHP brain homogenates. Trastuzumab levels presented as fold change relative to input reference positive control (10 ng trastuzumab). FIG. 37B shows trastuzumab expression in perfused brain tissue from female Rag1 KO mice 28 days post-ICV administration of 1×10¹⁰ GC/animal of AAV9.isotype control (n=8) or AAV9.UbC.trastuzumab (n=8), measured by ELISA. FIG. 37C shows trastuzumab protein expression in perfused brain regions by ELISA by determining the tissue levels of human IgG.

FIG. 37D shows quantification of total photon flux by IVIS imaging of adult female Rag1 KO mice treated ICV with 1×10¹⁰ GC/animal of AAV9.isotype control (n=8) or AAV9.UbC.trastuzumab (n=8) 3 days following intracranial tumor cell implantation of reporter MDA MB 453 breast cancer cells. FIG. 37E shows trastuzumab protein expression in spinal cord which were quantified by ELISA by determining the tissue levels of human IgG. FIG. 37F shows Kaplan Meier survival curves of adult female Rag1 KO mice treated ICV with 1×10¹⁰ GC/animal of AAV9.isotype control (n=8) or AAV9.UbC.trastuzumab (n=8) 3 days following intracranial tumor cell implantation of reporter MDA MB 453 breast cancer cells. ***p<0.0001

### DETAILED DESCRIPTION OF THE INVENTION

Provided herein are recombinant, replication-defective adeno-associated virus (rAAV) vectors having an AAV capsid and packaged therein a vector genome comprising at least one open reading frame comprising engineered nucleic acid sequence encoding heavy chain and light chain of anti-HER2 antibody (e.g., anti-neoplastic immunoglobulin construct, trastuzumab), and compositions containing same which are suitable for administration for treatment of anti-Her2 cancer, including metastatic cancers. Various methods of administration are provided, including systemic administration, direct administration to a primary to secondary tumor (metastatic cancer) (e.g., intratumoral), and administration to the central nervous system (e.g., intrathecal administration) for treatment of metastatic Her2-positive cancer, among others. Also provided are pharmaceutical compositions, formulations containing same, and in particularly, liquid aqueous suspension. Uses of these compositions are also provided. Also provided are method of compositions useful for the treatment and/or prevention of metastatic HER2-positive (HER2-human epidermal growth factor receptor 2; also referred to as Her2-positive, HER2+, or Her2+) cancers, e.g., in the brain.

In certain embodiments, the compositions and regimens described herein are useful for delivery of anti-neoplastic immunoglobulin constructs to the central nervous system.

Compositions described herein comprising AAV-1g are well suited for central nervous system (CNS) HER2-positive cancers (neoplasms), and particularly for those located in the brain. The compositions and regimens are also useful for treating primary and/or secondary Her2-positive breast cancer, primary and/or secondary Her2-positive gastric and/or primary and/or secondary Her2-positive gastric gastroesophageal junction cancer, and other HER-2 positive solid tumors and cancers.

As used herein, the term “CNS neoplasms” includes primary or metastatic cancers, which may be located in the brain (intracranial), meninges (connective tissue layer covering brain and spinal cord), or spinal cord. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

As used herein, an “anti-neoplastic” immunoglobulin construct (including antibody or antibody fragment as defined herein) encodes a polypeptide-based moiety which binds to a cell-surface antigen or receptor located on a cancer cell or solid tumor and which inhibits or prevents the growth and spread of tumors, or malignant cells in a non-solid tumor, and optionally, reduces the size of tumors. The anti-neoplastic immunoglobulin polypeptides can function by a number of mechanisms, e.g., inhibiting tumor cell growth by blocking a growth factor receptor, cross-linking cell membrane antigens to deliver signals that control the cell cycle, blocking angiogenesis, blocking DNA repair post chemotherapy, or even inducing cell death. Alternatively, they can influence tumor growth indirectly by activating host immune effector functions such as antibody-dependent and complement-mediated cell cytotoxicity. In one embodiment, the anti-neoplastic effect of the compositions and regimens described herein can be measured by reduction of tumor size and/or by an increased progression-free survival rate as compared to subjects which are untreated or treated with other regimens.

The term “immunoglobulin” is used herein to include antibodies, functional fragments thereof, and immunoadhesins. Antibodies may exist in a variety of forms including, for example, polyclonal antibodies, monoclonal antibodies, camelized single domain antibodies, intracellular antibodies (“intrabodies”), recombinant antibodies, multispecific antibody, antibody fragments, such as, Fv, Fab, F(ab)2, F(ab)3, Fab′, Fab′-SH, F(ab′)2, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), chimeric antibodies, chimeric antibodies comprising at least one human constant region, and the like. “Antibody fragment” refers to at least a portion of the variable region of the immunoglobulin that binds to its target, e.g., the tumor cell, tumor cell receptor.

The rAAV vectors, as described herein encode the anti-HER2 antibody (e.g., trastuzumab) heavy chain and light chain. See, e.g., drugbank.ca/drugs/DB00072. The encoded trastuzumab heavy chain amino acid sequence is:

EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK
(SEQ ID NO: 35; drugbank.ca/drugs/DB00072).

In certain embodiments, the encoded trastuzumab heavy chain amino acid sequence is: EVQLVESGGGLVQPGGSLRLSCAASGFNIKDTYIHWVRQAPGKGLEWVARIYPTNGYTR YADSVKGRFTISADTSKNTAYLQMNSLRAEDTAVYYCSRWGGDGFYAMDYWGQGTLV TVSSASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPCPAPEL LGGPSVFLFPPKPKDTLMISRTPEVTCVVVDVSHEDPEVKFNWYVDGVEVHNAKTKPRE EQYNSTYRVVSVLTVLHQDWLNGKEYKCKVSNKALPAPIEKTISKAKGQPREPQVYTLP PSREEMTKNQVSLTCLVKGFYPSDIAVEWESNGQPENNYKTTPPVLDSDGSFFLYSKLTV DKSRWQQGNVFSCSVMHEALHNHYTQKSLSLSPG (SEQ ID NO: 4; i.e., without terminal lysine (K)).

The trastuzumab light chain is a recombinant IgG1 kappa, humanized monoclonal antibody that selectively binds with high affinity in a cell-based assay (K_{d}=5 nM) to the extracellular domain of the human epidermal growth factor receptor protein. The encoded amino acid sequence of the trastuzumab light chain is:

DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 36 (drugbank_ca/drugs/
DB00072), also SEQ ID NO: 6)),

which sequences are incorporated herein by reference. See, also, 212-Pb-TCMC-trastuzumab [Areva Med, Bethesda, MD]. As described herein, a series of novel rAAV.anti-HER2-antibody (rAAV.trastuzumab or AAV.trastuzumab) constructs have been developed which have demonstrated high yield, expression levels, and/or activity. In certain embodiments, the sizes of the antibody (protein) chains can be confirmed by western blot. In certain embodiments, the sizes of the antibody (protein) chains can be confirmed by mass spectrometry.

Provided herein are coding sequence(s), expression cassette(s), and a vector genome(s) encoding the trastuzumab heavy chain (HC) amino acid sequence of SEQ ID NO: 4 (amino acids 1 to 449 of SEQ ID NO: 35) and a light chain (LC) amino acid sequence of SEQ ID NO: 6 (also SEQ ID NO: 36), each of which has been engineered to further have an exogenous leader sequence (also referred to as “leader peptide” or “signal peptide” or “signal sequence”) for each the heavy chain and the light chain (e.g., SEQ ID NO: 14 for heavy chain amino acid sequence comprising leader peptide, SEQ ID NO: 16 for light chain amino acid sequence comprising a leader peptide). In certain constructs illustrated herein, the leader sequence is derived from a human interleukin 2 (IL2 or IL-2) leader. In certain embodiments, the IL2 is same for each the heavy chain and the light chain. In certain embodiments, the IL2 is a modified IL2 for at least one of the heavy chain and the light chain. Further, in certain constructs illustrated in the working examples, the heavy and light chains are separated by a T2A linker which may result in one or more extra amino acids being added to the heavy chain [SEQ ID NO: 4 or SEQ ID NO: 14 (comprising leader peptide)]. Further, in certain constructs illustrated in the working examples, the heavy and light chains are separated by a furin/T2A linker which may result in one or more extra amino acids being added to the heavy chain [SEQ ID NO: 4 or SEQ ID NO: 14 (comprising leader peptide)]. In one embodiment, a single arginine [R] is added to the heavy chain. However, in certain embodiments, another linker may be selected and/or a different system may result in no additional amino acid, or one or more extra amino acids [e.g., R, Lys (K), RK, RKR, RKRR (SEQ ID NO: 49) among others]. In the constructs encoding the anti-Her2 antibody trastuzumab product, various designation following the term trastuzumab, e.g., trastuzumab-coGW, trastuzumab-coX, trastuzumab-coGY, trastuzumab, refers to different nucleic acid coding sequences for the open reading frame of the anti-Her2 antibody heavy chain and light chain. It will be understood that the resulting anti-Her2 antibody product, following cleavage of the leader peptides and assembly of the anti-Her2 light and heavy chains, optionally, with the mutant amino acid, is referred to herein alternatively as an “anti-Her2 antibody” or trastuzumab, but may contain a variant or mutant amino acid sequence as compared to the amino acid sequence of SEQ ID NO: 35 (drugbank.ca/drugs/DB00072; trastuzuamb heavy chain) and SEQ ID NO: 36 (drugbank_ca/drugs/DB00072; trastuzuamb light chain). In certain embodiments, anti-Her2 antibody may comprise one or more conservative, non-conservative amino acid substitutions, as well as insertions, truncations and/or deletions as compared to amino acid sequence of SEQ ID NO: 35 (drugbank.ca/drugs/DB00072; trastuzuamb heavy chain) and SEQ ID NO: 36 (drugbank_ca/drugs/DB00072; trastuzuamb light chain). In certain embodiments, the amino acid sequence of the anti-Her2 antibody comprises a truncation of the terminal Lysine (K) at the C-terminus of the amino acid sequence as compared to SEQ ID NO: 35.

In certain embodiments, the encoded amino acid sequence of the trastuzumab has 724 amino acids, including trastuzumab heavy chain and light chain separated by extra amino acids as a result of the linker. For example, while each of the described herein expression cassettes encodes the same trastuzumab heavy chain and light chain, in one embodiment, there may be one amino acid added to the last position of the heavy chain. In still other embodiments, there may be two, three, four or more extra amino acids attached to the heavy chain. For example, in certain embodiments, the nucleic acid sequences coding for the heavy and light chains of Trastuzumab are separated by a self-cleaving furin/T2A linker. A furin recognition site that consists of arginine-lysine-arginine-arginine amino acid sequence may be used. Due to the mechanism of furin-mediated cleavage, vector-expressed trastuzumab may contain an additional arginine (R) residue added to the last position of the heavy chain [SEQ ID NO: 4 or SEQ ID NO: 14 (comprising leader peptide)]. In other embodiments, the vector-expressed trastuzumab is modified to contain the dipeptide arginine-lysine at the end of the heavy chain, the tripeptide arginine-lysine-arginine at the end of the heavy chain, or the polypeptide arginine-lysine-arginine-arginine at the end of the heavy chain. In certain embodiments, the vector-expressed trastuzumab immunoglobulins are a heterogeneous mixture of two or more of these immunoglobulin products. Other furin cleavage sites can be used (arginine-X-X-arginine, or arginine-X-lysine or arginine-arginine), which can also generate C-terminal heterogeneity. In other words, other vector expressed trastuzumab immunoglobulins may be a heterogeneous population of the immunoglobulins in which the heavy chain has 0, 1, 2, 3, or 4 amino acids at its C-terminus as a result of the linker processing.

In addition, the light and heavy chain each contain a heterologous leader peptide which directs each of the immunoglobulin chains into appropriate cellular compartment where the leader peptide is processed away from the mature immunoglobulin chain by the host cellular machinery, e.g., HC with leader peptide comprises amino acid sequence of SEQ ID NO: 14, and LC with leader peptide comprises amino acid sequence of SEQ ID NO: 16 and the two chains are permitted to self-assemble in vivo into a recombinant anti-Her antibody.

In certain embodiments, the self-assemble in vivo into a recombinant trastuzumab antibody can be confirmed with native gel electrophoresis (e.g., polyacrylamide gel electrophoresis (native PAGE)) and denaturing gel electrophoresis (i.e., SDS-PAGE). In certain embodiments, the trastuzumab comprises no HC or LC leader peptide sequences (HC amino acid of SEQ ID NO: 4 and LC amino acid sequence of SEQ ID NO: 6).

As used herein, the term “trastuzumab” refers to an immunoglobulin construct (“anti-Her2 antibody”) comprising a heavy chain having an amino acid sequence of SEQ ID NO: 4 or a sequence about 95% to about 100% identical thereto, at least 95%, at least 96%, at least 97%, at least 98%, at least 99%, 99.9% identical thereto, and values therebetween, as determined over contiguous amino acid sequences (e.g., heavy chain has −1, 0, 1, 2, 3, or 4 amino acids at its C-terminus as a result of the linker processing), and comprising light chain having an amino acid sequence of SEQ ID NO: 6 or a sequence about 99% to about 100% identical thereto, or at least 99.9% identical thereto, and values therebetween (e.g., as a result of linker processing), as determined over contiguous amino acid sequences, which provide at least 10%, at least 20%, at least 30%, at least 40%, at least 50%, at least 60%, at least 70%, at least 75%, at least 80%, at least 90%, or a similar and/or same, or greater than 100% biological activity (e.g., binding) or function of trastuzumab immunoglobulin product. In certain embodiments, greater than 100%, e.g., about 105%, about 110%, about 115%, about 120%, about 1²⁵%, about 130%, or greater of trastuzumab as produced in vitro (e.g., in CHO cells) activity and/or function is achieved. This biological activity or function may be determined by any suitable means, e.g., in an in vitro assay, animal model or by monitoring patients post-treatment. In certain embodiments, the AAV-expressed trastuzumab immunoglobulin heavy chain and light chain provide advantages over protein-based trastuzumab compositions derived from in vitro-produced trastuzumab including, e.g., improved glycosylation, decreased deamidation, and/or improved stability.

In one embodiment, the engineered coding sequences for the heavy chain and light chain of trastuzumab are provided in SEQ ID NO: 17. In one embodiment, the engineered coding sequences for the heavy chain and light chain of trastuzumab are provided in SEQ ID NO: 29. In certain embodiments, provided herein is at least one ORF comprising a nucleic acid sequence encoding a leader peptide operably linked to trastuzumab heavy chain, a nucleic acid sequence comprising SEQ ID NO: 3 or a sequence at least 95% identical to SEQ ID NO: 3 encoding a trastuzumab heavy chain, a linker, a nucleic acid sequence encoding a leader peptide operably linked to a trastuzumab light chain, and a nucleic acid sequence comprising SEQ ID NO: 5 or a sequence at least 95% identical to SEQ ID NO: 5 encoding a trastuzumab light chain. In certain embodiments, provided herein is at least one ORF comprising a nucleic acid sequence encoding a leader peptide operably linked to trastuzumab heavy chain, a nucleic acid sequence comprising SEQ ID NO: 3 encoding a trastuzumab heavy chain, a linker, a nucleic acid sequence encoding a leader peptide operably linked to a trastuzumab light chain, and a nucleic acid sequence comprising SEQ ID NO: 5 encoding a trastuzumab light chain. In certain embodiments, the linker between the heavy chain and light chain is a Thosea asigna virus (T2A) linker. In certain embodiments, the T2A linker comprises nucleic acid sequence of SEQ ID NO: 32, or a sequence at least 95% identical to SEQ ID NO: 32. In certain embodiments, the T2A linker comprises nucleic acid sequence of SEQ ID NO: 32, or a sequence at least 95% identical to SEQ ID NO: 32 encoding amino acid sequence of SEQ ID NO: 52. In certain embodiments, the linker further comprises a furin cleavage at 5′ end of the T2A, optionally connected via a flexible linker (e.g., “GSG linker”). In certain embodiments, the furin cleavage sequence comprises nucleic acid sequence of SEQ ID NO: 31, or a sequence at least 95% identical to SEQ ID NO: 31. In certain embodiments, the furin/T2A linker comprises nucleic acid sequence of SEQ ID NO: 33, or a sequence at least 95% identical to SEQ ID NO: 33. In certain embodiments, the furin/T2A linker comprises nucleic acid sequence of SEQ ID NO: 33, or a sequence at least 95% identical to SEQ ID NO: 33 encoding amino acid sequence of SEQ ID NO: 53. In certain embodiments, provided herein is at least one ORF comprising a nucleic acid sequence encoding a leader peptide operably linked to trastuzumab heavy chain, a nucleic acid sequence comprising SEQ ID NO: 3 encoding a trastuzumab heavy chain, a linker comprising a furin cleavage and T2A, wherein furin cleavage site is at 5′ end of the T2A, optionally connected via a flexible linker (e.g., “GSG linker”), a nucleic acid sequence encoding a leader peptide operably linked to a trastuzumab light chain, and a nucleic acid sequence comprising SEQ ID NO: 5 encoding a trastuzumab light chain. In certain embodiments, the trastuzumab heavy chain has the amino acid sequence of SEQ ID NO: 4 with a leader sequence. In other embodiments, the trastuzumab light chain has the amino acid sequence of SEQ ID NO: 6 with a leader sequence. For example, the leader sequence may be from about to about 25 amino acids, preferably about 20 amino acids. In certain embodiments, the processing of trastuzumab heavy chain and light chain is directed by leader peptides that are derived from human IL2 protein. In one embodiment the leader sequence is an interleukin (IL) IL-2 leader sequence, which may be the wild-type human IL2, MYRMQLLSCIALSLALVTNS [SEQ ID NO: 8], or a mutated leader, such as MYRMQLLLLIALSLALVTNS [SEQ ID NO: 10] or MRMQLLLLIALSLALVTNS [SEQ ID NO: 12]. In certain embodiments, the leader peptide comprises nucleic acid sequence comprising SEQ ID NO: 7 or a sequence at least 95% identical to SEQ ID NO: 7. In certain embodiments, the leader peptide comprises nucleic acid sequence comprising SEQ ID NO: 7 or a sequence at least 95% identical to SEQ ID NO: 7 encoding SEQ ID NO: 8. In certain embodiments, the leader peptide comprises nucleic acid sequence comprising SEQ ID NO: 9 or a sequence at least 95% identical to SEQ ID NO: 9. In certain embodiments, the leader peptide comprises nucleic acid sequence comprising SEQ ID NO: 9 or a sequence at least 95% identical to SEQ ID NO: 9 encoding SEQ ID NO: 10. In certain embodiments, the leader peptide comprises nucleic acid sequence comprising SEQ ID NO: 11 or a sequence at least 95% identical to SEQ ID NO: 11. In certain embodiments, the leader peptide comprises nucleic acid sequence comprising SEQ ID NO: 11 or a sequence at least 95% identical to SEQ ID NO: 11 encoding SEQ ID NO: 12. In certain embodiments, the processing of trastuzumab heavy chain and light chain is directed by leader peptides that are same. In certain embodiments, the processing of trastuzumab heavy chain and light chain is directed by leader peptides that are different. In certain embodiments, the trastuzumab heavy chain is directed by the leader peptide comprising amino acid sequence of SEQ ID NO: 8. In certain embodiments, the trastuzumab heavy chain is directed by the leader peptide comprising amino acid sequence of SEQ ID NO: 8 encoded by nucleic acid sequence of SEQ ID NO: 7. In certain embodiments, the trastuzumab light chain is directed by the leader peptide comprising amino acid sequence of SEQ ID NO: 10. In certain embodiments, the trastuzumab light chain is directed by the leader peptide comprising amino acid sequence of SEQ ID NO: 10 encoded by nucleic acid sequence of SEQ ID NO: 9. In certain embodiments, the trastuzumab light chain is directed by the leader peptide comprising amino acid sequence of SEQ ID NO: 12. In certain embodiments, the trastuzumab light chain is directed by the leader peptide comprising amino acid sequence of SEQ ID NO: 12, encoded by nucleic acid sequence of SEQ ID NO: 11. Other leader sequences can be used, or other leaders exogenous to the heavy and light chain.

In certain embodiments, provided herein is at least one ORF comprising a nucleic acid sequence comprising SEQ ID NO: 7 encoding a leader peptide operably linked to trastuzumab heavy chain, a nucleic acid sequence comprising SEQ ID NO: 3 encoding a trastuzumab heavy chain, a linker comprising a furin cleavage site (SEQ ID NO: 31), “GSG” linker and T2A (SEQ ID NO: 32), wherein furin cleavage site is at 5′ end of the T2A, a nucleic acid sequence comprising SEQ ID NO: 9 encoding a leader peptide operably linked to a trastuzumab light chain, and a nucleic acid sequence comprising SEQ ID NO: 5 encoding a trastuzumab light chain.

In certain embodiments, provided herein is at least one ORF comprising a nucleic acid sequence comprising SEQ ID NO: 7 encoding a leader peptide operably linked to trastuzumab heavy chain, a nucleic acid sequence comprising SEQ ID NO: 3 encoding a trastuzumab heavy chain, a linker comprising a furin cleavage site (SEQ ID NO: 31), “GSG” linker and T2A (SEQ ID NO: 32), wherein furin cleavage site is at 5′ end of the T2A, a nucleic acid sequence comprising SEQ ID NO: 11 encoding a leader peptide operably linked to a trastuzumab light chain, and a nucleic acid sequence comprising SEQ ID NO: 5 encoding a trastuzumab light chain.

As used herein, the term “biological sample” refers to any cell, biological fluid or tissue. Suitable samples for use in this invention may include, without limitation, whole blood, leukocytes, fibroblasts, serum, urine, plasma, saliva, bone marrow, cerebrospinal fluid, amniotic fluid, and skin cells. Such samples may further be diluted with saline, buffer or a physiologically acceptable diluent. Alternatively, such samples are concentrated by conventional means.

“Patient” or “subject” as used herein means a male or female human, dogs, and animal models used for clinical research. In one embodiment, the subject of these methods and compositions is a human diagnosed with metastatic HER2-positive (HER2+) cancer in the brain, optionally wherein the metastatic HER2+ cancer in brain is breast cancer, optionally wherein the metastatic HER2+ cancer in brain is gastrointestinal cancer. In certain embodiments, the human subject of these methods and compositions is a prenatal, a newborn, an infant, a toddler, a preschool, a grade-schooler, a teen, a young adult or an adult.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### Open Reading Frame and Nucleic Acid Molecules

In one aspect, an engineered nucleic acid sequence which encodes an anti-Her2 antibody (i.e., trastuzumab immunoglobulin) is provided (also referred to as trastuzumab-coGW). Trastuzumab immunoglobulin comprises a heavy chain and a light chain. In one embodiment, the engineered nucleic acid sequence is useful to improve production, transcription, expression or safety in a subject. In another embodiment, the engineered sequence is useful to increase efficacy of the resulting therapeutic compositions or treatment. In a further embodiment, the engineered sequence is useful to increase the efficacy of the trastuzumab immunoglobulin being expressed, but may also permit a lower dose of a therapeutic reagent that delivers the immunoglobulin to increase safety. In certain embodiments, provided herein is a recombinant nucleic acid molecule comprising nucleic acid sequence encoding trastuzumab immunoglobulin. In certain embodiments, the recombinant nucleic acid molecule comprises nucleic acid sequence of SEQ ID NO: 17, or a sequence at least 95%, at least 96%, at least 97%, at least 98%, at least 99%, at least 99.9%, and including values therebetween, identical to SEQ ID NO: 17. In certain embodiments, the recombinant nucleic acid molecule comprises nucleic acid sequence of SEQ ID NO: 17. In certain embodiments, the nucleic acid molecule comprises nucleic acid sequence of SEQ ID NO: 29, or a sequence at least 95%, at least 96%, at least 97%, at least 98%, at least 99%, at least 99.9%, and including values therebetween, identical to SEQ ID NO: 29. In certain embodiments, the recombinant nucleic acid molecule comprises nucleic acid sequence of SEQ ID NO: 29.

In one embodiment, the engineered coding sequences for the heavy chain and light chain of trastuzumab are provided in SEQ ID NO: 17. In certain embodiments, the engineered coding sequences for the heavy chain and light chain of trastuzumab comprise nucleic acid sequence at least 95%, at least 96%, at least 97%, at least 98%, at least 99%, at least 99.9%, and including values therebetween, identical to SEQ ID NO: 17. In one embodiment, the engineered coding sequences for the heavy chain and light chain of trastuzumab are provided in SEQ ID NO: 29. In certain embodiments, the engineered coding sequences for the heavy chain and light chain of trastuzumab comprise nucleic acid sequence at least 95%, at least 96%, at least 97%, at least 98%, at least 99%, at least 99.9%, and including values therebetween, identical to SEQ ID NO: 29. In certain embodiments, provided herein is at least one open reading frame (ORF) comprising a nucleic acid sequence encoding a leader peptide operably linked to trastuzumab heavy chain, a nucleic acid sequence comprising SEQ ID NO: 3 encoding a trastuzumab heavy chain, a linker, a nucleic acid sequence encoding a leader peptide operably linked to a trastuzumab light chain, and a nucleic acid sequence comprising SEQ ID NO: 5 encoding a trastuzumab light chain. In certain embodiments, the nucleic acid sequence encoding a trastuzumab heavy chain comprises SEQ ID NO: 3, or a sequence at least 95%, at least 96%, at least 97%, at least 98%, at least 99%, at least 99.9%, and including values therebetween, identical to SEQ ID NO: 3. In certain embodiments, the nucleic acid sequence encoding a trastuzumab light chain comprises SEQ ID NO: 3, or a sequence at least 95%, at least 96%, at least 97%, at least 98%, at least 99%, at least 99.9%, and including values therebetween, identical to SEQ ID NO: 5. In certain embodiments, provided herein is at least one ORF comprising a nucleic acid sequence encoding a leader peptide operably linked to trastuzumab heavy chain, a nucleic acid sequence comprising SEQ ID NO: 3 encoding a trastuzumab heavy chain comprising amino acid sequence of SEQ ID NO: 4, a linker, a nucleic acid sequence encoding a leader peptide operably linked to a trastuzumab light chain, and a nucleic acid sequence comprising SEQ ID NO: 5 encoding a trastuzumab light chain comprising amino acid sequence of SEQ ID NO: 6. In certain embodiments, provided herein is at least one ORF comprising a nucleic acid sequence encoding a leader peptide operably linked to trastuzumab heavy chain, a nucleic acid sequence comprising SEQ ID NO: 3 or a sequence at least 95% identical to SEQ ID NO: 3 encoding a trastuzumab heavy chain comprising amino acid sequence of SEQ ID NO: 4, a linker, a nucleic acid sequence encoding a leader peptide operably linked to a trastuzumab light chain, and a nucleic acid sequence comprising SEQ ID NO: 5 or a sequence at least 95% identical to SEQ ID NO: 5 encoding a trastuzumab light chain comprising amino acid sequence of SEQ ID NO: 6.

In certain embodiments, the linker between the heavy chain and light chain is a Thosea asigna virus (T2A) linker. In certain embodiments, the T2A linker comprises nucleic acid sequence of SEQ ID NO: 32, or a sequence at least 95% identical to SEQ ID NO: 32. In certain embodiments, the linker further comprises a furin cleavage at 5′ end of the T2A, optionally connected via a flexible linker (e.g., “GSG linker”). In certain embodiments, the furin cleavage sequence comprises nucleic acid sequence of SEQ ID NO: 31, or a sequence at least 95% identical to SEQ ID NO: 31. In certain embodiments, the furin/T2A linker comprises nucleic acid sequence of SEQ ID NO: 33, or a sequence at least 95% identical to SEQ ID NO: 33.

In certain embodiments, the leader peptide is an interleukin (IL) IL-2 leader peptide. In certain embodiments, the nucleic acid sequence encoding a leader peptide operably linked to trastuzumab heavy chain comprises nucleic acid sequence of SEQ ID NO: 7. In certain embodiments, the nucleic acid sequence encoding a leader peptide operably linked to trastuzumab heavy chain comprises nucleic acid sequence of SEQ ID NO: 7 which encodes amino acid sequence of SEQ ID NO: 8 which is an IL2 leader peptide. In certain embodiments, the nucleic acid sequence encoding a leader peptide operably linked to trastuzumab heavy chain comprises nucleic acid sequence of SEQ ID NO: 9. In certain embodiments, the nucleic acid sequence encoding a leader peptide operably linked to trastuzumab heavy chain comprises nucleic acid sequence of SEQ ID NO: 9 which encodes amino acid sequence of SEQ ID NO:10 which is a modified IL2 leader peptide. In certain embodiments, the nucleic acid sequence encoding a leader peptide operably linked to trastuzumab heavy chain comprises nucleic acid sequence of SEQ ID NO: 11. In certain embodiments, the nucleic acid sequence encoding a leader peptide operably linked to trastuzumab heavy chain comprises nucleic acid sequence of SEQ ID NO: 11 which encodes amino acid sequence of SEQ ID NO: 12 which is a modified IL2 leader peptide. In certain embodiments, the nucleic acid sequence encoding a leader peptide operably linked to trastuzumab light chain comprises nucleic acid sequence of SEQ ID NO: 7. In certain embodiments, the nucleic acid sequence encoding a leader peptide operably linked to trastuzumab light chain comprises nucleic acid sequence of SEQ ID NO: 7 which encodes amino acid sequence of SEQ ID NO: 8 which is an IL2 leader peptide. In certain embodiments, the nucleic acid sequence encoding a leader peptide operably linked to trastuzumab light chain comprises nucleic acid sequence of SEQ ID NO: 9. In certain embodiments, the nucleic acid sequence encoding a leader peptide operably linked to trastuzumab light chain comprises nucleic acid sequence of SEQ ID NO: 9 which encodes amino acid sequence of SEQ ID NO:10 which is a modified IL2 leader peptide. In certain embodiments, the nucleic acid sequence encoding a leader peptide operably linked to trastuzumab light chain comprises nucleic acid sequence of SEQ ID NO: 11. In certain embodiments, the nucleic acid sequence encoding a leader peptide operably linked to trastuzumab light chain comprises nucleic acid sequence of SEQ ID NO: 11 which encodes amino acid sequence of SEQ ID NO: 12 which is a modified IL2 leader peptide.

In certain embodiments, the engineered nucleic acid sequence encoding trastuzumab immunoglobulin comprises, 5′ to 3′, an IL2 leader peptide, trastuzumab heavy chain, optionally a furin cleavage sequence, a T2A linker, an IL2 leader peptide, optionally which is a modified IL2 leader peptide, and a trastuzumab light chain. In certain embodiment, the engineered nucleic acid sequence encoding trastuzumab immunoglobulin comprises, 5′ to 3′, an IL2 leader peptide, trastuzumab heavy chain, a furin cleavage sequence, a T2A linker, an IL2 leader peptide which is a modified IL2 leader peptide, and a trastuzumab light chain.

In certain embodiment, the engineered nucleic acid sequence encoding trastuzumab heavy chain comprising an IL2 leader peptide comprises SEQ ID NO: 13. In certain embodiment, the engineered nucleic acid sequence encoding trastuzumab heavy chain comprising an IL2 leader peptide comprises a sequence at least 95% identical to SEQ ID NO: 13. In certain embodiment, the engineered nucleic acid sequence encoding trastuzumab heavy chain comprising an IL2 leader peptide comprises SEQ ID NO: 13 which encodes an amino acid sequence of SEQ ID NO: 14. In certain embodiment, the engineered nucleic acid sequence encoding trastuzumab light chain comprising a modified IL2 leader peptide comprises SEQ ID NO: 15. In certain embodiment, the engineered nucleic acid sequence encoding trastuzumab light chain comprising a modified IL2 leader peptide comprises a sequence at least 95% identical to SEQ ID NO: 15. In certain embodiment, the engineered nucleic acid sequence encoding trastuzumab light chain comprising a modified IL2 leader peptide comprises SEQ ID NO: 15 which encodes an amino acid sequence of SEQ ID NO: 16.

In certain embodiments, the engineered nucleic acid sequence encoding trastuzumab immunoglobulin comprising nucleic acid sequence of SEQ ID NO: 17, wherein the sequence comprises IL2 leader peptide sequence, a trastuzumab heavy chain encoding sequence, a T2A linker sequence, a modified IL2 leader peptide sequence, and a trastuzumab light chain encoding sequence. In certain embodiments, the engineered nucleic acid sequence encoding trastuzumab immunoglobulin comprising nucleic acid sequence at least 95% identical to SEQ ID NO: 17, wherein the sequence comprises IL2 leader peptide sequence, a trastuzumab heavy chain encoding sequence, a T2A linker sequence, a modified IL2 leader peptide sequence, and a trastuzumab light chain encoding sequence. In certain embodiments, the engineered nucleic acid sequence encoding trastuzumab immunoglobulin comprising nucleic acid sequence of SEQ ID NO: 29, wherein the sequence comprises IL2 leader peptide sequence, a trastuzumab heavy chain encoding sequence, a furin cleavage sequence, a T2A linker sequence, a modified IL2 leader peptide sequence, and a trastuzumab light chain encoding sequence. In certain embodiments, the engineered nucleic acid sequence encoding trastuzumab immunoglobulin comprising nucleic acid sequence at least 95% identical to SEQ ID NO: 29, wherein the sequence comprises IL2 leader peptide sequence, a trastuzumab heavy chain encoding sequence, a furin cleavage sequence, a T2A linker sequence, a modified IL2 leader peptide sequence, and a trastuzumab light chain encoding sequence.

In certain embodiments, provided herein is at least one open reading frame (ORF) comprising a nucleic acid sequence of SEQ ID NO: 7 encoding a leader peptide operably linked to trastuzumab heavy chain, a nucleic acid sequence comprising SEQ ID NO: 3 encoding a trastuzumab heavy chain, a nucleic acid sequence encoding a linker comprising nucleic acid sequence of SEQ ID NO: 31 encoding furin cleaveage site, nucleic sequence encoding “GSG” linker and nucleic sequence of SEQ ID NO: 32 encoding T2A, a nucleic acid sequence of SEQ ID NO: 7 encoding a leader peptide operably linked to a trastuzumab light chain, and a nucleic acid sequence comprising SEQ ID NO: 5 encoding a trastuzumab light chain.

In certain embodiments, provided herein is at least one open reading frame (ORF) comprising a nucleic acid sequence of SEQ ID NO: 7 encoding amino acid sequence of SEQ ID NO: 8 which is a leader peptide operably linked to trastuzumab heavy chain, a nucleic acid sequence comprising SEQ ID NO: 3 encoding amino acid sequence of SEQ ID NO: 4 which is a trastuzumab heavy chain, a nucleic acid sequence encoding a linker comprising nucleic acid sequence of SEQ ID NO: 31 encoding amino acid sequence of SEQ ID NO: 49 which is a furin cleaveage site, nucleic sequence encoding “GSG” linker and nucleic sequence of SEQ ID NO: 32 encoding amino acid sequence of SEQ ID NO: 51 which is a T2A, a nucleic acid sequence of SEQ ID NO: 7 encoding amino acid sequence of SEQ ID NO: 8 which is a leader peptide operably linked to a trastuzumab light chain, and a nucleic acid sequence comprising SEQ ID NO: 5 encoding amino acid sequence of SEQ ID NO: 6 which is a trastuzumab light chain.

In certain embodiments, provided herein is at least one open reading frame (ORF) comprising a nucleic acid sequence of SEQ ID NO: 7 encoding a leader peptide operably linked to trastuzumab heavy chain, a nucleic acid sequence comprising SEQ ID NO: 3 encoding a trastuzumab heavy chain, a nucleic acid sequence encoding a linker comprising nucleic acid sequence of SEQ ID NO: 31 encoding furin cleaveage site, nucleic sequence encoding “GSG” linker and nucleic sequence of SEQ ID NO: 32 encoding T2A, a nucleic acid sequence of SEQ ID NO: 9 encoding a leader peptide operably linked to a trastuzumab light chain, and a nucleic acid sequence comprising SEQ ID NO: 5 encoding a trastuzumab light chain.

In certain embodiments, provided herein is at least one open reading frame (ORF) comprising a nucleic acid sequence of SEQ ID NO: 7 encoding amino acid sequence of SEQ ID NO: 8 which is a leader peptide operably linked to trastuzumab heavy chain, a nucleic acid sequence comprising SEQ ID NO: 3 encoding amino acid sequence of SEQ ID NO: 4 which is a trastuzumab heavy chain, a nucleic acid sequence encoding a linker comprising nucleic acid sequence of SEQ ID NO: 31 encoding amino acid sequence of SEQ ID NO: 49 which is a furin cleaveage site, nucleic sequence encoding “GSG” linker and nucleic sequence of SEQ ID NO: 32 encoding amino acid sequence of SEQ ID NO: 51 which is a T2A, a nucleic acid sequence of SEQ ID NO: 9 encoding amino acid sequence of SEQ ID NO: 10 which is a leader peptide operably linked to a trastuzumab light chain, and a nucleic acid sequence comprising SEQ ID NO: 5 encoding amino acid sequence of SEQ ID NO: 6 which is a trastuzumab light chain.

In certain embodiments, provided herein is at least one open reading frame (ORF) comprising a nucleic acid sequence of SEQ ID NO: 7 encoding a leader peptide operably linked to trastuzumab heavy chain, a nucleic acid sequence comprising SEQ ID NO: 3 encoding a trastuzumab heavy chain, a nucleic acid sequence encoding a linker comprising nucleic acid sequence of SEQ ID NO: 31 encoding a furin cleaveage site, nucleic sequence encoding “GSG” linker and nucleic sequence of SEQ ID NO: 32 encoding T2A, a nucleic acid sequence of SEQ ID NO: 11 encoding a leader peptide operably linked to a trastuzumab light chain, and a nucleic acid sequence comprising SEQ ID NO: 5 encoding a trastuzumab light chain.

In certain embodiments, provided herein is at least one open reading frame (ORF) comprising a nucleic acid sequence of SEQ ID NO: 7 encoding amino acid sequence of SEQ ID NO: 8 which is a leader peptide operably linked to trastuzumab heavy chain, a nucleic acid sequence comprising SEQ ID NO: 3 encoding amino acid sequence of SEQ ID NO: 4 which is a trastuzumab heavy chain, a nucleic acid sequence encoding a linker comprising nucleic acid sequence of SEQ ID NO: 31 encoding amino acid sequence of SEQ ID NO: 49 which is a furin cleaveage site, nucleic sequence encoding “GSG” linker and nucleic sequence of SEQ ID NO: 32 encoding amino acid sequence of SEQ ID NO: 51 which is a T2A, a nucleic acid sequence of SEQ ID NO: 11 encoding amino acid sequence of SEQ ID NO: 12 which is a leader peptide operably linked to a trastuzumab light chain, and a nucleic acid sequence comprising SEQ ID NO: 5 encoding amino acid sequence of SEQ ID NO: 6 which is a trastuzumab light chain.

A “nucleic acid”, as described herein, can be RNA, DNA, or a modification thereof, and can be single or double stranded, and can be selected, for example, from a group including nucleic acid encoding a protein of interest, oligonucleotides, nucleic acid analogues, for example peptide-nucleic acid (PNA), pseudocomplementary PNA (pc-PNA), locked nucleic acid (LNA) etc. A nucleotide refers to a ribonucleotide, deoxynucleotide or a modified form of either type of nucleotide (e.g., a peptide nucleic acid oligomer). The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules. Such nucleic acid sequences include, for example, but are not limited to, In certain embodiments, the miRNA is a dorsal root ganglion (drg)-specific miRNA target sequence. In certain embodiments, the nucleic acid sequence further comprises at least one, at least two, at least three or preferably at least four tandem repeats of dorsal root ganglion (drg)-specific miRNA target sequences. In certain embodiments, the nucleic acid sequence further comprises at least one, at least two, at least three, at least four, at least five, at least six, at least seven or preferably at least eight tandem repeats of dorsal root ganglion (drg)-specific miRNA target sequences. See, e.g., PCT/US19/67872, filed Dec. 20, 2019, and now published as WO 2020/132455, which are incorporated herein by reference. See, also, International Patent Application No. PCT/US21/32003, filed May 12, 2021, and now published WO2021/231579A1, which are incorporated herein by reference. See also, U.S. Provisional Patent Application No. 63/279,561, filed Nov. 15, 2021, which is incorporated herein by reference in its entirety.

In certain embodiments, the recombinant nucleic acid molecules encoding a trastuzumab immunoglobulin, and other constructs encompassed by the present invention and useful in generating expression cassettes and vector genomes may be engineered for expression in yeast cells, insect cells or mammalian cells, such as human cells. Methods are known and have been described previously (e.g., WO 96/09378). A sequence is considered engineered if at least one non-preferred codon as compared to a wild type (WT) sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofns).

By “engineered” is meant that the nucleic acid sequences encoding a trastuzumab immunoglobulin described herein are assembled and placed into any suitable genetic element, e.g., naked DNA, phage, transposon, cosmid, episome, etc., which transfers the trastuzumab immunoglobulin carried thereon to a host cell, e.g., for generating non-viral delivery systems (e.g., RNA-based systems, naked DNA, or the like), or for generating viral vectors in a packaging host cell, and/or for delivery to a host cells in a subject. In one embodiment, the genetic element is a vector. In one embodiment, the genetic element is a plasmid. The methods used to make such engineered constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

The term “percent (%) identity”, “sequence identity”, “percent sequence identity”, or “percent identical” in the context of nucleic acid sequences refers to the residues in the two sequences which are the same when aligned for correspondence, subsequences that are the same or have a specified percentage of amino acid residues or nucleotides that are the same (i.e., about 70% identity, preferably 75%, 80%, 85%, 90%, 91%, 92%, 93%, 94%, 95%, 96%, 97%, 98%, 99%, or higher identity over a specified region (e.g., any one of the modified ORFs provided herein when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments are performed using any of a variety of publicly or commercially available Multiple Sequence Alignment Programs. Sequence alignment programs are available for amino acid sequences, e.g., the “Clustal X”, “Clustal Omega” “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “Clustal Omega”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

It should be understood that the compositions in the trastuzumab immunoglobulin and trastuzumab immunoglobulin coding sequence described herein are intended to be applied to other compositions, regiments, aspects, embodiments and methods described across the Specification.

### Expression Cassette

A gene therapy vector is provided herein which comprises an expression cassette comprising an engineered nucleic acid sequence comprising coding sequences for anti-Her2 antibody, i.e., trastuzumab immunoglobulin comprising heavy chain and light chain, operably linked to regulatory sequences which direct expression thereof. Provided herein also is a recombinant nucleic acid molecule comprising expression cassette comprising nucleic acid sequence encoding for a trastuzumab immunoglobulin, as described herein. Provided herein also is a recombinant nucleic acid molecule comprising vector genome comprising nucleic acid sequence encoding for a trastuzumab immunoglobulin, as described herein.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in cis or trans with nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (′3 UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell. Typically, such an expression cassette for generating a viral vector contains the coding sequence for the gene product described herein flanked by packaging signals of the viral genome and other expression control sequences such as those described herein. In certain embodiments, a vector genome may contain two or more expression cassettes.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g., a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector, indicates that the protein or the nucleic acid is present with another sequence or subsequence which with which the protein or nucleic acid in question is not found in the same relationship to each other in nature.

In certain embodiments, the regulatory sequences comprise a promoter. In certain embodiment, the regulatory sequences comprise one or more intron(s), one or more enhancer(s), and a polyadenylation (polyA) signal sequence.

In one embodiment, the regulatory sequence comprises a promoter. In one embodiment, the promoter is a chicken β-actin (also referred to as chicken beta-actin, CB or CBA) promoter. A variety of chicken beta-actin promoters have been described alone, or in combination with various enhancer elements (e.g., CB7 is a chicken beta-actin promoter with cytomegalovirus enhancer elements, a CAG promoter, which includes the promoter, the first exon and first intron of chicken beta actin, and the splice acceptor of the rabbit beta-globin gene), a CBh promoter [SJ Gray et al, Hu Gene Ther, 2011 Sep; 22(9): 143-1 153]. In certain embodiments, the promoter is cytomegalovirus (CMV) promoter. In certain, embodiments, the CB promoter comprises nucleic acid sequence of SEQ ID NO: 20.

In a further embodiment, the promoter is a CB7 (also referred to as hybrid CB7) promoter comprising a cytomegalovirus immediate-early (CMV IE) enhancer and the chicken R-actin promoter, optionally with spacer sequence, optionally with a chimeric intron comprising chicken beta actin intron and further comprising a chicken beta-actin splicing donor (including the exon sequence, chicken beta actin intron) and rabbit beta-globin splicing acceptor. In certain embodiments, the CMV IE enhancer comprises nucleic acid sequence of SEQ TD NO: 19. In certain embodiments, the CB7 promoter comprise nucleic acid sequence of SEQ ID NO: 21. See, e.g., cytomegalovirus (CMV) immediate early enhancer (260 bp, C4; GenBank #K03104.1). Chicken beta-actin promoter (281 bp; CB; GenBank #X00182.1). In other embodiments, the promoter is a ubiquitin C (UbC) promoter. See, e.g., WO 2001/091800. See, e.g., GenBank accession numbers AF232305 (rat) and D63791 (human), respectively. In certain embodiments, the UbC promoter comprises nucleic acid sequence of SEQ ID NO: 24. Still other promoters and/or enhancers may be selected. In another embodiment, a suitable promoter may include without limitation, an elongation factor 1 alpha (EF1 alpha) promoter (see, e.g., Kim D W et al, Use of the human elongation factor 1 alpha promoter as a versatile and efficient expression system. Gene. 1990 Jul. 16; 91(2):217-23), a Synapsin 1 promoter (see, e.g., Kügler S et al, Human synapsin 1 gene promoter confers highly neuron-specific long-term transgene expression from an adenoviral vector in the adult rat brain depending on the transduced area. Gene Ther. 2003 Feb; 10(4):337-47), a neuron-specific enolase (NSE) promoter (see, e.g., Kim J et al, Involvement of cholesterol-rich lipid rafts in interleukin-6-induced neuroendocrine differentiation of LNCaP prostate cancer cells. Endocrinology. 2004 Feb; 145(2):613-9. Epub 2003 Oct 16), or a CB6 promoter (see, e.g., Large-Scale Production of Adeno-Associated Viral Vector Serotype-9 Carrying the Human Survival Motor Neuron Gene, Mol Biotechnol. 2016 Jan; 58(1):30-6. doi: 10.1007/s12033-015-9899-5). In still other embodiments, multiple enhancers and/or promoters may be included.

In certain embodiments, an additional or alternative promoter sequence may be included as part of the expression control sequences (regulatory sequences), e.g., located between the selected 5′ ITR sequence and the coding sequence. Constitutive promoters, regulatable promoters [see, e.g., WO 2011/126808 and WO 2013/04943], tissue specific promoters, or a promoter responsive to physiologic cues may be utilized in the vectors described herein. The promoter(s) can be selected from different sources, e.g., human cytomegalovirus immediate-early (CMV IE) enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

In one embodiment, the expression cassette is designed for expression and secretion in a human subject. In one embodiment, the expression cassette is designed for expression in the central nervous system (CNS), including the cerebral spinal fluid and brain. In a further embodiment, the expression cassette is useful for expression in both the CNS and in the systemically. Suitable promoters may be selected, including but not limited to a constitutive promoter, a tissue-specific promoter or an inducible/regulatory promoter. Example of a constitutive promoter is chicken beta-actin promoter. See also, CB7, above. Examples of promoters that are tissue-specific are well known for liver (albumin, Miyatake et al., (1997) J. Virol., 71:5124 32; hepatitis B virus core promoter, Sandig et al., (1996) Gene Ther., 3:1002 9; alpha fetoprotein (AFP), Arbuthnot et al., (1996) Hum. Gene Ther., 7:1503 14), neuron (such as neuron specific enolase (NSE) promoter, Andersen et al., (1993) Cell. Mol. Neurobiol., 13:503 15; neurofilament light chain gene, Piccioli et al., (1991) Proc. Natl. Acad. Sci. USA, 88:5611 5; and the neuron-specific vgf gene, Piccioli et al., (1995) Neuron, 15:373 84), and other tissues. Alternatively, a regulatable promoter may be selected. See, e.g., WO 2011/126808B2, incorporated by reference herein.

In addition to a promoter, a vector may contain one or more other appropriate transcription initiation sequences, transcription termination sequences, enhancer sequences, efficient RNA processing signals such as splicing and polyadenylation (polyA) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an Alpha mic/bik enhancer or a CMV IE enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. In one embodiment, the intron is 875 bp (GenBank #X00182.1). In one embodiment, the intron is 973 bp intron from the chicken beta actin gene (GenBank #X00182.1). In certain embodiments, the chicken beta actin intron comprises nucleic acid sequence of SEQ ID NO: 34. In certain embodiments, the intron is a chimeric intron (CI)—a hybrid intron consisting of a human beta-globin splice donor and immunoglobulin G (IgG) splice acceptor elements. In certain embodiments, the intron is a chimeric intron comprising a chicken beta-actin splicing donor (including the exon sequence), chicken beta actin intron, and rabbit beta globin splicing acceptor. In certain embodiments, the intron is a chimeric intron comprising a chicken beta actin intron comprises nucleic acid sequence of SEQ ID NO: 51. In another embodiment, a chimeric intron available from Promega® is used. In certain embodiments, the chimeric intron is a Promega® (chimeric) intron comprising nucleic acid sequence of SEQ ID NO: 22. Other suitable introns include those known in the art may by a human β-globulin intron, and/or a commercially available intron, and those described in WO 2011/126808.

In one embodiment, the regulatory sequence further comprises a polyadenylation signal (polyA). Examples of suitable polyA sequences include, e.g., rabbit beta globin (RBG or rBG) poly A, SV40, SV50, bovine growth hormone (bGH), human growth hormone, and synthetic polyAs. Optionally, one or more sequences may be selected to stabilize mRNA. In certain embodiments, the polyA is a rabbit beta globin poly A (rabbit globin polyA or rBG). See, e.g., WO 2014/151341. In certain embodiments, a human growth hormone (hGH) polyadenylation sequence, an SV40 polyA, or a synthetic polyA may be included in an expression cassette. In certain embodiments, the SV40 polyA is selected. In certain embodiments, the SV40 polyA comprises nucleic acid sequence of SEQ ID NO: 23.

In certain embodiments, the expression cassette comprises a trastuzumab coding sequence and may include other regulatory sequences therefor. The regulatory sequences necessary are operably linked to the trastuzumab coding sequence in a manner which permits its transcription, translation and/or expression in target cell.

In certain embodiment, the target cell may be a central nervous system cell. In certain embodiments, the target cell is one or more of an excitatory neuron, an inhibitory neuron, a glial cell, a cortex cell, a frontal cortex cell, a cerebral cortex cell, a spinal cord cell. In certain embodiments, the target cell is in leptomeninges (LM) of the CNS. In certain embodiments, the target cell is in parenchyma of CNS.

In certain embodiments, the expression cassette comprises (i) a promoter and/or a promoter element which comprises an enhancer and a promoter, optionally with spacer sequences therebetween, (ii) an intron, (iii) at least one ORF which comprises: a nucleic acid sequence encoding a leader peptide operably linked to a trastuzumab heavy chain, (iv) a nucleic acid sequence comprising SEQ ID NO: 3 encoding a trastuzumab heavy chain, (v) optionally a furin cleavage site, (vi) a T2A element linker, (vii) a nucleic acid sequence encoding a leader peptide operably linked to a trastuzumab light chain, (viii) nucleic acid sequence comprising SEQ ID NO: encoding a trastuzumab light chain, and (ix) an SV40 polyadenylation (polyA) sequence.

In certain embodiments, the expression cassette comprises (i) a promoter and/or a promoter element which comprises an enhancer and a promoter, optionally with spacer sequences therebetween, (ii) an intron, (iii) Kozak sequence, (iv) at least one ORF which comprises: a nucleic acid sequence encoding a leader peptide operably linked to a trastuzumab heavy chain, (v) a nucleic acid sequence comprising SEQ ID NO: 3 encoding a trastuzumab heavy chain, (vi) a furin cleavage site, (vii) a T2A element linker, (viii) a nucleic acid sequence encoding a leader peptide operably linked to a trastuzumab light chain, (ix) nucleic acid sequence comprising SEQ ID NO: 5 encoding a trastuzumab light chain, and (x) an SV40 polyadenylation (polyA) sequence.

In certain embodiments, the expression cassette comprises (i) a promoter element comprising a chicken beta actin promoter (CB) and an enhancer, (ii) an intron which is a chicken beta actin intron (CT), (iii) at least one ORF which comprises: a nucleic acid sequence encoding a leader peptide operably linked to a trastuzumab heavy chain, (iv) a nucleic acid sequence comprising SEQ ID NO: 3 encoding a trastuzumab heavy chain, (v) a T2A element linker, (vi) a nucleic acid sequence encoding a leader peptide operably linked to a trastuzumab light chain, (vii) nucleic acid sequence comprising SEQ ID NO: 5 encoding a trastuzumab light chain, and (viii) an SV40 polyadenylation (polyA) sequence.

In certain embodiments, the expression cassette comprises (i) a promoter element comprising a chicken beta-actin promoter (CB) and an enhancer, (ii) an intron which is a chicken beta actin intron (CI), (iii) at least one ORF which comprises: a nucleic acid sequence encoding a leader peptide operably linked to a trastuzumab heavy chain, (iv) a nucleic acid sequence comprising SEQ ID NO: 3 or a sequence at least 95% identical to SEQ ID NO: 3 encoding a trastuzumab heavy chain, (v) a furin cleavage site, (vi) a T2A element linker, (vii) a nucleic acid sequence encoding a leader peptide operably linked to a trastuzumab light chain, (viii) nucleic acid sequence comprising SEQ ID NO: 5 or a sequence at least 95% identical to SEQ ID NO: 5 encoding a trastuzumab light chain, and (ix) an SV40 polyadenylation (polyA) sequence.

In certain embodiments, the expression cassette comprises (i) a promoter which is a CB7 hybrid promoter comprising a CMV IE enhancer, a chicken beta-actin promoter, and a chimeric intron comprising a chicken beta actin intron (CI), (ii) Kozak sequence, (iii) at least one ORF which comprises: a nucleic acid sequence encoding a leader peptide operably linked to a trastuzumab heavy chain, (iv) a nucleic acid sequence comprising SEQ ID NO: 3 or a sequence at least 95% identical to SEQ ID NO: 3 encoding a trastuzumab heavy chain, (v) optionally a furin cleavage site, (vi) a T2A element linker, (vii) a nucleic acid sequence encoding a leader peptide operably linked to a trastuzumab light chain, (viii) nucleic acid sequence comprising SEQ ID NO: or a sequence at least 95% identical to SEQ ID NO: 5 encoding a trastuzumab light chain, and (ix) an SV40 polyadenylation (polyA) sequence.

In certain embodiments, the expression cassette comprises (i) a promoter which is a CB7 hybrid promoter comprising a CMV IE enhancer, a chicken beta-actin promoter, and a chimeric intron comprising a chicken beta-actin splicing donor (including the exon sequence) chicken beta actin intron and rabbit b-globin splicing acceptor, (ii) Kozak sequence, (iii) at least one ORF which comprises: a nucleic acid sequence encoding a leader peptide operably linked to a trastuzumab heavy chain, (iv) a nucleic acid sequence comprising SEQ ID NO: 3 or a sequence at least 95% identical to SEQ ID NO: 3 encoding a trastuzumab heavy chain, (v) a furin cleavage site, (vi) a T2A element linker, (vii) a nucleic acid sequence encoding a leader peptide operably linked to a trastuzumab light chain, (viii) nucleic acid sequence comprising SEQ ID NO: 5 or a sequence at least 95% identical to SEQ ID NO: 5 encoding a trastuzumab light chain, and (ix) an SV40 polyadenylation (polyA) sequence.

In certain embodiments, the expression cassette comprises (i) a promoter which is a CB7 hybrid promoter comprising nucleic acid sequence of SEQ ID NO: 21, (ii) Kozak sequence, (iii) at least one ORF which comprises: a nucleic acid sequence encoding a leader peptide comprising nucleic acid sequence of SEQ ID NO: 7 operably linked to a trastuzumab heavy chain, (iv) a nucleic acid sequence comprising SEQ ID NO: 3 or a sequence at least 95% identical to SEQ ID NO: 3 encoding a trastuzumab heavy chain, (v) a furin cleaveage site comprising nucleic acid sequence of SEQ ID NO: 31, (vi) a T2A element linker comprising nucleic acid sequence of SEQ ID NO: 32, (vii) a nucleic acid sequence encoding a leader peptide comprising nucleic acid sequence of SEQ ID NO: 9 operably linked to a trastuzumab light chain, (viii) nucleic acid sequence comprising SEQ ID NO: 5 or a sequence at least 95% identical to SEQ ID NO: 5 encoding a trastuzumab light chain, and (ix) an SV40 polyadenylation (polyA) sequence comprising nucleic acid sequence of SEQ ID NO: 23.

In certain embodiments, the expression cassette comprises (i) a promoter which is a CB7 hybrid promoter comprising nucleic acid sequence of SEQ ID NO: 21, (ii) Kozak sequence, (iii) at least one ORF which comprises: a nucleic acid sequence encoding a leader peptide comprising nucleic acid sequence of SEQ ID NO: 7 operably linked to a trastuzumab heavy chain, (iv) a nucleic acid sequence comprising SEQ ID NO: 3 or a sequence at least 95% identical to SEQ ID NO: 3 encoding a trastuzumab heavy chain, (v) a furin cleavage site followed by T2A element linker connected via a “GSG” linker (furin/T2A) comprising nucleic acid sequence of SEQ ID NO: 33, (vi) a nucleic acid sequence encoding a leader peptide comprising nucleic acid sequence of SEQ ID NO: 9 operably linked to a trastuzumab light chain, (vii) nucleic acid sequence comprising SEQ ID NO: 5 or a sequence at least 95% identical to SEQ ID NO: 5 encoding a trastuzumab light chain, and (viii) an SV40 polyadenylation (polyA) sequence comprising nucleic acid sequence of SEQ ID NO: 23.

In certain embodiments, the expression cassette comprises (i) a promoter which is a CB7 hybrid promoter comprising a CMV IE enhancer and a chicken beta-actin promoter, and a chimeric intron composing a chicken beta actin intron (CI), (ii) Kozak sequence, (iii) at least one ORF which comprises: a nucleic acid sequence encoding a leader peptide operably linked to a trastuzumab heavy chain, (iv) a nucleic acid sequence comprising SEQ ID NO: 3 or a sequence at least 95% identical to SEQ ID NO: 3 encoding a trastuzumab heavy chain, (v) a furin cleavage site, (vi) a T2A element linker, (vii) a nucleic acid sequence encoding a leader peptide operably linked to a trastuzumab light chain, (viii) nucleic acid sequence comprising SEQ ID NO: 5 or a sequence at least 95% identical to SEQ ID NO: 5 encoding a trastuzumab light chain, and (ix) an SV40 polyadenylation (polyA) sequence.

In certain embodiments, the expression cassette comprises (i) a promoter which is a Ubiquitin C (UbC) promoter, (ii) an intron which is a chimeric intron comprising a Promega intron (PI), (iii) Kozak sequence, (iv) at least one ORF which comprises: a nucleic acid sequence encoding a leader peptide operably linked to a trastuzumab heavy chain, (v) a nucleic acid sequence comprising SEQ ID NO: 3 or a sequence at least 95% identical to SEQ ID NO: 3 encoding a trastuzumab heavy chain, (vi) optionally a furin cleavage site, (vii) a T2A element linker, (viii) a nucleic acid sequence encoding a leader peptide operably linked to a trastuzumab light chain, (ix) nucleic acid sequence comprising SEQ ID NO: 5 or a sequence at least 95% identical to SEQ ID NO: 5 encoding a trastuzumab light chain, and (x) an SV40 polyadenylation (polyA) sequence

In certain embodiments, the expression cassette comprises (i) a promoter which is a Ubiquitin C (UbC) promoter, (ii) an intron which is a chimeric intron comprising Promega intron (PI), (iii) Kozak sequence, (iv) at least one ORF which comprises: a nucleic acid sequence encoding a leader peptide operably linked to a trastuzumab heavy chain, (v) a nucleic acid sequence comprising SEQ ID NO: 3 or a sequence at least 95% identical to SEQ ID NO: 3 encoding a trastuzumab heavy chain, (vi) a furin cleavage site, (vii) a T2A element linker, (viii) a nucleic acid sequence encoding a leader peptide operably linked to a trastuzumab light chain, (ix) nucleic acid sequence comprising SEQ ID NO: 5 or a sequence at least 95% identical to SEQ ID NO: 5 encoding a trastuzumab light chain, and (x) an SV40 polyadenylation (polyA) sequence

In certain embodiments, the expression cassette comprises (i) a promoter which is a Ubiquitin C (UbC) promoter (SEQ ID NO: 24), (ii) an intron which is a chimeric intron comprising Promega intron (PI) (SEQ ID NO: 22), (iii) Kozak sequence, (iv) at least one ORF which comprises: a nucleic acid sequence encoding a leader peptide operably linked to a trastuzumab heavy chain (SEQ ID NO: 7), (v) a nucleic acid sequence comprising SEQ ID NO: 3 or a sequence at least 95% identical to SEQ ID NO: 3 encoding a trastuzumab heavy chain, (vi) a furin cleavage site (SEQ ID NO: 31), (vii) a T2A element linker (SEQ ID NO: 32), (viii) a nucleic acid sequence encoding a leader peptide operably linked to a trastuzumab light chain (SEQ ID NO: 11), (ix) nucleic acid sequence comprising SEQ ID NO: 5 or a sequence at least 95% identical to SEQ ID NO: 5 encoding a trastuzumab light chain, and (x) an SV40 polyadenylation (polyA) sequence (SEQ ID NO: 23).

In certain embodiments, the expression cassette comprises (i) a promoter which is a Ubiquitin C (UbC) promoter (SEQ ID NO: 24), (ii) an intron which is a chimeric intron comprising Promega intron (PI) (SEQ ID NO: 22), (iii) Kozak sequence, (iv) at least one ORF which comprises: a nucleic acid sequence encoding a leader peptide operably linked to a trastuzumab heavy chain (SEQ ID NO: 7), (v) a nucleic acid sequence comprising SEQ ID NO: 3 or a sequence at least 95% identical to SEQ ID NO: 3 encoding a trastuzumab heavy chain, (vi) a furin cleaveage site connected to T2A element linker via a “GSG” linker (furin/T2A) (SEQ ID NO: 33), (vii) a nucleic acid sequence encoding a leader peptide operably linked to a trastuzumab light chain (SEQ ID NO: 11), (viii) nucleic acid sequence comprising SEQ ID NO: 5 or a sequence at least 95% identical to SEQ ID NO: 5 encoding a trastuzumab light chain, and (ix) an SV40 polyadenylation (polyA) sequence (SEQ ID NO: 23).

In certain embodiments, the expression cassette comprises nucleic acid sequence of SEQ ID NO: 2 (CB.CI.IL2.V1.Trastuzumab-coGW.SV40) or a sequence at least 95%, at least 96%, at least 97%, at least 98%, at least 99 to at least 100% identical thereto. In certain embodiments, the expression cassette comprises nucleic acid sequence of SEQ ID NO: 2 (CB.CLIL2.V1.Trastuzumab-coGW.SV40) or a sequence at least 99% identical thereto. In certain embodiments, the expression cassette comprises nucleic acid sequence of SEQ ID NO: 26 (UbC.PI.IL2_Furin_V1.Trastuzumab-coGW.SV40) or a sequence at least 95%, at least 96%, at least 97%, at least 98%, at least 99 to at least 100% identical thereto. In certain embodiments, the expression cassette comprises nucleic acid sequence of SEQ ID NO: 26 (UbC.PI.IL2_Furin_V1.Trastuzumab-coGW.SV40) or a sequence at least 99% identical thereto. In certain embodiments, the expression cassette comprises nucleic acid sequence of SEQ ID NO: 28 (CB7.CI.IL2 Furin_V1.Trastuzumab-coGW.SV40) or a sequence at least 95%, at least 96%, at least 97%, at least 98%, at least 99 to at least 100% identical thereto. In certain embodiments, the expression cassette comprises nucleic acid sequence of SEQ ID NO: 28 (CB7.CI.IL2_Furin_V1. Trastuzumab-coGW.SV40) or a sequence at least 99% identical thereto.

It should be understood that the compositions in the expression cassette described herein are intended to be applied to other compositions, regiments, aspects, embodiments and methods described across the Specification.

Recombinant Adeno-Associated Virus (rAAV)

Provided herein is a recombinant adeno-associated virus (rAAV) comprising an engineered nucleic acid sequence encoding trastuzumab immunoglobulin (e.g., rAAV.Trastuzumab-coGW). Provided herein is a recombinant adeno-associated virus (rAAV) useful for treating of metastatic HER2-positive cancer in the brain. The rAAV comprises (a) an AAV capsid; and (b) a vector genome packaged in the AAV capsid of (a). Suitably, the AAV capsid selected targets the cells to be treated. In certain embodiments, the capsid is from Clade F. However, in certain embodiments, another AAV capsid source may be selected, i.e., Clade A. In certain embodiments, the AAV capsid is AAVhu68 capsid. In certain embodiments, the AAV capsid is AAVrh91 capsid. In certain embodiments, the AAV capsid is AAVhu95 capsid. In certain embodiments, the AAV capsid is AAVhu96 capsid. The vector genome comprises an AAV 5′ inverted terminal repeat (ITR), an expression cassette comprising at least one open reading frame (ORF) comprising a trastuzumab heavy chain and a trastuzumab light chain and nucleic acid sequences operably linked thereto which regulate expression of the trastuzumab heavy and light chains, and an AAV 3′ ITR.

As used herein, the term “vector genome” refers to a nucleic acid molecule which is packaged in a viral capsid, for example, an AAV capsid, and is capable of being delivered to a host cell or a cell in a patient. In certain embodiments, the vector genome comprises terminal repeat sequences (e.g., AAV inverted terminal repeat sequences (ITRs) necessary for packaging the vector genome into the capsid at the extreme 5′ and 3′ end and containing therebetween an expression cassette comprising the trastuzumab immunoglobulin gene (e.g., trastuzumab-coGW) as described herein operably linked to sequences which direct expression thereof.

The AAV sequences of the vector typically comprise the cis-acting 5′ and 3′ inverted terminal repeat (ITR) sequences (Sec, e.g., B. J. Carter, in “Handbook of Parvoviruses”, ed., P. Tijsser, CRC Press, pp. 155 168 (1990)). The ITR sequences are about 145 base pairs (bp) in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J. Virol., 70:520 532 (1996)). An example of such a molecule employed in the present invention is a “cis-acting” plasmid containing the transgene, in which the selected transgene sequence and associated regulatory elements are flanked by the 5′ and 3′ AAV ITR sequences. In one embodiment, the ITRs are from an AAV different than that supplying a capsid. In one embodiment, the ITR sequences are from AAV2. A shortened version of the 5′ ITR, termed ΔITR, has been described in which the D-sequence and terminal resolution site (trs) are deleted. In certain embodiments, the vector genome (e.g., of a plasmid) includes a shortened AAV2 ITR of 130 base pairs, wherein the external A elements is deleted. The shortened ITR may revert back to the wild-type length of 145 base pairs during vector DNA amplification using the internal A element as a template and packaging into the capsid to form the viral particle. In other embodiments, the full-length AAV 5′ and 3′ ITRs are used. However, ITRs from other AAV sources may be selected. Where the source of the ITRs is from AAV2 and the AAV capsid is from another AAV source, the resulting vector may be termed pseudotyped. However, other configurations of these elements may be suitable.

In one embodiment, rAAV.trastuzumab-coGW vector has an AAV capsid and a vector genome packaged therein which comprises at least one element heterologous to AAV capsid. In one embodiment, the vector genome contains, from 5′ to 3′: (a) an AAV 5′ ITR; (b) optionally an enhancer; (c) a promoter; (d) an intron; (e) a leader sequence and the trastuzumab heavy chain coding sequence; (f) optionally a furin cleavage sequence; (g) a T2A linker; (h) a leader sequence and the trastuzumab light chain coding sequence; (i) a polyA signal; and j) an AAV3′ ITR.

In one embodiment, the vector genome contains, from 5′ to 3′: (a) an AAV 5′ ITR; (b) a CB7 promoter comprising CMV IE enhancer, a chicken beta actin promoter, and a chimeric intron comprising a chicken beta-actin splicing donor (including the exon sequence) chicken beta actin intron and rabbit b-globin splicing acceptor optionally with spacer sequence; (c) Kozak sequence; (d) a leader sequence and the trastuzumab heavy chain coding sequence; (e) a furin cleavage sequence; (f) a T2A linker; (g) a leader sequence and the trastuzumab light chain coding sequence; (h) an SV40 polyA signal; and (i) an AAV3′ ITR. In one embodiment, the vector genome contains, from 5′ to 3′: (a) an AAV 5′ ITR; (b) a CB7 promoter comprising CMV IE enhancer, chicken beta actin promoter, and a chimeric intron comprising a chicken beta-actin splicing donor (including the exon sequence) chicken beta actin intron and rabbit b-globin splicing acceptor optionally with spacer sequence; (c) Kozak sequence; (d) a leader sequence and the trastuzumab heavy chain coding sequence; (e) a T2A linker; (f) a leader sequence and the trastuzumab light chain coding sequence; (g) an SV40 polyA signal; and (h) an AAV3′ ITR.

In one embodiment, the vector genome contains, from 5′ to 3′: (a) an AAV 5′ ITR; (b) a chicken beta actin promoter; (c) a chicken beta actin intron (CI); (d) a leader sequence and the trastuzumab heavy chain coding sequence; (e) a furin cleavage sequence; (f) a T2A linker; (g) a leader sequence and the trastuzumab light chain coding sequence; (h) an SV40 polyA signal; and (i) an AAV3′ ITR. In one embodiment, the vector genome contains, from 5′ to 3′: (a) an AAV 5′ ITR; (b) a chicken beta actin promoter; (c) a chicken beta actin intron (CI); (d) a leader sequence and the trastuzumab heavy chain coding sequence; (e) a T2A linker; (1) a leader sequence and the trastuzumab light chain coding sequence; (g) an SV40 polyA signal; and (h) an AAV3′ ITR.

In one embodiment, the vector genome contains, from 5′ to 3′: (a) an AAV 5′ ITR; (b) a Ubiquitin C (UbC) promoter; (c) a chimeric (Promega) intron (PI); (d) Kozak sequence; (e) a leader sequence and the trastuzumab heavy chain coding sequence; (f) a furin cleavage sequence; (g) a T2A linker; (h) a leader sequence and the trastuzumab light chain coding sequence; (i) an SV40 polyA signal; and (j) an AAV3′ ITR. In one embodiment, the vector genome contains, from 5′ to 3′: (a) an AAV 5′ ITR; (b) a Ubiquitin C (UbC) promoter; (c) a chimeric (promega) intron (PI); (d) Kozak sequence; (e) a leader sequence and the trastuzumab heavy chain coding sequence; (f) a T2A linker; (g) a leader sequence and the trastuzumab light chain coding sequence; (h) an SV40 polyA signal; and (i) an AAV3′ ITR.

In certain embodiments, the vector genome comprises nucleic acid sequence of SEQ ID NO: 1 (5′-ITR.CB.CI.IL2.V1.Trastuzumab-coGW.SV40.3′-ITR) or a sequence at least 95%, at least 96%, at least 97%, at least 98%, at least 99% to at least 100% identical thereto. In certain embodiments, the vector genome comprises nucleic acid sequence of SEQ ID NO: 1 (5′-ITR.CB.CI.IL2.V1.Trastuzumab-coGW.SV40.3′-ITR) or a sequence at least 99% identical thereto. In certain embodiments, the vector genome comprises nucleic acid sequence of SEQ ID NO: 25 (5′-ITR. UbC.PI.IL2_Furin_V1.Trastuzumab-coGW.SV40.3′-ITR) or a sequence at least 95%, at least 96%, at least 97%, at least 98%, at least 99% to at least 100% identical thereto. In certain embodiments, the vector genome comprises nucleic acid sequence of SEQ ID NO: 25 (5′-ITR. UbC.PI.IL2 Furin_V1.Trastuzumab-coGW.SV40.3′-ITR) or a sequence at least 99% identical thereto. In certain embodiments, the vector genome comprises nucleic acid sequence of SEQ ID NO: 27 (5′-ITR. CB7.CI.IL2_Furin_V1.Trastuzumab-coGW.SV40.3′-ITR) or a sequence at least 95%, at least 96%, at least 97%, at least 98%, at least 99% to at least 100% identical thereto. In certain embodiments, the vector genome comprises nucleic acid sequence of SEQ ID NO: 27 (5′-ITR. CB7.CI.IL2_Furin_V1.Trastuzumab-coGW.SV40.3′-ITR) or a sequence at least 99% identical thereto.

In certain embodiments, the AAV capsid is Clade F AAV capsid, wherein the Clade F AAV capsid is selected from an AAVhu68 capsid [See, e.g., US2020/0056159; PCT/US21/55436; SEQ ID NO: 37 and 38 for nucleic acid sequence; SEQ ID NO: 39 for amino acid sequence], an AAVhu95 capsid [See, e.g., U.S. Provisional Application No. 63/251,599, filed Oct. 2, 2201, International Patent Application No. PCT/US2022/077315, filed Sep. 30, 2022; SEQ ID NOs: 43 and 44 (hu95 nucleic acid sequence)] and SEQ ID NO: 45 (hu95 amino acid sequence), or an AAVhu96 capsid [See, e.g., U.S. Provisional Application No. 63/251,599, filed Oct. 2, 2201, and International Patent Application No. PCT/US2022/077315, filed Sep. 30, 2022; SEQ ID NOs: 46 and 47 (hu96 nucleic acid sequence) and SEQ ID NO: 48 (hu96 amino acid sequence)], AAV9 capsid (SEQ ID NO: 54 for nucleic acid sequence; SEQ ID NO: 55 for amino acid sequence). In certain embodiments, the AAV capsid is a Clade A capsid, such as AAVrh91 capsid (nucleic acid sequence of SEQ ID NOs: 40 and 41; amino acid sequence of SEQ ID NO: 42). See, PCT/US20/030266, filed Apr. 29, 2020, now published WO2020/223231, which is incorporated by reference herein and International Application No. PCT/US21/45945, filed Aug. 13, 2021 which are incorporated herein by reference.

In certain embodiment, the AAV capsid is an AAVhu68 capsid. In certain embodiments the AAV capsid is an AAV9 capsid. In certain embodiments the AAV capsid is an AAVhu95 capsid. In certain embodiments, the AAV capsid is an AAVhu96 capsid.

In certain embodiments, the AAV capsid for the compositions and methods described herein is chosen based on the target cell. In certain embodiment, the AAV capsid transduces a CNS cell and/or a PNS cell. In certain embodiments, other AAV capsid may be chosen, the AAV capsid is selected from a cy02 capsid, a rh43 capsid, an AAV8 capsid, a rh01 capsid, an AAV9 capsid, an rh8 capsid, a rh10 capsid, a bb01 capsid, a hu37 capsid, a rh02 capsid, a rh20 capsid, a rh39 capsid, a rh64 capsid, an AAV6 capsid, an AAV1 capsid, a hu44 capsid, a hu48 capsid, a cy05 capsid a hull capsid, a hu32 capsid, a pi2 capsid, or a variation thereof. In certain embodiments, the AAV capsid is a Clade F capsid, such as AAV9 capsid, AAVhu68 capsid, hu31 capsid, hu32 capsid, or a variation thereof. See, e.g., WO 2005/033321 published Apr. 14, 2015, WO 2018/160582, and US 2015/0079038, each of which is incorporated herein by reference in its entirety. In certain embodiments, the AAV capsid is a non-cade F capsid, for example a Clade A, B, C, D, or E capsid. In certain embodiment, the non-Clade F capsid is an AAV1 or a variation thereof. In certain embodiment, the AAV capsid transduces a target cell other than the nervous system cells. In certain embodiments, the AAV capsid is a Clade A capsid (e.g., AAV1, AAV6, AAVrh91), a Clade B capsid (e.g., AAV 2), a Clade C capsid (e.g., hu53), a Clade D capsid (e.g., AAV7), or a Clade E capsid (e.g., rh10).

An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein when used to refer to vp capsid proteins, the term “heterogeneous” or any grammatical variation thereof, refers to a population consisting of elements that are not the same, for example, having vp1, vp2 or vp3 (also referenced as VP1, VP2, VP3, or Vp1, Vp2, Vp3) monomers (proteins) with different modified amino acid sequences. The term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine—glycine pairs and optionally further comprising other deamidated amino acids, wherein the deamidation results in an amino acid change and other optional modifications.

In certain embodiments. AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart.

Additionally, provided herein is a recombinant nucleic acid molecule comprising (a) an AAV 5′ inverted terminal repeat (ITR), (b) an expression cassette comprising at least one open reading frame (ORF) comprising a trastuzumab heavy chain and a trastuzumab light chain and nucleic acid sequences operably linked thereto which regulate expression of the trastuzumab heavy chain and trastuzumab light chain, and (c) an AAV 3′ ITR, wherein the expression cassette comprises: (i) a promoter which is a Ubiquitin C (UbC) promoter, and (ii) a intron, which is a chimeric intron (e.g., such as available from Promega), and (iii) at least one ORF which comprises: a nucleic acid sequence encoding a leader peptide operably linked to a trastuzumab heavy chain, (iv) a nucleic acid sequence comprising SEQ ID NO: 3 encoding a trastuzumab heavy chain, (v) a furin cleaveage site, (vi) a T2A linker, (vii) a nucleic acid sequence encoding a leader peptide operably linked to a trastuzumab light chain, (viii) nucleic acid sequence comprising SEQ ID NO: 5 encoding a trastuzumab light chain, (ix) an SV40 polyadenylation (polyA) sequence, and wherein the expression cassette further comprises spacer sequences. In certain embodiments, a recombinant nucleic acid molecule comprises expression cassette comprising nucleic acid sequence of SEQ ID NO: 26. In certain embodiments, a recombinant nucleic acid molecule comprises vector genome comprising nucleic acid sequence of SEQ ID NO: 25. In certain embodiments, a recombinant nucleic acid molecule is for use in rAAV production system as described herein.

Additionally, provided herein, is an rAAV production system useful for producing a rAAV as described herein. The production system comprises a cell culture comprising (a) a nucleic acid sequence encoding an AAV capsid protein; (b) the vector genome; and (c) sufficient AAV rep functions and helper functions to permit packaging of the vector genome into the AAV capsid. In certain embodiments, the vector genome is SEQ ID NO: 1. In certain embodiments, the vector genome is SEQ ID NO: 25. In certain embodiments, the vector genome is SEQ ID NO: 27. In certain embodiments, the cell culture is bacterial cell culture. In certain embodiments, the cell culture is mammalian cell culture. In certain embodiments, the cell culture is a human embryonic kidney 293 (HEK293) cell culture. In certain embodiments, the cell culture is a suspension cell culture. In certain embodiments, the AAV rep is from a different AAV. In certain embodiments, wherein the AAV rep is from AAV2. In certain embodiments, the AAV rep coding sequence and cap genes are on the same nucleic acid molecule, wherein there is optionally a spacer between the rep sequence and cap gene.

For use in producing an AAV viral vector (e.g., a recombinant (r) AAV), the vector genomes can be carried on any suitable vector, e.g., a plasmid, which is delivered to a packaging host cell. The plasmids useful in this invention may be engineered such that they are suitable for replication and packaging in vitro in prokaryotic cells, insect cells, mammalian cells, among others. Suitable transfection techniques and packaging host cells are known and/or can be readily designed by one of skill in the art.

In certain embodiments, a recombinant nucleic acid molecule is a plasmid. In certain embodiments, a recombinant nucleic acid molecule (e.g., plasmid) is useful in rAAV production.

In certain embodiments, a recombinant nucleic acid molecule (e.g., a plasmid) useful in rAAV production comprises a vector genome comprising a 5′-ITR.CB.CI.IL2.V1.Trastuzumab-coGW.SV40.3′-ITR. In certain embodiments, recombinant a nucleic acid molecule (e.g., a plasmid) useful in rAAV production comprises a vector genome comprising a nucleic acid sequence of SEQ ID NO: 1. In certain embodiments, a recombinant nucleic acid molecule (e.g., a plasmid) useful in rAAV production comprises a vector genome comprising a 5′-ITR. UbC.PJ.IL2_Furin_V1.Trastuzumab-coGW.SV40.3′-ITR. In certain embodiments, recombinant a nucleic acid molecule (e.g., a plasmid) useful in rAAV production comprises a vector genome comprising a nucleic acid sequence of SEQ ID NO: 25. In certain embodiments, a recombinant nucleic acid molecule (e.g., a plasmid) useful in rAAV production comprises a vector genome comprising a 5′-ITR.CB7.CJ.IL2_Furin_V1.Trastuzumab-coGW.SV40.3′-ITR. In certain embodiments, recombinant a nucleic acid molecule (e.g., a plasmid) useful in rAAV production comprises a vector genome comprising a nucleic acid sequence of SEQ ID NO: 27.

Methods for generating and isolating AAVs suitable for use as vectors are known in the art. See generally, e.g., Grieger & Samulski, 2005, Adeno-associated virus as a gene therapy vector: Vector development, production and clinical applications, Adv. Biochem. Engin/Biotechnol. 99: 119-145; Buning et al., 2008, Recent developments in adeno-associated virus vector technology, J. Gene Med. 10:717-733; and the references cited below, each of which is incorporated herein by reference in its entirety. As used herein, a gene therapy vector refers to a rAAV as described herein, which is suitable for use in treating a patient. For packaging a gene into virions, the ITRs are the only AAV components required in cis in the same construct as the nucleic acid molecule containing the gene. The cap and rep genes can be supplied in trans.

In one embodiment, the expression cassettes described herein are engineered into a genetic element (e.g., a shuttle plasmid) which transfers the immunoglobulin construct sequences carried thereon into a packaging host cell for production of a viral vector. In one embodiment, the selected genetic element may be delivered to an AAV packaging cell by any suitable method, including transfection, electroporation, liposome delivery, membrane fusion techniques, high velocity DNA-coated pellets, viral infection and protoplast fusion. Stable AAV packaging cells can also be made. The methods used to make such constructs are known to those with skill in nucleic acid manipulation and include genetic engineering, recombinant engineering, and synthetic techniques. See, e.g., Molecular Cloning: A Laboratory Manual, ed. Green and Sambrook, Cold Spring Harbor Press, Cold Spring Harbor, NY (2012).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product. These empty capsids are non-functional to transfer the gene of interest to a host cell.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid protein; a functional rep gene; an expression cassette composed of, at a minimum, AAV inverted terminal repeats (ITRs) and a transgene; and sufficient helper functions to permit packaging of the expression cassette into the AAV capsid protein. Methods of generating the capsid, coding sequences therefor, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, a production cell culture useful for producing a recombinant AAV having a capsid selected from AAVhu68, AAVrh91, AAVhu95 or AAVhu96 is provided. Such a cell culture contains a nucleic acid which expresses the AAVhu68 capsid protein in the host cell (e.g., SEQ ID NO: 37 or SEQ ID NO: 38; a nucleic acid molecule suitable for packaging into the AAVhu68 capsid, e.g., a vector genome which contains AAV ITRs and a non-AAV nucleic acid sequence encoding a gene operably linked to regulatory sequences which direct expression of the gene in a host cell; and sufficient AAV rep functions and adenovirus helper functions to permit packaging of the vector genome into the recombinant AAVhu68, or AAVrh91 capsid (e.g., SEQ ID NO: 40 or SEQ ID NO: 41), AAVhu95 capsid (e.g., SEQ ID NO: 43 or SEQ ID NO: 44), AAVhu96 capsid (e.g., SEQ ID NO: 47 or SEQ ID NO: 48). In one embodiment, the cell culture is composed of mammalian cells (e.g., human embryonic kidney 293 cells, among others) or insect cells (e.g., Spodoptera frugiperda (SfD) cells). In certain embodiments, baculovirus provides the helper functions necessary for packaging the vector genome into the recombinant AAVhu68, AAVrh91, AAVhu95 or AAVhu96 capsid.

Optionally the rep functions are provided by an AAV other than AAV2, selected to complement the source of the ITRs.

In one embodiment, cells are manufactured in a suitable cell culture (e.g., HEK 293 or Sf9) or suspension. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV vector genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Zhang et al., 2009, Adenovirus-adeno-associated virus hybrid for large-scale recombinant adeno-associated virus production, Human Gene Therapy 20:922-929, the contents of each of which is incorporated herein by reference in its entirety. Methods of making and using these and other AAV production systems are also described in the following US patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. These methods are described in more detail in WO2017/160360, filed Dec. 9, 2016, entitled “Scalable Purification Method for AAV9”, which is incorporated by reference. Purification methods for AAV8, WO2017/100676, filed Dec. 9, 2016, and rh10, WO2017/100704, filed Dec. 9, 2016, entitled “Scalable Purification Method for AAVrh10”, also filed Dec. 11, 2015, and for AAV1, WO2017/100674, filed Dec. 9, 2016 for “Scalable Purification Method for AAV1”, filed Dec. 11, 2015, are all incorporated by reference herein. Other suitable methods may be selected.

To calculate empty and full particle content, VP3 band volumes for a selected sample (e.g., in examples herein an iodixanol gradient-purified preparation where #of genome copies (GC)=#of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles.

Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-old or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb 14.

In certain embodiments, the manufacturing process for rAAV as described herein involves method as described in U.S. Provisional Patent Application No. 63/371,597, filed Aug. 16, 2022, and U.S. Provisional Patent Application No. 63/371,592, filed Aug. 16, 2022, which are incorporated herein by reference in their entirety.

In brief, the method for separating rAAVhu68 (or AAVrh91, AAVhu95 or AAVhu96) particles having packaged genomic sequences from genome-deficient AAVhu68 (or AAVrh91 or AAVhu95 or AAVhu96) intermediates involves subjecting a suspension comprising recombinant AAVhu68 (or AAVrh91) viral particles and AAVhu68 (or AAVrh91 or AVhu95 or AAVhu96) capsid intermediates to fast performance liquid chromatography, wherein the AAVhu68 (or AAVrh91 or AAVhu95 or AAVhu96) viral particles and AAVhu68 intermediates are bound to a strong anion exchange resin equilibrated at a pH of about 10.2 (or about 9.8 for AAVrh91), and subjected to a salt gradient while monitoring eluate for ultraviolet absorbance at about 260 nanometers (nm) and about 280 nm. Although less optimal for rAAVhu68 and AAVrh91, the pH may be in the range of about 10 to 10.4. In this method, the AAV full capsids are collected from a fraction which is eluted when the ratio of A260/A280 reaches an inflection point. In one example, for the Affinity Chromatography step, the diafiltered product may be applied to an affinity resin (Life Technologies) that efficiently captures the AAV serotype. Under these ionic conditions, a significant percentage of residual cellular DNA and proteins flow through the column, while AAV particles are efficiently captured.

The rAAV.Trastuzumab-coGW (e.g., rAAV.UbC.PI.IL2_Furin_V1.Trastuzumab-coGW.SV40, rAAV.UbC.PJ.IL2.V1.Trastuzumab-coGW.SV40, rAAV.CB7.CI.IL2_Furin_V1.Trastuzumab-coGW.SV40 or rAAV.CB7.CI.IL2.V1.Trastuzumab-coGW.SV40) is suspended in a suitable physiologically compatible composition (e.g., a buffered saline). This composition may be frozen for storage, later thawed and optionally diluted with a suitable diluent. Alternatively, the vector may be prepared as a composition which is suitable for delivery to a patient without proceeding through the freezing and thawing steps.

As used herein, the term “clade” as it relates to groups of AAV refers to a group of AAV which are phylogenetically related to one another as determined using a Neighbor-Joining algorithm by a bootstrap value of at least 75% (of at least 1000 replicates) and a Poisson correction distance measurement of no more than 0.05, based on alignment of the AAV vp1 amino acid sequence. The Neighbor-Joining algorithm has been described in the literature. See, e.g., M. Nei and S. Kumar, Molecular Evolution and Phylogenetics (Oxford University Press, New York (2000). Computer programs are available that can be used to implement this algorithm. For example, the MEGA v2.1 program implements the modified Nei-Gojobori method. Using these techniques and computer programs, and the sequence of an AAV vp1 capsid protein, one of skill in the art can readily determine whether a selected AAV is contained in one of the clades identified herein, in another clade, or is outside these clades. See, e.g., G Gao, et al, J Virol, 2004 Jun; 78(10): 6381-6388, which identifies Clades A, B, C, D, E and F, and provides nucleic acid sequences of novel AAV, GenBank Accession Numbers AY530553 to AY530629. See, also, WO 2005/033321.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

As used herein, the terms “recombinant AAV”, “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, NY (2012).

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

The term “heterogeneous” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine—glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins is at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine—glycine pairs.

Unless defined otherwise in this specification, technical and scientific terms used herein have the same meaning as commonly understood by one of ordinary skill in the art and by reference to published texts, which provide one skilled in the art with a general guide to many of the terms used in the present application.

### Pharmaceutical Composition

In one aspect, provided herein is a pharmaceutical composition comprising a rAAV as described herein in a formulation buffer.

In one embodiment, the rAAV is formulated at about 1×10⁹ genome copies (GC)/mL to about 1×10¹⁴ GC/mL. In a further embodiment, the rAAV is formulated at about 3×10⁹ GC/mL to about 3×10¹³ GC/mL. In yet a further embodiment, the rAAV is formulated at about 1×10⁹ GC/mL to about 1×10¹³ GC/mL. In one embodiment, the rAAV is formulated at least 1×10¹¹ GC/mL.

Provided herein also is a composition comprising an rAAV as described herein and an aqueous suspension media. In certain embodiments, the suspension is formulated for intravenous delivery, intrathecal administration, or intracerebroventricular administration. In one aspect, the compositions contain at least one rAAV stock and an optional carrier, excipient and/or preservative.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host. Delivery vehicles such as liposomes, nanocapsules, nanoparticles, lipid nanoparticle (LNP), microparticles, microspheres, lipid particles, vesicles, and the like, may be used for the introduction of the compositions of the present invention into suitable host cells. In particular, the rAAV vector delivered vector genomes may be formulated for delivery either encapsulated in a lipid particle, a liposome, a vesicle, a nanosphere, or a nanoparticle or the like.

In one embodiment, a composition includes a final formulation suitable for delivery to a subject, e.g., is an aqueous liquid suspension buffered to a physiologically compatible pH and salt concentration. Optionally, one or more surfactants are present in the formulation. In another embodiment, the composition may be transported as a concentrate which is diluted for administration to a subject. In other embodiments, the composition may be lyophilized and reconstituted at the time of administration.

A suitable surfactant, or combination of surfactants, may be selected from among non-ionic surfactants that are nontoxic.

In one embodiment, the composition includes a carrier, diluent, excipient and/or adjuvant. Suitable carriers may be readily selected by one of skill in the art in view of the indication for which the transfer virus is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The buffer/carrier should include a component that prevents the rAAV, from sticking to the infusion tubing but does not interfere with the rAAV binding activity in vivo. A suitable surfactant, or combination of surfactants, may be selected from among non-ionic surfactants that are nontoxic. In one embodiment, a difunctional block copolymer surfactant terminating in primary hydroxyl groups is selected, e.g., such as Poloxamer 188 (also known under the commercial names Pluronic® F68 [BASF], Lutrolk F68, Synperonic® F68, Kolliphor® P188) which has a neutral pH, has an average molecular weight of 8400. Other surfactants and other Poloxamers may be selected, i.e., nonionic triblock copolymers composed of a central hydrophobic chain of polyoxypropylene (poly(propylene oxide)) flanked by two hydrophilic chains of polyoxyethylene (poly(ethylene oxide)), SOLUTOL HS 15 (Macrogol-15 Hydroxystearate), LABRASOL (Polyoxy capryllic glyceride), polyoxy-oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

In certain embodiments, the composition containing the rAAV.Trastuzumab-coGW is delivered at a pH in the range of 6 to 8, or 7.2 to 7.8, or 7.5 to 8. For intrathecal delivery, a pH above 7.5 may be desired, e.g., 7.5 to 8, or 7.8. For intravenous delivery, a pH of about 6.8 to about 7.2 may be desired.

In certain embodiments, the formulation may contain a buffered saline aqueous solution not comprising sodium bicarbonate. Such a formulation may contain a buffered saline aqueous solution comprising one or more of sodium phosphate, sodium chloride, potassium chloride, calcium chloride, magnesium chloride and mixtures thereof, in water, such as a Harvard's buffer. In one embodiment, the buffer is PBS. In another embodiment, the buffer is an artificial cerebrospinal fluid (aCSF), e.g., Eliott's formulation buffer; or Harvard apparatus perfusion fluid (an artificial CSF with final Ion Concentrations (in mM): Na 150; K 3.0; Ca 1.4; Mg 0.8; P 1.0; Cl 155). The aqueous solution may further contain Kolliphor® P188, a poloxamer which is commercially available from BASF which was formerly sold under the trade name Lutrol® F68. The aqueous solution may have a pH of 7.2.

In another embodiment, the formulation may contain a buffered saline aqueous solution comprising 1 mM Sodium Phosphate (Na₃PO₄), 150 mM sodium chloride (NaCl), 3 mM potassium chloride (KCl), 1.4 mM calcium chloride (CaCl₂)), 0.8 mM magnesium chloride (MgCl₂), and 0.001% poloxamer (e.g., Kolliphor®) 188, pH 7.2. See, e.g., harvardapparatus.com/harvard-apparatus-perfusion-fluid.html. In certain embodiments, Harvard's buffer is preferred due to better pH stability observed with Harvard's buffer.

In certain embodiments, the formulation buffer is artificial CSF with Pluronic F68. In other embodiments, the formulation may contain one or more permeation enhancers. Examples of suitable permeation enhancers may include, e.g., mannitol, sodium glycocholate, sodium taurocholate, sodium deoxycholate, sodium salicylate, sodium caprylate, sodium caprate, sodium lauryl sulfate, polyoxyethylene-9-laurel ether, or EDTA.

Optionally, the compositions of the invention may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The compositions according to the present invention may comprise a pharmaceutically acceptable carrier, such as defined above. Suitably, the compositions described herein comprise an effective amount of one or more AAV suspended in a pharmaceutically suitable carrier and/or admixed with suitable excipients designed for delivery to the subject via injection, osmotic pump, intrathecal catheter, or for delivery by another device or route. In certain embodiments, an ommaya reservoir is used for delivery. In one example, the composition is formulated for intrathecal delivery. In one example, the composition is formulated for intravenous (iv) delivery.

In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

As used herein, the term “dosage” or “amount” can refer to the total dosage or amount delivered to the subject in the course of treatment, or the dosage or amount delivered in a single unit (or multiple unit or split dosage) administration.

In certain embodiments, the compositions can be formulated in dosage units to contain an amount of rAAV that is in the range of about 1×10⁹ GC per gram of brain mass to about 1×10¹³ genome copies (GC) per gram (g) of brain mass, including all integers or fractional amounts within the range and the endpoints. In another embodiment, the dosage is 1×10¹⁰ GC per gram of brain mass to about 1×10¹³ GC per gram of brain mass. In specific embodiments, the dose of the vector administered to a patient is at least about 1.0×10⁹ GC/g, about 1.5×10⁹ GC/g, about 2.0×10⁹ GC/g, about 2.5×10⁹ GC/g, about 3.0×10⁹ GC/g, about 3.5×10⁹ GC/g, about 4.0×10⁹ GC/g, about 4.5×10⁹ GC/g, about 5.0×10⁹ GC/g, about 5.5×10⁹ GC/g, about 6.0×10⁹ GC/g, about 6.5×10⁹ GC/g, about 7.0×10⁹ GC/g, about 7.5×10⁹ GC/g, about 8.0×10⁹ GC/g, about 8.5×10⁹ GC/g, about 9.0×10⁹ GC/g, about 9.5×10⁹ GC/g, about 1.0×10¹⁰ GC/g, about 1.5×10¹⁰ GC/g, about 2.0×10¹⁰ GC/g, about 2.5×10¹⁰ GC/g, about 3.0×10¹⁰ GC/g, about 3.5×10¹⁰ GC/g, about 4.0×10¹⁰ GC/g, about 4.5×10¹⁰ GC/g, about 5.0×10¹⁰ GC/g, about 5.5×10¹⁰ GC/g, about 6.0×10¹⁰ GC/g, about 6.5×10¹⁰ GC/g, about 7.0×10¹⁰ GC/g, about 7.5×10¹⁰ GC/g, about 8.0×10¹⁰ GC/g, about 8.5×10¹⁰ GC/g, about 9.0×10¹⁰ GC/g, about 9.5×10¹⁰ GC/g, about 1.0×10¹¹ GC/g, about 1.5×10¹¹ GC/g, about 2.0×10¹¹ GC/g, about 2.5×10¹¹ GC/g, about 3.0×10¹¹ GC/g, about 3.5×10¹¹ GC/g, about 4.0×10¹¹ GC/g, about 4.5×10¹¹ GC/g, about 5.0×10¹¹ GC/g, about 5.5×10¹¹ GC/g, about 6.0×10¹¹ GC/g, about 6.5×10¹¹ GC/g, about 7.0×10¹¹ GC/g, about 7.5×10¹¹ GC/g, about 8.0×10¹¹ GC/g, about 8.5×10¹¹ GC/g, about 9.0×10¹¹ GC/g, about 9.5×10¹¹ GC/g, about 1.0×10¹² GC/g, about 1.5×10¹² GC/g, about 2.0×10¹² GC/g, about 2.5×10¹² GC/g, about 3.0×10¹² GC/g, about 3.5×10¹² GC/g, about 4.0×10¹² GC/g, about 4.5×10¹² GC/g, about 5.0×10¹² GC/g, about 5.5×10¹² GC/g, about 6.0×10¹² GC/g, about 6.5×10¹² GC/g, about 7.0×10¹² GC/g, about 7.5×10¹² GC/g, about 8.0×10¹² GC/g, about 8.5×10¹² GC/g, about 9.0×10¹² GC/g, about 9.5×10¹² GC/g, about 1.0×10¹³ GC/g, about 1.5×10¹³ GC/g, about 2.0×10¹³ GC/g, about 2.5×10¹³ GC/g, about 3.0×10¹³ GC/g, about 3.5×10¹³ GC/g, about 4.0×10¹³ GC/g, about 4.5×10¹³ GC/g, about 5.0×10¹³ GC/g, about 5.5×10¹³ GC/g, about 6.0×10¹³ GC/g, about 6.5×10¹³ GC/g, about 7.0×10¹³ GC/g, about 7.5×10¹³ GC/g, about 8.0×10¹³ GC/g, about 8.5×10¹³ GC/g, about 9.0×10¹¹ GC/g, about 9.5×10¹¹ GC/g, or about 1.0×10¹⁴ GC/g brain mass.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹ 7×10¹¹, 8×10¹¹ or 9×10¹¹ GC per dose including all integers or fractional amounts within the range. In another embodiment, the compositions are formulated to contain at least 1×10¹² 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In certain embodiments, the composition is delivered intrathecally, optionally via intra-cisterna magna (ICM) injection. In certain embodiments, the composition is delivered via intraparenchymal administration. In certain embodiments, the composition is delivered via Ommaya Reservoir delivery system. In certain embodiments, the composition is delivered via direct injection into tumor or tumor bed.

In one embodiment, the pharmaceutical composition comprising a rAAV as described herein is administrable at a dose of about 1×10⁹ GC per gram of brain mass to about 1×10¹³ GC per gram of brain mass.

The rAAV, preferably suspended in a physiologically compatible carrier, may be administered to a human or non-human mammalian patient.

It should be understood that the compositions in the pharmaceutical composition described herein are intended to be applied to other compositions, regiments, aspects, embodiments and methods described across the Specification.

### Uses and Regimens

In one aspect, a method is provided herein is a method of treating a human subject diagnosed with a HER2-positive cancer. The method comprises administering to a subject a suspension of an rAAV vector as described herein. In one embodiment, the method comprises administering to a subject having a metastatic HER2-positive cancer in the brain, a suspension of a rAAV as described herein in a formulation buffer at a dose of about 1×10⁹ GC per gram of brain mass to about 1×10¹⁴ GC per gram of brain mass. In another embodiment, the method comprises administering to a subject a suspension of a rAAV as described herein in a formulation buffer at a dose of about 1×10⁹ CC per kg of body weight to about 1×10¹⁴ CC per kg of body weight. Suitable doses for systemic administration and/or intratumoral administration can be determined by one of skill in the art.

In some embodiments, the method comprises treating a subject diagnosed with a HER2-positive cancer which is a refractory and/or resistant cancer. In some embodiments, the method comprises treating a subject diagnosed with a HER2-positive breast cancer which is a refractory and/or resistant cancer. In some embodiments, the method comprises treating a subject diagnosed with a metastatic HER2-positive breast cancer in the brain which is a refractory and/or resistant cancer. In some embodiments, the method comprises treating a subject diagnosed with a HER2-positive cancer which is trastuzumab-resistant. In some embodiments, the method comprises treating a subject having a metastatic HER2-positive breast cancer in the brain. In some embodiments, the method comprises treating a subject diagnosed with a HER2-positive breast cancer which is trastuzumab-resistant (e.g., estrogen receptor (ER)-positive, progesterone receptor (PR)-positive, and Her2-positive). In certain embodiments, the method comprises treating a subject diagnosed with an ER-negative, PR-negative, and Her2-positive cancer. In certain embodiments, the method comprises treating a subject diagnosed with HER2/low Tumor.

As used herein, “refractory cancer” and/or “resistant cancer” refers to a cancer which is refractory or resistant to one or more cancer therapies, for example a cancer chemotherapy (cytotoxic chemotherapy). In certain embodiment, the refractory and/or resistant cancer is not amendable to surgical intervention. In certain embodiment, the refractory and/or resistant cancer is initially unresponsive to chemotherapy or radiation therapy. In certain embodiment, the refractory and/or resistant cancer becomes unresponsive to cancer therapeutics over time.

As used herein “trastuzumab-resistant” refers to a cancer which is refractory or resistant to trastuzumab treatment. In certain embodiments, “refractory” or “resistant” means that the cancer (i.e., HER2-positive) is non-responsive to trastuzumab following a standard course of treatment, e.g., the cancer continues to progress even after the trastuzumab treatment. In certain embodiments, the trastuzumab-resistant cancer is inherently resistant to trastuzumab treatment. In certain embodiments, the trastuzumab-resistant cancer acquires resistance, wherein cancer cells initially responded to treatment, but after some period of time no longer responded to trastuzumab treatment (i.e., refractory to treatment). In certain embodiments, the resistance is developed to a late stage therapeutic, wherein the HER2-positive tumors and non-responsive or become resistant to the trastuzumab therapy.

When assessed in vitro, in certain embodiments, the trastuzumab-resistant cancer cell is from a parental cell, which was trastuzumab sensitive, and which was treated with trastuzumab-comprising composition and/or solution either as a prior treatment or as a means of exerting selective pressure. See also, Pohlman, P. R., et al., Resistance to Trastuzumab in Breast Cancer, 2009, Clin. Cancer Res. 15(24):7479-7491; Vu, T., and Claret, F. X., Trastuzumab: updated mechanisms of action and resistance in breast cancer, 2012, Front. Oncol. 2(62); Rimawi, M. F., et al., Resistance to Anti-HER2 Therapies in Breast Cancer, 2015, American Society of Clinical Oncology Educational Book 35 (May 14, 2015) c157-c164, which are incorporated herein by reference.

In some embodiments, the method comprises administering to a subject a suspension of a rAAV as described herein in a formulation buffer at a dose of 1×10¹¹ to 1×10¹⁴ GC/kg. In certain embodiments, the method comprises intravenous administration at a dose ranging from about 1×10¹² genome copies (GC)/kg of rAAV to about 1×10¹⁴ GC of rAAV per kg. In certain embodiments, a dose is about 1×10¹³ GC/kg to about 1×10¹⁴ GC of rAAV per patient, or about 3×10¹³ GC/kg. In certain embodiments, delivery via intravenous administration is contemplated with a dose of about 3×10¹² GC/kg to about 1×10¹⁴ GC/kg, further including doses of about 3.0×10¹³ GC/kg and about 1.0×10¹³ GC/kg.

In one embodiment, the subject is delivered a therapeutically effective amount of the rAAV described herein. As used herein, a “therapeutically effective amount” refers to the amount of the composition comprising the nucleic acid sequence encoding trastuzumab immunoglobulin which delivers and expresses in the target cells an amount of immunoglobulin sufficient to achieve efficacy. The dosage is adjusted to balance the therapeutic benefit against any side effects and such dosages may vary depending upon the therapeutic application for which the recombinant vector is employed. The levels of expression of the transgene product can be monitored to determine the frequency of dosage resulting in viral vectors, preferably AAV vectors containing the transgene. Optionally, dosage regimens similar to those described for therapeutic purposes may be utilized for immunization using the compositions described herein.

Suitable, conventional, and pharmaceutically acceptable routes of administration include, but are not limited to, direct delivery to a desired organ (e.g., brain, CSF, heart), intranasal, intrathecal, intratracheal, intraarterial, intraocular, intravenous, intramuscular, subcutaneous, intradermal, intraparenchymal, intracerebroventricular, intrathecal, ICM, lumbar puncture and other parenteral routes of administration. Routes of administration may be combined, if desired.

Suitable volumes for delivery of these doses and concentrations may be determined by one of skill in the art. For example, volumes of about 1 μL to 150 mL may be selected for CNS delivery, with the higher volumes being selected for adults. Typically, for newborn infants a suitable volume is about 0.5 mL to about 10 mL, for older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other embodiments, a patient may receive an intrathecal administration in a volume of about 5 mL to about 15 mL are selected, or about 7.5 mL to about 10 mL. Other suitable volumes and dosages may be determined for CNS delivery, intratumoral delivery, and/or for systemic delivery (e.g., IV). The dosage will be adjusted to balance the therapeutic benefit against any side effects and such dosages may vary depending upon the therapeutic application for which the recombinant vector is employed.

In one embodiment, the method comprises administering rAAV as described herein is to the subject in need. The above-described recombinant vectors may be administered or delivered to host cells according to published methods. The rAAV, preferably suspended in a physiologically compatible carrier, may be administered to a subject, human or non-human mammalian patient. In certain embodiments, for administration to a human patient, the rAAV is suitably suspended in an aqueous solution containing saline, a surfactant, and a physiologically compatible salt or mixture of salts. Suitably, the formulation is adjusted to a physiologically acceptable pH, e.g., in the range of pH 6 to 9, or pH 6.5 to 7.5, pH 7.0 to 7.7, or pH 7.2 to 7.8. As the pH of the cerebrospinal fluid is about 7.28 to about 7.32, for intrathecal delivery, a pH within this range may be desired; whereas for intravenous delivery, a pH of about 6.8 to about 7.2 may be desired. For intravenous delivery, a pH of about 6.8 to about 7.2 may be desired. However, other pHs within the broadest ranges and these subranges may be selected for other route of delivery.

Suitably, the pharmaceutical compositions, as described herein, and the uses thereof comprise delivering to the subject via injection, osmotic pump, intrathecal catheter, or for delivery by another device or route. In one example, the composition is formulated for intrathecal delivery.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna. In certain embodiment, a rAAV, vector, or composition as described herein is administrated to a subject in need via the intrathecal administration. In certain embodiments, the intrathecal administration is performed as described in US Patent Publication No. 2018/0339065 A1, published Nov. 29, 2019, which is incorporated herein by reference in its entirety. In certain embodiments, the CNS administration is performed using Ommaya Reservoir (also referred to as Ommaya device or Ommaya system).

As used herein, the terms “intracisternal delivery” or “intracisternal administration” refer to a route of administration for drugs directly into the cerebrospinal fluid of the cisterna magna cerebellomedularis, more specifically via a suboccipital puncture or by direct injection into the cisterna magna or via permanently positioned tube.

As used herein, the term “intraparenchymal”, “dentate nucleus” or IDN refers to a route of administration of a composition directly into dentate nuclei. IDN allows for targeting of dentate nuclei and/or cerebellum. In certain embodiments, the IDN administration is performed using ClearPoint® Neuro Navigation System (MRI Interventions, Inc., Memphis, TN) and ventricular cannula, which allows for MRI-guided visualization and administration. Alternatively, other devices and methods may be selected.

Suitably, the pharmaceutical compositions, as described herein, and the uses thereof comprise delivering to tumor bed to the subject via surgical and non-surgical techniques. See also, Gilbert, A., and Machluf, M., Nano to micro delivery systems: targeting angiogenesis in brain tumors, J Angiogenes Res. 2010 Oct. 8; 2(1):20; Balyasnikova, I V., et al., Intranasal Delivery of Mesenchymal Stem Cells Significantly Extends Survival of Irradiated Mice with Experimental Brain Tumors, Molecular Therapy, 2014, 22(1):P140-P148, epub Jan. 1, 2014, which are incorporated herein by reference in its entirety. In certain embodiments, the pharmaceutical compositions, as described herein, and the uses thereof comprise directly delivering to the tumor or tumor bed in the CNS. In certain embodiments, the pharmaceutical compositions, as described herein, and the uses thereof comprise delivering via a systemic route. In certain embodiments, the pharmaceutical compositions, as described herein, and the uses thereof comprise delivering directly into a Her2-positive tumor located outside of the CNS (e.g., in the breast or a metastatic Her2-positive cancer, or gastric gastroesophageal junction cancer). See also, Ma, H., et al., Intratumoral gene therapy of malignant brain tumor in a rat model with angiostatin delivered by adeno-associated viral (AAV) vector, 2002, Gene Therapy, 9:2-11; Liang, C., et al., Local Expression of Secondary Lymphoid Tissue Chemokine Delivered by Adeno-Associated Virus within the Tumor Bed Stimulates Strong Anti-Liver Tumor Immunity, Journal of Virology, 2007, 81(17):9502-9511; Xu, X., et al., Adeno-associated virus (AAV)-based gene therapy for glioblastoma, Cancer Cell International, 2021, 21:76, epub 26 Jan. 2021; Reul, J., et al., Tumor-Specific Delivery of Immune Checkpoint Inhibitors by Engineered AAV Vectors, Methods (Frontiers in Oncology), 2019, 9:52, epub Feb. 14, 2019; Sheth, R A., et al., Assessment of Image-Guided Intratumoral Delivery of Immunotherapeutics in Patients With Cancer, JAMA Netw Open. 2020; 3(7):e207911, epub Jul. 29, 2020; Tchou, J., et al., Safety and Efficacy of Intratumoral Injections of Chimeric Antigen Receptor (CAR) T Cells in Metastatic Breast Cancer, Cancer Immunol Res, 2017, 5(12): 1152-1161, epub Nov. 6, 2017, which are all incorporated herein by reference in its entirety.

In a further aspect, suitably, the compositions of the invention are designed so that rAAV vectors carry the nucleic acid expression cassettes encoding the trastuzumab immunoglobulin construct and regulatory sequences which direct expression of the trastuzumab immunoglobulin thereof in the selected cell. Following administration of the vectors into the CNS, the vectors deliver the expression cassettes to the CNS and express the proteinaceous immunoglobulin constructs in vivo. The use of compositions described herein in an anti-neoplastic method are described, as are uses of these compositions in anti-neoplastic regimens, which may optionally involve delivery of one or more other anti-neoplastic or other active agents.

As stated above, a composition may contain a single type of AAV vector as described herein which contains the expression cassette for delivering the anti-neoplastic trastuzumab immunoglobulin construct in vivo. Alternatively, a composition may contain two or more different AAV vectors, each of which has packaged therein different expression cassettes. For example, the two or more different AAV may have different expression cassettes which express immunoglobulin polypeptides which assemble in vivo to form a single functional immunoglobulin construct. In another example, the two or more AAV may have different expression cassettes which express immunoglobulin polypeptides for different targets, e.g., two provide for two functional immunoglobulin constructs (e.g., an anti-Her2 immunoglobulin construct and a second anti-neoplastic immunoglobulin construct). I

A regimen as described herein may comprise, in addition to one or more of the combinations described herein, further combination with one or more of an anti-neoplastic biological drug, an anti-neoplastic small molecule drug, a chemotherapeutic agent, immune enhancers, radiation, surgery, and the like. A biological drug as described herein, is based on a peptide, polypeptide, protein, enzyme, nucleic acid molecule, vector (including viral vectors), or the like.

In one embodiment, the method further comprises the subject receives an immunosuppressive co-therapy. Immunosuppressants for such co-therapy include, but are not limited to, a glucocorticoid, corticosteroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent.

In certain embodiments, the immunosuppressive therapy may be started 0, 1, 2, 7, or more days prior to the gene therapy administration. Such therapy may involve co-administration of two or more drugs, the (e.g., prednelisone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected.

In one embodiment, the method further comprises administering to a subject anti-AAV neutralizing antibodies (NAb) to reduce peripheral transduction, and mitigate the potential risk of trastuzumab-induced cardiotoxicity. In certain embodiments, the method further comprises detect the presence of systemic AAV NAb prior to treating with anti-AAV NAb, wherein patients with levels of anti-AAV NAb in excess of a predetermined level against the rAAV capsid (or a sero-crossreactive capsid) do not require pretreatment. Such levels may be, e.g., in excess of about 1:10, about 1:20, about 1:50, about 1:100, about 1:250, or higher or lower levels. In certain embodiments, the method further comprises intravenously administering human anti-AAV polyclonal antibodies (e.g., plasma-derived, pooled human immunoglobulin (IVIG)), an anti-AAV monoclonal antibody, or a cocktail of anti-AAV antibodies, to a patient about 1 day to about 2 hours before treatment with a rAAV-trastuzumab, e.g., rAAV.trastuzumab-coGW.

In certain embodiments, a combination regimen is provided for preventing off-target delivery rAAV, the regimen comprising (a) pretreating the patient by systemically administering a composition comprising anti-AAV capsid neutralizing antibodies directed against an AAV capsid in a recombinant AAV vector, and (b) administering to the central nervous system (CNS) rAAV as described herein (e.g., rAAV.trastuzumab-coGW). See also, U.S. Provisional Patent Application No. 63/328,227, filed Apr. 6, 2022, which is incorporated herein by reference in its entirety.

As used herein, a “neutralizing antibody” or “NAb” binds specifically to a viral capsid or envelope and interferes with the infectivity of the virus or a recombinant viral vector having the viral capsid or envelope, thus preventing the recombinant viral vector from delivering effective amounts of a gene product encoded by an expression cassette in its vector genome. Various methods for assessing neutralizing antibodies in a patient's sera may be utilized. The term method and assay may be used interchangeably. As used herein, the term “neutralization assay” and “serum virus neutralization assay” refers to a serological test to detect the presence of systemic antibodies that may prevent infectivity of a virus. Such assays may also qualitatively or quantitatively discern the binding capacity (e.g., magnitude) or efficiency of the antibodies to neutralize a target. Immunological assays may include enzyme immunoassay (EIA), radioimmunoassay (RIA), which uses radioactive isotopes, fluoroimmunoassay (FIA) which uses fluorescent materials, chemiluminescent immunoassay (CLIA) which uses chemiluminescent materials and counting immunoassay (CIA) which employs particle-counting techniques, other modified assays such as western blot, immunohistochemistry (IHC) and agglutination. One of the most common enzyme immunoassays is enzyme-linked immunosorbent assay (ELISA).

Example of suitable methods include those described, e.g., R Calcedo, et al, Journal Infectious Diseases, 2009, 199:381-290; GUO, et al., “Rapid AAV_Neutralizing Antibody Determination with a Cell-Binding Assay”, Molecular Therapy: Methods & Clinical Development Vol. 13 Jun. 2019, T. Ito et al, “A convenient enzyme-linked immunosorbent assay for rapid screening of anti-adeno-associated virus neutralizing antibodies”, Ann Clin Biochem 2009; 46: 508-510; US 2018/0356394A2 (Voyager Therapeutics). Additionally, commercial kits exist (see, e.g., Athena Diagnostics, Invitrogen, ThermoFisher.com; Covance).

The neutralization ability of an antibody is usually measured via the expression of a reporter gene such as luciferase or GFP. In order to determine and compare the activity of a neutralizing antibody, the antibody tested should display a neutralizing activity of 50% or more in one of the neutralization assays described herein. In some examples, neutralizing capacity is determined by measuring the activity of a reporter gene product (e.g., luciferase, GFP). The neutralizing capacity of an antibody to a specific viral vector may be at least 50%, e.g., at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

Still other co-therapeutics may include, e.g., anti-IgG enzymes, which have been described as being useful for depleting anti-AAV antibodies (and thus may permit administration to patients testing above a threshold level of antibody for the selected AAV capsid), and/or delivery of anti-FcRN antibodies and/or one or more of a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon. anti-FcRN antibodies include, e.g., rozanolixizumab (UCB7665) (UCB SA); IMVT-1401, RVT-1401 (HL161), HBM9161 (all form HanAll BioPhrma Co. Ltd), Nipocalimab (M281) (Momenta Phannaceuticals Inc), ARGX-113 (efgartigimod) (Argenx S.E.), orilanolimab (ALXN 1830, SYNT001, Alexion Pharmaceuticals Inc), SYNT002, ABY-039 (Affibody AB), or DX-2507 (Takeda Pharmaceutical Co. Ltd). In certain embodiments, a combinations of anti-FcRN antibodies is administered. In certain embodiments, an anti-FcRN antibody is administered in combination with a suitable anti-FcRn ligand (i.e., a peptide or protein construct binding human FcRn so as to inhibit IgG binding).

In one embodiment, a combination regimen for treating a patient with Her2-positive tumor or Her2-positive metastatic tumor is provided, wherein the regiment includes administering a vector describe herein in combination with a ligand which inhibits binding of human FcRn and pre-existing patient neutralizing antibodies (e.g., lgG). In certain embodiments, the patient may be naïve to any therapeutic treatment with a vector and may have pre-existing immunity due to prior infections with a wild-type virus. In other embodiments, the patient may have neutralizing antibodies as a result of a prior treatment or vaccination. In certain embodiments, the patient may have neutralizing antibodies 1:1 to 1:20, or in excess of 1:2, in excess of 1:5, in excess of 1:10, in excess of 1:20, in excess of 1:50, in excess of 1:100, in excess of 1:200, in excess of 1:300 or higher. In certain embodiments, a patient has neutralizing antibodies in the range of 1:1 to 1:200, or 1:5 to 1:100, or 1:2 to 1:20, or 1:5 to 1:50, or 1:5 to 1:20. In certain embodiments, a patient receives a single anti-FcRn ligand (e.g., anti-FcRn antibody) as the sole agent to modulate FcRn-IgG binding and to permit effective vector delivery. In other embodiments, a patient may receive a combination of one or more anti-FcRn ligands and a second component (e.g., an Fc receptor down-regulator (e.g., interferon gamma), an IgG enzyme, or another suitable component). Such combinations may be particularly desirable for patients having particularly high neutralizing antibody levels (e.g., in excess of 1:200).

In certain embodiments, an anti-FcRn ligand(s) (e.g., antibodies) is administered to a patient having neutralizing antibodies prior to and, optionally, concurrently with a selected viral vector. In certain embodiments, continued expression of an anti-FcRn ligand post administration of the gene therapy vector may desired on a short-term (transient basis), e.g., until such time as the viral vector clears from the patient. In certain embodiments, persistent expression of an anti-FcRn ligand may be desired. Optionally, in this embodiment, the ligand may be delivered via a viral vector, including, e.g., in the viral vector expressing the therapeutic transgene. However, this embodiment is not desirable where the therapeutic gene being delivered is an antibody or antibody construct or another construct comprising an IgG chain. In such embodiments, where an antibody construct having an IgG chain is being delivered via a viral vector to a patient having pre-existing immunity, the anti-FcRn ligand is delivered or dosed transiently so that the amount of anti-FcRn ligand in the circulation is cleared from the sera before effective levels of vector-mediated transgene product are expressed.

In certain embodiments, the FcRn ligand is delivered one to seven days prior to administration of the vector (e.g., rAAV). In certain embodiments, the FcRn ligand is delivered daily. In certain embodiments, the FcRn ligand (e.g., immunoglobulin construct(s)) is delivered on the same day as the vector is administered. In certain embodiments, the FcRn ligand (e.g., immunoglobulin construct(s)) is delivered at least one day to four weeks post-rAAV administration. In certain embodiments, the ligand is delivered for four weeks to six months post-rAAV administration. In certain embodiments, the ligand is dosed via a different route of administration than the rAAV. In certain embodiments, the ligand is dosed orally, intravenously, or intraperitoneally. See also, International Patent Application No. PCT/US2021/037575, filed Jun. 16, 2021, and now published WO 2021/257668 A1, which is incorporated herein by reference in its entirety.

In one embodiment, the compositions described herein are used in a method for retarding the growth of a tumor, wherein the tumor is a metastatic HER2+ cancer in brain. In one embodiment, the compositions described herein are used in a method for retarding the growth of a tumor, wherein the tumor is a metastatic HER2+ breast cancer in brain. In one embodiment, the compositions described herein are used in a method for retarding the growth of a tumor, wherein the tumor is a metastatic HER2+ gastrointestinal cancer in brain. In still another embodiment, the compositions described herein are useful for decreasing tumor size in a subject. In a further embodiment, the compositions described herein are useful in reducing the number of cancer cells in a non-solid tumor cancer. In another embodiment, the compositions described herein are useful in prophylaxis. In one embodiment, the compositions described herein are used in a method for preventing development of metastasis in patient with HER2+ tumors, which patients are at risk of developing metastasis. In one embodiment, the compositions described herein are used in a method for preventing development of brain metastasis in patient with advanced HER2+ tumors, which patients are at risk of developing brain metastasis. In one embodiment, the compositions described herein are used in a method for preventing development of brain metastasis in patient with d HER2+ tumors following stereotactic radiosurgery, i.e., prevent recurrence in patients with HER2+ breast cancer brain metastasis. In another embodiment, a composition as provided herein is used in a method for increasing overall survival and/or progression-free survival in a patient. In certain embodiments, the compositions described herein are used in a method for treatment to delay brain metastasis progression in patients that achieved stable disease with standard of care therapies. In certain embodiments, the compositions described herein are used in a method for treatment to manage brain metastasis and mitigate symptoms in patients who have progressed.

Optionally, the AAV compositions as described herein are administered in the absence of an additional extrinsic pharmacological or chemical agent, or other physical disruption of the blood brain barrier.

In a combination therapy, the AAV-delivered immunoglobulin construct described herein is administered before, during, or after commencing therapy with another agent, as well as any combination thereof, i.e., before and during, before and after, during and after, or before, during and after commencing the anti-neoplastic therapy. For example, the AAV can be administered between 1 and 30 days, preferably 3 and 20 days, more preferably between 5 and 12 days before commencing radiation therapy. In another embodiment of the invention, chemotherapy is administered concurrently with or, more preferably, subsequent to AAV-mediated immunoglobulin (antibody) therapy. In still other embodiments, the compositions of the invention may be combined with other biologics, e.g., recombinant monoclonal antibody drugs, antibody-drug conjugates, or the like. Further, combinations of different AAV-delivered immunoglobulin constructs such as are discussed above may be used in such regimens.

Any suitable method or route can be used to administer an AAV-containing composition as described herein, and optionally, to co-administer anti-neoplastic agents and/or antagonists of other receptors. The anti-neoplastic agent regimens utilized according to the invention, include any regimen believed to be optimally suitable for the treatment of the patient's neoplastic condition. Different malignancies can require use of specific antitumor antibodies and specific anti-neoplastic agents, which will be determined on a patient-to-patient basis. Routes of administration include, for example, systemic, oral, intravenous, intraperitoneal, subcutaneous, or intramuscular administration. The dose of antagonist administered depends on numerous factors, including, for example, the type of antagonists, the type and severity tumor being treated and the route of administration of the antagonists.

### Kit

In certain embodiments, a kit is provided which includes a concentrated vector suspended in a formulation (optionally frozen), optional dilution buffer, and devices and components required for intrathecal, intracerebroventricular or intracisternal administration. In another embodiment, the kit may additional or alternatively include components for intravenous delivery. In one embodiment, the kit provides sufficient buffer to allow for injection. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

It should be understood that the compositions in kit described herein are intended to be applied to other compositions, regiments, aspects, embodiments and methods described across the Specification.

### Apparatus and Method for Delivery of a Pharmaceutical Composition

In one aspect, the vectors, rAAV or compositions thereof provided herein may be administered intrathecally via the method and/or the device provided in this section and described in WO 2017/136500 and WO 2018/160582, which are incorporated by reference herein. Alternatively, other devices and methods may be selected. In certain embodiments, the method comprises the steps of CT-guided sub-occipital injection via spinal needle into the cisterna magna of a patient. As used herein, the term Computed Tomography (CT) refers to radiography in which a three-dimensional image of a body structure is constructed by computer from a series of plane cross-sectional images made along an axis. In certain embodiments, the apparatus is described in US Patent Publication No. 2018-0339065 A1, published Nov. 29, 2019, which is incorporated herein by reference in its entirety. In certain embodiments, the vectors, rAAV or compositions thereof provided herein may be administered using Ommaya Reservoir.

It should be understood that the compositions in the device described herein are intended to be applied to other compositions, regiments, aspects, embodiments and methods described across the Specification.

As used herein, the phrases “ameliorate a symptom”, “improve a symptom” or any grammatical variants thereof, refer to reversal of a metastatic Her2+ cancer in brain-related symptoms. In one embodiment, the amelioration or improvement refers to the total number of symptoms in a patient after administration of the described composition(s) or use of the described method, which is reduced by about 5%, about 10%, about 20%, about 30%, about 40%, about 50%, about 60%, about 70%, about 80%, about 90%, about 95% compared to that before the administration or use. In another embodiment, the amelioration or improvement refers to the severity or progression of a symptom after administration of the described composition(s) or use of the described method, which is reduced by about 5%, about 10%, about 20%, about 30%, about 40%, about 50%, about 60%, about 70%, about 80%, about 90%, about 95% compared to that before the administration or use.

As used herein, the “conservative amino acid replacement” or “conservative amino acid substitutions” refers to a change, replacement or substitution of an amino acid to a different amino acid with similar biochemical properties (e.g., charge, hydrophobicity and size), which is known by practitioners of the art. Also see, e.g., FRENCH et al. What is a conservative substitution?Journal of Molecular Evolution, March 1983, Volume 19, Issue 2, pp 171-175 and YAMPOLSKY et al. The Exchangeability of Amino Acids in Proteins, Genetics. 2005 August; 170(4): 1459-1472, each of which is incorporated herein by reference in its entirety.

As used herein, the term “administration” or any grammatical variations thereof refers to delivery of composition described herein to a subject.

The words “comprise”, “comprises”, and “comprising” are to be interpreted inclusively rather than exclusively. The words “consist”, “consisting”, and its variants, are to be interpreted exclusively, rather than inclusively. While various embodiments in the specification are presented using “comprising” language, under other circumstances, a related embodiment is also intended to be included and described using “consisting of” or “consisting essentially of” language. As used throughout this specification and the claims, the terms “comprising”, “containing”, “including”, and its variants are inclusive of other components, elements, integers, steps and the like. Conversely, the term “consisting” and its variants are exclusive of other components, elements, integers, steps and the like.

It is to be noted that the term “a” or “an” refers to one or more. As such, the terms “a” (or “an”), “one or more,” and “at least one” are used interchangeably herein.

As used herein, the term “about” or “˜” refers to a variant of 10% from the reference integer and values therebetween, unless otherwise specified. For example, “about” 500 μM includes ±50 (i.e., 450-550, which includes the integers therebetween). For other values, particularly when reference is to a percentage (e.g., 90% of taste), the term “about” is inclusive of all values within the range including both the integer and fractions.

As described above, the term “about” when used to modify a numerical value means a variation of 10%, (±10%, e.g., ±1, ±2, 3, ±4, 5, 6, ±7, ±8, ±9, ±10, or values therebetween) from the reference given, unless otherwise specified.

In certain instances, the term “E+#” or the term “e+#” is used to reference an exponent. For example, “5E10” or “5e10” is 5×10¹⁰. These terms may be used interchangeably.

With regard to the description of various embodiments herein, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

### EXAMPLES

The following examples are provided to illustrate certain aspects of the claimed invention. The invention is not limited to these examples.

Breast cancer is the most common cause of brain metastases in women, affecting 15-30% of all patients. Despite the success of systemic Her2-targeted therapies, up to half of patients with Her2-positive (HER2-positive, HER2+, or her2+) primary breast cancer develop breast cancer brain metastases (BCBM). Subtype switching from Her2-primary breast tumors account for an additional 15% of Her2+ BCBM, bringing total Her2 positivity in the BCBM setting to nearly 65% of cases. HER2-positive BCBM can manifest in the leptomeninges (LM) or parenchyma. The LM lesions have direct access to cerebrospinal fluid (CSF). LM disease represents 8% of the total cases of HER2-positive BCBM. Even though direct delivery of Her2-targeted therapies to the central nervous system (CNS) has shown promising clinical results, long-term feasibility is limited, at least in part, by the need for repeated dosing to overcome the shorter drug half-life in the cerebrospinal fluid vs. systemic circulation (e.g., about 1 vs. 5 days, respectively).

Trastuzumab, also referred to as Herceptin, is a humanized monoclonal antibody (mAb) against HER2, developed by Genentech and commercialized by Genentech/Roche. Trastuzumab is indicated for treatment of HER2+ breast cancer and for metastatic breast cancer, both in combination with chemotherapy or as a single agent (after having received chemotherapy). Trastuzumab plus chemotherapy increase both progression-free survival and overall survival for patients with metastatic breast cancer. Systemic administration of trastuzumab is not indicated for the treatment of BCBM as it cannot cross the blood-brain-barrier.

To achieve sustained CNS expression of trastuzumab (Herceptin) without frequent dosing, we developed a gene therapy approach to treat BCBM comprising a single local administration of AAV vector encoding trastuzumab. Previous work has shown that intracerebroventricular (ICV) injection of trastuzumab-expressing AAV doubled the life expectancy of Rag1 knockout (KO) mice bearing BT-474 breast cancer brain tumors. Rag1 KO mice who received AAV-trastuzumab prior to implantation of BT-474 tumor cells into the brain showed a dose-dependent delay in tumorigenesis. See also, International Patent Application No. PCT/US2015/027491, filed Apr. 24, 2015, published Oct. 29, 2015, as WO 2015/164723A1.

In current studies, we performed extensive gene therapy vector engineering and in vivo evaluation. The concentration of trastuzumab achieved in the brain parenchyma after a single ICV administration of the engineered vectors into Rag1 KO mice ranged from 10-165 ng/mg of brain protein by day 28.

Previous studies utilizing CNS delivery of gene therapy vectors have shown vector leakage into the systemic circulation, which results in detectable transgene expression in the blood following peripheral transduction. To mitigate the potential risk of trastuzumab-induced cardiotoxicity, we investigated using intravenously administered, plasma-derived, pooled human immunoglobulin (IVIG) as a source of anti-AAV neutralizing antibodies to reduce peripheral transduction. Compared to controls that received naïve mouse serum, Rag1 KO mice receiving IVIG two hours prior to ICV administration of AAV-trastuzumab exhibited similar levels of trastuzumab within the brain while displaying a 10-fold reduction in serum trastuzumab concentrations. Vector biodistribution analysis suggested that reduced serum trastuzumab levels resulted from effective blockade of liver transduction following IVIG pre-treatment.

These data suggest that AAV vector administration to the CNS to achieve sustained expression of trastuzumab may effectively treat Her2+ BCBM. Additionally, patients with pre-existing neutralizing antibodies to the AAV vector capsid need not be excluded from future clinical trials.

### Example 1: Production of rAAV Comprising Engineered Trastuzumab

In the studies herein, various engineered sequences encoding trastuzumab were generated, and comparative studies were performed. The rAAV are generated using triple transfection techniques, utilizing (1) a cis plasmid encoding AAV2 rep proteins and the AAVhu68 VP1 cap gene (alternatively AAVrh91, AAVhu95, or AAV9), (2) a cis plasmid comprising adenovirus helper genes not provided by the packaging cell line which expresses adenovirus Ela, and (3) a trans plasmid containing the vector genome for packaging in the AAV capsid. See, e.g., US 2020/0056159. The trans plasmid is designed to contain either the vector genome comprising engineered trastuzumab sequence comprising IL-2 signal peptide and T2A element, optionally comprising a furin cleavage sequence. The vector genomes include (among others): (i) (SEQ ID NO: 1); (ii) (SEQ ID NO: 25); (iii) (SEQ ID NO: 27); or (iv) (SEQ ID NO: 50).

The vector genome contains an AAV 5′ inverted terminal repeat (ITR) and an AAV 3′ ITR at the extreme 5′ and 3′ end, respectively. The ITRs flank the sequences of the expression cassette packaged into the AAV capsid which have sequence encoding a trastuzumab. The expression cassette further comprises regulatory sequences operably linked to the fusion protein coding sequences, including a promoter (chicken beta actin promoter, CB7 hybrid promoter comprising CMV IE enhancer, chicken beta actin promoter, and a chimeric intron comprising chicken beta actin intron, or a Ubiquitin C (UbC) promoter), an intron (chicken beta actin intron or chimeric intron comprising a Promega intron), SV40 polyA.

AAV vector technology allows for sufficient expression of the engineered trastuzumab transgene within the brain as directed using a variety of promoters. Expression of trastuzumab from an AAV vector provide an effective one-time gene therapy approach for the prevention and/or treatment of HER2+ BCBM. The current treatment strategies for HER2+ BCBM uses different combinations of HER2 modulatory agents (anti-HER2 monoclonal antibodies and kinase inhibitors), as well as chemotherapeutic drugs. Moreover, intracranial delivery of AAV-trastuzumab in HER2+ breast cancer patients at high risk for the development of BCBM could prevent or significantly delay the emergence of tumor lesions. CNS delivery of AAV-trastuzumab (engineered trastuzumab coding sequence) alone or in combination with standard-of-care systemic HER2 kinase inhibitors could be used as an alternative approach to achieve disease remission or stabilization since it could avoid treatment scape and result in long lasting trastuzumab expression.

### Example 2: rAAV Evaluation in Mice

### Introduction

The human epidermal growth factor receptor (EGFR) tyrosine kinase family is composed of four closely related membrane-bound cell surface receptors: EGFR, HER2, HER3 and HER4. Ligand-dependent homo or heterodimerization of these receptors results in phosphorylation of intracellular tyrosine kinase domains and activation of signaling pathways that promote cell proliferation, migration, and differentiation. Among the EGFR family proteins, HER2 is the only constitutively active orphan receptor, for which no ligand is known; HER2 also has the strongest catalytic activity and is the preferred dimerization partner for all other members of the EGFR family. While HER2 expression is required for normal mammalian embryonic development, aberrant HER2 signaling is a known oncogenic driver. Amplification/overexpression of HER2 promotes tumorigenesis by hyperactivating oncogenic RAS-MAPK and PI3/AKT/mTOR signaling pathways. HER2 has been found to be amplified/overexpressed in several types of tumors (e.g., breast, gastric, esophageal, and ovarian cancers), and is often associated with aggressive tumor phenotypes and higher recurrence risk.

HER2 positivity is observed in 15-20% of all invasive breast cancer cases. Up to half of patients with HER2+ breast cancer will go on to develop breast cancer brain metastases (BCBM). Subtype switching arising from HER2-primary breast tumors accounts for an additional 15% of HER2+ BCBM, bringing total HER2 positivity in the BCBM setting to nearly 65% of cases. Targeting of advanced HER2+ breast cancer using regimens containing trastuzumab (Herceptin®), an anti-HER2 humanized monoclonal antibody, have been associated with remarkable extension of survival (1.5 years in 2001 vs. about 5 years currently); however, median overall survival of patients diagnosed with HER2+ BCBM is only 29.1 months despite up-to-date treatment (trastuzumab+ tucatinib (HER2 tyrosine kinase inhibitor)+ capecitabine). This discrepancy may be explained, at least in part, by the presence of an intact blood brain barrier, which blocks large molecular weight biologics such as trastuzumab from entering the brain parenchyma (420:1 ratio of trastuzumab levels in the serum compared to the cerebrospinal fluid (CSF)). Due to subtherapeutic drug concentrations in the brain, HER2+ breast cancer patients receiving systemic antibody therapy are at an increased risk of developing BCBM.

Alternatively, direct delivery of HER2-targeted therapies into the central nervous system (CNS) has shown promising clinical results. A case report presented stabilization of brain and epidural metastatic lesions (>6 months) in a 34-year-old patient with HER2+ BCBM treated with repeated intraventricular injections of trastuzumab. Another case report has shown significant improvement of general health condition (˜11 months) in a 39-year-old patient with HER2+ breast cancer and leptomeningeal carcinomatosis following repeated intrathecal infusions of trastuzumab [Bousquet, G., et al., Intrathecal Trastuzumab Halts Progression of CNS Metastases in Breast Cancer, Journal of Clinical Oncology, 2016, 34(16):e151-e154, epub Jun. 1, 2016]. Nonetheless, long-term feasibility of this approach is limited by the need for repeated drug administrations to overcome its dramatically shorter half-life in the CSF vs. systemic circulation (about 1 vs. 5 days, respectively).

### Trastuzumab Expression Studies

To achieve sustained CNS expression of trastuzumab, we developed a gene therapy approach to treat HER2+ BCBM comprising a single local administration of an adeno-associated viral (AAV) vector encoding for trastuzumab (engineered coding sequence, i.e., Trastuzumab-coGW). To evaluate trastuzumab expression efficiency within the CNS, we engineered 16 new AAV-trastuzumab vectors. All vector iterations were packaged into AAV rhesus 91 (AAVrh91) capsid and injected via the intracerebroventricular (ICV) route in 7-8-week-old adult female Rag1 knockout (KO) mice (N=4 to 5 mice/vector group) for subsequent gene expression studies. Phosphate buffered saline (PBS) injected Rag1 KO mice were used as vehicle sham control group (N=5 mice). The Rag1 KO mouse model was chosen for this purpose since it is the preferred model for tumor challenge studies to confirm HER2 targeting using human-derived xenograft tumor models. Sub-M blood collection was performed on days −1, 7, 14, 21 and 28. Euthanasia was performed on day 28 via whole body perfusion using PBS.

To quantify trastuzumab expression, we conducted a 28-day time-course ELISA (enzyme linked immunosorbent assay) analysis study using serum and brain homogenates (at endpoint) from whole-body-perfused mice treated ICV with 1×10¹¹ genome copies (GC)/mouse of each AAVrh91-trastuzumab vector or PBS (Table 1 below).

TABLE 1

| | Trastuzumab | Trastuzumab |
|---|---|---|
| | (μg/mL) | (ng/mg protein) |
| Vector | in serum | in brain |
| V1 | 25.2917 | 9.1174 |
| AAVrh91.CB-CI.IL2_V1_Trastuzumab- | 12.1915 | 25.9944 |
| coGW.SV40 | | |
| V2 | 32.2827 | 11.5211 |
| V3 | 30.0614 | 4.0954 |
| V4 | 17.6228 | 2.4468 |
| AAVrh91.CB-CI.IL2_V2_Trastuzumab- | 22.8378 | 5.3583 |
| coGW.SV40 | | |
| V5 | 31.6432 | 0.0709 |
| V6 | 36.4897 | 2.6925 |
| PBS | — | — |

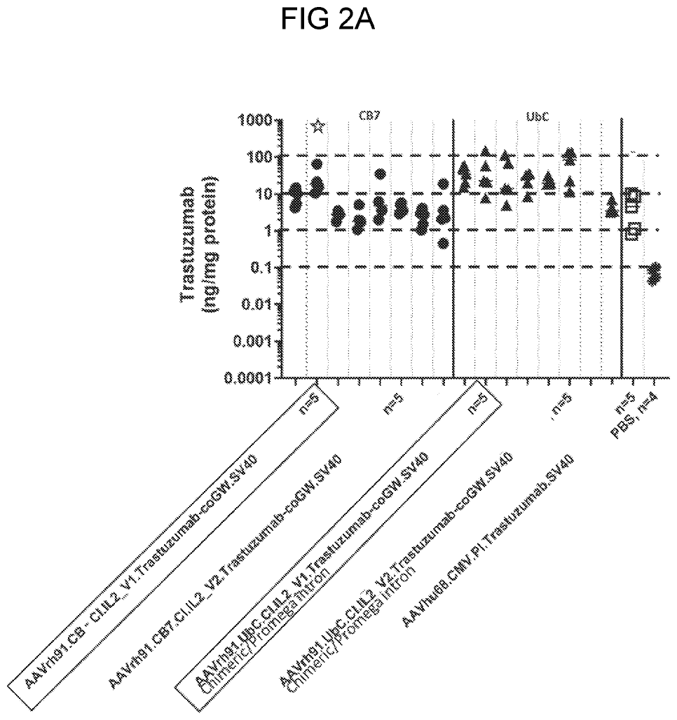

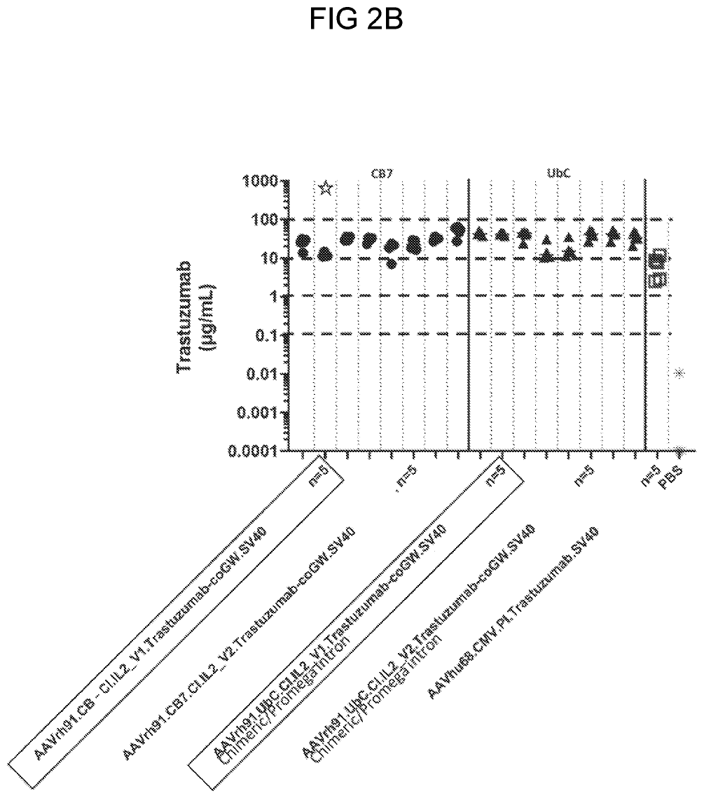

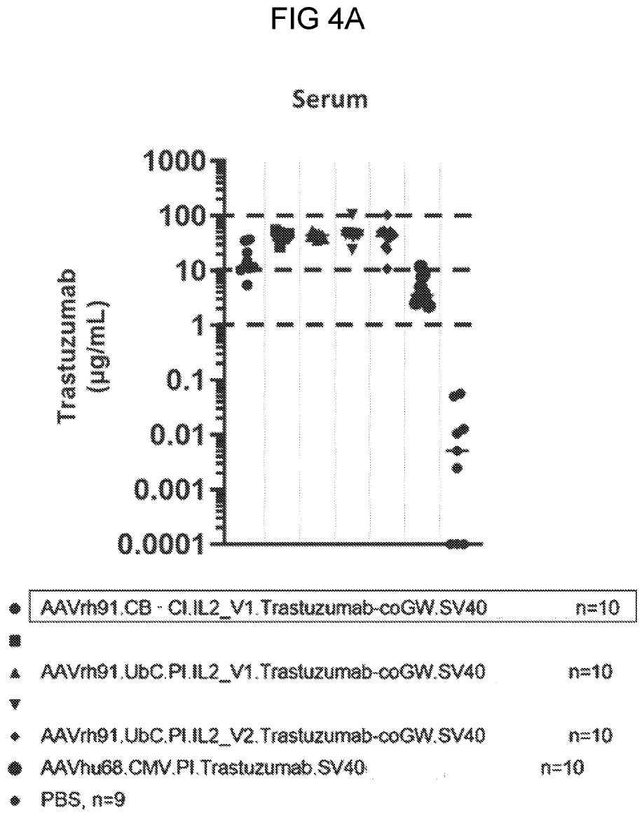

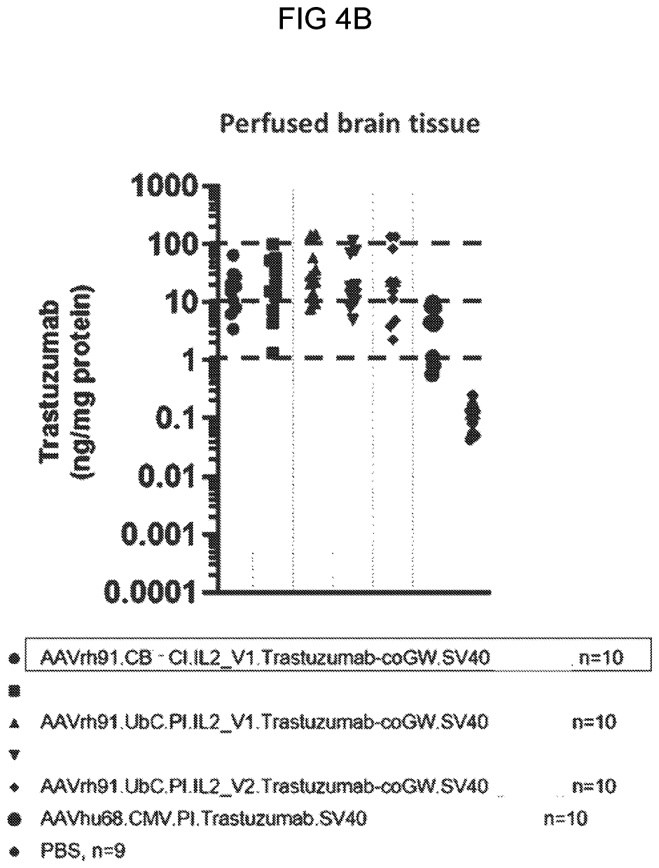

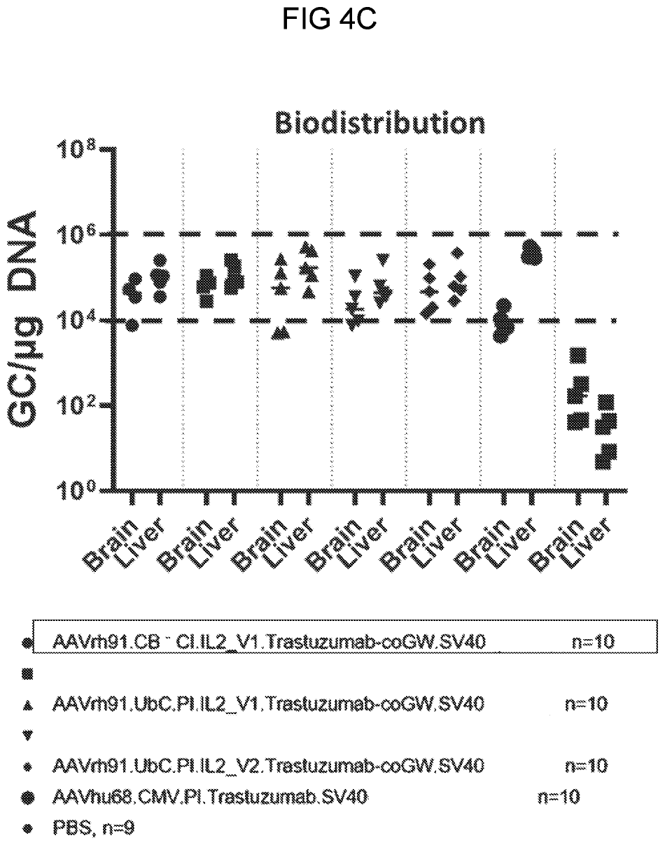

FIG. 2A shows results of the ELISA with plotted measurements of trastuzumab concentration (ng/mg protein) in the perfused brain tissue samples collected post administration with AAVrh91.CB-CI.IL2_V1_Trastuzumab-coGW.SV40 and AAVrh91.UbC.CI.IL2 V1 Trastuzumab-coGW.SV40 (Promega intron) in mice as compared to AAVhu68.CMV.PLTrastuzumab.SV40 (previously examined construct) and PBS. FIG. 2B shows results of the ELISA with plotted measurements of trastuzumab concentration (g/mL) in the serum samples collected post administration with AAVrh91.CB-CI.IL2_V1_Trastuzumab-coGW.SV40 and AAVrh91.UbC.CI.IL2_V1_Trastuzumab-coGW.SV40 (Promega intron) in mice as compared to AAVhu68.CMV.PI.Trastuzumab.SV40 (previously examined construct) and PBS. Mean peak serum concentration in patients receiving 500 mg (highest dose) were 377 μg/mL. FIG. 4A shows further data of measurements of trastuzumab concentration in collected serum samples at day 28 post administration with AAVrh91.CB-CI.IL2_V1_Trastuzumab-coGW.SV40, AAVrh91.UbC.Pl.IL2_V1_Trastuzumab-coGW.SV40 and AAVrh91.UbC.PI.IL2_V2_Trastuzumab-coGW.SV40 in mice as compared to AAVhu68.CMV.PI.Trastuzumab.SV40 (previously examined construct) and PBS. FIG. 4B shows further data of measurements of trastuzumab concentration in collected perfused brain tissue samples at day 28 post administration with AAVrh91.CB-CI.IL2_V1_Trastuzumab-coGW.SV40, AAVrh91.UbC.PI.IL2_V1_Trastuzumab-coGW.SV40 and AAVrh91.UbC.PI.IL2_V2_Trastuzumab-coGW.SV40 in mice as compared to AAVhu68.CMV.PLTrastuzumab.SV40 (previously examined construct) and PBS.

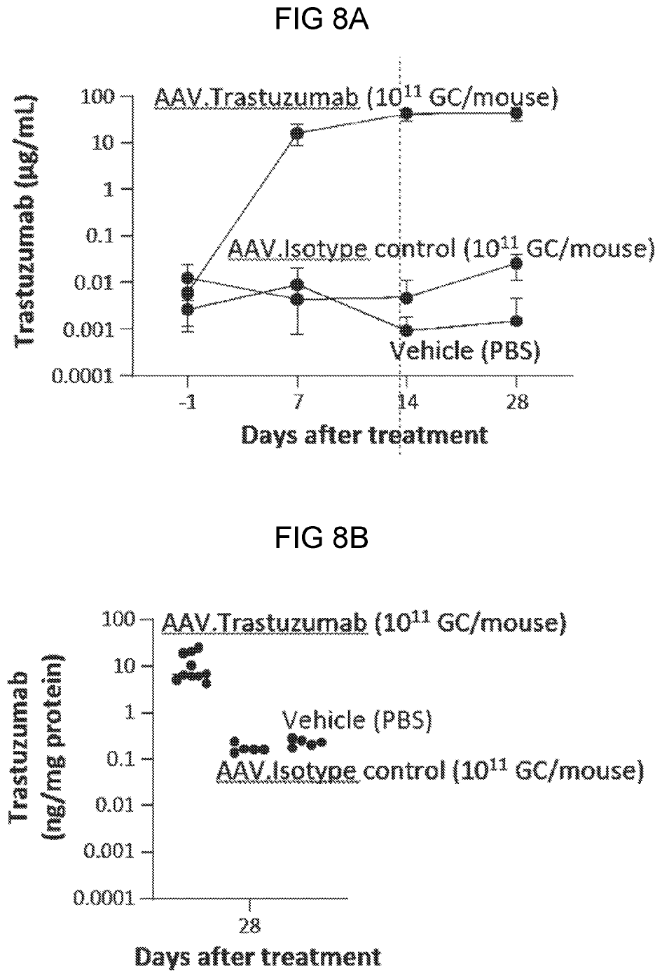

Additionally, FIG. 8A shows that ICV Injection of AAV Resulted in Sustained Expression of Trastuzumab in Rag11KO Mice as measured with ELISA in serum samples. FIG. 8B shows that ICV Injection of AAV Resulted in Sustained Expression of Trastuzumab in Rag1KO Mice as measured with ELISA in brain homogenate (perfused) samples.

Furthermore, we assessed vector biodistribution within the brain and liver to help determine the extent of brain and peripheral transduction following ICV administration (Table 2, below).

TABLE 2

| | | | | | Total |
|---|---|---|---|---|---|
| | | Trastuzumab | | GC per | RNA |
| | Mouse | ng/mg | GC/μg | diploid | (copies/100 |
| Treatment group | ID | protein | DNA | cell | ng) |
| AAVrh91.CB.CI.IL2_V1.Trastuzumab- | 641 | 15.28 | 1.75E+05 | 0.876 | 2.20E+02 |
| coGW.SV40 | 642 | 64.56 | 1.11E+04 | 0.055 | 2.33E+04 |
| | 643 | 20.94 | 1.38E+04 | 0.069 | 1.94E+03 |
| | 644 | 18.62 | 1.91E+05 | 0.953 | 6.85E+05 |
| | 645 | 10.58 | 4.55E+05 | 2.274 | 4.14E+02 |
| AAVrh91.UbC.CI.IL2_V1.Trastuzumab- | 2901 | 57.36 | 2.71E+04 | 0.136 | 1.19E+03 |
| coX.SV40 | 2902 | 34.88 | 1.17E+04 | 0.059 | 6.76E+04 |
| (Chimeric/Promega | 2903 | 20.77 | 2.92E+04 | 0.146 | 5.44E+04 |
| intron) | 2904 | 52.94 | 2.95E+05 | 1.476 | 3.29E+03 |
| | 2905 | 15.30 | 8.50E+04 | 0.425 | 5.05E+03 |
| AAVrh91.UbC.CI.IL2_V1.Trastuzumab- | 2316 | 45.26 | 1.47E+06 | 7.359 | 4.34E+04 |
| coGW.SV40 | 1247 | 44.05 | 1.95E+05 | 0.976 | 8.30E+02 |
| (Chimeric/Promega | 1218 | 37.85 | 9.36E+04 | 0.468 | 5.09E+04 |
| intron) | 2319 | 41.80 | 8.07E+04 | 0.404 | 1.14E+04 |
| | 2320 | 40.99 | 1.63E+05 | 0.813 | 1.83E+04 |
| AAVrh91.UbC.CI.IL2_V1.Trastuzumab- | 2906 | 14.18 | 6.40E+05 | 3.198 | 3.02E+05 |
| coY.SV40 | 2907 | 112.62 | 3.05E+04 | 0.153 | 3.56E+03 |
| (Chimeric/Promega | 2908 | 69.00 | 4.72E+04 | 0.236 | 6.56E+05 |
| intron) | 2909 | 13.68 | 9.08E+03 | 0.045 | 1.39E+03 |
| | 2910 | 5.06 | 3.52E+03 | 0.018 | 1.08E+05 |
| AAVrh91.UbC.CI.IL2_V2.Trastuzumab- | 2321 | 51.61 | 3.04E+04 | 0.152 | 8.87E+05 |
| coGW.SV40 | 1222 | 49.94 | 3.80E+04 | 0.190 | 5.29E+04 |
| (Chimeric/Promega | 1223 | 41.56 | 3.22E+04 | 0.161 | 3.21E+04 |
| intron) | 1224 | 27.12 | 5.29E+04 | 0.264 | 4.47E+05 |
| | 2325 | 38.24 | 5.15E+04 | 0.258 | 9.36E+03 |
| AAVhu68.CMV.PI.Trastuzumab.SV40 | 2921 | 4.61 | 7.15E+03 | 0.036 | 4.90E+04 |
| | 2922 | 1.19 | 1.93E+04 | 0.096 | 3.24E+03 |
| | 2923 | 0.88 | 3.17E+05 | 1.586 | 9.55E+02 |
| | 2924 | 8.64 | 1.36E+06 | 6.793 | 4.94E+01 |
| | 2925 | 10.10 | 5.46E+03 | 0.027 | 1.11E+03 |

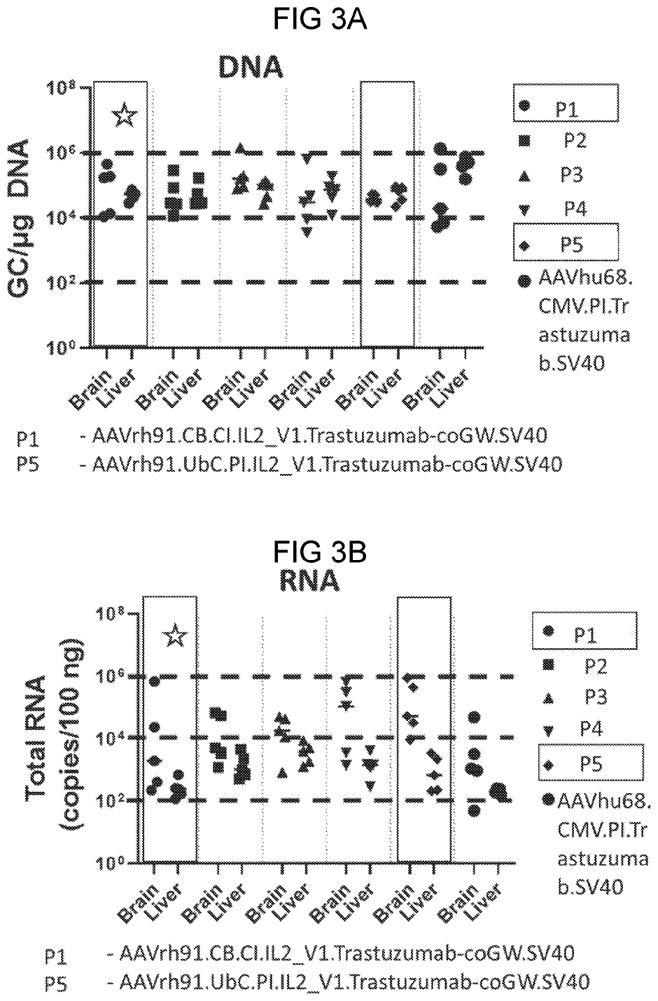

FIGS. 3A and 3B show a summarized view of data in Table 1. FIG. 3A shows results of a biodistribution study with plotted measurements of DNA concentration (GC/μg) in the collected liver and brain tissue. FIG. 3B shows results of a biodistribution study with plotted measurements of RNA concentration (copies/100 ng of RNA) in the collected liver and brain tissue. FIG. 4C shows further data of measurements of DNA biodistribution in collected brain and liver tissue samples at day 28 post administration with AAVrh91.CB-CI.IL2_V1_Trastuzumab-coGW.SV40, AAVrh91.UbC.PI.IL2_V1_Trastuzumab-coGW.SV40 and AAVrh91.UbC.PI.IL2_V2_Trastuzumab-coGW.SV40 in mice as compared to AAVhu68.CMV.PI.Trastuzumab.SV40 (previously examined construct) and PBS. All vectors tested were well tolerated in mice and raised no safety concerns.

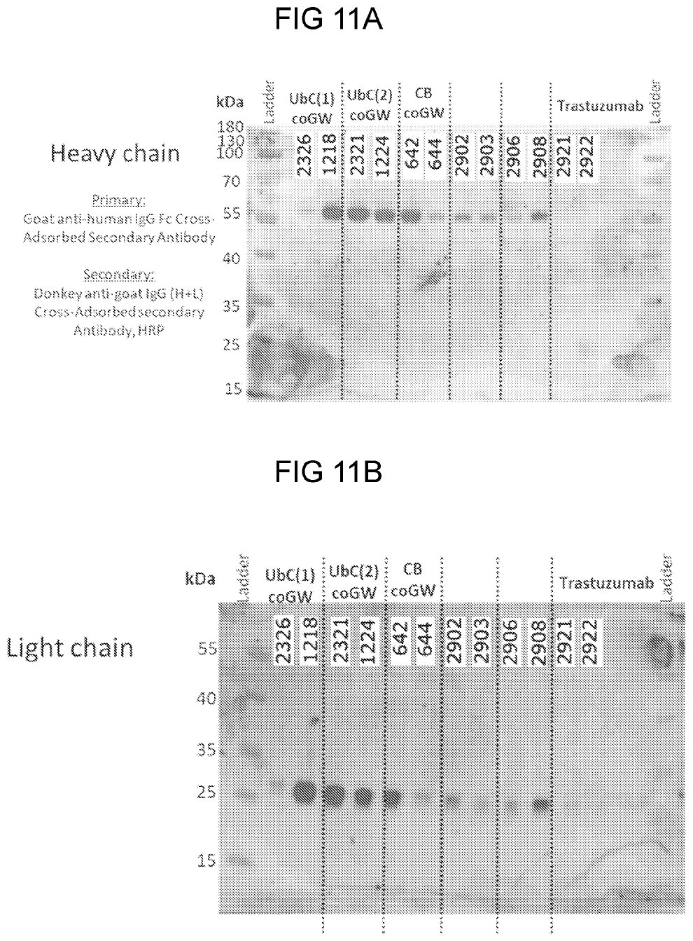

Trastuzumab was detected by ELISA in the serum and brain of mice following single ICV delivery of all 16 AAVrh91-trastuzumab vectors (FIGS. 2A and 2B). Among the CB promoter cohort, Trastuzumab-coGW construct encoding for a human engineered trastuzumab sequence (AAVrh91.CB7.CI.IL2_V1.Trastuzumab-coGW.SV40) resulted in highest expression in the brain and lowest systemic concentration in the serum at 28 days after ICV delivery (average of 26 ng of antibody per mg of protein). Western blot analysis suggests equimolar ratio of antibody heavy and light chain, suggesting accurate assembly (FIGS. 11A and 11B). FIGS. 11A and 11B shows a representative western blot confirming expression of the Trastuzumab heavy and light chains in brain lysates (e.g., FIGS. 2A and 2B).

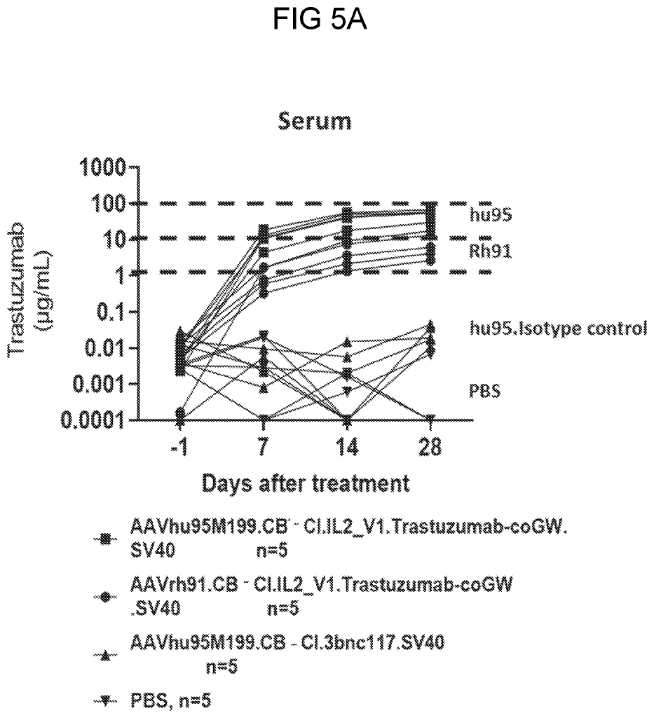

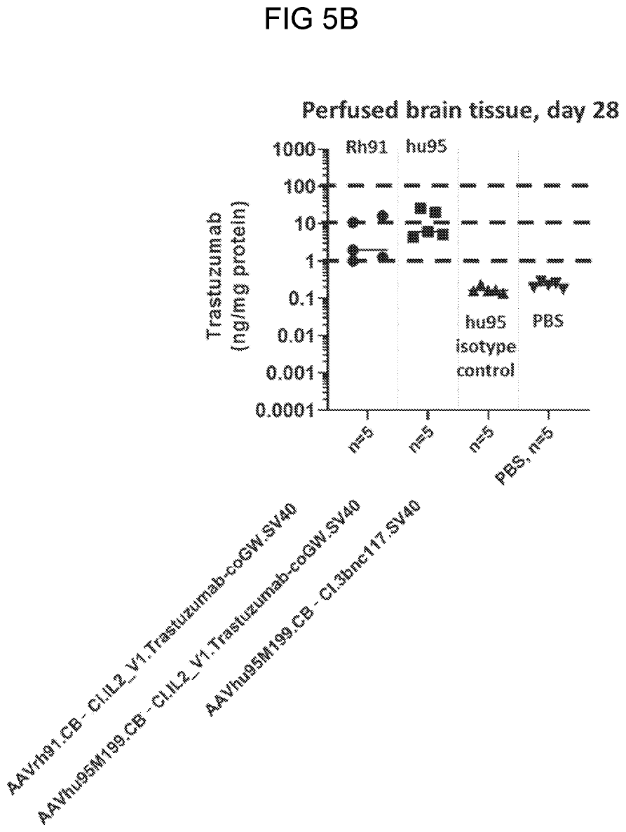

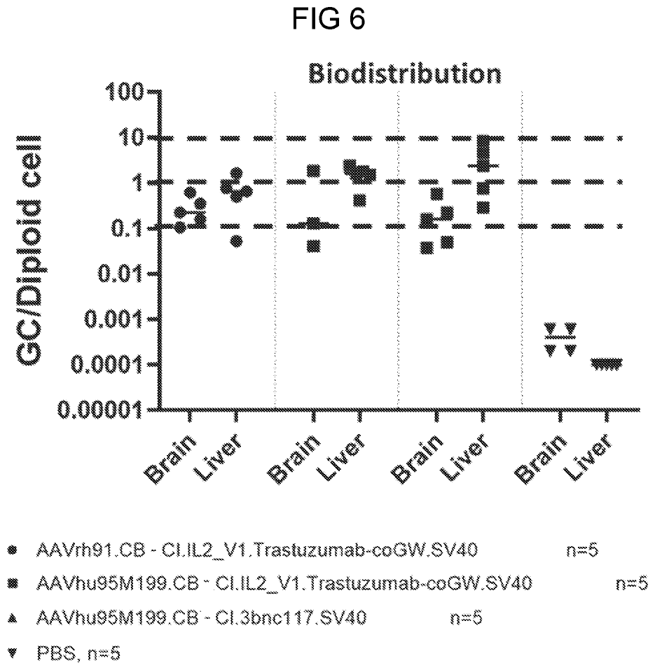

To further improve upon the current lead engineered vector, CB.CI.IL2_V1. Trastuzumab-coGW.SV40, we conducted studies to evaluate transduction efficiency of CB.CI.IL2_V1.Trastuzumab-coGW.SV40 package into AAVhu95 capsid. Briefly, in AAVhu95 capsid evaluation study, CB.CI.IL2_V1.Trastuzumab-coGW.SV40 and isotype control vector (i.e., 3bnc117 antibody encoding control vector) were packaged into AAVhu95 capsid and administered to mice via ICV injection. AAVhu95 transduction efficiency was evaluated via Trastuzumab expression using ELISA, and via fluorescent imaging. FIG. 5A shows expression levels of trastuzumab (g/mL) as measured in serum samples at day −1, 7, 14, and 28 post administration with AAVhu95.CB.CLIL2_V1.Trastuzumab-coGW.SV40, AAVrh91.CB.CI.IL2_V1.Trastuzumab-coGW.SV40 in comparison with capsid control and PBS. FIG. 5B shows expression levels of trastuzumab (g/mL) as measured in brain tissue samples at day −1, 7, 14, and 28 post administration with AAVhu95.CB.CI.IL2_V1.Trastuzumab-coGW.SV40, AAVrh91.CB.CI.IL2_V1. Trastuzumab-coGW.SV40 in comparison with capsid control and PBS. FIG. 6 shows vector biodistribution (GC/diploid cell) samples at day −1, 7, 14, and 28 post administration with AAVhu95.CB.CI.IL2 V1.Trastuzumab-coGW.SV40, AAVrh91.CB.CI.IL2_V1.Trastuzumab-coGW.SV40 in comparison with capsid control and PBS.

For the examination of rAAV.Trastuzumab-coGW (also referred to as AAVhu95.CB.CLIL2_V1.Trastuzumab-coGW.SV40 or AAV.Trastuzumab) preclinical activity, we used the mouse models as described in the Table 3, immediately below, which includes trastuzumab sensitivity and anticipated results. Briefly, the xenograft implantation procedure includes guide screw implantation, and tumor cell engraftment (at >1 week later), see also, Lal, et al., J Neurosurg 92:326-333, 2000.

TABLE 3

| BC xenograft | HR/HER2 | Trastuzumab | Anticipated |
|---|---|---|---|
| model | Status | sensitivity | results |
| BT-474 | ER+/PR+/HER2+ | Y | Remission |
| BT-474 Clone 5 | ER+/PR+/HER2+ | Y in vivo/N | Remission |
| (Trastuzumab | | in vitro | |
| resistant) | | | |
| MDA-MB-453 | ER−/PR−/HER2+ | Y | Remission |

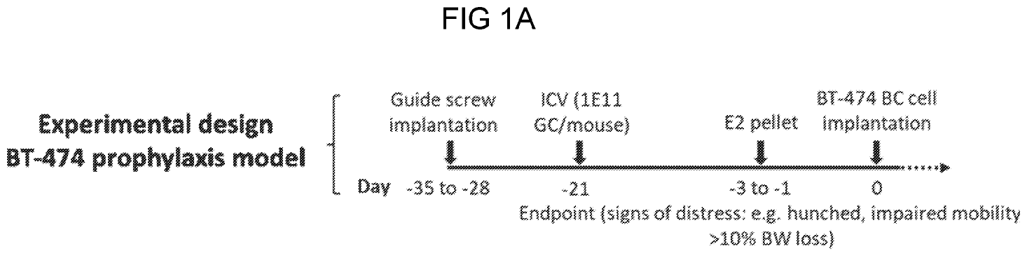

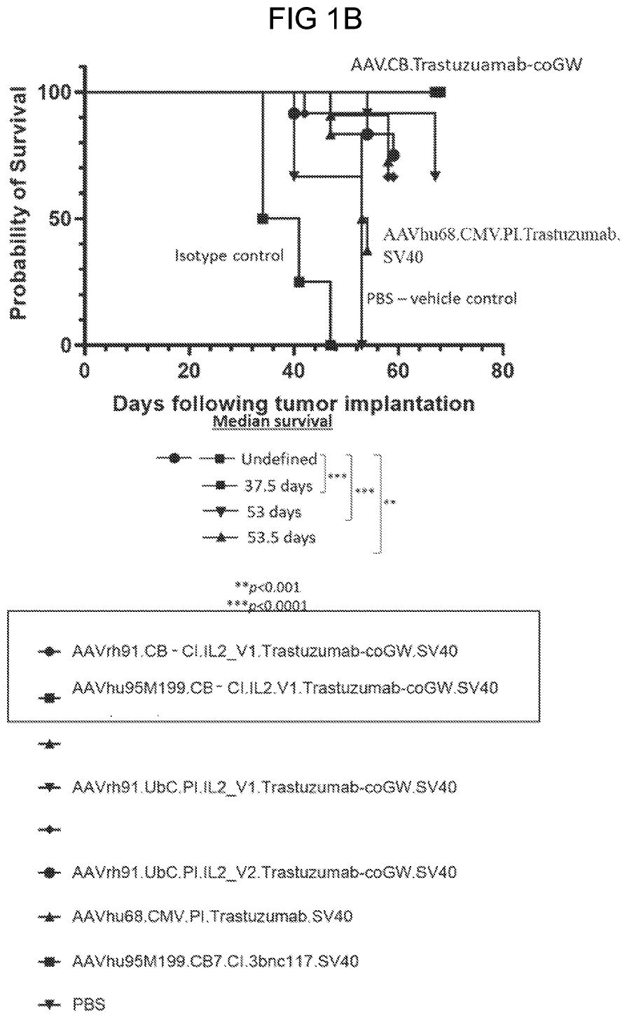

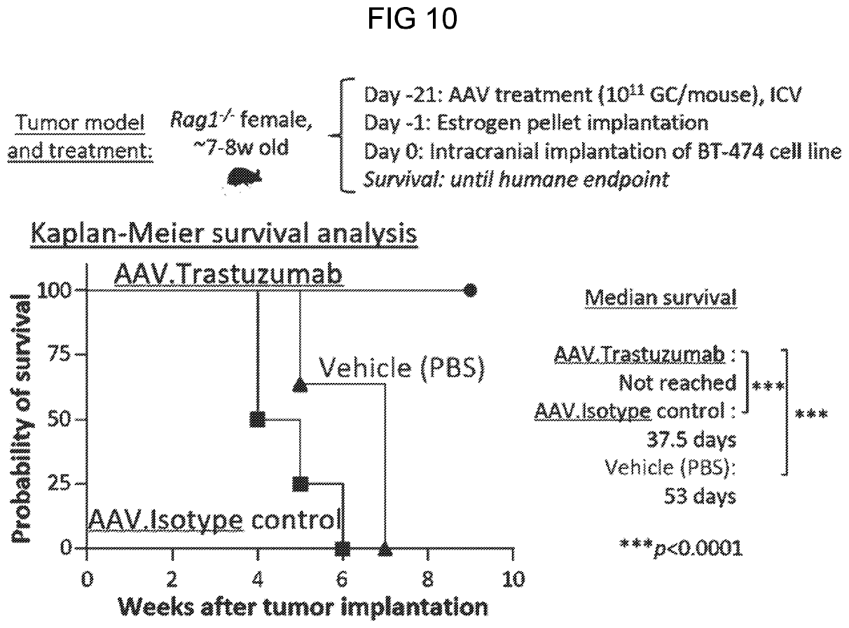

We examined preclinical activity in the prophylactic model and treatment model (also referred to as disease remission model). FIG. 1A shows a schematic representation for a designed study examining preclinical activity in mice. Briefly, in a prophylaxis model (prophylactic model with BT-474 (ER+/PR+/HER2+) cell line), at day −35 to −28 a guide screw was implanted, at day −21 (at 21 days prior to cell implantation), Rag1-KO female, about 7-8 weeks-old mice were treated with AAV at a dose of 10¹¹ GC/mouse via ICV injection, at day −1 the estrogen pellet was implanted, and at day 0 the intracranial implantation of BT-474 cell line was performed. The survival was monitored until a human endpoint. FIG. 1B shows results demonstrating preclinical activity of the engineered trastuzumab (anti-tumor activity against breast cancer brain metastasis) shown as a plot of the probability of survival in mice after tumor implantation. FIG. 10 shows Kaplan-Meier survival analysis (prophylactic treatment) of probability of survival in tumor bearing mice treated with AAVhu95.CB.CI.IL2.V1.Trastuzumab-coGW.SV40. For the endpoint, the signs of disease progression used included reduced mobility, hunched posture, weight loss. The median survival in the indicated groups were determined: PBS, n=12, 7 weeks (53 days); Isotype control, n=12, 5 weeks (37.5 days); AAV.Trastuzumab, n=12, not reached. These results show that a prophylactic treatment with AAVrh91.CB.CI.IL2_V1.Trastuzumab-coGW.SV40 and AAVhu95.CB.CI.IL2_V1.Trastuzumab-coGW.SV40 protected against HER2+ brain tumor development. Pre-treatment with AAV.Trastuzumab prevented brain xenograft progression with 100% survival beyond 10 weeks.

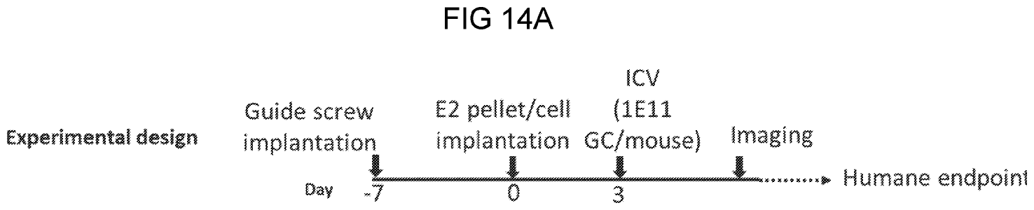

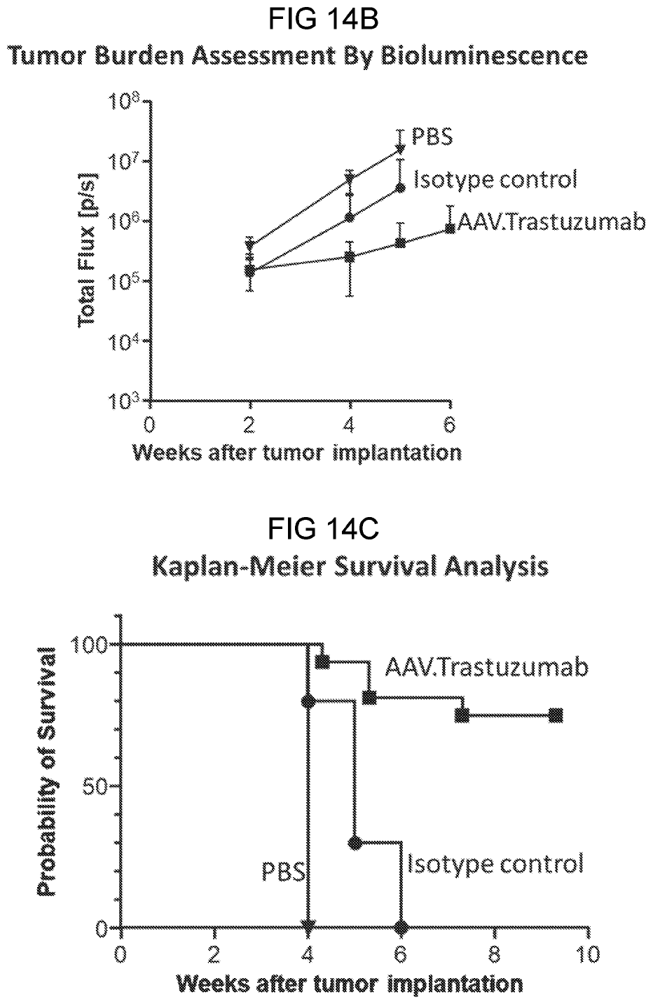

Furthermore, we examined preclinical activity (i.e., treatment of breast cancer brain xenografts with AAV.Trastuzumab) in a BT-474 (ER+/PR+/HER2+) mouse model. FIG. 14A shows a schematic representation for an experimental design of a study examining preclinical activity in mice. Briefly, on day −7 (i.e., 7 days prior to tumor cell implantation) a guide screw was implanted, on day 0 tumor cells were implanted, on day 3 (post tumor cell implantation) AAVhu95.CB.CI.IL2_V1.Trastuzumab-coGW.SV40 was administered ICV at a dose 1e11 (1×10¹¹ GC/mouse), following which imaging was performed to monitor tumor burden until humane endpoint. FIG. 14B shows quantified results of the tumor burden assessment (bioluminescence assessment) in mice xenograft (BT-474 (ER+/PR+/HER2+) xenograft) post treatment with AAVhu95.CB.CI.IL2.V1.Trastuzumab-coGW.SV40 in comparison with PBS, and isotype control. FIG. 14C shows Kaplan-Meier survival analysis of probability of survival in tumor bearing mice (BT-474 Clone 5 Trastuzumab Resistant (ER+/PR+/HER2+) Xenograft) treated with AAVhu95.CB.CJ.IL2.V1.Trastuzumab-coGW.SV40. Briefly, the human endpoint included signs of disease progression such as reduced mobility, hunched posture, weight loss. The median survival in the indicated groups were determined: PBS, n=8, 4 weeks; Isotype control, n=10, 5 weeks; AAV.Trastuzumab, n=14, Not reached. These results show that treatment with AAV.Trastuzumab on day +3 inhibited tumor progression.

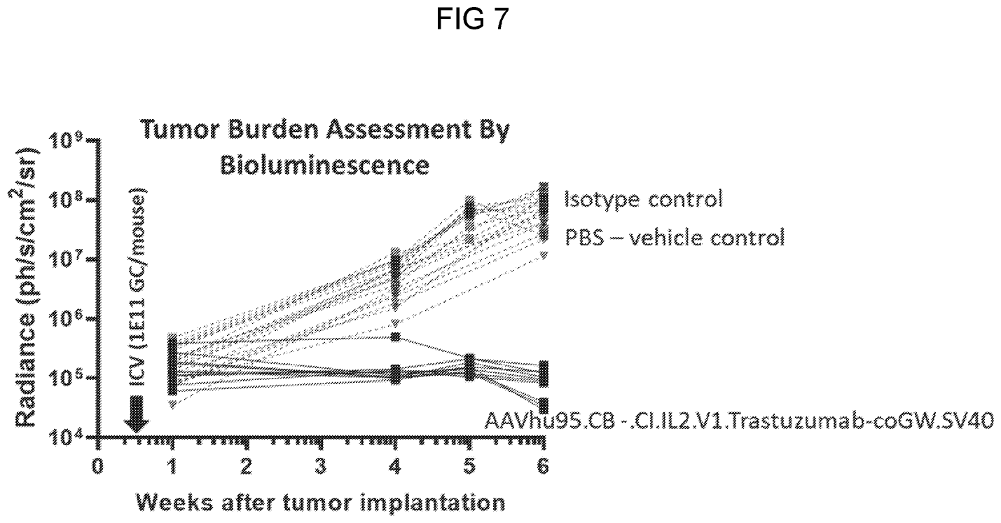

Furthermore, we examined preclinical activity in an MDA-MB-453 (ER−/PR−/HER2+) (also referred to as MDA-453; highly sensitive to trastuzumab) mouse model. Briefly, in disease remission model on day 0 an intracranial implantation of MDA-453 luciferase+ cell line was performed, and on day 3 the AAV treatment (10¹¹ GC/mouse) via ICV injection, wherein survival was examined until humane endpoint. FIG. 7 shows quantified results of the tumor bioluminescence assessment in mice xenograft (MDA-MB-453 (ER−/PR−/HER2+)) post treatment with AAVhu95.CB.CI.IL2.V1.Trastuzumab-coGW.SV40 in comparison with isotype control (Isotype control: 47 days; PBS: 44 days; rAAV.Trastuzumab-coGW: >6 weeks). Efficacy of rAAV.Trastuzumab-coGW injected following establishment of Her2+ cancer in mouse brain.

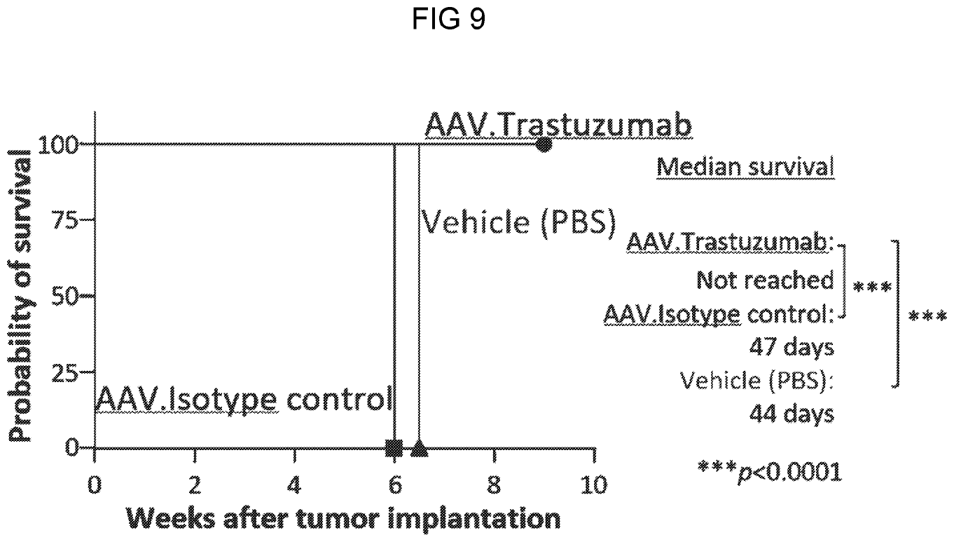

Complete tumor remission was observed, as measured by tumor growth (chart) and survival (inset). Representative bioluminescent images were taken at weeks 4, 5, and 6 for quantification analysis of the results of the tumor burden assessment by IVIS (bioluminescence assessment) in mice xenograft (MDA-MB-453 (ER−/PR−/HER2+)) post treatment with AAVhu95.CB.CI.IL2.V1.Trastuzumab-coGW.SV40 in comparison with isotype control (data not shown). Additionally, representative bioluminescent images were taken for quantification analysis of the results of the tumor bioluminescence assessment in mice xenograft (MDA-MB-453 (ER−/PR−/HER2+)) post treatment with AAVhu95.CB.CLIL2.V1.Trastuzumab-coGW.SV40 in comparison with isotype control. Further, FIG. 9 shows Kaplan-Meier survival analysis (disease remission) of probability of survival in tumor-bearing mice treated with AAVhu95.CB.CI.IL2.V1.Trastuzumab-coGW.SV40. The median survival in the indicated groups were determined: PBS, 6 weeks (44 days); Isotype control, 6 weeks (47 days); AAV.Trastuzumab, not reached. These results show that a complete disease remission with 100% survival beyond 10 weeks was achieved in tumor bearing mice treated with AAVhu95.CB.CI.IL2.V1.Trastuzumab-coGW.SV40.

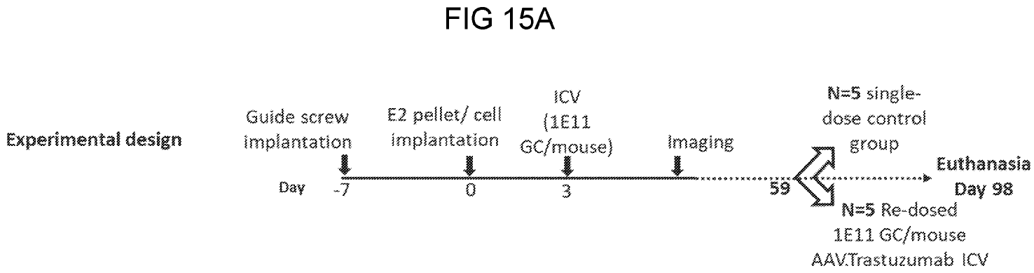

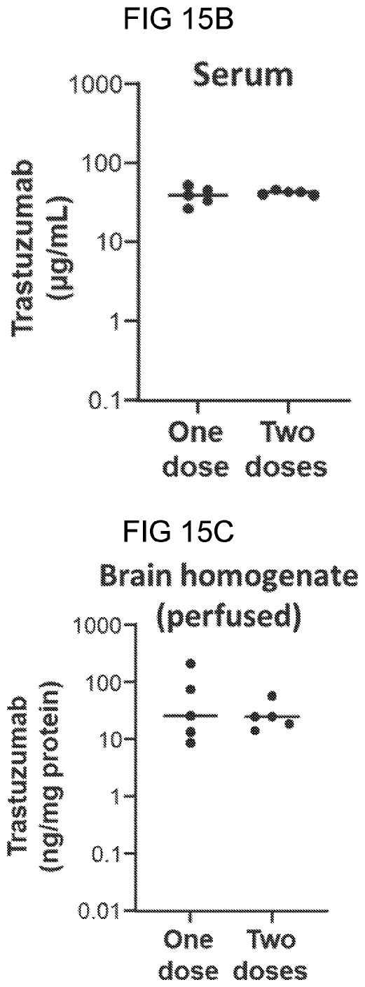

Furthermore, we performed a dose-comparison study using MDA-MB-453 mouse model to examine long-term transgene expression after a single (or double; re-dosed on day 59 post tumor cell implantation) ICV administration of AAV.Trastuzumab. FIG. 15A shows a schematic representation for an experimental design of a study examining long-term transgene expression after a single or double (re-dose) administration of AAV.Trastuzumab. FIG. 15B show trastuzumab expression levels measured by ELISA using the serum samples harvested on day 98, plotted as trastuzumab gg/mL. FIG. 15C show trastuzumab expression levels measured by ELISA using the brain samples (perfused brain homogenate) harvested on day 98, plotted as trastuzumab gg/mL. These results show that trastuzumab is stably expressed for >98 days after a single ICV injection.

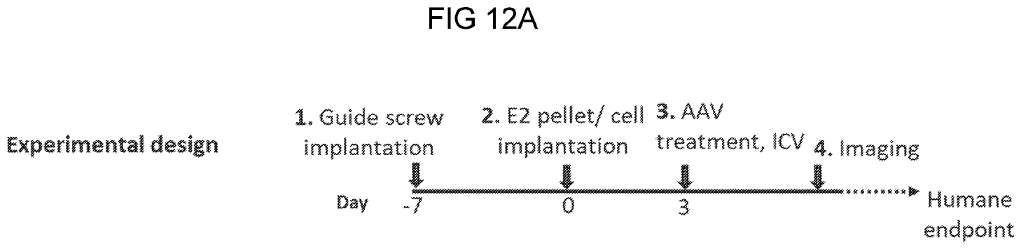

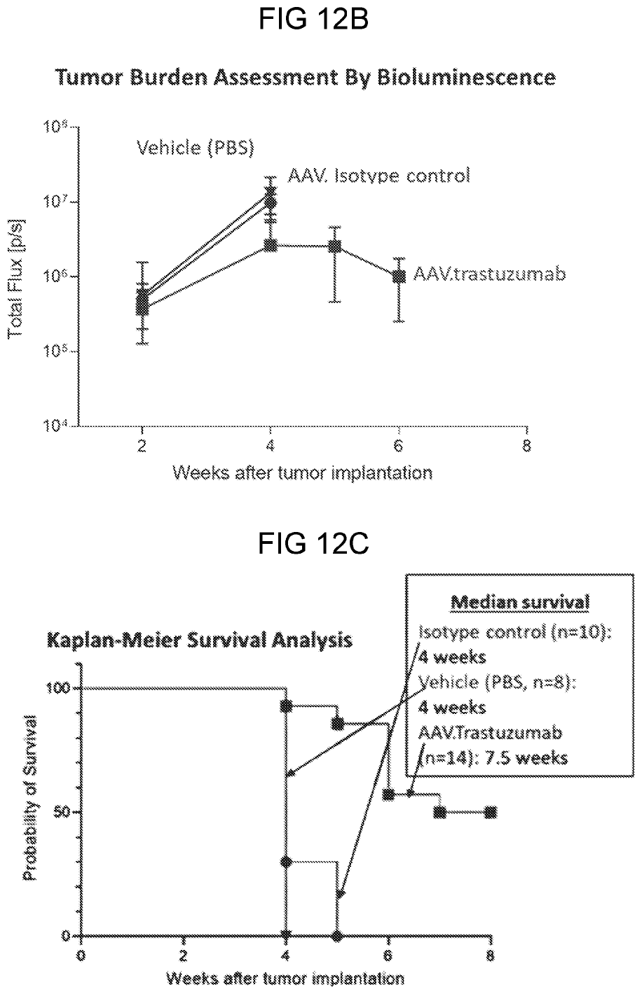

Furthermore, we examined preclinical activity in the BT-474 Clone 5 Trastuzumab Resistant (ER+/PR+/HER2+) xenograft, which provides for an evaluation in a more stringent model. FIG. 12A shows a schematic representation for an experimental design of a study examining preclinical activity in mice. FIG. 12B shows quantified results of the tumor bioluminescence assessment in mice xenograft (BT-474 Clone 5 Trastuzumab Resistant (ER+/PR+/HER2+) xenograft) post treatment with AAVhu95.CB.CI.IL2.V1.Trastuzumab-coGW.SV40 in comparison with isotype control. FIG. 12C shows Kaplan-Meier survival analysis of probability of survival in tumor bearing mice (BT-474 Clone 5 Trastuzumab Resistant (ER+/PR+/HER2+) xenograft) treated with AAVhu95.CB.CI.IL2.V1.Trastuzumab-coGW.SV40. These results show that treatment of BT-474 Clone 5 trastuzumab resistant (ER+/PR+/HER2+) xenograft with AAV.Trastuzumab on day +3 delayed tumor progression, with a median survival observed to be 4 weeks for isotype control, 4 weeks for vehicle control, and 7.5 weeks for AAV.Trastuzumab (i.e., approximately two-fold longer survival).

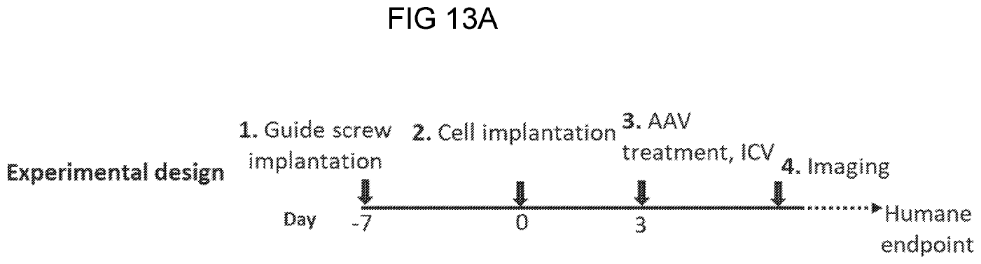

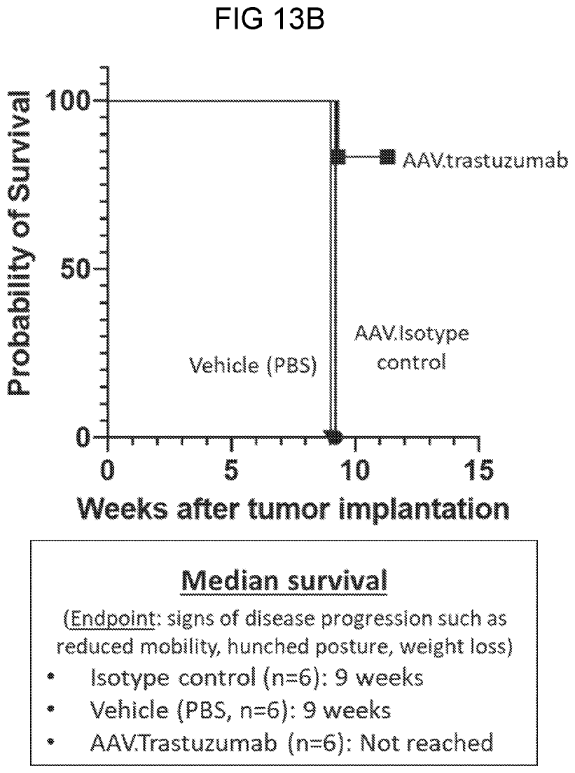

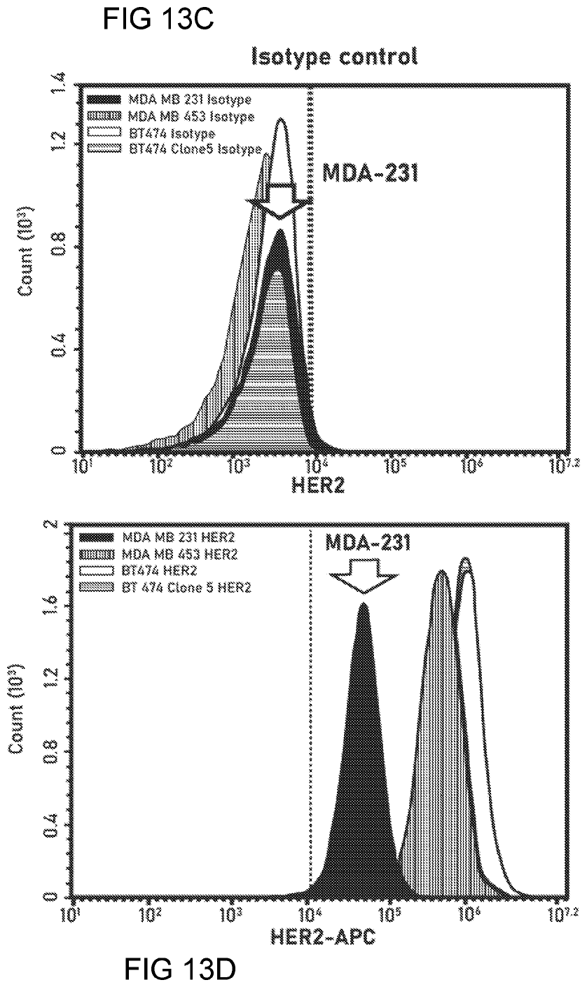

Furthermore, we examined preclinical activity in the MDA-MB-231-HER2/low tumor models. FIG. 13A shows a schematic representation for an experimental design of a study examining preclinical activity in mice. FIG. 13B shows Kaplan-Meier survival analysis of probability of survival in tumor bearing mice (MDA-MB-231HER2/low Tumors) treated with AAVhu95.CB.CI.IL2.V1.Trastuzumab-coGW.SV40. FIG. 13C shows Her2 expression levels in MDA-MB-231 cells as measured via flow cytometry following surface staining with isotype control antibody. FIG. 14D shows Her2 expression levels in MDA-MB-231 cells as measured via flow cytometry following surface staining with Her2 antibody. These results show that treatment with AAV.Trastuzumab ICV on day +3 inhibited progression of MDA-MB-231HER2/low tumors.

Overall, single dose ICV administration of AAV.CB.CI.IL2_V1.Trastuzumab-coGW.SV40 resulted in robust anti-tumor responses in the xenograft mouse models of HER2+ breast to brain metastases tested.

Treatment with AAV-trastuzumab vectors driven by CB or UbC promoters resulted in sustained antibody expression after 28 days. This gene therapy approach has the potential to provide and maintain long-lasting steady-state levels of the drug in the brain following single treatment of local delivery of trastuzumab to prevent and/or treat HER2+ metastatic brain lesions following a single administration, and for an effective one-time gene therapy approach for the prevention and/or treatment of HER2+ BCBM. The current treatment strategies for HER2+ BCBM uses different combinations of HER2 modulatory agents (anti-HER2 monoclonal antibodies and kinase inhibitors), as well as chemotherapeutic drugs. Moreover, intracranial delivery of AAV-trastuzumab in HER2+ breast cancer patients at high risk for the development of BCBM could prevent or significantly delay the emergence of tumor lesions. CNS delivery of AAV-trastuzumab alone or in combination with standard-of-care systemic HER2 kinase inhibitors could be used as an alternative approach to achieve disease remission or stabilization since it could avoid treatment scape and result in long lasting trastuzumab expression.

### Natural History Tumor Progression Study

In this study we evaluate the validity of the selected tumor model approach. Briefly, in this study green fluorescent protein positive (GFP+) breast cancer cells (e.g., BT-474, BT-474 clone 5 (trastuzumab resistant and aggressive cell line in vivo), MDA-MB-453, MDA-MB-231 cells) were sorted and expanded post-sorting. The cells were then implanted at 1.5×10⁵ cells in PBS/Matrigel (50:50) using the guide screw method (e.g., guide screw implantation followed by tumor cell engraftment >1 week later). Advantages of the guide screw method are that it does not require a stereotactic frame, it is streamlined (i.e., more animals per procedure (˜6 min/mouse)), screws can be implanted even weeks before tumor cell implantation, it allows for intra-tumoral delivery of therapeutics (i.e., screws can be re-opened several times). Hematoxylin and eosin (H&E) histology so far have demonstrated 100% tumor take (data not shown). Furthermore, we generated GFP/luciferase dual reporter breast cancer cell lines which are also used in studies herein.

Additionally, we develop patient-derived xenograft models using HER2+ breast cancer fragments from (DFCI), which were further implanted in a tumor slurry in 6-8 weeks-old NOD scid gamma mice (NSG™ mice, Jackson Laboratory), with time to progression between 6-12 months.

Furthermore, studies in mice are expanded to include tumor challenge experiments using HER2+ and HER2-xenograft mouse models to confirm anti-tumor efficacy and target specificity, as well as a combined therapy approach including clinical HER2 kinase inhibitor(s) to maximize antitumor effects. Further studies include evaluation of AAV-trastuzumab vector packaged into AAVhu95 capsids. The AAV-trastuzumab vector is then further evaluated for toxicology in rhesus macaques.

### Example 3: Further Evaluation of rAAV.Trastuzumab in Mice

First, we evaluated expression levels of revised rAAV.trastuzumab vectors in mice. The revised rAAV.trastuzmnab include vectors comprising furin cleavage site in the linker sequence (Furin/T2A) between the heavy chain and light chain of trastuzumab coding sequence: AAVhu95M199.IE.CB7.CJ.IL2_Furin_V1_Trastuzumab-coGW.SV40, and AAVhu95M199.UbC.PI.IL2_Furin_V1_Trastuzumab-coGW.SV40.

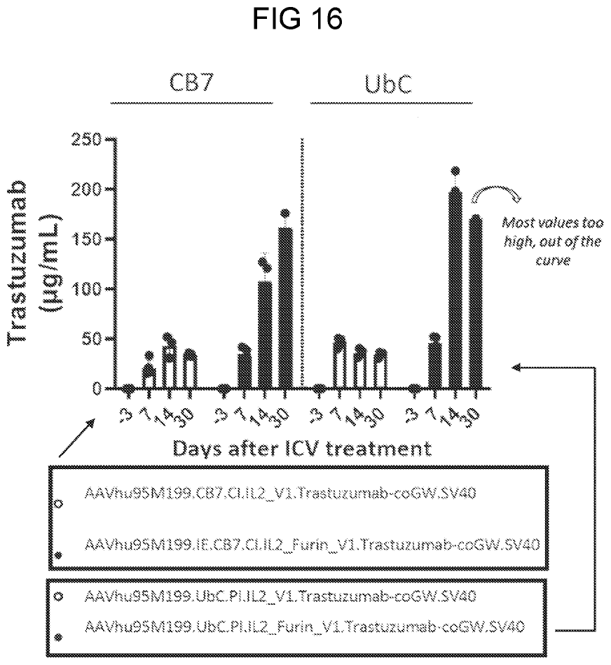

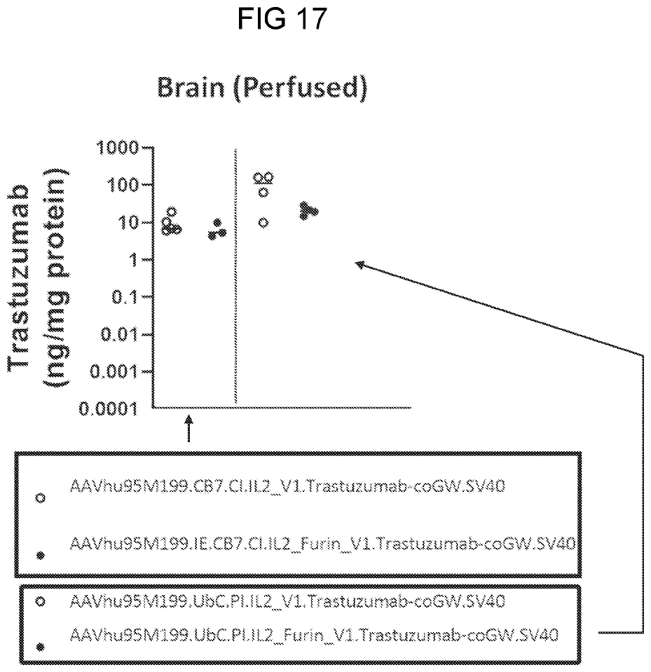

FIGS. 16 and 17 show comparison of evaluation of expression levels of trastuzumab in mice following rAAV.Trastuzumab and revised rAAV.Trastuzumab administration. FIG. 16 shows trastuzumab expression levels in serum following administration of AAVhu95M199.CB7.CI.IL2 Trastuzumab-coGW.SV40, AAVhu95M199.IE.CB7.CI.IL2_Furin_V1_Trastuzumab-coGW.SV40, AAVhu95M199.UbC.PI.IL2_Furin_V1_Trastuzumab-coGW.SV40, AAVhu95M199.UbC.PI.IL2_Trastuzumab-coGW.SV40 in mice. FIG. 17 shows trastuzumab expression levels in brain (perfused) following administration of AAVhu95M199.CB7.CI.IL2_Trastuzumab-coGW.SV40, AAVhu95M199.IE.CB7.CI.IL2_Furin_V1_Trastuzumab-coGW.SV40, AAVhu95M199.UbC.PI.IL2_Furin_V1_Trastuzumab-coGW.SV40, AAVhu95M199.UbC.Pi.IL2_Trastuzumab-coGW.SV40 in mice.

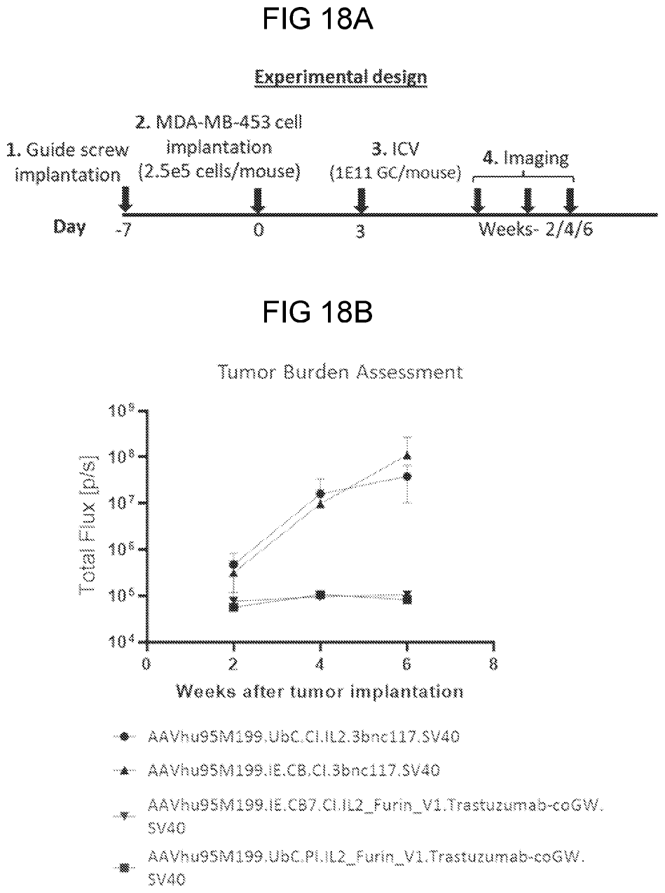

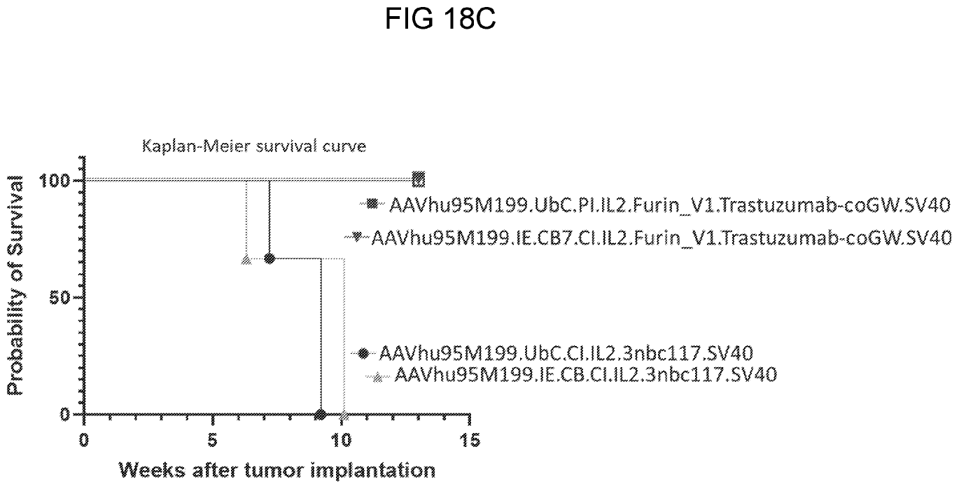

Next, performed a study to examine the lowest effective dose which confirms therapeutic effect of rAAV.trastuzunab. FIG. 18A shows a schematic of the experimental design of the study for assessing tumor burden. Briefly, seven days before cell implantation (D-7) a guide screw was implanted, on day 0 MDA-MB-453 cells were implanted (2.5×10⁵ cells/mouse), on day 3 post cell implantation mice were administered with rAAV.trastuzumab at a dose of 1×10¹¹ GC/mouse intracranially (ICV), and imaging was performed at 2, 4, and 6 weeks post cell implantation. FIG. 18B shows results of the tumor burden assessment plotted as Total Flux (p/s) at 2, 4 and 6 weeks post tumor cell implantation in mice administered with AAVhu95M199.UbC.CT.IL2.3bnc117.SV40, AAVhu95M199.TE.CB.CT.3bnc117.SV40, AAVhu95M199.IE.CB7.CI.IL2_Furin_V1.Trastuzumab-coGW.SV40, AAVhu95M199.UbC.PI.IL2_Furin_V1.Trastuzumab-coGW.SV40. These results confirm therapeutic effect of rAAV.Trastuzumab vectors. FIG. 18C shows Kaplan-Meier survival curve in mice following administration with AAVhu95M199.UbC.CI.IL2.3bnc117.SV40, AAVhu95M199.IE.CB.CI.3bnc117.SV40, AAVhu95M199.IE.CB7.CI.IL2_Furin_V1.Trastuzumab-coGW.SV40, AAVhu95M199.UbC.PI.IL2_Furin_V1.Trastuzumab-coGW.SV40.

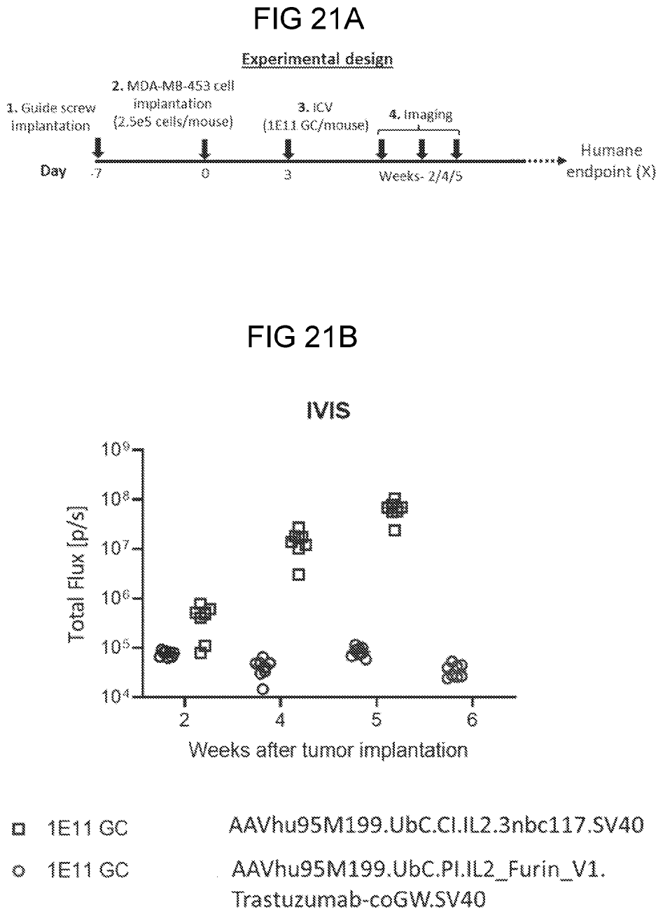

Next, we performed tumor challenge and tumor remission study in MDA-MB-453 xenograft mice model administered with AAVhu95M199.UbC.CI.IL2.3bnc117.SV40, AAVhu95M199.UbC.PI.IL2_Furin_V1.Trastuzumab-coGW.SV40 at a dose 1×10¹⁰ GC/mouse and 1×10¹¹ GC/mouse. FIG. 21A shows a schematic of the experimental design of the study for assessing tumor challenge. Briefly, seven days before cell implantation (D-7) a guide screw was implanted, on day 0 MDA-MB-453 cells were implanted (2.5×10⁵ cells/mouse), on day 3 post cell implantation mice were administered with rAAV.trastuzumab at a dose of 1×10¹¹ GC/mouse intracranially (ICV), and imaging was performed at 2, 4, and 5 weeks post cell implantation. FIG. 21B shows tumor growth plotted as measured total flux (p/s) at 2, 4, and 5 weeks post tumor implantation in mice administered with AAVhu95M199.UbC.CI.IL2.3bnc117.SV40, AAVhu95M199.UbC.PI.IL2_Furin_V1.Trastuzumab-coGW.SV40 at a dose 1×10¹¹ GC/mouse.

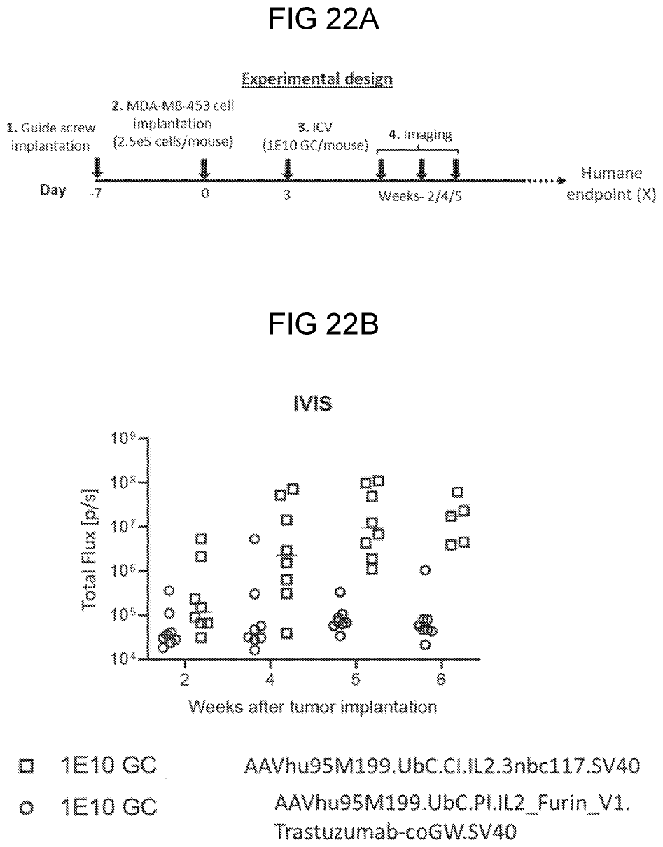

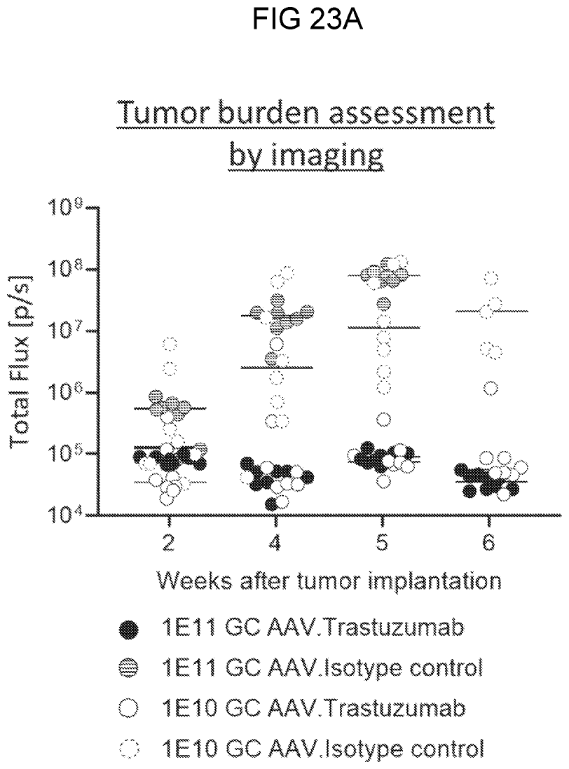

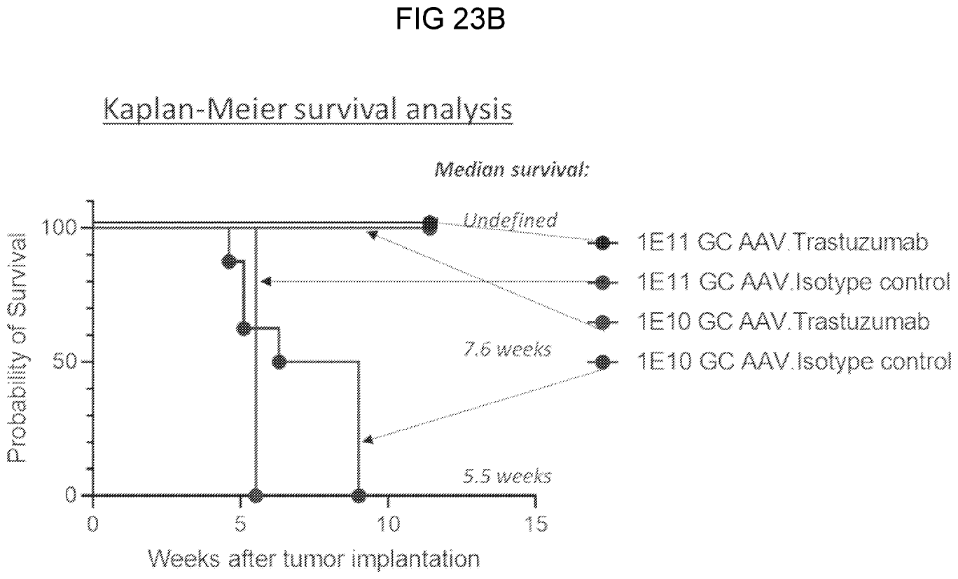

FIG. 22A shows a schematic of the experimental design of the study for assessing tumor challenge. Briefly, seven days before cell implantation (D-7) a guide screw was implanted, on day 0 MDA-MB-453 cells were implanted (2.5×10⁵ cells/mouse), on day 3 post cell implantation mice were administered with rAAV.trastuzumab at a dose of 1×10¹⁰ GC/mouse intracranially (ICV), and imaging was performed at 2, 4, and 5 weeks post cell implantation. FIG. 22B shows tumor growth plotted as measured total flux (p/s) at 2, 4, and 5 weeks post tumor implantation in mice administered with AAVhu95M199.UbC.CI.IL2.3bnc117.SV40, AAVhu95M199.UbC.PI.IL2_Furin_V1.Trastuzumab-coGW.SV40 at a dose 1×10¹⁰ GC/mouse. FIG. 23A shows results of tumor burden assessment as examined by imaging plotted as total flux (p/s) in mice administered with AAVhu95M199.UbC.CI.IL2.3bnc117.SV40, AAVhu95M199.UbC.PI.IL2_Furin_V1. Trastuzumab-coGW.SV40 at a dose 1×10¹⁰ GC/mouse and 1×10¹¹ GC/mouse. FIG. 23B shows results of Kaplan-Meier survival analysis plotted as probability of survival in mice administered with AAVhu95M199.UbC.CI.IL2.3bnc117.SV40, AAVhu95M199.UbC.PI.IL2_Furin_V1.Trastuzumab-coGW.SV40 at a dose 1×10¹⁰ GC/mouse and 1×10¹¹ GC/mouse.

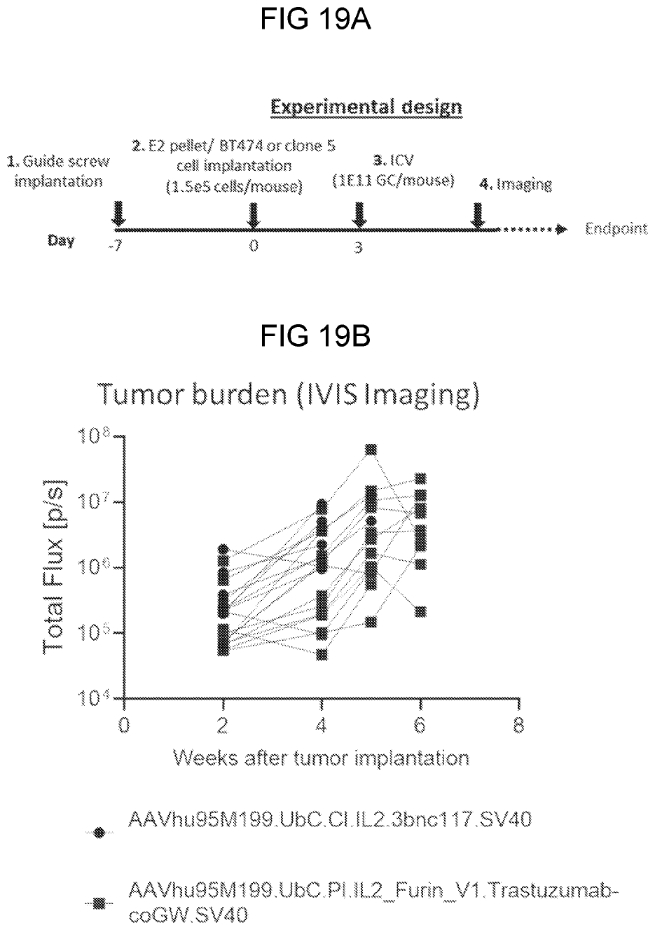

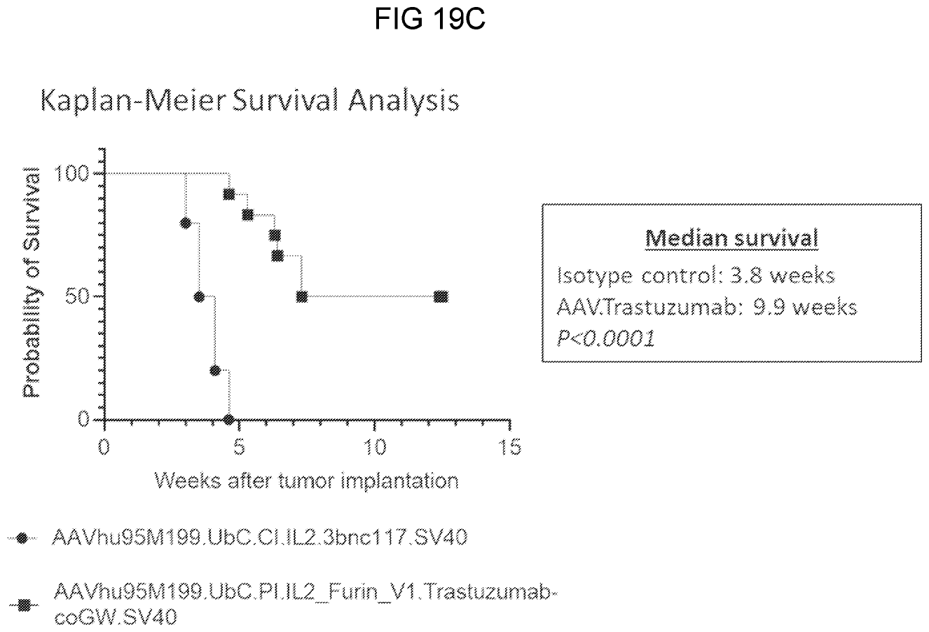

Additionally, we performed study assessing antitumor activity in BT474 clone 5 cell xenograft tumor model. FIG. 19A shows a schematic of the experimental design of the study for assessing antitumor activity. Briefly, seven days before cell implantation (D-7) a guide screw was implanted, on day 0 E2 pellets with BT474 clone 5 cells were implanted (1.5×10⁵ cells/mouse), on day 3 post cell implantation mice were administered with rAAV.trastuzumab at a dose of 1×10¹¹ GC/mouse intracranially (ICV), and imaging was performed at 2 and weeks post cell implantation. FIG. 19B shows results of the tumor burden assessment plotted as Total Flux (p/s) at 2, 4 and 6 weeks post tumor cell implantation in mice administered with AAVhu95M199.UbC.CJ.IL2.3bnc117.SV40, AAVhu95M199.UbC.PI.IL2 Furin_V1.Trastuzumab-coGW.SV40. FIG. 19C shows Kaplan-Meier survival curve in mice (with BT474 Xenografts) following administration with AAVhu95M199.UbC.C.IL2.3bnc117.SV40, AAVhu95M199.IE.CB.CJ.3bnc117.SV40, AAVhu95M199.IE.CB7.CI.IL2_Furin_V1.Trastuzumab-coGW.SV40, AAVhu95M199.UbC.PI.IL2_Furin_V1.Trastuzumab-coGW.SV40.

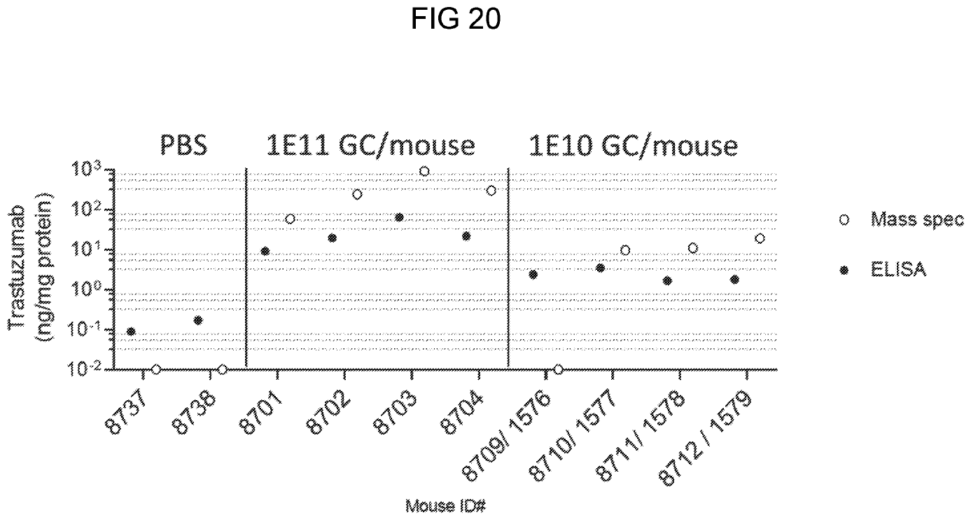

Next, we performed rAAV vector titration study and examined trastuzumab expression levels. Detection and quantification of trastuzumab was performed by Mass Spectrometry (LC-MS) and ELISA in perfused mouse brain homogenates FIG. 20 shows results of the vector titration study plotted as trastuzumab expression levels a as measured by ELISA and Mass Spectrometry in mice administered with rAAV.Trastuzumab (AAVhu95M199.UbC.PI.IL2_Furin_V1.Trastuzumab-coGW.SV40) at a dose of 1×10¹¹ GC/mouse and 1×10¹⁰ GC/mouse. These results show that a dose of 1×10¹⁰ GC/Mouse administered ICV results in transgene levels like found in NHP brain and is sufficient to induce sustained tumor remission in the MDA-MB-453 brain xenografts. Low dose of rAAV.Trastuzumab inhibits tumor progression in HER2-cell line-derived xenograft model.

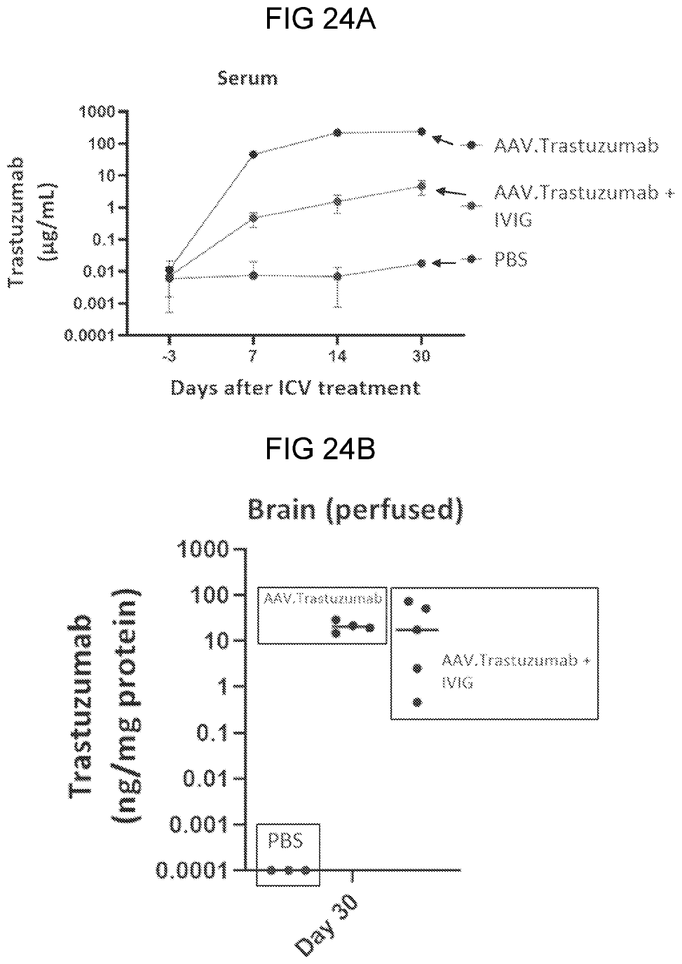

Additionally, we performed a study examining the effect of IVIG pre-treatment in mice followed by ICV administration of rAAV.Trastuzumab (AAVhu95M199.UbC.PI.IL2_Furin_V1.Trastuzumab-coGW.SV40). Briefly, healthy TAG1KO female mice were administered via ICV with rAAV.Trastuzumab at a dose of 1×10¹¹ GC/mouse, and samples of blood for analysis were taken on day 3 before days 7, 14, 21, and 29 after rAAV administration. FIG. 24A shows trastuzumab expression levels in serum following administration with rAAV.Trastuzumab with and without IVIG pre-treatment. FIG. 24B shows trastuzumab expression levels in brain (perfused) at day 30 following administration with rAAV.Trastuzumab with and without IVIG pre-treatment. These results show that pre-treatment with IVIG reduced systemic levels of trastuzumab but not in brain.

Overall, the results above confirm that engineered trastuzumab construct in AAV. Trastuzumab inhibited tumor progression in all cell line-derived tumor xenograft models tested (BT-474 S and R, MDA-MB-453) at dose of 1×10¹ GC/mouse. Additionally, these data show that a lower dose (i.e., 1×10¹⁰ GC/mouse) was still sufficient to induce tumor remission in MDA-MB-453 xenografts (HR−/HER2+).

### Example 4: rAAV Evaluation in Non-Human Primates (NHPs)

In this study, we examined rAAV.Trastuzumab-coGW vector efficacy in non-human primates (NHPs; Nab+ Female Indian Rhesus). Briefly, at day −5 baseline measurements were taken of clinical pathology samples, neutralizing antibodies (NAb) levels, neurological exam, and Nerve Conduction Velocity (NCV) test is also performed. At day 0, NHPs were dosed with specified AAV vectors (via intra-cisterna magna (ICM) injection), at which time further clinical pathology samples were taken, serum and cerebrospinal fluid (CSF) samples were collected for analysis, and NAb were evaluated. At days 7 and 21, further serum and cerebrospinal fluid (CSF) samples were collected for analysis. At day 14, further serum and cerebrospinal fluid (CSF) sample were collected for analysis and further neurological exam is performed. At days 36-37 post ICM, necropsy is performed, and further clinical pathology sample are taken, and serum and CSF fluid samples were collected, NAb/PBMC were evaluated, and further neuro-exam and NCV test was performed prior to necropsy. Throughout duration of the study, ELISA for trastuzumab and measurements for humoral immune response (Anti-Drug Antibody (ADA)) were performed to evaluate expression, and to further guide the duration of the study. Table 3, below, shows a summarized view of the study design.

TABLE 3

| | | |
|---|---|---|
| Group | 1 | 2 |
| Designation | | |
| Number of | 2 | 2 |
| macaques | | |
| Sex/age | F/adult | F/adult |
| Test article | AAVhu95.CB7.CI.IL2_{—} | AAVhu95.UbC.CI.IL2_{—} |
| | V1.Trastuzumab-coGW.SV40 | V1.Trastuzumab-coGW.SV40 |
| | (AAVhu95M199.IE.CB7.CI.IL2_{—} | (AAVhu95M199.UbC.PI.IL2_{—} |
| | Furin_V1.Trastuzumab- | Furin_V1.Trastuzumab- |
| | coGW.SV40) | coGW.SV40) |
| Route of | ICM | ICM |
| administration | | |
| Vector Dose | 3 × 10¹³ GC | 3 × 10¹³ GC |
| (total dose) | | |
| Necropsy Day | 35 | 35 |

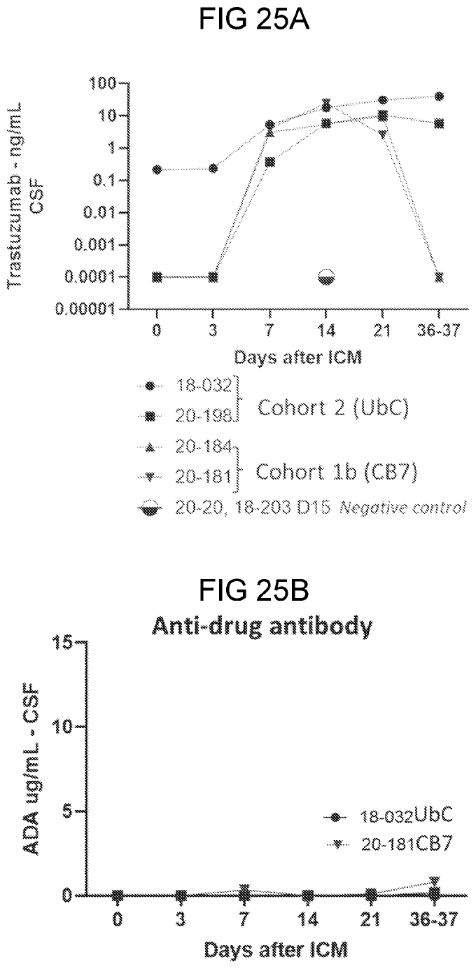

FIG. 25A shows trastuzumab expression levels in CSF as measured by ELISA on days 0. 3, 7, 14, 21 and 36-37 following rAAV administration in NHPs of cohort 1b ((AAVhu95M199.IE.CB7.CI.IL2_Furin_V1.Trastuzumab-coGW.SV40)), cohort 2 (AAVhu95M199.UbC.PI.IL2 Furin_V1.Trastuzumab-coGW.SV40), and negative control on day 14. FIG. 25B shows levels of anti-drug antibodies (ADA) as measured in collected samples of CSF in NHPs on days 0. 3, 7, 14, 21 and 36-37 following rAAV administration (AAVhu95M199.IE.CB7.CI.IL2_Furin_V1.Trastuzumab-coGW.SV40 and AAVhu95M199.UbC.PI.IL2_Furin_V1.Trastuzumab-coGW.SV40. These results show that trastuzumab is detected in the CSF to the terminal study timepoint, and that no ADAs against trastuzumab are detected in CSF.

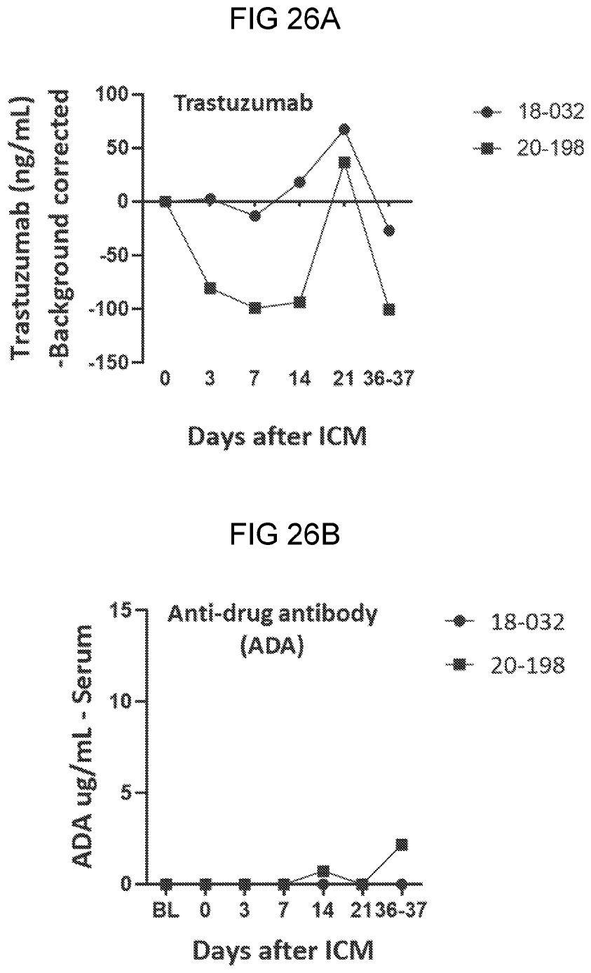

FIG. 26A shows trastuzumab expression levels in serum as measured by ELISA on days 0. 3, 7, 14, 21 and 36-37 following rAAV administration in NHPs of cohort 1b ((AAVhu95M199.IE.CB7.CI.IL2_Furin_V1.Trastuzumab-coGW.SV40)), cohort 2 (AAVhu95M199.UbC.PI.IL2_Furin_V1.Trastuzumab-coGW.SV40). FIG. 26B shows levels of anti-drug antibodies as measured in collected samples of serum in NHPs on days 0. 3, 7, 14, 21 and 36-37 following rAAV administration (AAVhu95M199.IE.CB7.CI.IL2_Furin_V1.Trastuzumab-coGW.SV40 and AAVhu95M199.UbC.PI.IL2_Furin_V1.Trastuzumab-coGW.SV40. These results show that there is low systemic trastuzumab is detected in NHP serum by ELISA following ICM treatment with AAV.Trastuzumab.

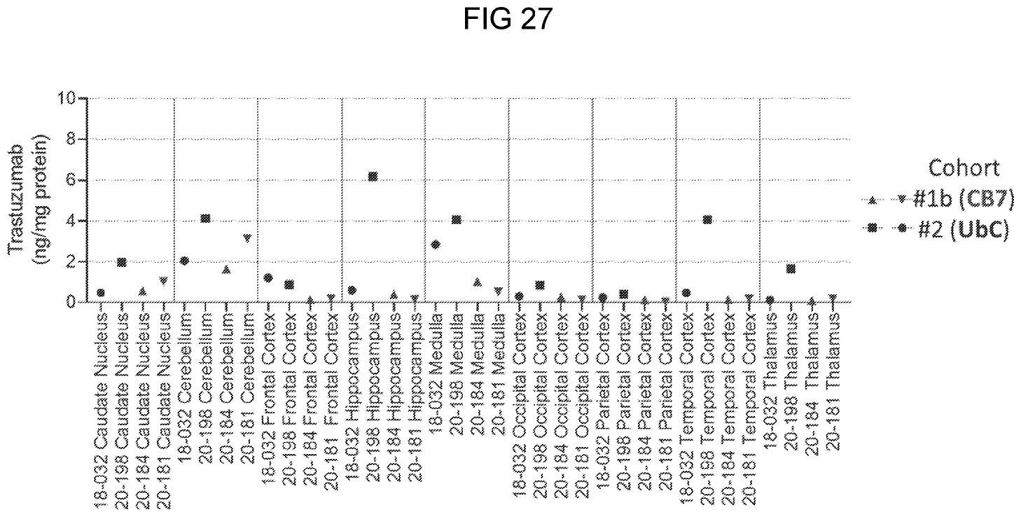

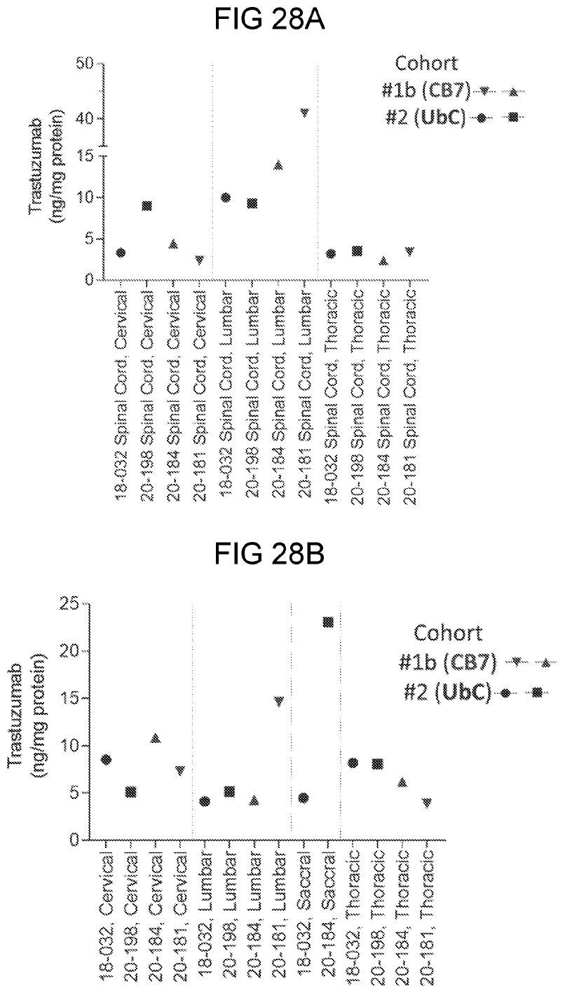

FIG. 27 shows quantification of trastuzumab protein in NHP brain tissue following ICM administration of AAV.Trastuzumab (AAVhu95M199.IE.CB7.CI.IL2_Furin_V1.Trastuzumab-coGW.SV40 and AAVhu95M199.UbC.PI.IL2 Furin_V1.Trastuzumab-coGW.SV40). FIG. 28A shows quantification of trastuzumab protein in NHP spinal cord following ICM administration of AAV.Trastuzumab (AAVhu95M199.IE.CB7.CI.IL2_Furin_V1.Trastuzumab-coGW.SV40 and AAVhu95M199.UbC.PI.IL2_Furin_V1.Trastuzumab-coGW.SV40). FIG. 28B shows quantification of trastuzumab protein in NHP Dorsal Root Ganglion (DRG) following ICM administration of AAV.Trastuzumab (AAVhu95M199.IE.CB7.CI.IL2_Furin_V1. Trastuzumab-coGW.SV40 and AAVhu95M199.UbC.PI.IL2_Furin_V1.Trastuzumab-coGW.SV40).

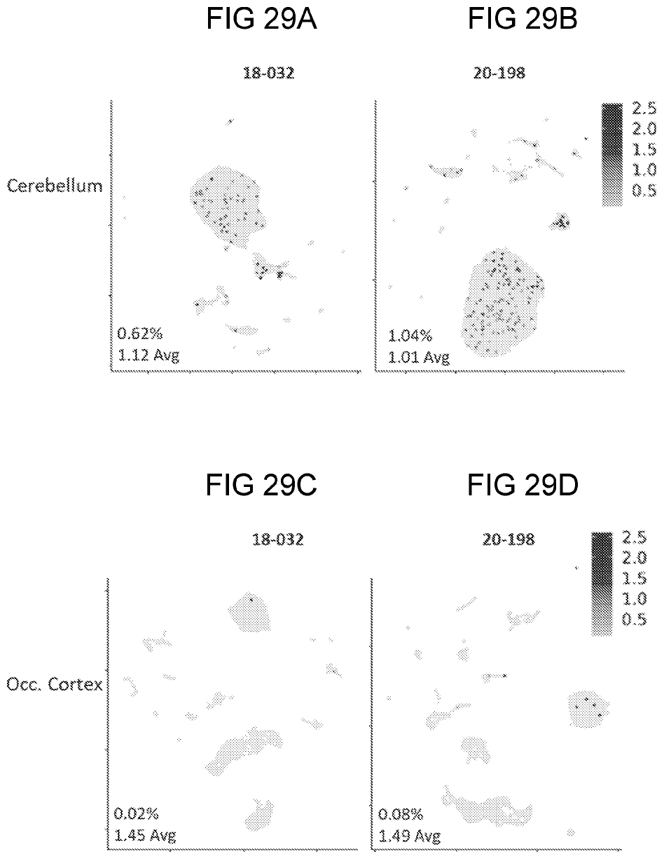

FIGS. 29A to 29D show results of trastuzumab mRNA Detection in NHP brain tissues (cerebellum and occipital lobe cortex) by 10×GENOMICS (final sequencing depth average reads −883×10⁶ reads/sample). There was observed no background detection of trastuzumab in untreated monkeys. FIG. 29A shows results of trastuzumab mRNA Detection in cerebellum in NHP 18-032 administered with AAVhu95M199.UbC.PI.IL2_Furin_V1.Trastuzumab-coGW.SV40. FIG. 29B shows results of trastuzumab mRNA Detection in cerebellum in NHP 20-198 administered with AAVhu95M199.UbC.PI.IL2 Furin_V1.Trastuzumab-coGW.SV40. FIG. 29C shows results of trastuzumab mRNA Detection in occipital lobe cortex in NHP 18-032 administered with AAVhu95M199.UbC.PI.IL2 Furin_V1.Trastuzumab-coGW.SV40. FIG. 29D shows results of trastuzumab mRNA Detection in occipital lobe cortex in NHP 20-198 administered with AAVhu95M199.UbC.PI.IL2_Furin_V1.Trastuzumab-coGW.SV40.

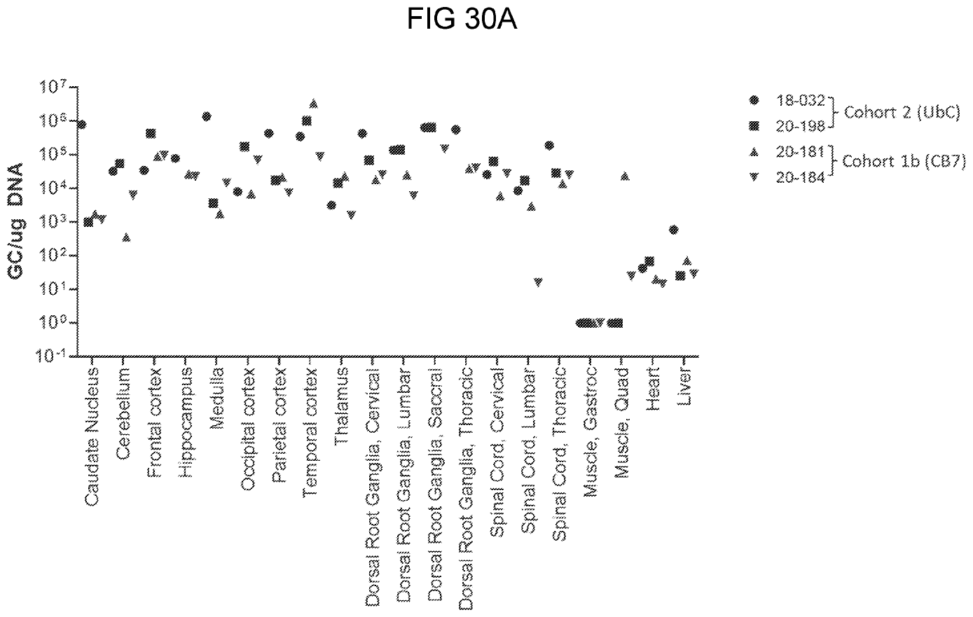

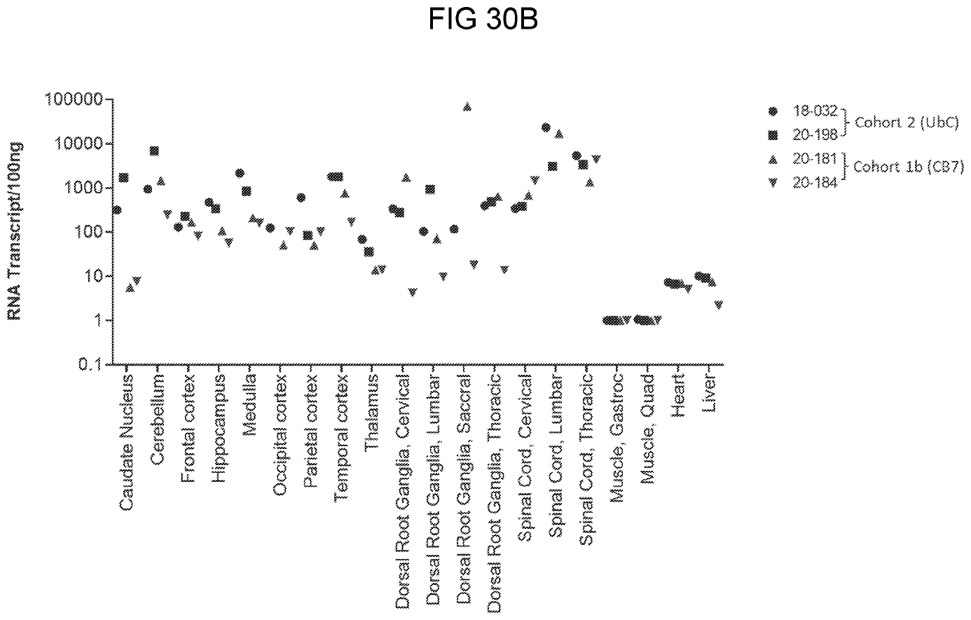

FIGS. 30A and 30B show biodistribution of AAV.Trastuzumab (AAVhu95M199.IE.CB7.CJ.IL2_Furin_V1.Trastuzumab-coGW.SV40 and AAVhu95M199.UbC.PI.IL2_Furin_V1. Trastuzumab-coGW.SV40) in NHP in various tissues following ICM administration. FIG. 30A shows DNA biodistribution of AAV.Trastuzumab in NHP in various tissues following ICM administration. FIG. 30B shows RNA biodistribution of AAV.Trastuzumab in NHP in various tissues following ICM administration.

Overall, these results show treatment with AAV.Trastuzumab can both prevent the development of HER2+ CNS tumors and drive tumor regression in the tumor animal models evaluated so far.

### Example 5: Outcomes of Adeno-Associated Viral (AAV) Vector-Mediated Delivery of Trastuzumab to the Central Nervous System of Xenograft Models of Her2+ Breast Cancer Brain Metastasis and Nonhuman Primates

### Translational Relevance

The incidence of breast cancer metastasis to the central nervous system (CNS) is rising due to advances in systemic therapy that better controls extra-cranial disease but potentially enables metastatic cells within the CNS to exit latency. Breast cancer patients with symptomatic brain and/or leptomeningeal metastases have a poor prognosis because current adjuvant therapies fail to prevent CNS recurrence. Repeated intrathecal (IT) delivery of trastuzumab safe and provides clinical benefits to patients with HER2+ CNS disease. Trastuzumab (Herceptin®) is approved in protein form as a gold standard treatment for human epidermal growth factor receptor 2 (HER2)-positive breast cancer. To overcome the treatment burden associated with serial IT drug administration, we developed an adeno-associated virus (AAV) vector to persistently express trastuzumab within the CNS after a single IT injection. Preclinical studies demonstrated that AAV-trastuzumab induced remissions in HER2+ orthotopic models of brain metastasis and was well-tolerated in nonhuman primates. These encouraging preclinical findings warrant future clinical investigation.

### Abstract

A single-dose gene therapy which expresses a trastuzumab-like anti-Her antibody within the CNS represents a therapeutic option for disease prevention or treatment.

We developed an AAV vector expressing a engineered trastuzumab sequence driven by the ubiquitin C (UbC) promoter (AAV9.UbC.trastuzumab) for IT administration. CNS transgene expression was evaluated in adult Rag1 knockout mice and rhesus nonhuman primates (NHPs) after a single intracerebroventricular (ICV) or intra-cisterna magna (ICM) AAV9.UbC.trastuzumab injection, respectively. Real-time PCR, ELISA, Western blot, in situ hybridization, immunohistochemistry, single nuclei RNA-sequencing, and liquid chromatography-mass spectrometry were employed. HER2+ breast cancer cell line-derived brain xenografts (BT-474 and MDA-MB-453) were used to determine the efficacy of AAV9.UbC.trastuzumab.

Transgene expression was detected in brain homogenates and serum of Rag1 knockout mice following ICV injection of AAV9.UbC.trastuzumab (1×10¹¹ vector genome copies (GC)/mouse), with levels plateauing at 28-days post administration. A single AAV9.UbC.trastuzumab administration inhibited tumor progression in the xenograft models tested, compared to AAV9.UbC.isotype control. In NHPs, ICM delivery of AAV9.UbC.trastuzumab (3×10¹³ GC/animal) was well-tolerated and resulted in detectable transgene expression in CNS tissues and cerebrospinal fluid after 36-37 days.

These results demonstrate that AAV9.UbC.trastuzumab exhibited robust activity against HER2+ brain orthotopic xenografts. With AAV9's proven clinical safety record, this gene therapy may represent a viable approach for targeting HER2+ CNS malignancies.

To meet the need for novel therapies to target HER2+ CNS metastases, we developed a gene therapy approach comprising a recombinant adeno-associated virus (AAV) vector engineered to constitutively express trastuzumab within the CNS. An effective single-dose gene therapy would provide a continuous local source of trastuzumab, improving patient outcomes.

Here, we demonstrate that a single IT administration of AAV9 encoding an engineered trastuzumab driven by the human ubiquitin C (UbC) promoter (AAV9.UbC.trastuzumab) resulted in robust anti-tumor responses in the treatment setting against HER2+ xenograft BCBM models. We also show that intra-cisterna magna injection of AAV9.UbC.trastuzumab in rhesus NHPs was well-tolerated and led to detectable transgene protein expression at levels anticipated to exert a therapeutic effect against orthotopic cell line-derived xenograft BCBM mouse models. Altogether, AAV-mediated expression of trastuzumab within the CNS may represent a viable strategy to treat and prevent the emergence of HER2+ CNS metastases in breast cancer patients.

### Materials and Methods

### Vector Construction

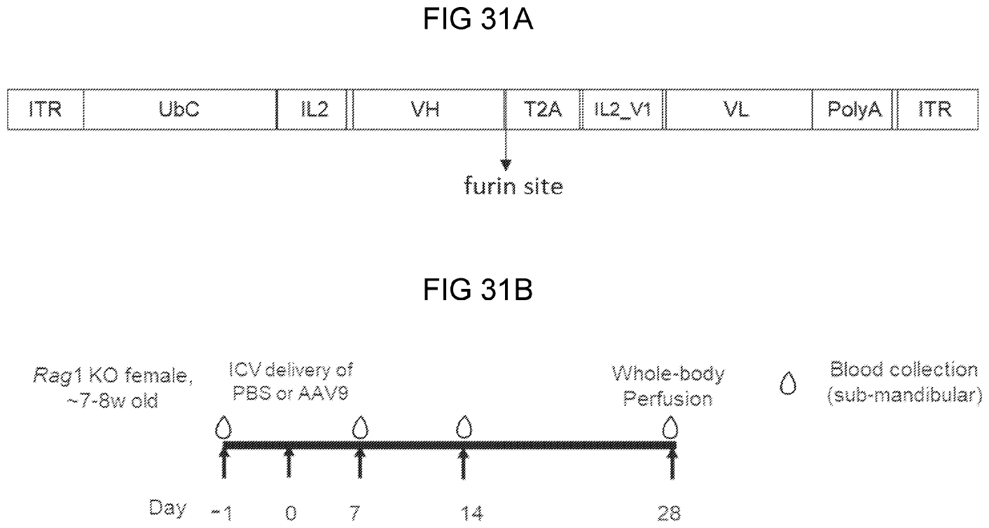

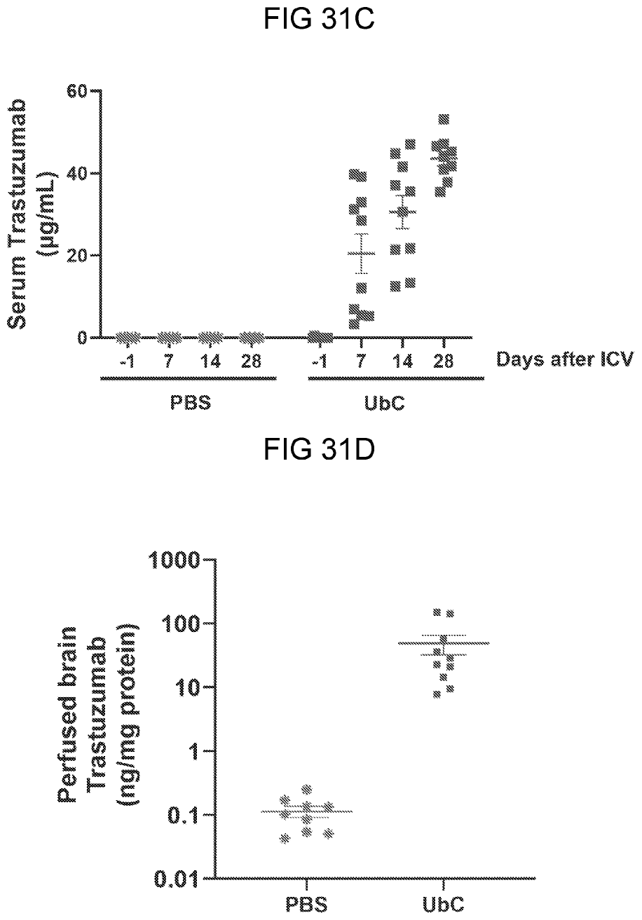

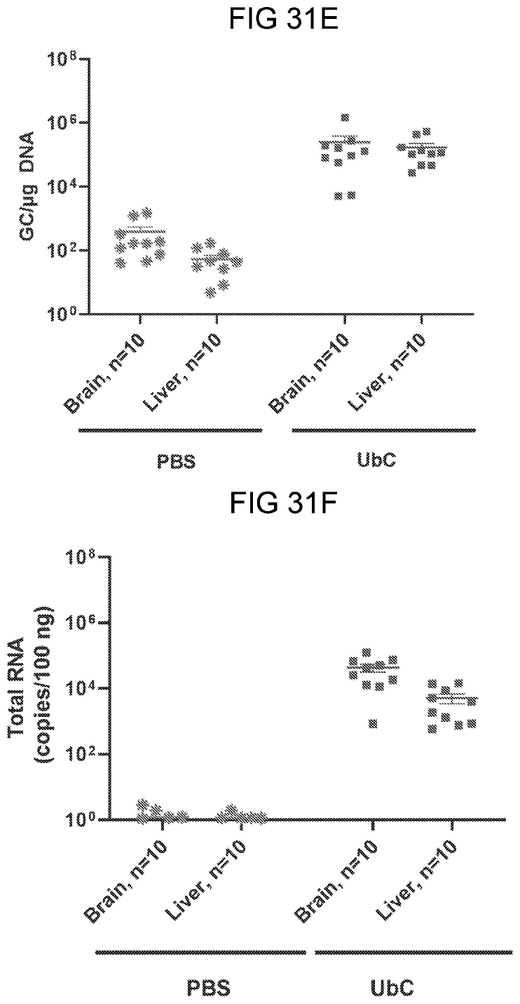

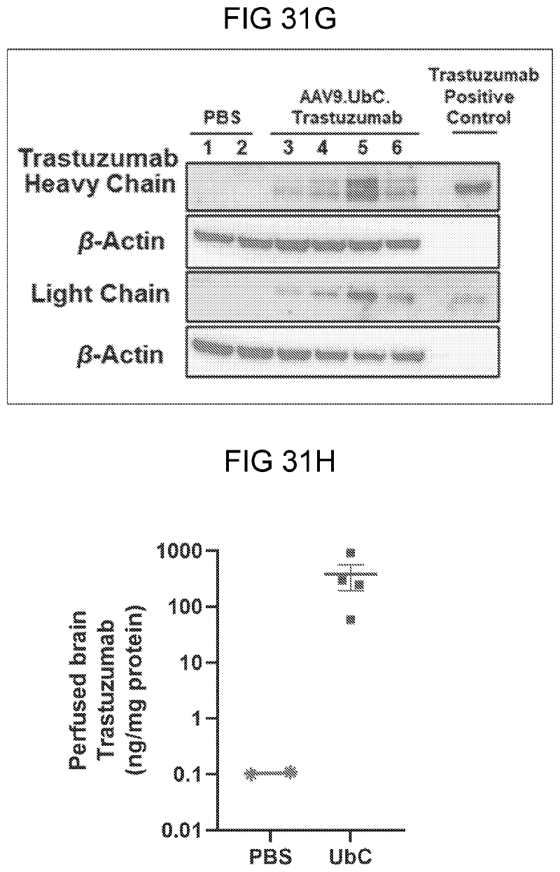

The transgene expression cassette comprising engineered trastuzumab coding sequence was synthesized by GeneArt (ThermoFisher) and engineered as follows: heavy and light chains were preceded by a human interleukin 2 (IL2) signal peptide (MYRMQLLSCIALSLALVTNS) and separated by a furin cleavage site, followed by a T2A self-cleavage peptide linker and a mutant IL2 signal peptide (MYRMQLLLLIALSLALVTNS). The UbC promoter was used to drive transgene expression (FIG. 31A).

The AAV9.isotype vector control was engineered to express 3BNC117, a fully human IgG1 kappa monoclonal antibody against the HIV envelope. All vectors were packaged in an AAV9 capsid, produced, and purified by the Penn Vector Core, as previously described [Lock, M., et al., Rapid, simple, and versatile manufacturing of recombinant adeno-associated viral vectors at scale. Hum Gene Ther, 2010. 21(10): p. 1259-71].

Cell lines and Generation of GFP-luciferase+ breast cancer reporter cell lines Human breast cancer cell lines MDA-MB-453 (HTB-131) and BT 474 (HTB-20) were obtained from ATCC and maintained according to vendor's instructions.

Cell lines were transduced with CMV-Luciferase-EF1α-copGFP BLIV 2.0 Lentivector lentiviral vector using TransDux MAX lentivirus transduction reagent, according to vendor instructions (Systems Biosciences). Transduced cells were then sorted for the top 50% brightest cells expressing green fluorescent protein (GFP).

### Animal Procedures

All animal procedures were approved by the Institutional Animal Care and Use Committee of the University of Pennsylvania. Animals were monitored daily by the animal care and/or veterinary staff for any signs of distress requiring intervention.

### Vector Administration

Mice: AAV vector was administered intracerebroventricularly (ICV) in 7-to-8-week-old female B6.129S7-Rag1tm1Mom/J knockout mice (Rag1 KO, Jackson Laboratories Strain #002216) by trained laboratory animal technicians or veterinarians (success rate >95%). Blood was collected from the submandibular vein at scheduled time points (FIG. 31B).

Nonhuman primates (NHPs): Adult female Indian rhesus macaques (3-4 years old) were dosed via the intra-cisterna magna (ICM) with AAV vector in 1 mL, as previously described [Katz, N., et al., Standardized Method for Intra-Cisterna Magna Delivery Under Fluoroscopic Guidance in Nonhuman Primates. Hum Gene Ther Methods, 2018. 29(5): p. 212-219]. Serum, CSF and tissue samples from an Indian rhesus macaque previously treated ICM with AAV9.GFP was used as a negative control in transgene expression assays. Blood was collected from the peripheral vein; up to 1 mL CSF was collected via suboccipital puncture (FIG. 34A). Animals were clinically monitored by veterinarians throughout the study, main parameters assessed were vital signs, blood and CSF clinical pathology, and comprehensive neurological evaluation (mentation, posture, proprioception, gait, reflexes, nerve conduction velocity tests).

### Necropsy

Mice: For vector expression studies, animals were deeply anesthetized and whole-body perfusions were performed with saline through the left ventricle at scheduled study endpoints. Brain, heart, and liver tissues were snap frozen for analysis.

NHPs: At days 36-37 post-vector administration, animals were deeply anesthetized and euthanized by intravenous (i.v.) pentobarbital overdose followed by brain perfusion through injection of 600 mL saline into the jugular vein. Various tissues were collected for histopathological analysis including Adrenal Gland, Aorta, Bone marrow, femur, Bone, sternum, Brain, Right hemisphere, Brain, caudate nucleus, Brain, cerebellum, Brain, frontal cortex, Brain, hippocampus, Brain, medulla, Brain, occipital cortex, Brain, parietal cortex, Brain, temporal cortex, Brain, thalamus. Brain, olfactory bulb, Chest tattoo, Deep cervical lymph node, Dorsal root ganglia cervical, Dorsal root ganglia thoracic, Dorsal root ganglia lumbar, Dorsal root ganglia sacral, Esophagus, Eyes, Gall Bladder, Heart, Intestine-Large, colon, Intestine-Small, duodenum, Intestine-Small, jejunum, Kidney (left), Kidney (right), Liver, left lobe, Liver, right lobe, Liver, middle lobe, Liver, caudate lobe, Lung, left, Lung, right, Lymph nodes: axillary, Lymph nodes: cervical, Lymph nodes: inguinal, Lymph nodes: mesenteric. Muscle, quadriceps femoris, Muscle, gastrocnemius, Nerve, optic (cranial nerve II), Nerve, sciatic, Nerve, sural, Nerve, median, proximal, Nerve, median, distal, Nerve, median, epon, Nerve, tibial, Ovaries, Pancreas, Salivary Gland, submandibular, Injection site, skin and surrounding tissue, Injection site, spinal cord, Spinal cord, cervical, Spinal cord, thoracic, Spinal cord, lumbar, Spleen, Thymus, Thyroid gland w/parathyroid, Trachea, Trigeminal nerve+ ganglia, Urinary bladder, Gross Lesions.

### Tumor Models

Breast cancer cell lines (250,000 cells/mouse) were resuspended in 1:1 DPBS/Matrigel and implanted in 7-to-8 week old female Rag1 KO mice through a guide screw, as previously described [Lal, S., et al., An implantable guide-screw system for brain tumor studies in small animals. J Neurosurg, 2000. 92(2): p. 326-33]. Three to four days post engraftment, mice were randomized to the treatment groups using an online randomization tool (random.org) and treated with AAV vectors via ICV injection. Tumor progression was monitored using IVIS Spectrum In Vivo Imaging System (PerkinElmer). Animals were euthanized at humane endpoints (signs of cachexia, lethargy, hunched posture).

Estrogen receptor (ER), progesterone receptor (PR) and HER2 receptor tumor statuses was confirmed by immunohistochemistry in formalin fixed brain sections.

Histopathology Histopathological evaluations of NHP tissues were conducted by veterinary pathologist that is board certified by the American College of Veterinary Pathologist, using formalin fixed paraffin embedded tissues stained with hematoxylin & eosin.

### Immunohistochemistry (IHC)

Immunohistochemistry was performed on sections from formalin-fixed paraffin embedded tissues on a Leica Bond Rx autostainer following a standard IHC protocol with the Bond polymer detection system (Leica Biosystems, DS9800) and DAB as chromogen. Primary antibodies were applied with 30 min incubation time using the indicated conditions to detect estrogen receptor (ER), progesterone receptor (PR) and HER2. After the staining, procedure slides were dehydrated through ethanol and xylene and cover-slipped.

### Vector DNA and RNA Biodistribution

DNA and RNA was extracted from snap-frozen tissue samples using QIAmp mini extraction kits (Qiagen). Vector GC levels were determined by qPCR using vector-specific primers and probes.

### Single Nuclei RNA Sequencing

Nuclei Isolation and snRNA-Seq

To isolate nuclei from frozen tissue samples, a modified version of previously published nuclei isolation procedures was used [Zhu, Y., et al., Spatiotemporal transcriptomic divergence across human and macaque brain development. Science, 2018. 362(6420); Kati J. Ernst, K. O., Josephine Bageritz, Jan-Philipp Mallm, Andrea Wittmann, Kendra K. Maaß, Svenja Leible, Michael Boutros, Stefan M. Pfister, Marc Zuckermann, David T. W. Jones, Establishment of a simplified preparation method for single-nucleus RNA-sequencing and its application to long-term frozen tumor tissues. bioRxiv, 2020]. For these isolations, all buffers and samples were maintained on ice throughout the procedure to maintain nuclei integrity. Buffers were typically made as described below and cooled in advance, and then supplemented immediately prior to use to make “complete” buffers at a final concentration of 1 mM DTT, 0.8 U/μl RNase Inhibitor (Protector RNase Inhibitor, Roche), and 1× protease inhibitor (complete Mini EDTA-free, Roche). To isolate nuclei, ˜25 mg of frozen tissue was minced with a scalpel and transferred to a pre-chilled 2 ml Dounce homogenizer with 1 ml cold complete lysis buffer (0.32 M sucrose, 5 mM CaCl₂), 3 mM magnesium acetate, 0.1 mM EDTA, 10 mM Tris-HCl pH 8.0, and 0.1% Triton X-100). The tissue was homogenized with 10 strokes each of pestle A then B, then sequentially passed through pre-wet 100 μM and 30 μM filters, collecting filtered sample in a sterile tube. The homogenizer and filters were washed with an additional 3 volumes of complete lysis buffer, collected with the filtered sample. Two volumes of sample were then layered on top of one volume of cold complete isolation buffer (1.8 M sucrose, 3 mM magnesium acetate, and 10 mM Tris-HCl pH 8.0) and spun at 21,000×g for 45 mins at 4° C. Supernatant was then carefully removed, and 100 μl complete resuspension buffer (250 mM sucrose, 25 mM KCl, 5 mM MgCl2, 20 mM Tris-HCl pH 7.2) was added to the tube without mixing. The samples were incubated on ice for 10-15 min, and then the nuclei were fully resuspended by gently pipetting up and down 20-30 times and counted with an automated cell counter (Countess 3, Thermo Fisher). For submission of single nuclei transcriptomics samples, the concentration was adjusted to ˜1×10⁶ nuclei/ml (range of 0.7-1.2×10⁶ nuclei per ml) as per the manufacturer's sample loading guidelines. To identify single nuclei transcriptomes, the manufacturer's protocol was followed to achieve ˜10,000 partitioned nuclei per sample using the 10× Genomics Chromium Controller and 3′ Gene Expression Assay (ver. 3). Nuclei partitioning, reverse transcription and cleanup, cDNA amplification, and library construction and cleanup were all performed as described in the manufacturer's protocol, and libraries were sequenced on an Illumina NextSeq2000. Final sequencing depth with average reads was 883×10⁶ reads/sample.

snRNA-Seq Analysis

After sequencing, demultiplexed Fastq files were passed through the Cell Ranger count pipeline (10× Genomics), aligning against a custom reference genome consisting of the rhesus macaque reference (Mmul 10) and the complete annotated plasmid sequence used in the generation of the rAAV vector. Cell Ranger-generated count matrices were then further analyzed within R using the package Seurat (ver. 4) and as described (Hao, Y. et al. Cell 184, 3573-3587 e3529 (2021)). Each individual sample dataset was normalized using the sctransform function, and principal component analysis (PCA), uniform manifold approximation and projection (UMAP), and nuclei clustering were all performed using standard functions within Seurat for each individual dataset.

### Antibodies

Manufacturer information for all antibodies are listed in table immediately below.

| Assay type | Species | Antibody or antigen | | Manufacturer | Catalog code |
|---|---|---|---|---|---|
| Trastuzumab | Mouse | Capture | ErbB2/Her2 Fc | Novus | 1129-ER |
| ELISA | | | Chimera Protein | Biologicals | |
| | | Detection | HRP conjugated | Sigma- | A7164 |
| | | | mouse anti-human | Aldrich | |
| | | | kappa light chain | | |
| | | | antibody | | |
| | NHP | Capture | NHP pre-adsorbed | Bio-Rad | MAS- |
| | | | mouse anti-human | | 16929 |
| | | | IgG antibody, Clone | | |
| | | | R10Z8E9 | | |
| | | Detection | Mouse pre-adsorbed | Abcam | ab98624 |
| | | | HRP conjugated Goat | | |
| | | | Anti-Human IgG Fc | | |
| | N/A | Standard | anti-HER2-Tra-hIgG1 | InvivoGen | her2tra- |
| | | curve | antibody | | mab 1 |
| ADA | Mouse | Capture | anti-HER2-Tra-hIgG1 | InvivoGen | her2tra- |
| ELISA | and | | antibody | | mab 1 |
| | NHP | Detection | HRP conjugated | Novus | NBP2- |
| | | | ErbB2/Her2 | Biologicals | 75895H |
| | | | Antibody, Clone | | |
| | | | 4D58 | | |
| | N/A | Standard | anti-Trastuzumab | Genescript | A02033 |
| | | curve | Antibody, Clone | | |
| | | | 15H2 | | |
| Western | Mouse | Primary | goat anti-human IgG | ThermoFisher | 31125 |
| Blot | | | FC antibody | | |
| | | Primary | goat anti-human | ThermoFisher | A18861 |
| | | | kappa light chain | | |
| | | | antibody | | |
| | | Primary | Beta-actin polyclonal | ThermoFisher | PA1- |
| | | | antibody | | 46296 |
| | | Secondary | Goat anti-rabbit | ThermoFisher | 31460 |
| | | | IgG HRP | | |
| IHC | Human | Estrogen | Recombinant Anti- | Abcam | ab108398 |
| | | receptor | Estrogen Receptor | | |
| | | | alpha antibody, Clone | | |
| | | | EPR4097 | | |
| | | Progesterone | Anti-progesterone | Abcam | ab32085 |
| | | receptor | Receptor antibody, | | |
| | | | Clone YR85 | | |
| | | Her2 | Recombinant Anti- | Abcam | ab134182 |
| | | | ErB2/HER2 Receptor | | |
| | | | antibody, Clone | | |
| | | | EP1045Y | | |

### In Situ Hybridization (ISH)

ISH was performed on sections from formalin-fixed paraffin embedded tissues. Sections were processed manually with an RNAscope 2.5 HD Detection kit (Advanced Cell Diagnostics, ACD) following the manufacturer's instructions. A probe consisting of ZZ probe pairs was manufactured by ACD and designed not to detect endogenous sequences in NHP tissues. Fast Red was used as chromogen to show sites of bound probes. Sections were mounted in Fluoromount with DAPI to counterstain nuclei and imaged with rhodamine and DAPI filter sets.

### Protein Extraction for ELISA and Western Blot Assays

Snap-frozen brain tissue was homogenized in lysis buffer (20 mM Tris-HCl pH: 8.0, 137 mM NaCl, 10% glycerol, 1% Triton X-100, 2 mM EDTA, IX Protease Inhibitor EDTA-free tablet) with 5 mm stainless steel beads using a bead mill homogenizer. Lysates were clarified by centrifugation and protein concentration determined via BCA assay.

Enzyme-linked immunosorbent assay (ELISA) Samples and reagents were diluted in DPBS for capture or blocking buffer (5% BSA, 0.05% Tween-20, DPBS) for detection. CSF was diluted 1:5 in blocking buffer. Brain homogenates were diluted to 2 mg total protein/mL in blocking buffer. ELISA assays were performed as previously described [Maple, L., et al., Development and validation of ELISA for Herceptin detection in human serum. J Immunol Methods, 2004. 295(1-2): p. 169-82].

### Trastuzumab

Mouse: 2 μg/mL human ErbB2/Her2 FEc Chimera Protein were first coated into high-binding 96 well plates. Trastuzumab was detected using HRP-conjugated anti-human kappa light chain antibody.

NHP: 1 pg/mL NHP pre-adsorbed mouse anti-human IgG antibody was used for capture. Mouse pre-adsorbed HRP-conjugated Goat Anti-Human IgG FEc antibody was used for detection.

0.5 μg/mL anti-HER2-Tra-hIgG1 antibody was used to generate a standard curve.

### Anti-Drug Antibody (ADA)

1 μg/mL anti-HER2-Tra-hIgG1 antibody was used for capture. 1 μg/mL anti-trastuzumab antibody was used to generate a standard curve. 2 μg/mL HRP-conjugatcd ErbB2/Her2 antibody was used for detection.

### Western Blot

Brain lysates (25 μg/lane total protein) were heat denatured in reducing LDS sample buffer, separated by SDS-PAGE, and transferred to a PVDF membrane. Electroblotted membrane was blocked in 5% skim milk. Goat anti-human IgG FC antibody was used to probe for the trastuzumab heavy chain or goat anti-human kappa light chain antibody to probe for the trastuzumab light chain. Heavy and light chains were detected using HRP-conjugated donkey anti-goat IgG antibody. Blots were developed using chemiluminescent substrate.

### Liquid Chromatography Mass Spectrometry (LC-MS)

Detection of trastuzumab by LC-MS in mouse and NHP brain samples were also performed by LC-MS.

### LC-MS Analysis

### Mouse Brain Lysates

Signature peptides for LCMS analysis and multiple reaction monitoring (MRM) transitions were chosen from previous literature [Russo, R., et al., Ultra-performance liquid chromatography/multiple reaction monitoring mass spectrometry quantification of trastuzumab in human serum by selective monitoring of a specific peptide marker from the antibody complementarity-determining regions. Rapid Communications in Mass Spectrometry, 2017. 31(14): p. 1184-1192]. Tissue samples were homogenized in lysis buffer (50 mM ammonium bicarbonate, 6M urea, 0.15% sodium deoxycholate) at a ratio of 7 μL buffer to ˜1 mg of tissue. Homogenate was clarified and protein concentration of clarified lysate was determined by BCA. A trastuzumab calibration curve ranging from 68-7277 ng/mL was created in clarified lysate from a control mouse. Twenty L of calibration standard, animal samples, clarified lysate blank, and clarified lysate double blanks were plated in duplicate in a 1 mL 96-well plate. 50:50 methanol:acetone was added to each well, the plate was vortexed and dried in a Eppendorf Vaccufuge using no heat. Samples were reconstituted in 6M urea, reduced in 8 mM 2-carboxyethyl) phosphine at 37° C. for 1 hour shaking at 500 rpm, followed by alkylation in 30 mM iodoacetamide at room temperature in the dark for 30 minutes. Standards, samples, and blanks were spiked with 20 μL of 10 nM heavy labeled internal peptide standard solution (DTYIHWVR; SEQ ID NO: 56). Samples were diluted 1:3 with 0.1 mg/mL trypsin prepared in 50 mM ammonium bicarbonate. Samples were digested overnight at 37° C. shaking at 500 rpm. Digestion was quenched to a final concentration of 0.7% formic acid. Solid phase extraction was performed using a Waters Oasis HLB sorbent. Eluted peptides were dried in an Eppendorf Vaccufuge using no heat and reconstituted in 5:95 acetonitrile: water and 0.1% formic acid. LCMS analysis was performed using an Agilent 1260 Infinity II HPLC and Agilent TQ 6495 mass spectrometer. Mobile phase A was 0.1% formic acid in water and mobile phase B was 0.1% formic acid in acetonitrile. The stationary phase was a Waters Premier HSS T3 1.8 m 2.1×100 mm using a gradient from 15% mobile phase B to 21% mobile phase B over 8 minutes. Data was analyzed and quantitated using Skyline by the ratio to heavy normalization method and a 1/x*x regression weighting [MacLean, B., et al., Skyline: an open source document editor for creating and analyzing targeted proteomics experiments. Bioinformatics, 2010. 26(7): p. 966-968]. Calculated concentrations were normalized against BCA protein content.

### NHP Brain Lysates

Trastuzumab pull-down: Human HER2-coupled magnetic beads were reconstituted and washed according to vendor's recommendations (Acro Biosystems MBS-K006). One hundred μL of beads per sample were pelleted in a microcentrifuge tube using a magnetic separator and the supernatant was discarded. Protein lysate samples from NHP brain homogenates were prepared as described in the LC-MS analysis of mouse brain lysate section. Samples were diluted to 2 mg/mL in PBS-T and 1 ml of diluted lysate was added to tubes with pelleted beads. Samples were incubated overnight at 4° C. on a tube rotator. Following incubation, beads were pelleted using a magnetic separator and the supernatant was discarded. Beads were washed with PBS-T by vortexing the tube for 20 seconds, pelleting the beads on a magnetic separator, and discarding the supernatant a total of 4 times.

Protein bound beads were prepared according to the SP3 protocol [Hughes, C. S., et al., Single-pot, solid-phase-enhanced sample preparation for proteomics experiments. Nature Protocols, 2019. 14(1): p. 68-85]. A heavy labeled internal standard peptide (DTYIHWVR; SEQ ID NO: 56) was spiked into solution for retention time reference and normalization. Each sample was split into two vials for targeted and untargeted LCMS analysis.

Vial one was analyzed using the targeted MRM acquisition method, as discussed in the LC-MS analysis of mouse brain lysate section. Data was analyzed and quantitated using Skyline by the ratio to heavy normalization method and a linear through zero single point calibration regression, using the 10 ng/mL positive control as a reference [MacLean, B., et al., Skyline: an open source document editor for creating and analyzing targeted proteomics experiments. Bioinformatics, 2010. 26(7): p. 966-968].

Vial two was analyzed using a Thermo UltiMate 3000 RSLNano LC system and Thermo Q-Exactive HF Orbitrap mass spectrometer. Mobile phase A was 0.1% formic acid in water and mobile phase B was 0.1% formic acid in acetonitrile. The stationary phase was an Easy-Spray PepMap Neo 2 μm C18 75 μm×150 mm using a gradient from 4% mobile phase B to 50% mobile phase B over 65 minutes, followed by a high organic wash and re-equilibration. Data acquisition was performed using a top 10 data dependent acquisition method. MS1 resolution was set to 60,000 with the AGC target set to 1 e6 with maximum IT at 60 ms scanning from 300-1500 m/z and an isolation window of 2 m/z. MS2 resolution was set to 30,000 with the AGC target set to 1e6 with maximum IT at 120 ms scanning from 50-2000 m/z and an NCE of 27. The minimum intensity threshold was set at 8.3e3. Charge state exclusion was set to unassigned, 1, and >7 and dynamic exclusion was set to 5 seconds. Data was analyzed using Skyline with peptide settings set to a maximum of 1 tryptic missed cleavage site and fixed carbamidomethyl cysteine. Skyline transition settings were set to charge states of 2-4, ion charge of 1, and ion types of y, b, and p. Skyline product ion was set from ion 1 to last ion. Peptide mapping coverage was reviewed manually for accuracy. Heavy chain to light chain relative quantitation was calculated using average MS1 peak areas from the 3 most abundant peptides in each chain.

### Statistical Analyses

Differences in trastuzumab expression levels between treated and control groups were analyzed using unpaired two-tailed student t-tests in GraphPad Prism 9.5.1. Comparison of number of viral genome copies between different treatment groups were calculated using t-tests with log transformed values. Estimated Kaplan-Meier survival probabilities for tumor challenge experiments in mice were plotted using GraphPad Prism 9.5.1 and differences between groups compared using the Log-rank test.

### Results

ICV delivery of AAV9.UbC.trastuzumab vector resulted in transgene expression in Rag1

### KO Mice

To achieve stable trastuzumab expression in vivo, healthy young adult Rag1 KO female mice were ICV injected (1×10n GC/mouse) with AAV9.UbC.trastuzumab and trastuzumab levels were quantified in the systemic circulation and brain parenchyma (FIGS. 31A and 31B). ICV delivery of AAV9.UbC.trastuzumab vector (1×10¹¹ GC/mouse) in Rag1 KO mice resulted in robust systemic and local transgene expression after 4 weeks (100-1000-fold signal increase over background, FIGS. 31C and 31D). Biodistribution studies of brain and liver tissue confirmed that vector delivery (FIG. 31E) and transgene expression (FIG. 31F) was successful in AAV-treated mice (AAV genome copies per microgram of DNA: 600-3,000 fold signal increase over background; transgene RNA transcript: 5,000-42,000 fold signal increase over background). Western blot analysis confirmed the expression of human IgG antibody heavy and light chains in whole brain lysates from mice receiving AAV-trastuzumab via ICV injection (FIG. 31G). The identity of the expressed transgene was confirmed to be trastuzumab by LC-MS with multiple reaction monitoring transition via quantification of a unique peptide (DTYIHWVR; SEQ ID NO: 56) present in brain tissue homogenates of ICV-treated mice receiving AAV9.UbC.trastuzumab (signal≥588-fold above background compared to PBS control; FIG. 31H).

FIGS. 31A-31H shows single ICV administration of AAV9 vector encoding engineered version of trastuzumab resulted in robust transgene expression in RAG1 KO Mice. FIG. 31A shows a schematic of AAV vector genome. FIG. 31B shows schematic for evaluation of in vivo transgene expression following ICV delivery of AAV9 vector encoding trastuzumab (1×10¹¹ GC/mouse) in healthy adult Rag1 KO female mice. FIG. 31C shows longitudinal quantification of trastuzumab by ELISA in serum from Rag1 KO mice treated ICV with AAV9.UbC.trastuzumab vector (1×10¹¹ GC/mouse; n=10) or PBS (n=10). FIG. 31D shows longitudinal quantification of trastuzumab by ELISA in perfused brain tissue from Rag1 KO mice treated ICV with AAV9.UbC.trastuzumab vector (1×10¹¹ GC/mouse; n=10) or PBS (n=10). FIG. 34E shows DNA biodistribution and FIG. 31F shows RNA biodistribution analysis by qPCR of brain and liver tissue from Rag1 KO mice treated ICV with AAV9.UbC.Trastuzumab or PBS. FIG. 31G shows Western blot analysis of Trastuzumab heavy and light chains in brain lysates from Rag1 KO mice treated ICV with AAV9.UbC.trastuzumab (n=4) or PBS (n=2). Purified trastuzumab (10 ng/lane) and β-actin were used as a positive control and loading control, respectively. FIG. 31H shows trastuzumab identity was confirmed by LC-MS using brain homogenates from Rag1 KO mice treated ICV with AAV9.UbC.trastuzumab (n=4) or PBS (n=2). Data shown as individual data points and mean±SEM.

Preventing Off-Target Transduction does not Affect Trastuzumab Expression in the Brains of Rag1 KO Mice

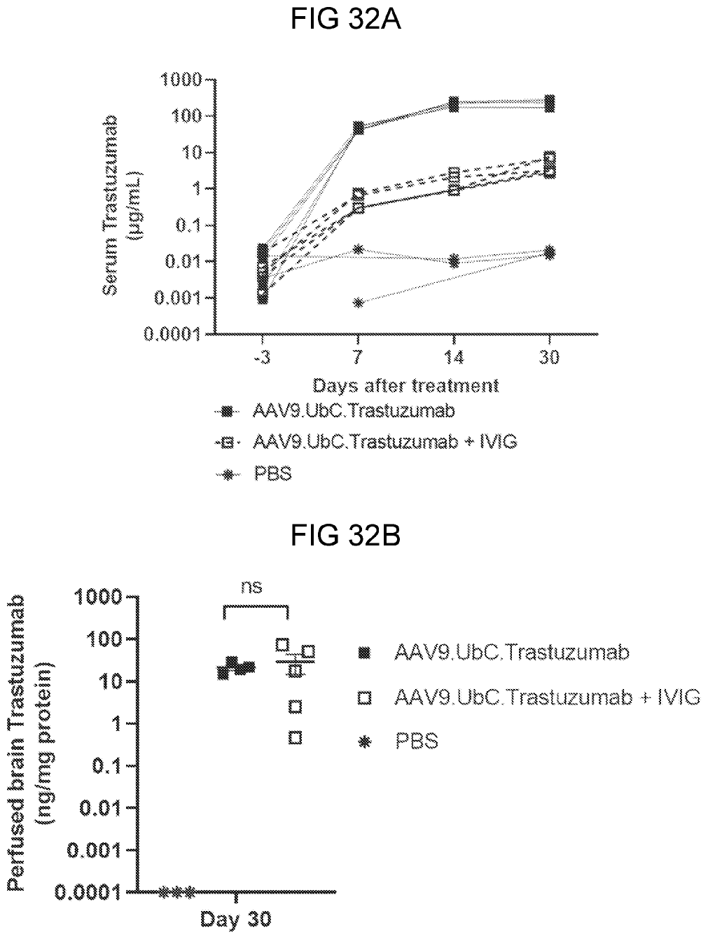

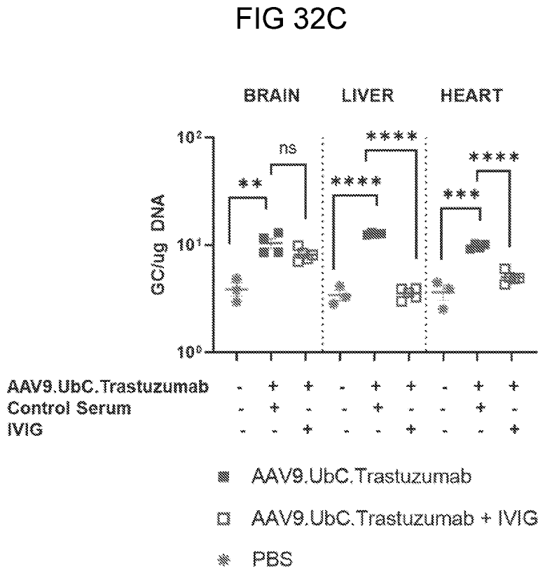

Cardiotoxicity is a significant but largely reversible complication of systemic trastuzumab treatment observed in a subset of patients with metastatic HER2+ breast cancer. The incidence of trastuzumab-related cardiotoxicity varies greatly according to risk factors like pre-existing cardiac dysfunction; the likelihood of developing any type of cardiotoxicity peaks at 11 months following treatment commencement and decreases over time [Bouwer, N. I., et al., Cardiotoxicity during long-term trastuzumab use in patients with HER2-positive metastatic breast cancer: who needs cardiac monitoring?Breast Cancer Res Treat, 2021. 186(3): p. 851-862]. As AAV vector can leak into the general circulation from the CSF to transduce off-target organs following IT delivery, we assessed whether intravenous plasma-derived pooled human immunoglobulin (TVTg; a source of anti-AAV9 neutralizing antibodies) could reduce extra-cranial organ transduction following ICV AAV vector delivery in mice [Boutin, S., et al., Prevalence of serum IgG and neutralizing factors against adeno-associated virus (AAV) types 1, 2, 5, 6, 8, and 9 in the healthy population: implications for gene therapy using AAV vectors. Hum Gene Ther, 2010. 21(6): p. 704-12]. Compared to the control group that received naïve serum, mice that received IVIg 2 hours prior to ICV injection of AAV9.UbC.trastuzumab (1×10 GC/mouse) showed a 40-fold reduction in serum trastuzumab concentrations (FIG. 32A) while levels in the brain remained unaffected (FIG. 32B). Vector biodistribution analyses corroborated these findings (FIG. 32C) showing that the reduced systemic, but not brain levels of trastuzumab, resulted from effective blockade of only liver and heart transduction following IVIg pre-treatment. In humans, pre-existing immunity against recombinant AAV vectors following natural exposure to wild type AAV can greatly reduce tissue transduction. However, as the CSF contains only a fraction of NAbs compared to blood [Horiuchi, M., et al., Intravenous immunoglobulin prevents peripheral liver transduction of intrathecally delivered AAV vectors. Mol Ther Methods Clin Dev, 2022. 27: p. 272-280; Verdera, H. C., K. Kuranda, and F. Mingozzi, AAV Vector Immunogenicity in Humans: A Long Journey to Successful Gene Transfer. Mol Ther, 2020. 28(3): p. 723-746], pre-existing immunity to AAV would likely not contraindicate this gene therapy approach for most potential patients.

FIGS. 32A to 32C show AAV9.UbC.trastuzumab CNS transduction efficiency is not affected by pre-treatment with intravenous immunoglobulin (IVIg) containing broad neutralizing antibodies against AAV9. Adult female Rag1 KO mice were pre-treated with IVIg or control serum from C57BL6/J donor mice 2 hours prior to ICV administration of AAV9.UbC.Trastuzumab (1×10¹¹ GC/mouse; n=5). Mice treated ICV with PBS (n=3) were used as controls. FIG. 32A shows trastuzumab levels in serum over time and FIG. 32B shows trastuzumab levels in perfused brain tissue (day 30) from ICV treated mice were measured by ELISA. FIG. 32C shows DNA biodistribution analysis of AAV9.UbC.Trastuzumab vector by qPCR in brain, liver and heart 30 days post-vector administration. Data shown as individual data points and mean±SEM. ***p<0.0001; ns, not significant.

Treatment with AAV9.UbC.Trastuzumab Inhibited Tumor Progression in Orthotopic Xenograft Mouse Models of HER2+ Breast Cancer Brain Metastasis

We next sought to evaluate anti-tumor efficacy of AAV9.UbC.trastuzumab ICV administration in an immunocompromised mouse model that retains NK cell-mediated ADCC activity. Orthotopic xenograft Rag1 KO mouse models were generated by engrafting GFP/luciferase-reporter HER2+ cell lines (BT-474 or MDA-MB-453) in the right frontal lobe using a guide-screw system [Lal, S., et al., An implantable guide-screw system for brain tumor studies in small animals. J Neurosurg, 2000. 92(2): p. 326-33]. Hormone receptor and HER2 status for each cell line was confirmed by IHC staining of tumor-bearing brain sections from Rag1 KO mice (data not shown). Three days after tumor cell implantation, animals were randomized to treatment groups and received AAV9.UbC.trastuzumab or AAV9.UbC.isotype control vector (1×10¹¹ GC/mouse) via ICV injection; tumor growth was monitored by bioluminescence imaging (data not shown).

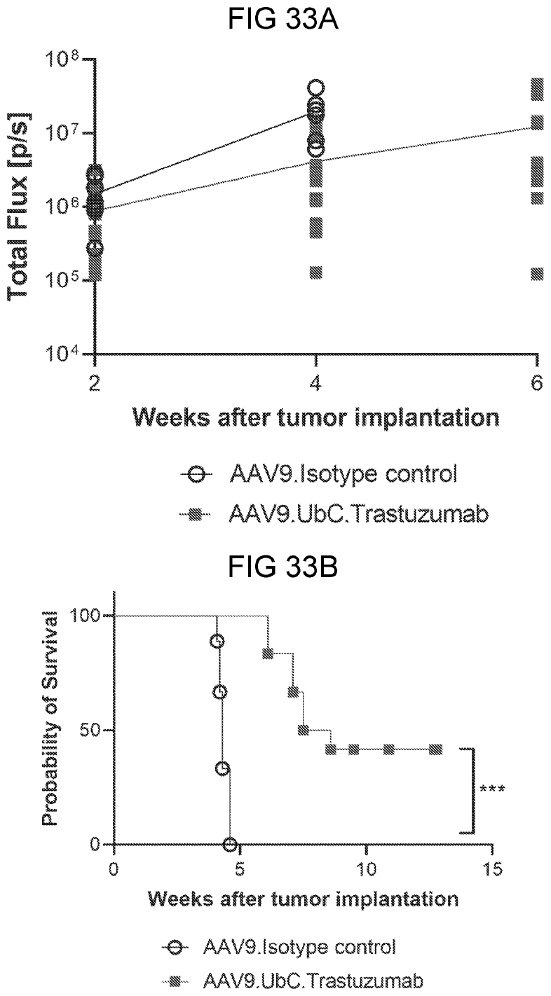

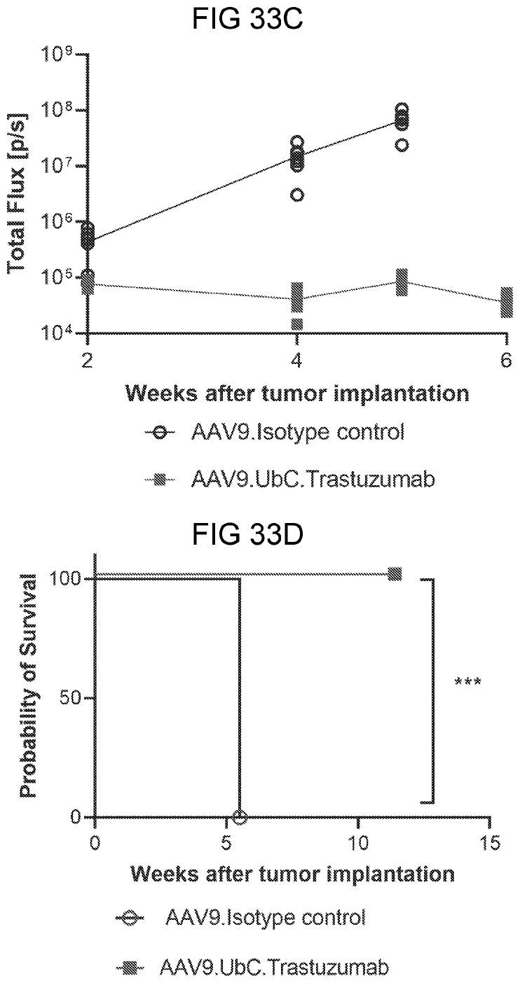

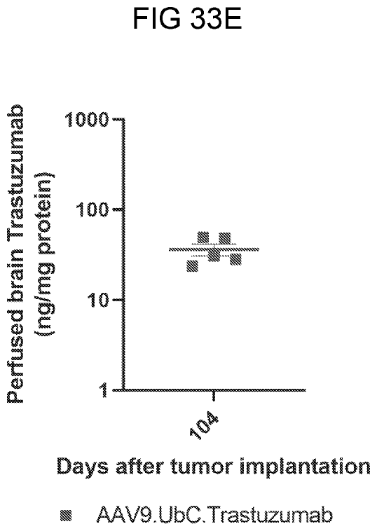

Compared to AAV9.UbC.isotype control-treated mice, AAV9.UbC.trastuzumab administration delayed tumor progression (FIG. 33A) and extended the OS (FIG. 33B) of BT-474 tumor-bearing mice (3.8 weeks vs 9.9 weeks, respectively). Six of the 12 mice treated with AAV9.UbC.trastuzumab remained asymptomatic for >12 weeks and were considered to be in complete remission. ICV treatment of Rag1 KO mice bearing MDA-MB-453 xenograft tumors with AAV9.UbC.trastuzumab achieved a 100% response rate (FIG. 33C) and all animals remained in remission >12 weeks post treatment; the OS of mice treated ICV with AAV9.UbC.isotype control vector was 5.5 weeks (FIG. 33D). Analysis of brain homogenates from Rag1 KO mice orthotopically implanted with MDA-MB-453 104 days after ICV administration of AAV9.UbC.trastuzumab showed persistent trastuzumab expression (ranging from 23.7-49.5 ng trastuzumab/mg total protein; FIG. 33E). Combined, these results provide proof-of-principle evidence which supports the use of direct CNS delivery of AAV9.UbC.trastuzumab vector to target HER2+ BCBM.

FIGS. 33A to 33E show that a single dose ICV administration of AAV9.UbC.Trastuzumab led to tumor regression in xenograft mouse models of HER2+ breast to brain metastases. Three days post-intracranial implantation of BT-474 (ER+/PR+/HER2+) or MDA-MB-453 (ER−/PR−/HER2+) human breast cancer cell lines, adult female Rag1 KO mice were ICV injected with 1×10¹¹ GC/animal of AAV9.UbC.trastuzumab or AAV9.isotype control. FIG. 33A shows quantification of total photon flux via bioluminescent in vivo imaging system (IVIS) and FIG. 33B shows a Kaplan-Meier survival curve of mice bearing BT-474 cell line-derived orthotopic brain tumors and treated ICV with AAV vectors (AAV9.Isotype control: n=9; or AAV9.UbC.trastuzumab: n=12). FIG. 33C shows representative bioluminescent IVIS images (at week 4, left panel), total photon flux signal quantification (right panel), and FIG. 33D shows a Kaplan-Meier survival curve of mice bearing MDA-MB-453 cell line-derived orthotopic brain tumors following ICV treatment (AAV9.isotype control: n=8; or AAV9.UbC.trastuzumab: n=7). FIG. 33E shows a long-term trastuzumab expression quantified in perfused brain tissue by ELISA in a subset of mice showcased in FIG. 33C and FIG. 33D (n=5) at day 104 post-ICV administration of AAV9.UbC.trastuzumab. ***p<0.0001

Successful Expression of Therapeutic Antibody Protein in the CSF of NHPs Treated with AAV9.UbC.Trastuzumab

NHPs are more comparable in size and have greater immunological similarity to humans, which makes them the gold standard model species for assessing AAV safety and CNS vector distribution [Gopinath, C., et al., Contemporary Animal Models For Human Gene Therapy Applications. Curr Gene Ther, 2015. 15(6): p. 531-40]. To achieve broad vector distribution within the CNS, AAV9.UbC.trastuzumab was delivered into the CSF of adult female Indian rhesus macaques with pre-existing AAV9-neutralizing antibodies via ICM injection (3×10¹³ GC/animal, n=2/group), as previously described [Hinderer, C., et al., Widespread gene transfer in the central nervous system of cynomolgus macaques following delivery of AAV9 into the cisterna magna. Mol Ther Methods Clin Dev, 2014. 1: p. 14051]. Body weights and comprehensive serum chemistry remained within normal parameters compared to baseline measurements. No physical deficits or clinical signs of toxicity were observed throughout the study and animals were euthanized at their scheduled endpoint on days 36-37. No gross lesions or histopathological abnormalities were observed following necropsy.

We then aimed to assess whether ICM injection of AAV9.UbC.trastuzumab vector led to detectable transgene expression in the serum and CSF of treated animals as a longitudinal surrogate marker of CNS gene transfer. Samples were collected at baseline and weekly intervals until scheduled euthanasia (FIG. 34A). Serum and CSF samples collected from a NHP previously treated ICM with AAV9.GFP was used as a negative control. Quantification of trastuzumab levels demonstrated evidence of drug accumulation in the CSF (FIG. 34B), but not serum (FIG. 34C), in 2/2 NHPs that received AAV9.UbC.trastuzumab via ICM injection as compared to the control NHP. The development of anti-drug antibodies (ADA) against trastuzumab, which were detected in later time-points in the CSF (FIG. 34D) and serum (FIG. 34E) of 1/2 NHPs treated with AAV9.UbC.trastuzumab likely contributed to CSF drug clearance (FIGS. 34D and 34E). Overall, these results confirm successful expression of Trastuzumab in the CSF of NHPs post ICM administration of AA9.UbC.Trastuzumab.

FIGS. 34A to 34E show detection of trastuzumab in the CSF of female NHPs following ICM administration of AAV9 vectors encoding trastuzumab. FIG. 34A sows schematic design of experimental ICM delivery of 3×10¹³ GC/animal of AAV9.UbC.trastuzumab (n=2) vector in NHPs to inform in-life blood and CSF collection schedules and scheduled end-of-study time points. NHP #3 was injected ICM with 3×10¹³ GC of an AAV9 vector expressing GFP and was used as a negative control. Detection of trastuzumab, a humanized IgG1 antibody, in the CSF (FIG. 34B) and serum (FIG. 34C) of treated animals was determined by ELISA following quantification of human IgG and anti-drug antibodies against trastuzumab in CSF (FIG. 34D) and serum (FIG. 34E).

### ICM Delivery of AAV Vectors Encoding Trastuzumab Resulted in Widespread Transduction and Transgene Expression in CNS Tissues

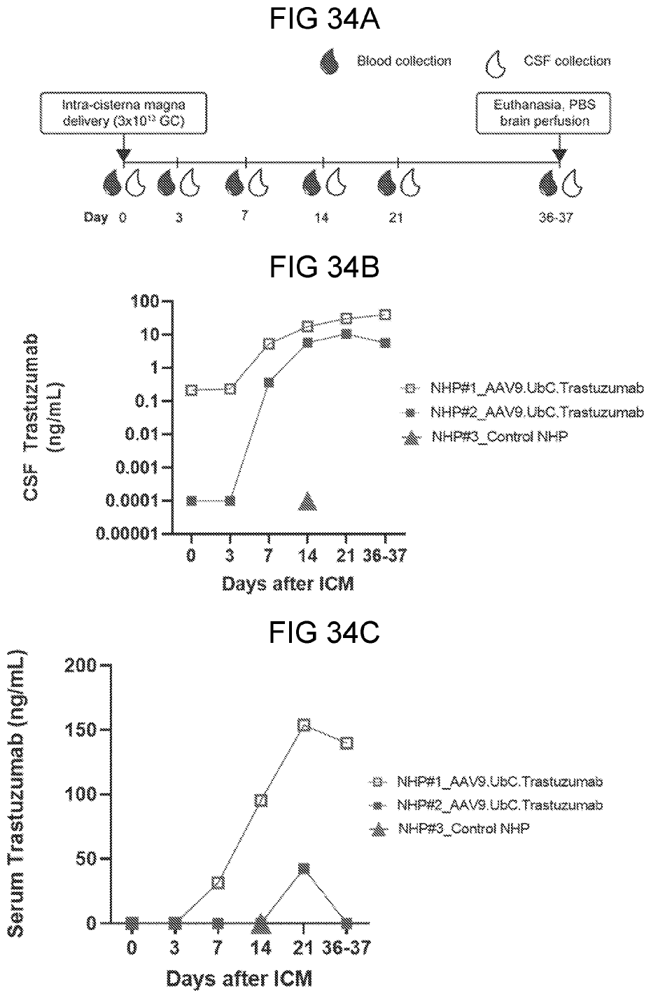

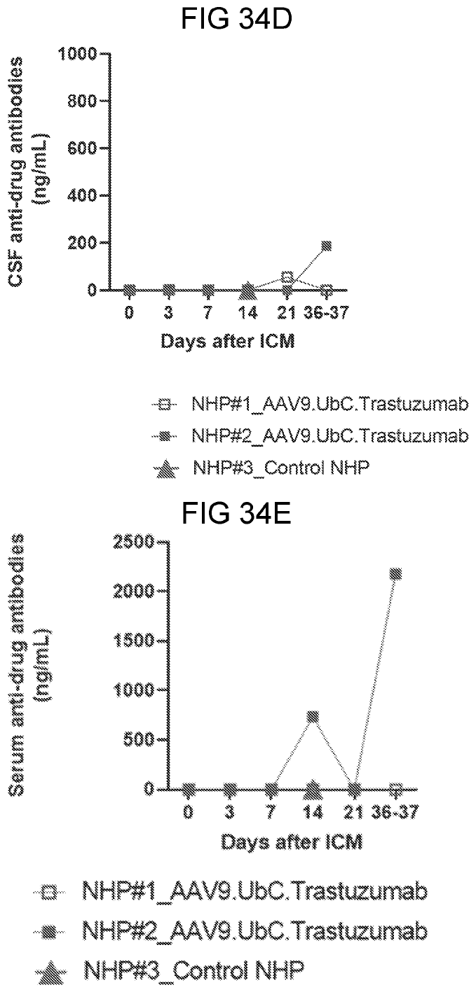

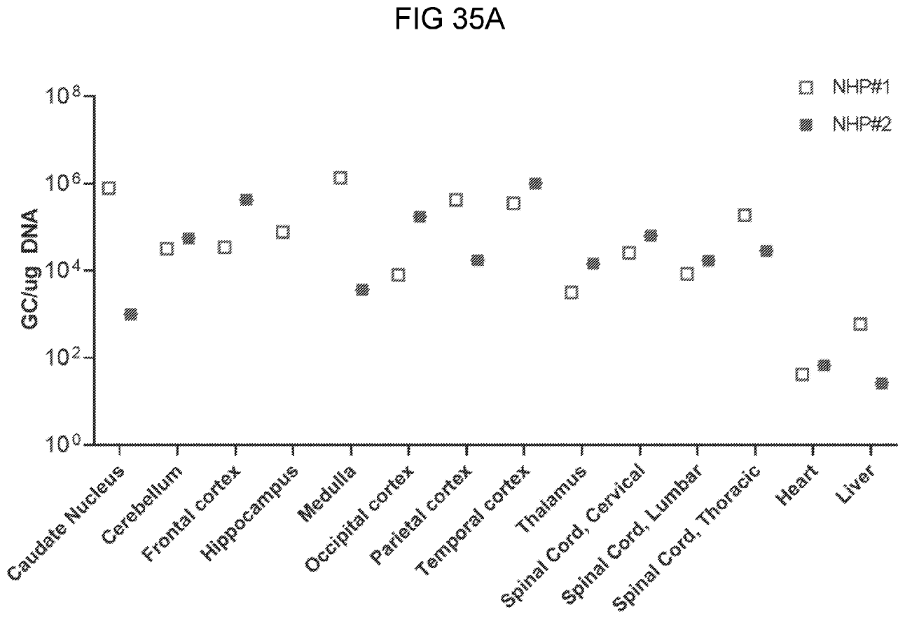

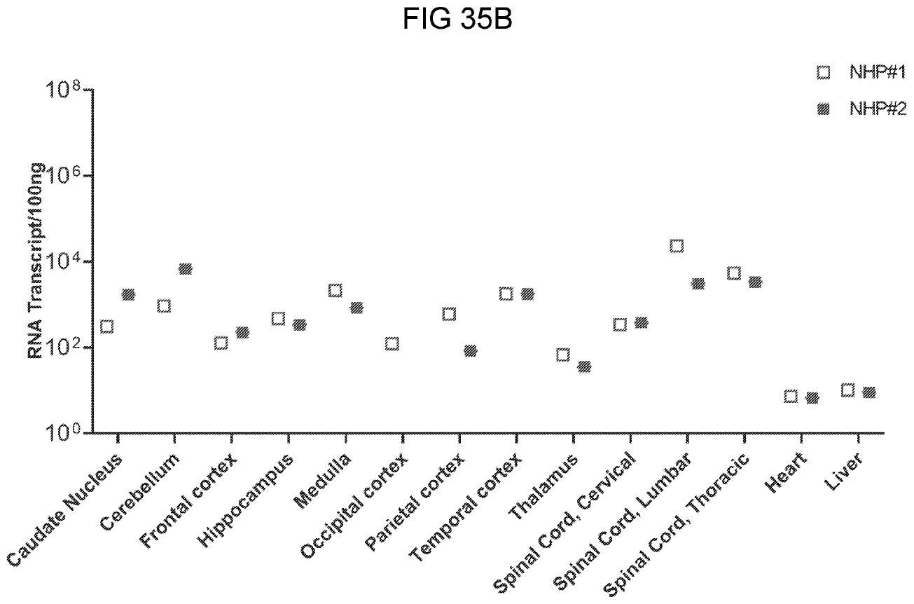

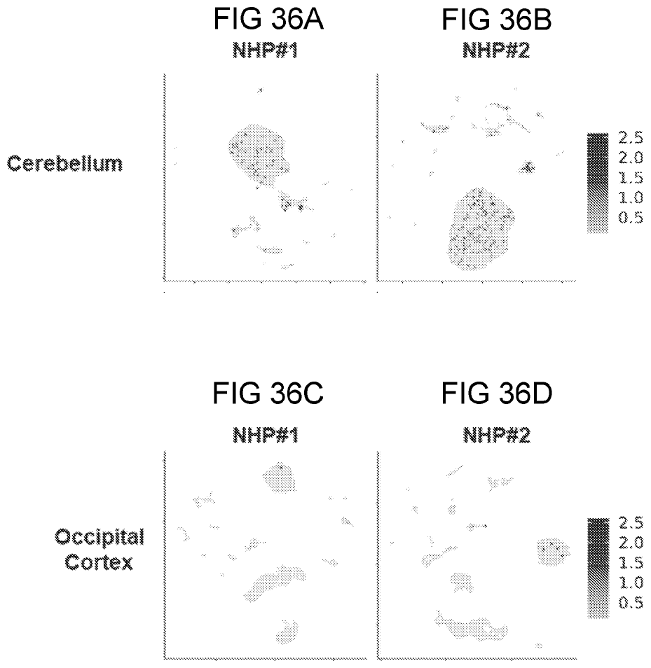

DNA biodistribution analyses (FIG. 35A) showed 100-10,000-fold higher vector GC in the brain and spinal cord compared to the heart and liver of NHPs that received ICM AAV9.UbC.trastuzumab (3×10¹³ GC/animal). The presence of transgene transcript following transduction was evaluated in CNS, heart, and liver. Consistent with the DNA biodistribution data, transgene mRNA expression was 100-1,000-fold higher in brain and spinal cord regions compared to the heart and liver of these animals (FIG. 35B). The presence of trastuzumab transcripts in the CNS of NIHPs treated ICM with AAV9.UbC.trastuzumab (3×10¹³ GC/animal) was confirmed via in situ hybridization using a probe specific for the engineered trastuzumab version in paraffin-embedded sections from the cerebellum and occipital cortex (data not shown). Single-nuclei RNA sequencing further confirmed the presence of transgene transcripts in the cerebellum and occipital cortex from NHPs that received ICM AAV9.UbC.trastuzumab, although differential vector transduction levels were observed in these regions (0.6 to 1% total trastuzumab positive cells in the cerebellum vs. 0.02-0.08% total trastuzumab positive cells in the occipital cortex; FIGS. 36A to 36D). These data suggest that extra-cranial transduction by AAV leaked from the CSF was largely prevented in these animals, since they had pre-existing levels of anti-AAV9 neutralizing antibodies prior to ICM treatment.

FIGS. 35A, 35B, and 36A to 36D show CNS-wide gene delivery and transgene transcription following ICM treatment with 3×10¹³ GC/animal of AAV9.UbC.trastuzumab. FIG. 35A shows absolute quantification of AAV vector GC in NHPs on day 36-37 post-ICM administration of AAV9.UbC.Trastuzumab (n=2) vector. FIG. 35B shows transcripts in selected tissues by qPCR in NHPs on day 36-37 post-ICM administration of AAV9.UbC.Trastuzumab (n=2) vector.

FIGS. 36A to 36D show results of single-nuclei RNA sequencing showing the presence of trastuzumab mRNA transcripts in the cerebellum and occipital cortex from animals injected ICM with AAV9.UbC.Trastuzumab vector.

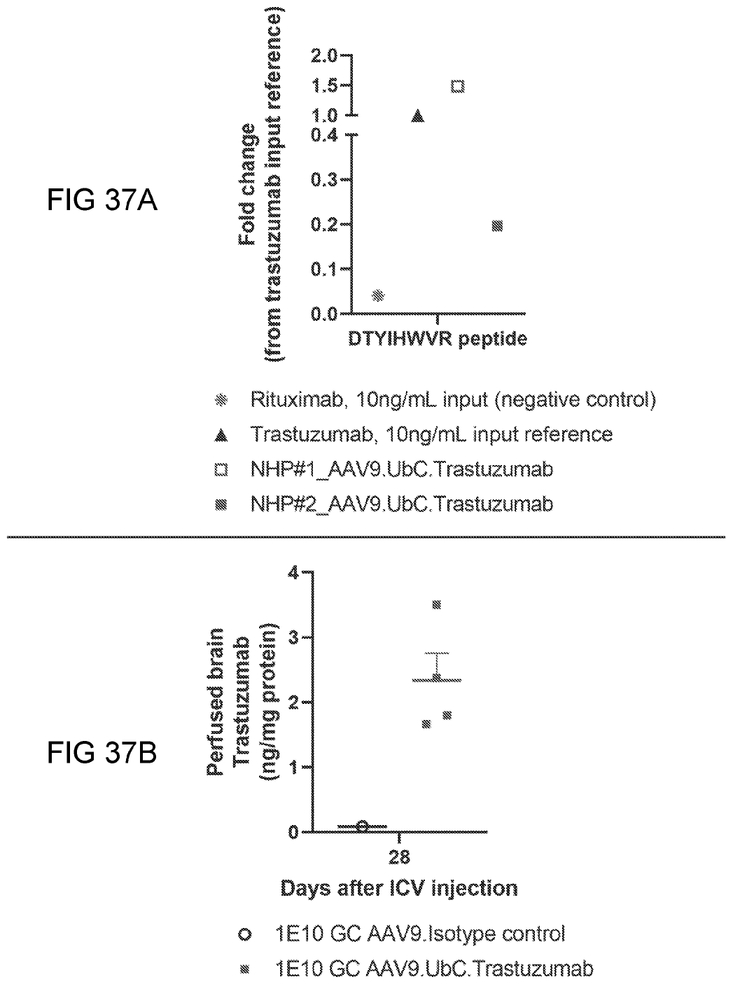

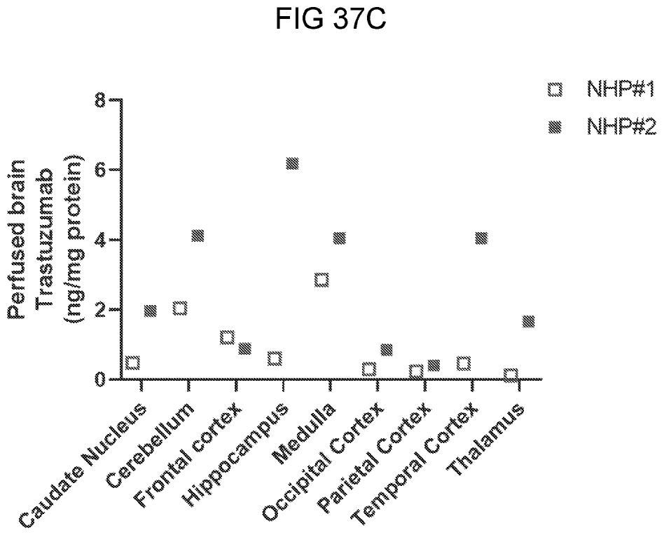

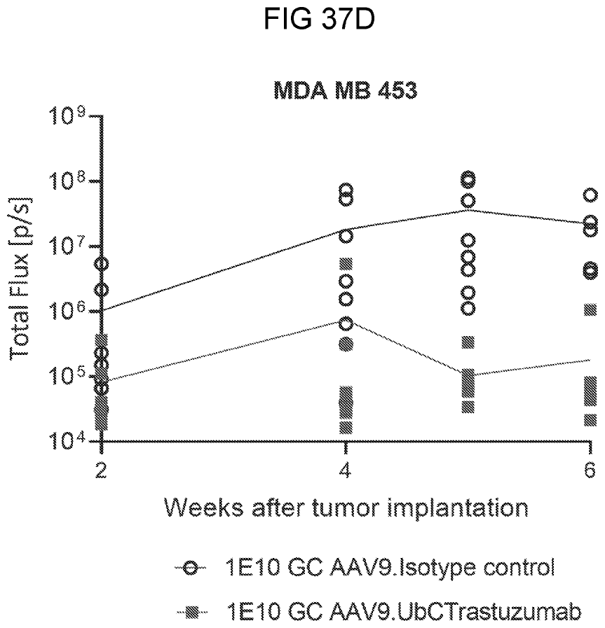

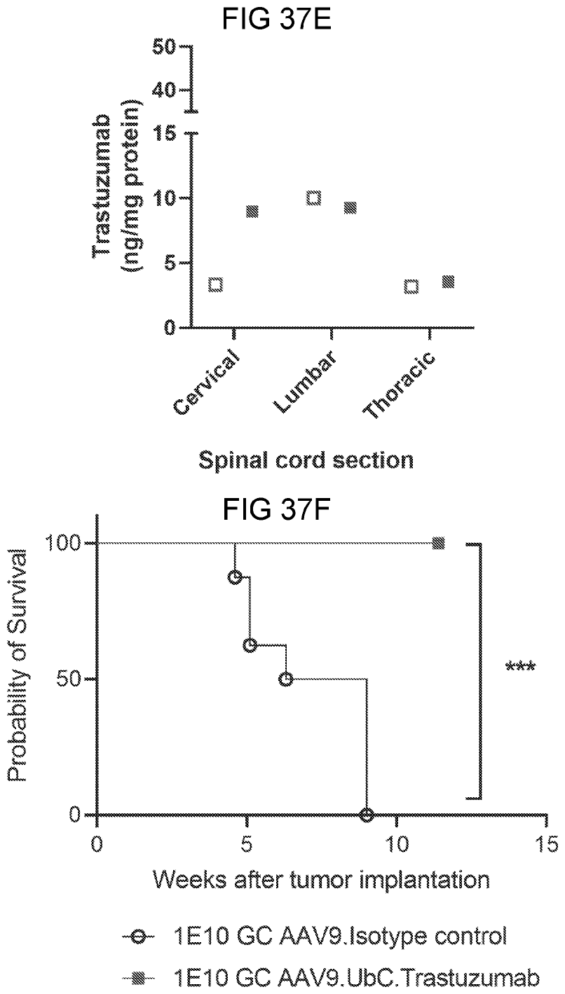

Next, the identity of AAV-encoded trastuzumab in NHP CNS tissues was analyzed using LC-MS targeted and untargeted proteomic approaches (based on the detection of a unique trastuzumab-derived peptide and peptide mapping analysis, respectively). To reduce the matrix effect and improve assay sensitivity, NHP brain homogenates were prepared under non-denaturing conditions and subjected to trastuzumab pull down using HER2-coated magnetic beads. Ten nanograms of purified research-grade trastuzumab or rituximab (anti-human CD20) antibodies were added to brain homogenates consisting of a 1 mL solution containing 1 mg total protein from an untreated NHP control to provide a positive or negative input control for the pull-down assays, respectively. After incubation and recovery of the HER2-coated magnetic beads, bead-bound antibodies were released by proteolysis and the peptide fragments were analyzed by LC-MS. For the targeted proteomics approach, the amount of trastuzumab recovered from the positive input control were used for normalization; as expected, antibody binding to HER2 was specific, as essentially no rituximab was recovered in the pull down. The presence of trastuzumab in the brain homogenates from NHPs receiving ICM AAV9.UbC.trastuzumab was confirmed by the detection of a unique trastuzumab-derived peptide and by peptide mapping of trastuzumab heavy and light chains (FIG. 37A).

ELISA were used to further determine the abundance of human IgG protein (as a surrogate for trastuzumab) in multiple brain regions and in the spinal cord from NHPs that received ICM AAV9.UbC.trastuzumab. CNS tissue samples harvested from a NHP previously treated ICM with AAV9.GFP was used as a negative control. In the brain parenchyma (FIG. 37C), levels of human IgG appeared to be consistently higher among both AAV9.UbC.trastuzumab treated NHPs in the cerebellum and medulla (2-4 ng of trastuzumab/mg total protein). The cerebellum is a hotspot for HER2+ BCBM, and sustained trastuzumab expression in this region could efficiently target the tumor bed and slow disease progression [Kyeong, S., et al., Subtypes of breast cancer show different spatial distributions of brain metastases. PLoS One, 2017. 12(11): p. e0188542]. Trastuzumab levels appeared to be slightly higher in the spinal cord compared to brain in these animals (3-10 ng/mg total protein; FIG. 37E). Our choice to employ this route of administration is further validated by reports that ICM vector delivery results in higher vector transduction of the spinal cord compared to lumbar puncture, meaning leptomeningeal metastases lining the spinal cord could also be a feasible target using this approach [Hinderer, C., et al., Widespread gene transfer in the central nervous system of cynomolgus macaques following delivery of AAV9 into the cisterna magna. Mol Ther Methods Clin Dev, 2014. 1: p. 14051].

Tumor Remission in Mice Treated with Low Dose AAV9.UbC.Trastuzumab ICV Achieve CNS Transgene Expression Levels Similar to NHPs

Since the trastuzumab expression levels achieved in the NHP brain parenchyma were lower than the levels observed in the tumor challenge experiments in mice presented above, we interrogated whether treatment of tumor-bearing mice with a dose of AAV9.UbC.trastuzumab that recapitulated the level of transgene expression in the CNS of NHPs would retain sufficient anti-tumor activity. A dose-down titration and ICV delivery of AAV9.UbC.trastuzumab from 1×10¹¹ GC/mouse to 1×10¹⁰ GC/mouse resulted in trastuzumab accumulation in the brain parenchyma of healthy female Rag1 KO mice similar to that observed in the cerebellum and medulla of ICM-treated NHPs (1.6-3.5 ng/mg total protein; FIG. 37B). ICV treatment of Rag1 KO mice bearing MDA-MB-453 cell line-derived orthotopic brain xenografts with 1×¹⁰′° GC/mouse AAV9.UbC.trastuzumab resulted in a 100% response rate, with all mice in complete remission beyond 12 weeks after engraftment compared to animals treated with AAV9.UbC.Isotype control (OS 7.6 weeks; FIGS. 37D and 37F). These results demonstrate that local expression of AAV-encoded trastuzumab in the CNS of tumor-bearing mice provided a robust antitumor response even at the lower therapeutic drug concentration levels observed in ICM-treated NHPs. Altogether, ICM delivery of AAV9.UbC.trastuzumab in NHPs was well tolerated and resulted in CNS transgene expression at levels exhibiting therapeutic effects.

FIGS. 37A to 37F show that ICM delivery of 3×10¹³ GC/animal AAV9.UbC.trastuzumab results in transgene protein expression in NHP CNS tissues at levels sufficient to induce complete antitumor responses in tumor bearing mice. Experimental scheme is shown in FIG. 34A. FIG. 37A shows results of LC-MS analysis to detect a unique AAV-encoded, trastuzumab-derived peptide (DTYIHWVR; SEQ ID NO: 56) in NHP brain homogenates. Trastuzumab levels presented as fold change relative to input reference positive control (10 ng trastuzumab). FIG. 37B shows trastuzumab expression in perfused brain tissue from female Rag1 KO mice 28 days post-ICV administration of 1×10¹⁰ GC/animal of AAV9.isotype control (n=8) or AAV9.UbC.trastuzumab (n=8), measured by ELISA. FIG. 37C shows trastuzumab protein expression in perfused brain regions by ELISA by determining the tissue levels of human IgG. FIG. 37D shows quantification of total photon flux by IVIS imaging of adult female Rag1 KO mice treated ICV with 1×10¹⁰ GC/animal of AAV9.isotype control (n=8) or AAV9.UbC.trastuzumab (n=8) 3 days following intracranial tumor cell implantation of reporter MDA MB 453 breast cancer cells. FIG. 37E shows trastuzumab protein expression in spinal cord which were quantified by ELISA by determining the tissue levels of human IgG. FIG. 37F shows Kaplan Meier survival curves of adult female Rag1 KO mice treated ICV with 1×10¹⁰ GC/animal of AAV9.isotype control (n=8) or AAV9.UbC.trastuzumab (n=8) 3 days following intracranial tumor cell implantation of reporter MDA MB 453 breast cancer cells. ***p<0.0001

### Discussion

The mechanisms contributing to the transition from tumor cell quiescence to symptomatic CNS metastatic onset are unclear, but they typically manifest in the brain parenchyma and leptomeninges around 2-3 years after an initial breast cancer diagnosis (80% vs. 11-20% of cases, respectively) [Pestalozzi, B. C., et al., Identifying breast cancer patients at risk for Central Nervous System (CNS) metastases in trials of the International Breast Cancer Study Group (IBCSG). Ann Oncol, 2006. 17(6): p. 935-44; Scott, B. J. and S. Kesari, Leptomeningeal metastases in breast cancer. Am J Cancer Res, 2013. 3(2): p. 117-26]. Overall prognosis and survival of patients diagnosed with BCBM are contingent upon independent factors including tumor molecular subtype, Karnofsky performance status, and the size and number of tumors present; for instance, having >3 foci of BCBM at the time of diagnosis is correlated with worse outcomes [Griguolo, G., et al., External validation of Modified Breast Graded Prognostic Assessment for breast cancer patients with brain metastases: A multicentric European experience. Breast, 2018. 37: p. 36-41]. In this study, we establish AAV9.UbC.trastuzumab as a therapeutic strategy to target HER2+ tumor cells to delay recurrence of CNS metastasis in patients with HER2+ breast cancer.

Here, we show that AAV9.UbC.trastuzumab produced durable antitumor responses in the treatment setting using aggressive cell line-derived orthotopic xenograft models of HER2+ BCBM (BT-474 and MDA-MB-453 cell lines). The most striking effect was observed against MDA-MB-453 cell line-derived orthotopic brain xenografts, where ICV treatment with AAV9.UbC.trastuzumab led to complete disease remission in 100% of the mice for the duration of the study (>12 weeks). While treatment outcomes may partly reflect intrinsic differences in cell line sensitivity, all BT-474 and MDA-MB-453 engrafted tumor-bearing control mice reached humane endpoint at ˜5-7 weeks post-tumor engraftment, suggesting that ICV treatment with AAV9.UbC.trastuzumab interfered with the growth of aggressive tumors. These results may also suggest that persistent expression of trastuzumab following IT administration of AAV9.UbC.trastuzumab could target quiescent HER2+ CNS disseminated tumor cells and prevent relapse in new sites.

A direct comparison between CSF trastuzumab levels achieved via IT administration of prior products versus tissue expression achieved by AAV9.UbC.trastuzumab cannot be established, because in essence, the drug is expected to follow opposite paths. Indeed, while IT trastuzumab injection may result in a higher transient concentration in the CSF, continuous drug diffusion from the CSF to the brain parenchyma is required to exert its function. On the contrary, trastuzumab produced within the brain parenchyma following gene transfer may result in stable tissue expression, with only a fraction of the drug diffusing to the CSF. Clearance kinetics of trastuzumab in the CSF also appears to be faster at lower concentrations, adding further complexity to the drug concentration dynamics [Braen, A. P., et al., A 4-week intrathecal toxicity and pharmacokinetic study with trastuzumab in cynomolgus monkeys. Int J Toxicol, 2010. 29(3): p. 259-67; Crowley, A. R. and M. E. Ackerman, Mind the Gap: How Interspecies Variability in IgG and Its Receptors May Complicate Comparisons of Human and Non-human Primate Effector Function. Front Immunol, 2019. 10: p. 697].

Single-dose ICM treatment with AAV9.UbC.trastuzumab can result in the transduction of cells across brain and spinal cord regions that serve as a drug source that could potentially achieve therapeutic concentrations near to the tumor bed [Hinderer, C., et al., Translational Feasibility of Lumbar Puncture for Intrathecal AAV Administration. Mol Ther Methods Clin Dev, 2020. 17: p. 969-974; Hordeaux, J., et al., Efficacy and Safety of a Krabbe Disease Gene Therapy. Hum Gene Ther, 2022. 33(9-10): p. 499-517; Kyeong, S., et al., Subtypes of breast cancer show different spatial distributions of brain metastases. PloS One, 2017. 12(11): p. e0188542; Hinderer, C., et al., Evaluation of Intrathecal Routes of Administration for Adeno-Associated Viral Vectors in Large Animals. Hum Gene Ther, 2018. 29(1): p. 15-24]. Since the presence of pre-existing antibodies against AAV9 in the general circulation does not appear to impact CNS transduction, ICM vector re-dosing or even delivery to the tumor bed following debulking surgery may represent alternatives to further increase trastuzumab expression and achieve targeted local distribution. The feasibility of delivering AAV via the ICM delivery method is supported by a CT fluoroscopy protocol we developed in collaboration with the Neuroradiology department at the University of Pennsylvania [Hinderer, C., et al., Widespread gene transfer in the central nervous system of cynomolgus macaques following delivery of AAV9 into the cisterna magna. Mol Ther Methods Clin Dev, 2014. 1: p. 14051]. This protocol is safe; correct placement of the needle in the suboccipital portion of the cisterna magna, away from the brain stem, is verified by fluoroscopy and can be done without contrast agents [Katz, N., et al., Standardized Method for Intra-Cisterna Magna Delivery Under Fluoroscopic Guidance in Nonhuman Primates. Hum Gene Ther Methods, 2018. 29(5): p. 212-219; Hinderer, C., et al., Adeno-associated virus serotype 1-based gene therapy for FTD caused by GRN mutations. Ann Clin Transl Neurol, 2020. 7(10): p. 1843-1853].

We anticipate being able to treat any patient with HER2+ CNS metastasis, regardless of their anti-AAV antibody titer status (in contrast to systemic vector delivery) via ICM administration of AAV9 into the CNS, an immune-privileged compartment. Indeed, we detected transgene expression in the CSF and brain parenchyma of rhesus NHPs with pre-existing anti-AAV9 antibody titers >1:20 receiving 3×10¹³ GC/animal AAV9.UbC.trastuzumab ICM. Moreover, the presence of ADA against trastuzumab in the CSF of NHP #2 did not appear to impact the brain parenchymal trastuzumab levels of this animal compared to NHP #1, which received the same vector ICM but did not develop ADA by day 36-37 post treatment. Our preclinical findings support these observations, as AAV brain transduction and trastuzumab expression levels remained unaffected in the brain parenchyma of mice pre-treated with IVIg, while peripheral transduction was blocked resulting in lower levels of systemic trastuzumab.

These favorable preclinical data indicate that AAV-trastuzumab represents a promising new gene therapy approach to target HER2+ breast-to-CNS metastases.

### REFERENCES

1. Allemani, C., et al., Global surveillance of trends in cancer survival 2000-14 (CONCORD-3): analysis of individual records for 37 513 025 patients diagnosed with one of 18 cancers from 322 population-based registries in 71 countries. Lancet, 2018. 391(10125): p. 1023-1075.
2. American Cancer Society: Survival Rates for Breast Cancer. Mar. 1, 2022; Available from: https://www.cancer.org/cancer/breast-cancer/understanding-a-breast-cancer-diagnosis/breast-cancer-survival-rates.html
3. Slamon, D., et al., Adjuvant trastuzumab in HER2-positive breast cancer. N Engl J Med, 2011. 365(14): p. 1273-83.
4. Vu, T. and F. X. Claret, Trastuzumab: updated mechanisms of action and resistance in breast cancer. Front Oncol, 2012. 2: p. 62.
5. Cho, H. S., et al., Structure of the extracellular region of HER2 alone and in complex with the Herceptin Fab. Nature, 2003. 421(6924): p. 756-60.
6. DeBusk, K., et al., Real-world outcomes among patients with HER2+ metastatic breast cancer with brain metastases. J Manag Care Spec Pharm, 2022. 28(6): p. 657-666.
7. Barnholtz-Sloan, J. S., et al., Incidence proportions of brain metastases in patients diagnosed (1973 to 2001) in the Metropolitan Detroit Cancer Surveillance System. J Clin Oncol, 2004. 22(14): p. 2865-72.
8. Pardridge, W. M., CSF, blood-brain barrier, and brain drug delivery. Expert Opin Drug Deliv, 2016. 13(7): p. 963-75.
9. Stemmler, H. J., et al., Ratio of trastuzumab levels in serum and cerebrospinal fluid is altered in HER2-positive breast cancer patients with brain metastases and impairment of blood-brain barrier. Anticancer Drugs, 2007. 18(1): p. 23-8.
10. Mills, M. N., et al., Evolving management of HER2+ breast cancer brain metastases and leptomeningeal disease. J Neurooncol, 2022. 157(2): p. 249-269.
11. Bousquet, G., et al., Intrathecal Trastuzumab Halts Progression of CNS Metastases in Breast Cancer. J Clin Oncol, 2016. 34(16): p. e151-5.
12. Bonneau, C., et al., Phase I feasibility study for intrathecal administration of trastuzumab in patients with HER2 positive breast carcinomatous meningitis. Eur J Cancer, 2018. 95: p. 75-84.
13. Delhaas, E. M. and F. Huygen, Complications associated with intrathecal drug delivery systems. BJA Educ, 2020. 20(2): p. 51-57.
14. Naso, M. F., et al., Adeno-Associated Virus (AAV) as a Vector for Gene Therapy. BioDrugs, 2017. 31(4): p. 317-334.
15. Hinderer, C., et al., Translational Feasibility of Lumbar Puncture for Intrathecal AAV Administration. Mol Ther Methods Clin Dev, 2020. 17: p. 969-974.
16. Hordeaux, J., et al., Toxicology Study of Intra-Cisterna Magna Adeno-Associated Virus 9 Expressing Human Alpha-L-Iduronidase in Rhesus Macaques. Mol Ther Methods Clin Dev, 2018. 10: p. 79-88.
17. Hordeaux, J., et al., Efficacy and Safety of a Krabbe Disease Gene Therapy. Hum Gene Ther, 2022. 33(9-10): p. 499-517.
18. Kang, L., et al., AAV vectors applied to the treatment of CNS disorders: Clinical status and challenges. J Control Release, 2023. 355: p. 458-473.
19. Keeler, A. M. and T. R. Flotte, Recombinant Adeno-Associated Virus Gene Therapy in Light of Luxturna (and Zolgensma and Glybera): Where Are We, and How Did We Get Here?Annu Rev Virol, 2019. 6(1): p. 601-621.
20. George, L. A., et al., Multiyear Factor VIII Expression after AAV Gene Transfer for Hemophilia A. N Engl J Med, 2021. 385(21): p. 1961-1973.
21. Kuzmin, D. A., et al., The clinical landscape for AAV gene therapies. Nat Rev Drug Discov, 2021. 20(3): p. 173-174.
22. Rothwell, W. T., et al., Intrathecal Viral Vector Delivery of Trastuzumab Prevents or Inhibits Tumor Growth of Human HER2-Positive Xenografts in Mice. Cancer Res, 2018. 78(21): p. 6171-6182.
23. Lock, M., et al., Rapid, simple, and versatile manufacturing of recombinant adeno-associated viral vectors at scale. Hum Gene Ther, 2010. 21(10): p. 1259-71.
24. Katz, N., et al., Standardized Method for Intra-Cisterna Magna Delivery Under Fluoroscopic Guidance in Nonhuman Primates. Hum Gene Ther Methods, 2018. 29(5): p. 212-219.
25. Lal, S., et al., An implantable guide-screw system for brain tumor studies in small animals. J Neurosurg, 2000. 92(2): p. 326-33.
26. Maple, L., et al., Development and validation of ELISA for herceptin detection in human serum. J Immunol Methods, 2004. 295(1-2): p. 169-82.
27. Bouwer, N. I., et al., Cardiotoxicity during long-term trastuzumab use in patients with HER2-positive metastatic breast cancer: who needs cardiac monitoring?Breast Cancer Res Treat, 2021. 186(3): p. 851-862.
28. Boutin, S., et al., Prevalence of serum IgG and neutralizing factors against adeno-associated virus (AAV) types 1, 2, 5, 6, 8, and 9 in the healthy population: implications for gene therapy using AAV vectors. Hum Gene Ther, 2010. 21(6): p. 704-12.
29. Horiuchi, M., et al., Intravenous immunoglobulin prevents peripheral liver transduction of intrathecally delivered AAV vectors. Mol Ther Methods Clin Dev, 2022. 27: p. 272-280.
30. Verdera, H. C., K. Kuranda, and F. Mingozzi, AAV Vector Immunogenicity in Humans: A Long Journey to Successful Gene Transfer. Mol Ther, 2020. 28(3): p. 723-746.
31. Gopinath, C., et al., Contemporary Animal Models For Human Gene Therapy Applications. Curr Gene Ther, 2015. 15(6): p. 531-40.
32. Hinderer, C., et al., Widespread gene transfer in the central nervous system of cynomolgus macaques following delivery of AAV9 into the cisterna magna. Mol Ther Methods Clin Dev, 2014. 1: p. 14051.
33. Kyeong, S., et al., Subtypes of breast cancer show different spatial distributions of brain metastases. PloS One, 2017. 12(11): p. e0188542.
34. Pestalozzi, B. C., et al., Identifying breast cancer patients at risk for Central Nervous System (CNS) metastases in trials of the International Breast Cancer Study Group (IBCSG). Ann Oncol, 2006. 17(6): p. 935-44.
35. Scott, B. J. and S. Kesari, Leptomeningeal metastases in breast cancer. Am J Cancer Res, 2013. 3(2): p. 117-26.
36. Griguolo, G., et al., External validation of Modified Breast Graded Prognostic Assessment for breast cancer patients with brain metastases: A multicentric European experience.

Breast, 2018. 37: p. 36-41.
37. Lu, N. T., et al., Intrathecal trastuzumab: immunotherapy improves the prognosis of leptomeningeal metastases in HER-2+ breast cancer patient. J Immunother Cancer, 2015. 3: p. 41.
38. Figura, N. B., et al., Clinical outcomes of breast leptomeningeal disease treated with intrathecal trastuzumab, intrathecal chemotherapy, or whole brain radiation therapy. Breast Cancer Res Treat, 2019. 175(3): p. 781-788.
39. Wolak, D. J., M. E. Pizzo, and R. G. Thorne, Probing the extracellular diffusion of antibodies in brain using in vivo integrative optical imaging and ex vivo fluorescence imaging. J Control Release, 2015. 197: p. 78-86.
40. Sykova, E. and C. Nicholson, Diffusion in brain extracellular space. Physiol Rev, 2008. 88(4): p. 1277-340.
41. Braen, A. P., et al., A 4-week intrathecal toxicity and pharmacokinetic study with trastuzumab in cynomolgus monkeys. Int J Toxicol, 2010. 29(3): p. 259-67.
42. Crowley, A. R. and M. E. Ackerman, Mind the Gap: How Interspecies Variability in IgG and Its Receptors May Complicate Comparisons of Human and Non-human Primate Effector Function. Front Immunol, 2019. 10: p. 697.
43. Hinderer, C., et al., Evaluation of Intrathecal Routes of Administration for Adeno-Associated Viral Vectors in Large Animals. Hum Gene Ther, 2018. 29(1): p. 15-24.
44. Hinderer, C., et al., Adeno-associated virus serotype 1-based gene therapy for FTD caused by GRN mutations. Ann Clin Transl Neurol, 2020. 7(10): p. 1843-1853.
45. Gao, G., et al., Clades of Adeno-associated viruses are widely disseminated in human tissues. J Virol, 2004. 78(12): p. 6381-8.
46. Gao, G. P., et al., Novel adeno-associated viruses from rhesus monkeys as vectors for human gene therapy. Proc Natl Acad Sci USA, 2002. 99(18): p. 11854-9.
47. Gao, G., L. H. Vandenberghe, and J. M. Wilson, New recombinant serotypes of AAV vectors. Curr Gene Ther, 2005. 5(3): p. 285-97.
48. Mendell, J. R., et al., Single-Dose Gene-Replacement Therapy for Spinal Muscular Atrophy. N Engl J Med, 2017. 377(18): p. 1713-1722.
49. Ponde, N. F., M. Lambertini, and E. de Azambuja, Twenty years of anti-HER2 therapy-associated cardiotoxicity. ESMO Open, 2016. 1(4): p. e000073.
50. Cameron, D., et al., 11 years' follow-up of trastuzumab after adjuvant chemotherapy in HER2-positive early breast cancer: final analysis of the HERceptin Adjuvant (HERA) trial. Lancet, 2017. 389(10075): p. 1195-1205.
51. Zhu, Y., et al., Spatiotemporal transcriptomic divergence across human and macaque brain development. Science, 2018. 362(6420).
52. Kati J. Ernst, K. O., Josephine Bageritz, Jan-Philipp Mallm, Andrea Wittmann, Kendra K. Maaß, Svenja Leible, Michael Boutros, Stefan M. Pfister, Marc Zuckermann, David T. W. Jones, Establishment of a simplified preparation method for single-nucleus RNA-sequencing and its application to long-term frozen tumor tissues. bioRxiv, 2020.
53. Russo, R., et al., Ultra-performance liquid chromatography/multiple reaction monitoring mass spectrometry quantification of trastuzumab in human serum by selective monitoring of a specific peptide marker from the antibody complementarity-determining regions. Rapid Communications in Mass Spectrometry, 2017. 31(14): p. 1184-1192.
54. MacLean, B., et al., Skyline: an open source document editor for creating and analyzing targeted proteomics experiments. Bioinformatics, 2010. 26(7): p. 966-968.
55. Hughes, C. S., et al., Single-pot, solid-phase-enhanced sample preparation for proteomics experiments. Nature Protocols, 2019. 14(1): p. 68-85.

All documents cited in this specification are incorporated herein by reference, as is the Sequence Listing filed herewith, labelled “UPN-22-9834PCT_Scq_List.xml”, and the sequences and text therein are incorporated by reference. U.S. Provisional Patent Application No. 63/328,225, filed Apr. 6, 2022, U.S. Provisional Patent Application No. 63/343,326, filed May 18, 2022, and U.S. Provisional Patent Application No. 63/344,905, filed May 23, 2022 are incorporated herein by reference. While the invention has been described with reference to particular embodiments, it will be appreciated that modifications can be made without departing from the spirit of the invention. Such modifications are intended to fall within the scope of the appended claims.

1. A recombinant adeno-associated virus (rAAV) comprising an adeno-associated virus (AAV) capsid containing a vector genome, wherein the vector genome comprises:
(a) an AAV-5′ inverted terminal repeat (ITR),
(b) an expression cassette comprising a coding sequence for an anti-Her2 antibody having a heavy chain and a light chain, said expression cassette comprising:
(i) a nucleic acid sequence encoding an IL2 leader peptide operably linked to an anti-Her2 antibody heavy chain,
(ii) a nucleic acid sequence comprising SEQ ID NO: 3 or a sequence at least 95% identical to SEQ ID NO: 3 encoding an anti-Her2 antibody heavy chain,
(iii) a furin cleavage site,
(iv) a T2A element linker,
(v) a nucleic acid sequence encoding an IL2 leader peptide operably linked to an anti-Her2 antibody light chain,
(vi) nucleic acid sequence comprising SEQ ID NO: 5 or a sequence at least 95% identical to SEQ ID NO: 5 encoding an anti-Her2 antibody light chain, and
regulatory control sequences operably linked to the sequences in the expression cassette, wherein the regulatory sequences comprise a ubiquitin C (UbC) promoter, an optional enhancer, an intron, and a polyadenylation (polyA) sequence, and
(c) an AAV-3′ITR.

2. The rAAV according to claim 1, wherein the nucleic acid sequence encoding a leader peptide of (i) comprises SEQ ID NO: 7.

3. The rAAV according to claim 1, wherein the nucleic acid sequence encoding a leader peptide of (v) comprises SEQ ID NO: 9.

4. The rAAV according to claim 1, wherein the anti-Her2 antibody coding sequence comprises nucleic acid sequence of SEQ ID NO: 29, or a sequence at least 95% identical to SEQ ID NO: 29.

5. The rAAV according to claim 1, wherein the UbC promoter has a nucleic acid sequence of SEQ ID NO: 24.

6. The rAAV according to claim 1, wherein the polyA sequence is SV40 polyA sequence having a nucleic acid sequence of SEQ ID NO: 23.

7. The rAAV according to claim 1, wherein the intron is a chimeric intron having a nucleic acid sequence of SEQ ID NO: 22.

8. The rAAV according to claim 1, wherein the AAV ITRs are from AAV2.

9. The rAAV according to claim 1, wherein the expression cassette comprises nucleic acid sequence of SEQ ID NO: 26, or a nucleic acid sequence at least 99% identical to SEQ ID NO: 26.

10. The rAAV according to claim 1, wherein the vector genome comprises nucleic acid sequence of SEQ ID NO: 25 or a sequence at least 99% identical to SEQ ID NO: 25.

11. A recombinant adeno-associated virus (rAAV) comprising an adeno-associated virus (AAV) capsid containing a vector genome, wherein the vector genome comprises:
(a) an AAV-5′ inverted terminal repeat (ITR),
(b) an expression cassette comprising a coding sequence for an anti-Her2 antibody having a heavy chain and a light chain, said expression cassette comprising:
(i) a nucleic acid sequence encoding an IL2 leader peptide operably linked to an anti-Her2 antibody heavy chain,
(ii) a nucleic acid sequence comprising SEQ ID NO: 3 or a sequence at least 95% identical to SEQ ID NO: 3 encoding an anti-Her2 antibody heavy chain,
(iii) a furin cleavage site,
(iv) a T2A element linker,
(v) a nucleic acid sequence encoding an IL2 leader peptide operably linked to an anti-Her2 antibody light chain,
(vi) nucleic acid sequence comprising SEQ ID NO: 5 or a sequence at least 95% identical to SEQ ID NO: 5 encoding an anti-Her2 antibody light chain, and
regulatory control sequences operably linked to the sequences in the expression cassette, wherein the regulatory sequences comprise one or more of: a promoter which is a chicken beta actin promoter or a CB7 hybrid promoter comprising cytomegalovirus immediate early (CMV IE) enhancer, a chicken beta actin promoter, and a chimeric intron comprising chicken beta actin intron, and a polyA sequence which is a SV40 polyA sequence, and
(c) AAV-3′ ITR.

12. The rAAV according to 11, wherein the promoter is the CB7 hybrid promoter having a nucleic acid sequence of SEQ ID NO: 21.

13. The rAAV according to claim 11, wherein the expression cassette comprises nucleic acid sequence of SEQ ID NO: 28, a nucleic acid sequence at least 99% identical to SEQ ID NO: 28, a nucleic acid sequence of SEQ ID NO: 2 or a nucleic acid sequence at least 99% identical to SEQ ID NO: 2.

14. The rAAV according to claim 11, wherein the vector genome comprises nucleic acid sequence of SEQ ID NO: 27, a sequence at least 99% identical to SEQ ID NO: 27, SEQ ID NO: 50, a sequence at least 99% identical to SEQ ID NO: 50, SEQ ID NO: 1, or a sequence at least 99% identical to SEQ ID NO: 1.

15. The rAAV according to claim 11, wherein the AAV capsid is a clade F AAV capsid.

16. The rAAV according to claim 1, wherein the AAV capsid is AAVhu68 capsid.

17. The rAAV according to claim 1, wherein the AAV capsid is an AAVhu95 capsid, an AAVhu96 capsid, an AAV9 capsid, or an AAVrh91 capsid.

18. The rAAV according to claim 1, which is for use in treating one or more of a HER2-positive cancer, a HER2-positive metastatic breast cancer in the brain, or HER2-positive trastuzumab resistant cancer.

19. The rAAV according to claim 1, wherein the rAAV is formulated for systemic, central nervous system and/or intratumoral delivery.

20. A composition comprising a stock of rAAV according to claim 1 and an aqueous suspension media.

21. The composition according to claim 20 wherein the suspension is formulated for an intrathecal delivery, optionally wherein the intrathecal delivery is an intracerebroventricular (ICV) injection or an intracisternal magna (ICM) injection.

22. A pharmaceutical composition comprising a rAAV according to claim 1 and an aqueous formulation buffer.

23. The pharmaceutical composition according to claim 22, which is formulated for intrathecal delivery.

24. The pharmaceutical composition according to claim 22, which is formulated for intracerebroventricular (ICV) injection or intracisternal magna (ICM) injection.

25. A recombinant nucleic acid molecule comprising:
(a) an AAV-5′ inverted terminal repeat (ITR),
(b) an expression cassette comprising at least one open reading frame (ORF) comprising an anti-Her2 antibody heavy chain and an anti-Her2 antibody light chain and nucleic acid sequences operably linked thereto which regulate expression of the anti-Her2 heavy chain and anti-Her2 light chain, and
(c) an AAV-3′ ITR,
wherein the expression cassette comprises:
(i) a promoter which is:
(A) a Ubiquitin C (UbC) promoter, or
(B) a CB7 hybrid promoter comprising a CMV IE enhancer, a chicken beta-actin promoter, and a chimeric intron comprising chicken beta actin splicing donor including chicken beta actin intron and rabbit beta globin splicing acceptor, and/or
(ii) an intron, which is a chimeric intron, and
(iii) at least one ORF which comprises: a nucleic acid sequence encoding a leader peptide operably linked to an anti-Her2 heavy chain,
(iv) a nucleic acid sequence comprising SEQ ID NO: 3 encoding an anti-Her2 heavy chain,
(v) a furin cleavage site,
(vi) a T2A linker,
(vii) a nucleic acid sequence encoding a leader peptide operably linked to an anti-Her2 light chain,
(viii) nucleic acid sequence comprising SEQ ID NO: 5 encoding an anti-Her2 light chain,
(ix) an SV40 polyadenylation (polyA) sequence, and
wherein the expression cassette further comprises spacer sequences.

26. The recombinant nucleic acid molecule according to claim 25, wherein the expression cassette comprises a nucleic acid sequence of SEQ ID NO: 26.

27. The recombinant nucleic acid molecule according to claim 25, which comprises a nucleic acid sequence of SEQ ID NO: 25.

28. A packaging host cell in culture comprising a recombinant nucleic acid molecule according to claim 25.

29. The packaging host cell in culture according to claim 28, which further comprises AAV rep coding sequences operably linked to sequences which express rep protein in the packaging host cell, an AAV capsid coding sequences operably linked to sequences which express AAV capsid proteins in the packaging host cell, and helper virus functions necessary to permit packaging of the expression cassette and AAV ITRs into the AAV capsid.

30.-31. (canceled)

32. An rAAV production system useful for producing the rAAV according to claim 1.

33.-34. (canceled)

35. The rAAV production system according to claim 32, wherein the vector genome comprises nucleic acid sequence of SEQ ID NO: 25.

36. (canceled)

37. A method for treating metastatic HER2-positive cancer in the brain, said method comprising administrating to the subject a suspension of a rAAV according to claim 1 in a formulation buffer.

38. A method for treating metastatic breast cancer in the brain, said method comprising administrating to the subject a suspension of a rAAV according to claim 1 in a formulation buffer.

39. The method according to claim 37, wherein the suspension is administered intrathecally.

40. (canceled)

41. The method according to claim 37, wherein the suspension is administered via direct injection into tumor bed or via an Ommaya device.

42. (canceled)

43. An anti-neoplastic regimen comprising administering rAAV according to claim 1 in combination with a biologic drug, a small molecule, anti-neoplastic agent, radiation, and/or chemotherapeutic agent.

44. A method for treating HER2-positive cancer which is trastuzumab-resistant, said method comprising administrating to the subject a suspension of a rAAV according to claim 1 in a formulation buffer.
